(19) United States
(12) Patent Application Publication
(10) Pub. No.: US 2024/0091345 A1
(43) Pub. Date: Mar. 21, 2024

(54) Therapeutic Viral Vaccine

(71) Applicant: GlaxoSmithKline Biologicals SA, Rixensart (BE)

(72) Inventors: Johann MOLS, Rixensart (BE); Marie TOUSSAINT, Rixensart (BE)

(73) Assignee: GlaxoSmithKline Biologicals SA, Rixensart (BE)

(21) Appl. No.: 18/262,073

(22) PCT Filed: Jan. 18, 2022
(86) PCT No.: PCT/EP2022/051030

§ 371 Date: Jul. 19, 2023

(30) Foreign Application Priority Data
Jan. 20, 2021 (EP) 21152601.7

(51) Int. Cl. A61K 39/245; A61P 31/22

(52) U.S. Cl. CPC A61K 39/245; A61P 31/22; A61K 2039/53

(57) ABSTRACT

The present invention relates to a nucleic acid encoding a HSV2 Fc receptor or immunogenic fragment or variant thereof for use in generating a cross reactive immune response against HSV1 in a subject. Also provided is a nucleic acid encoding a HSV1 Fc receptor or immunogenic fragment or variant thereof for use in generating a cross reactive immune response against HSV2 when administered to a subject.

### FIELD OF THE INVENTION

The present invention relates to a Herpes Simplex Virus (HSV, including HSV1 and HSV2) Fc receptor or an immunogenic fragment or variant thereof for treating a HSV infection in a subject. In particular, the invention provides a nucleic acid encoding a HSV2 or HSV1 Fc receptor or an immunogenic fragment or variant thereof that can induce a cross reactive immune response against HSV-1 or HSV-2 in a subject.

### BACKGROUND

Herpes Simplex Viruses (HSVs, including HSV1 and HSV2) are members of the subfamily Alphaherpesvirinae (α-herpesvirus) in the family Herpesviridae. They are enveloped, double-stranded DNA viruses containing at least 74 genes encoding functional proteins. HSV1 and HSV2 infect mucosal epithelial cells and establish lifelong persistent infection in sensory neurons innervating the mucosa in which the primary infection had occurred. Both HSV1 and HSV2 can reactivate periodically from latency established in neuronal cell body, leading to either herpes labialis (cold sores) or genital herpes (GH).

The global prevalence of genital herpes is estimated at 417 million in individuals between the ages of 15 and 49, with a disproportionate burden of disease in Africa. HSV1 is approximately as common as HSV2 as the cause of first time genital herpes in resource-rich countries. Recurrent infections are less common after HSV1 than HSV2 genital infections; therefore, HSV2 remains the predominant cause of recurrent genital herpes. Some infected individuals have severe and frequent outbreaks of genital ulcers, while others have mild or subclinical infections, yet all risk transmitting genital herpes to their intimate partners.

Recurrent GH is the consequence of reactivation of HSV2 (and to some extent of HSV1) from the sacral ganglia, followed by an anterograde migration of the viral capsid along the neuron axon leading to viral particles assembly, cell to cell fusion, viral spread and infection of surrounding epithelial cells from the genital mucosa.

Antivirals such as acyclovir; valacyclovir and famciclovir are used for the treatment of GH, both in primary or recurrent infections and regardless of the HSV1 or HSV2 origin. These drugs do not eradicate the virus from the host, as their biological mechanism of action blocks or interferes with the viral replication machinery. Randomized controlled trials demonstrated that short-term therapy with any of these three drugs reduced the severity and duration of symptomatic recurrences by one to two days when started early after the onset of symptoms or clinical signs of recurrence. However, such intermittent regimen does not reduce the number of recurrences per year.

Current treatment options for HSV recurrences have limitations, including incomplete antiviral efficacy, short term efficacy, compliance to treatment regimen, appearance of antiviral resistance, cost of treatment, and side effects. Presently, no strategies that result in long term prevention of symptomatic recurrences are known.

Accordingly, there is a need in the art for improved treatment of recurrent herpes virus infections, in particular HSV2 and HSV1 infections.

### SUMMARY OF THE INVENTION

Provided herein are the following aspects of the invention:
a nucleic acid encoding a HSV2 Fc receptor or an immunogenic fragment or variant thereof for use in inducing a cross reactive immune response against HSV1 when administered to a subject.
a nucleic acid encoding a HSV1 Fc receptor or an immunogenic fragment or variant thereof for use in inducing a cross reactive immune response against HSV2 when administered to a subject.
a pharmaceutical composition comprising the nucleic acid encoding a HSV2 Fc receptor or immunogenic fragment or variant thereof described above and a pharmaceutically acceptable carrier, for use in inducing a cross reactive immune response against HSV1 when administered to a subject.
a pharmaceutical composition comprising the nucleic acid encoding a HSV1 Fc receptor or an immunogenic fragment or variant thereof and a pharmaceutically acceptable carrier, for use in inducing a cross reactive immune response against HSV2 when administered to a subject.
a method of treating a herpes virus infection or herpes virus related disease in a subject in need thereof comprising administering an immunologically effective amount of a nucleic acid encoding a HSV2 Fc receptor or immunogenic fragment or variant thereof, to the subject wherein the HSV2 Fc receptor or immunogenic fragment or variant thereof can induce a cross reactive immune response against HSV1 when administered to a subject.
a method of treating a herpes virus infection or herpes virus related disease in a subject in need thereof comprising administering an immunologically effective amount of a nucleic acid encoding a HSV1 Fc receptor or immunogenic fragment or variant thereof, to the subject wherein the HSV1 Fc receptor or immunogenic fragment or variant thereof can induce a cross reactive immune response against HSV2 when administered to a subject.

### BRIEF DESCRIPTION OF THE DRAWINGS

FIG. 1—Total HSV-2 gE- or gI-specific IgG antibody titers measured in serum samples collected after immunizations with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 14 (14PI), 28 (14PII) & 42 (14PIII), serum samples were collected to evaluate the total HSV-2 gE- (FIG. 1A) or gI- (FIG. 1B) specific IgG antibody titer by ELISA. Each symbol represents individual animal at 14PI (dot), 14PII (diamond) or 14PIII (triangle) while the black bars represents the Geometric mean (GM) of each group. GM and number of animals (N) for each group is indicated on the x axis.

FIG. 2—Head to head comparison of HSV-2 gE- or gI-specific IgG antibody responses between vaccinated and unvaccinated groups 14 days after the third immunization in CB6F1mice. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On day 42 (14PIII), serum sample were collected to evaluate the total HSV-2 gE- (FIG. 2A) or gI- (FIG. 2B) specific IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 3—Timepoint comparison of HSV-2 gE- or gI-specific IgG antibody responses in each vaccinated group of CB6F1mice. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 14 (14PI), 28 (14PII) & 42 (14PIII), serum samples were collected to evaluate the total HSV-2 gE-specific IgG antibody titer by ELISA. The black error bar represents the 95% Confidence Interval (CI) of each geometric mean ratio (GMR). The geometric means (GMs) of the two time points and GMRs are indicated at the right of the graph. FIG. 3A illustrates PII over PI & FIG. 3B illustrates PIII over PII for HSV2 gE-specific response, whereas FIG. 3C illustrates PII over PI & FIG. 3D illustrates PIII over PII for HSV2 gI-specific response.

FIG. 4—Head to head comparison of HSV-2 gE- or gI IgG antibody responses between vaccinated groups 14 days after the third immunization of CB6F1 mice. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On day 42 (14PIII), serum samples were collected to evaluate the total HSV-2 gE- (FIG. 4A) or gI- (FIG. 4B) specific IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 5—Total HSV-1 gE/gI cross-reactive IgG antibody titers measured in serum samples collected 14 days after the third immunization with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On day 42 (14PIII), serum samples were collected to evaluate the total HSV-1 gE/gI cross-reactive IgG antibody titer by ELISA. Each symbol represents individual animal at 14PIII while the black bars represents the Geometric mean (GM) of each group. GM and number of animals (N) for each group is indicated on the x axis.

FIG. 6—Head to head comparison of HSV-1 gE/gI cross-reactive IgG antibody response between vaccinated and unvaccinated groups 14 days after the third immunization in CB6F1mice. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On day 42 (14PIII), serum sample were collected to evaluate the total HSV-1 gE/gI cross-reactive IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 7—Head to head comparison of HSV-1 gE/gI cross-reactive antibody responses between vaccinated groups 14 days after the third immunization of CB6F1 mice. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On day 42 (14PIII), serum samples were collected to evaluate the total HSV-1 gE/gI cross-reactive IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 8—HSV-2 MS-specific neutralizing antibody titers measured in serum samples collected 14 days after the third immunization with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII), serum samples were collected to evaluate the titer of neutralizing antibodies towards HSV-2 MS strain (400 TCID₅₀). FIG. 8A: Each dot represents individual animal titer. The positivity threshold value corresponds to the 1^{st} sample dilution. Negative samples are illustrated by the 1^{st} samples dilution/2 (neutralizing titer=5). The number of mice by group (N) and the Geometric mean (GM) for each group are indicated below the x axis of the graph. FIG. 8B: GMR with 95% of confidence interval (CI) of HSV-2-specific neutralizing antibody titers. The black error bar represents the 95% Confidence Interval (CI) of each geometric mean ratio (GMR). The GMs of the two groups compared and GMRs are indicated at the right of the graph.

FIG. 9—HSV-1 cross-reactive neutralizing antibody titers measured in serum samples collected 14 days after the third immunization with different mutated versions of AS01-adjuvanted HSV-2 gE/gI heterodimer protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII), serum samples were collected to evaluate the titer of cross-neutralizing antibodies towards HSV-1 VR1789 strain (400 TCID₅₀). FIG. 9A: Each dot represents individual animal titer. The positivity threshold value corresponds to the 1^{st} sample dilution. Negative samples are illustrated by the 1^{st} samples dilution/2 (neutralizing titer=5). The number of mice by group (N) and the Geometric mean (GM) for each group are indicated below the x axis of the graph. FIG. 9B: GMR with 95% of confidence interval (CI) of HSV-1 cross-reactive neutralizing antibody titers. The black error bar represents the 95% Confidence Interval (CI) of each geometric mean ratio (GMR). The GMs of the two groups compared and GMRs are indicated at the right of the graph.

FIG. 10—Evaluation of the ability of vaccine-specific antibodies to decrease, in-vitro, human IgG Fc binding by HSV-2 gE/gI antigen 14 days after third immunizations with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII), serum samples were collected to evaluate the ability of vaccine-specific antibodies to compete and decrease hIgG Fc binding by HSV-2 gE/gI protein. Each curve represents individual mice data. FIG. 10A: AS01/HSV-2 gE/gI V340W over NaCl; FIG. 10B: AS01/HSV-2 gE/gI A248T over NaCl; FIG. 10C: AS01/HSV-2 gE/gI A246W over NaCl; FIG. 10D: AS01/HSV-2 gE/gI P318I over NaCl; FIG. 10E: AS01/HSV-2 gE/gI A248T_V340W over NaCl.

FIG. 11—Comparison of the ability of vaccine-specific antibodies to decrease, in-vitro, human IgG Fc binding by HSV-2 gE/gI antigen 14 days after third immunizations with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant proteins adjuvanted in AS01 (5 μg/each). An additional group of mice was i.m injected with a saline solution (NaCl 150 mM) following the same schedule of immunization and used as negative control group (n=4/gr6). On days 42 (14PIII), serum samples were collected to evaluate the ability of vaccine-specific antibodies to compete and decrease hIgG Fc binding by HSV-2 gE/gI protein. FIG. 11A: Each dot represents ED50 titer with 95% CIs from individual mice. The positivity threshold value corresponds to the 1^{st} sample dilution. Negative samples are illustrated by the 1^{st} samples dilution/2. The number of mice by group (N) and the Geometric mean (GM) for each group is indicated below the x axis of the graph. FIG. 11B: GMR with 95% of confidence interval (CI). The black error bar represents the 95% Confidence Interval (CI) of each geometric mean ratio (GMR). The GMs of the two groups compared and GMRs are indicated at the right of the graph.

FIG. 12—Evaluation of the ability of HSV-2 gE/gI antibodies to bind and activate mFcγRIII after incubation with HSV-2 gE/gI positive cells 14 days after third immunizations with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII), serum samples were collected to evaluate the ability of vaccine-specific antibodies to bind HSV-2 gE/gI positive 3T3 cells and activate the mouse F_{c}γRIII expressed by modified Jurkat reporter cells. FIG. 12A-E each curve illustrate data from pools of 2 mouse sera immunized with different AS01-HSV-2 gE/gI mutants over NaCl while FIG. 12F illustrates Geoemetric mean of each AS01-HSV-2 gE/gI vaccinated group over NaCl.

FIG. 13—Percentage of vaccine-specific CD4+/CD8+ T cell response induced in CB6F1 mice 14 days after third immunizations with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII), serum sample were collected to evaluate vaccine-specific CD4+/CD8+ T cell response in the spleen. Splenocytes were stimulated ex-vivo during 6 hours with pools of 15mer peptides covering the whole amino acids sequence of the gE or gI protein from HSV-2. β-actin peptides pool stimulation was used to evaluate non-specific response. The frequencies of CD4+/CD8+ T cells secreting IL-2, IFN-γ and/or TNF-α were measured by intracellular cytokine staining. Circle, triangle and diamond shapes represent individual % of CD4+(FIG. 13A)/CD8+(FIG. 13B) T cell response detected for each antigen (HSV-2 gE or HSV-2 gI antigens, or human (3-actin). Black line represents the geometric mean (GM) of the response and black dotted line indicates the percentile 95th (P95) obtained with all the stimulations in the saline group. The number of animals/group with valid result (N) and GM of each group are indicated under the graph.

FIG. 14—Head to head comparison of % of vaccine-specific CD4+/CD8+ T cell responses between vaccinated and unvaccinated groups 14 days after third immunizations in CB6F1mice. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII) mice were culled to evaluate vaccine-specific CD4+/CD8+ T cell response in the spleen. The black error bar represents the 95% Confidence Interval (CI) of each geometric mean ratio (GMR). The geometric means (GMs) of the two groups compared and GMRs are indicated at the right of the graph. FIG. 14A: GMRs with 95% CIs of HSV-2 gE CD4+ T cells; FIG. 14B: GMRs with 95% CIs of HSV-2 gI specific CD4+ T cells; FIG. 14C: GMRs with 95% CIs of HSV-2 gE specific CD8+ T cells.

FIG. 15—Head to head comparison of % of vaccine-specific CD4+/CD8+ T cell responses between vaccinated groups 14 days after the third immunization of CB6F1 mice. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII) mice were culled to evaluate vaccine-specific CD4/CD8+ T cell response in the spleen. The black error bar represents the 95% Confidence Interval (CI) of each geometric mean ratio (GMR). The geometric means (GMs) of the two groups compared and GMRs are indicated at the right of the graph. FIG. 15A: GMRs with 95% CIs of HSV-2 gE-specific CD4+ T cells; FIG. 15B: GMRs with 95% CIs of HSV-2 gI-specific CD4+ T cells; FIG. 15C: GMRs with 95% CIs of HSV-2 gE- specific CD8+ T cells.

FIG. 16—Total HSV-2 gE- or gI-specific IgG antibody titers measured in serum samples collected after one, two or three immunizations with different mutated versions of SAM HSV-2 gE/gI vector formulated in Lipid nanoparticles (LNP). At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 21 (21PI), 42 (21PII) & 63 (21PIII), serum sample from each animal was collected to evaluate the total HSV-2 gE- (FIG. 16A) or gI- (FIG. 16B) specific IgG antibody titer by ELISA. Each symbol represents individual animal at 21PI (dot), 21PII (triangle) or 21PIII (diamond) while the black bars represents the Geometric mean (GM) of each group. GM and number of animals (N) for each group is indicated on the x axis.

FIG. 17—Head to head comparison of HSV-2 gE- or gI-specific IgG antibody responses between vaccinated and unvaccinated groups 21 days after the third immunization in CB6F1mice. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On day 63 (21PIII), serum sample from each animal was collected to evaluate the total HSV-2 gE-(fig2A) or gI- (fig2B) specific IgG antibody titer by ELISA. The black error bar represents the 95% Confidence Interval (CI) of each geometric mean ratio (GMR). The geometric means (GMs) of the two groups compared and GMRs are indicated at the right of the graph. FIG. 17A: GMR with 95% CIs of HSV-2 gE-specific antibody titers (EU/mL); FIG. 17B: GMR with 95% CIs of HSV-2 gI-specific antibody titers (EU/mL).

FIG. 18—Timepoint comparison of HSV-2 gE- or gI-specific IgG antibody responses between each LNP-SAM HSV-2 gE/gI vaccinated group of CB6F1mice. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl150 mM) following the same schedule of immunization and used as negative control group (n=4/gr7). On days 21 (21PI), 42 (21PII) & 63 (21PIII), serum sample from each animal was collected to evaluate the total HSV-2 gE- or gI-specific IgG antibody titer by ELISA. The black error bar represents the 95% Confidence Interval (CI) of each geometric mean ratio (GMR). The geometric means (GMs) of the two time points and GMRs are indicated at the right of the graph. FIG. 18A illustrates PII over PI & FIG. 18B illustrates PIII over PII for HSV-2 gE-specific response, whereas FIG. 18C illustrates PII over PI & FIG. 18D illustrates PIII over PII for HSV-2 gI-specific response.

FIG. 19—Head to head comparison of HSV-2 gE- or gI- IgG antibody responses between vaccinated groups 21 days after the third immunization of CB6F1 mice. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On day 63 (21PIII), serum sample from each animal was collected to evaluate the total HSV-2 gE- (FIG. 19A) or gI- (FIG. 19B) specific IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 20—Total HSV-1 gE/gI cross-reactive IgG antibody titers measured in serum samples collected 21 days after the third immunization with different SAM HSV-2 gE/gI mutants formulated in Lipid nanoparticles (LNP). At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On day 63 (21PIII), serum sample from each animal was collected to evaluate the total HSV-1 gE/gI cross-reactive IgG antibody titer by ELISA. Each symbol represents individual animal at 21PIII while the black bars represents the Geometric mean (GM) of each group. GM and number of animals (N) for each group is indicated on the x axis.

FIG. 21—Head to head comparison of HSV-1 gE/gI cross-reactive IgG antibody response between vaccinated and unvaccinated groups 21 days after the third immunization in CB6F1mice. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On day 63 (21PIII), serum sample from each animal was collected to evaluate the total HSV-1 gE/gI cross-reactive IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 22—Head to head comparison of HSV-1 gE/gI cross-reactive IgG antibody response between vaccinated groups 21 days after the third immunization of CB6F1 mice. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On day 63 (21PIII), serum sample from each animal was collected to evaluate the total HSV-1 gE/gI cross-reactive IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 23—HSV-2 MS-specific neutralizing antibody titer measured in serum samples collected 21 days after the third immunization with different LNP-formulated SAM-HSV-2 gE/gI mutants. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII), serum sample from each animal was collected to evaluate the titer of neutralizing antibodies towards HSV-2 MS strain (400 TCID₅₀). FIG. 23A: Each symbol represents individual animal titer while each bar represents Geomean (GM)+95% Confidence intervals (CIs). The positivity threshold value corresponds to the 1^{st} sample dilution. Negative samples are illustrated by the 1^{st} samples dilution/2 (neutralizing titer=5). The number of mice by group (N) and the GM for each group are indicated below the x axis of the graph. FIG. 23B: The black error bar represents the 95% Confidence Interval (CI) of each geometric mean ratio (GMR). The GMs of the two groups compared and GMRs are indicated at the right of the graph.

FIG. 24—HSV-1 cross-reactive neutralizing antibody titer measured in serum samples collected 21 days after the third immunization with different LNP-formulated SAM-HSV-2 gE/gI mutants. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII), serum sample from each animal was collected to evaluate the titer of neutralizing antibodies towards HSV-1 VR1789 strain (400 TCID₅₀). FIG. 24A: Each symbol represents individual animal titer while each bar represents Geomean (GM)+95% Confidence intervals (CIs). The positivity threshold value corresponds to the 1^{st} sample dilution. Negative samples are illustrated by the 1^{st} samples dilution/2 (neutralizing titer=5). The number of mice by group (N) and the GM for each group are indicated below the x axis of the graph. FIG. 24B: The black error bar represents the 95% Confidence Interval (CI) of each geometric mean ratio (GMR). The GMs of the two groups compared and GMRs are indicated at the right of the graph.

FIG. 25—Evaluation of the ability of vaccine-specific antibodies to decrease, in-vitro, hIgG Fc binding by HSV-2 gE/gI antigen 21 days after the third immunization with different LNP-formulated SAM-HSV-2 gE/gI mutants. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII), serum sample from each animal was collected to evaluate the ability of vaccine-specific antibodies to compete and decrease hIgG Fc binding by HSV-2 gE/gI protein. Each curve represents individual mice. LNP/SAM-HSV-2 gE/gI V340W over NaCl group (FIG. 25A); LNP/SAM-HSV-2 gE/gI A248T over NaCl group (FIG. 25B); LNP/SAM-HSV-2 gE/gI A246W over NaCl group (FIG. 25C); LNP/SAM-HSV-2 gE/gI P318I over NaCl group (FIG. 25D); LNP/SAM-HSV-2 gE/gI A248T_V340W over NaCl group (FIG. 25E); LNP/SAM-HSV-2 gE/gI insert ARAA over NaCl group; (FIG. 25F).

FIG. 26—Comparison of the ability of vaccine-specific antibodies to decrease, in-vitro, human IgG Fc binding by HSV-2 gE/gI antigen 21 days after third immunizations with different LNP-formulated SAM-HSV-2 gE/gI mutants in CB6F1 mice. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII), serum sample from each animal was collected to evaluate the ability of vaccine-specific antibodies to compete and decrease hIgG Fc binding by HSV-2 gE/gI protein. Each dot represents ED50 titer with 95% CIs from individual mice. The positivity threshold value corresponds to the 1^{st} sample dilution. Negative samples are illustrated by the Pt samples dilution/2 (titer=5). The number of mice by group (N) and the Geometric mean (GM) for each group is indicated below the x axis of the graph.

FIG. 27—Evaluation of the ability of HSV-2 gE/gI antibodies to bind and activate mFcγRIII after incubation with HSV-2 gE/gI positive cells 21 days after three immunizations with different mutated versions of LNP-formulated SAM-HSV-2 gE/gI protein. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII), serum samples were collected to evaluate the ability of vaccine-specific antibodies to bind HSV-2 gE/gI positive 3T3 cells and activate the mouse F_{c}γRIII expressed by modified Jurkat reporter cells. FIG. 27A-F: each curve illustrates pools of 2 mouse sera immunized with different LNP-SAM HSV-2 gE/gI mutants over NaCl while FIG. 27G: illustrates Geoemetric mean of each LNP-SAM HSV-2 gE/gI vaccinated group over NaCl.

FIG. 28—Percentage of vaccine-specific or HSV-1 gE cross reactive CD4+/CD8+T cell responses induced in CB6F1 mice 21 days after third immunization with different SAM HSV-2 gE/gI mutants formulated in Lipid nanoparticles (LNP). At days 0, 21 & 42, CB6F1 mice (n=6/gr1-6) were intramuscularly (i.m) immunized with 0.8 μg of different mutated versions of SAM HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII) mice were culled to evaluate HSV-2 gE and HSV-2 gI-specific or HSV-1 gE cross reactive CD4+/CD8+T cell responses in the spleen. Splenocytes were stimulated ex-vivo during 6 hours with pools of 15mer peptides covering the whole amino acids sequence of the gE or gI protein from HSV-2, as well as gE protein from HSV-1. Human β-actin peptides pool stimulation was used to evaluate non-specific response. The frequencies of CD4+(FIG. 28A)/CD8+(FIG. 28B) T cells secreting IL-2, IFN-γ and/or TNF-α were measured by intracellular cytokine staining. Circle, triangle, square and diamond shapes represent individual % of CD4+/CD8+ T cell responses detected for each antigen (HSV-1 or 2 gE or HSV-2 gI antigens, or human β-actin). Black line represents the geometric means (GM) of the response and black dotted line indicates the percentile 95th (P95) obtained in the saline group when combining the four antigens (HSV-1 or 2 gE or HSV-2 gI antigens or Human β-actin). The number of animals/group with valid result (N) and the GM of each group are indicated under the graph.

FIG. 29—Head to head comparison of vaccine-specific CD4+T cell response between vaccinated and unvaccinated groups 21 days after third immunization in CB6F1 mice. At days 0, 21 & 42, CB6F1 mice (n=6/gr1-6) were intramuscularly (i.m) immunized with 0.8 μg of different mutated versions of SAM HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII) mice were culled to evaluate HSV-2 gE (FIG. 29A) & HSV-2 gI (FIG. 29B)-specific CD4+T cell responses in the spleen. The black error bar represents the 95% Confidence Interval (CI) of each the geometric mean ratio (GMR). The geometric means (GMs) of the two groups compared and GMRs are indicated at the right of the graph.

FIG. 30—Head to head comparison of HSV-2 gE-specific or HSV-1 gE cross reactive CD8+T cell response between vaccinated and unvaccinated groups 21 days after third immunization in CB6F1 mice. At days 0, 21 & 42, CB6F1 mice (n=6/gr1-6) were intramuscularly (i.m) immunized with 0.8 μg of different mutated versions of SAM HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII), mice were culled to evaluate HSV-2 gE-specific (FIG. 30A) or HSV-1 gE cross reactive (FIG. 30B) CD8+ T cell responses in the spleen. The black error bar represents the 95% Confidence Interval (CI) of each geometric mean ratio (GMR). The geometric means (GMs) of the two groups compared and GMRs are indicated at the right of the graph.

FIG. 31—Head to head comparison of vaccine-specific CD4+T cell response between vaccinated groups 21 days after third immunization in CB6F1 mice. At days 0, 21 & 42, CB6F1 mice (n=6/gr1-6) were intramuscularly (i.m) immunized with 0.81 μg of different mutated versions of SAM HSV-2 gE/gI vector formulated in Lipid nanoparticles (LNP). An additional group of mice was i.m injected with a saline solution (NaCl150 mM) following the same schedule of immunization and used as negative control group (n=4/gr7). On days 63 (21PIII) mice were culled to evaluate HSV-2 gE- (FIG. 31A) & HSV-2 gI- (FIG. 31B) specific CD4+T cell responses in the spleen. The black error bar represents the 95% Confidence Interval (CI) of each geometric mean ratio (GMR). The geometric means (GMs) of the two groups compared and GMRs are indicated at the right of the graph.

FIG. 32—Head to head comparison of HSV-2 gE-specific or HSV-1 gE cross reactive CD8+T cell response between vaccinated groups 21 days after third immunization in CB6F1 mice. At days 0, 21 & 42, CB6F1 mice (n=6/gr1-6) were intramuscularly (i.m) immunized with 0.8 μg of different mutated versions of SAM HSV-2 gE/gI vector formulated in Lipid nanoparticles (LNP). An additional group of mice was i.m injected with a saline solution (NaCl 150 mM) following the same schedule of immunization and used as negative control group (n=4/gr7 On days 63 (21PIII), mice were culled to evaluate HSV-2 gE-specific (FIG. 32A) or HSV-1 gE cross reactive (FIG. 32B) CD8+T cell responses in the spleen. The black error bar represents the 95% Confidence Interval (CI) of each geometric mean ratio (GMR). The geometric means (GMs) of the two groups compared and GMRs are indicated at the right of the graph.

FIG. 33—Anti-HSV-1-specific gE/gI IgG antibody response measured in serum samples after immunizations with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 13 (13PI), 27 (13PII) & 42 (14PIII), serum sample from each animal was collected to evaluate the total anti-HSV-1 gE/gI-specific IgG antibody titer by ELISA. Each shape represents individual animal at different timepoints (dot=13PI; triangle=13PII; diamond=14PIII) while the black bars represents the Geometric mean of each group. Geometric mean (GM) and number of animals (N) for each group is indicated on the x axis.

FIG. 34—Head to head comparison of anti-HSV-1-specific gE/gI-specific IgG antibody response between vaccinated and unvaccinated groups 14 days after the third immunization in CB6F1mice. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with saline solution (NaCl 150 mM) following the same schedule of immunization and used as negative control group (n=4/gr7). On days 42 (14PIII), serum sample from each animal was collected to evaluate the total anti-HSV-1 gE/gI-specific IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 35—Time point comparison of anti-HSV-1 gE/gI-specific IgG antibody response in each vaccinated group of CB6F1 mice. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with saline solution (NaCl 150 mM) following the same schedule of immunization and used as negative control group (n=4/gr7). On days 13 (13PI), 27 (13PII) & 42 (14PIII), serum sample from each animal was collected to evaluate the total anti-HSV-1 gE/gI-specific IgG antibody titer by ELISA. The black error bar represents the 95% Confidence Interval (CI) of each geometric mean ratio (GMR). The geometric means (GMs) of the two time points and GMRs are indicated at the right of the graph. FIG. 35A=PII over PI & FIG. 35B=PIII over PII.

FIG. 36—Head to head comparison of anti-HSV-1 gE/gI-specific IgG antibody response between vaccinated groups 14 days after the third immunization in CB6F1mice. At days 0, 14 & 28, CB6F1 mice (n=6/gr1-6) were intramuscularly (i.m) immunized with different versions of HSV-1 gE/gI protein) adjuvanted with AS01 (5 μg/each). An additional group of mice was i.m injected with a saline solution (NaCl 150 mM) following the same schedule of immunization and used as negative control group (n=4/gr7). On days 42 (14PIII), serum sample from each animal was collected to evaluate the total anti-HSV-1 gE/gI- specific IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 37—Anti-HSV-2 gE or HSV-2 gI cross-reactive IgG antibody response measured in serum samples after immunizations with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On day 42 (14PIII), serum sample from each animal was collected to evaluate the total anti-HSV-2 gE (FIG. 37A) or HSV-2 gI (FIG. 37B) cross-reactive IgG antibody titer by ELISA. Each shape represents individual animal at timepoint 14PIII while the black bars represents the Geometric mean of each group. Geometric mean (GM) and number of animals (N) for each group is indicated on the x axis.

FIG. 38—Head to head comparison of anti-HSV-2 gE or HSV-2 gI cross-reactive IgG antibody response between vaccinated and unvaccinated groups 14 days after the third immunization in CB6F1mice. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with saline solution (NaCl 150 mM) following the same schedule of immunization and used as negative control group (n=4/gr7). On days 42 (14PIII), serum sample from each animal was collected to evaluate the total anti-HSV-2 gE (FIG. 38A) or HSV-2 gI (FIG. 38B) cross-reactive IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 39—Head to head comparison of anti-HSV-2 gE or HSV-2 gI cross-reactive IgG antibody response between vaccinated groups 14 days after the third immunization in CB6F1mice. At days 0, 14 & 28, CB6F1 mice (n=6/gr1-6) were intramuscularly (i.m) immunized with different versions of HSV-1 gE/gI protein) adjuvanted with AS01 (5 μg/each). An additional group of mice was i.m injected with a saline solution (NaCl 150 mM) following the same schedule of immunization and used as negative control group (n=4/gr7). On days 42 (14PIII), serum sample from each animal was collected to evaluate the total anti-HSV-2 gE (FIG. 39A) or HSV-2 gI (FIG. 39B) cross-reactive IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 40—HSV-1-specific neutralizing antibody titers measured in serum samples collected 14 days after the third immunization with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 42 (14PIII), serum sample was collected to evaluate the titer of neutralizing antibodies towards HSV-1 VR-1789 strain (400 TCID₅₀). FIG. 40A: Each dot represents individual animal titer. The positivity threshold value corresponds to the 1^{st} sample dilution. Negative samples are illustrated by the 1^{st} samples dilution/2 (neutralizating titer=5). The number of mice by group (N) and the Geometric mean (GM) for each group are indicated below the x axis of the graph. FIG. 40B: GMR with 95% of confidence interval (CI). The black error bar represents the 95% Confidence Interval (CI) of each GMR. The GMs of the two groups compared and GMRs are indicated at the right of the graph.

FIG. 41—HSV-2 cross-reactive neutralizing antibody titers measured in serum samples collected 14 days after the third immunization with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 42 (14PIII), serum sample was collected to evaluate the titer of cross-reactive neutralizing antibodies towards HSV-2 MS strain (400 TCID₅₀). Each dot represents individual animal titer. The positivity threshold value corresponds to the 1^{st} sample dilution. Negative samples are illustrated by the 1^{st} samples dilution/2 (neutralizating titer=5). The number of mice by group (N) and the Geometric mean (GM) for each group are indicated below the x axis of the graph.

FIG. 42—Evaluation of the ability of vaccine-specific antibodies to decrease, in-vitro, hIgG Fc binding by HSV-1 gE/gI antigen 14 days after the third immunization with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 42 (14PIII), serum sample was collected to evaluate the ability of vaccine-specific antibodies to compete and decrease hIgG Fc binding by HSV-1 gE/gI protein. FIG. 42A=AS01-HSV-1 gE/gI unmutated over NaCl; FIG. 42B=AS01-HSV-1 gE_P319R/gI over NaCl; FIG. 42C=AS01-HSV-1 gE_P321D/gI over NaCl; FIG. 42D=AS01-HSV-1 gE_R322D/gI over NaCl; FIG. 42E=AS01-HSV-1 gE_N243A_R322D/gI over NaCl; FIG. 42F=AS01-HSV-1 gE_A340G_S341G_V342G/gI over NaCl.

FIG. 43—Comparison of the ability of vaccine-specific antibodies to decrease, in-vitro, hIgG Fc binding by HSV-1 gE/gI antigen 14 days after the third immunization with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted in AS01 (5 μg/each). An additional group of mice was i.m injected with saline solution (NaCl 150 mM) following the same schedule of immunization and used as negative control group (n=4/gr7). On days 42 (14PIII), serum sample was collected to evaluate the ability of vaccine-specific antibodies to compete and decrease hIgG Fc binding by HSV-1 gE/gI protein. FIG. 43A: Each dot represents ED50 value from individual mice while each bar represents GMT+95% CIs. The positivity threshold value corresponds to the 1^{st} sample dilution. Negative samples are illustrated by the 1^{st} samples dilution/2 (ED50 value=5). The number of mice by group (N) and the Geometric mean (GM) for each group is indicated below the x axis of the graph. FIG. 43B: GMR with 95% CIs of inhibition of hIgG Fc binding on HSV-1 gE/gI protein (ED50). The black error bar represents the 95% Confidence Interval (CI) of each GMR. The GMs of the two groups compared and GMRs are indicated at the right of the graph.

FIG. 44—Evaluation of the ability of vaccine-specific antibodies to decrease, in-vitro, hIgG Fc binding by HSV-2 gE/gI antigen 14 days after the third immunization with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 42 (14PIII), serum sample was collected to evaluate the ability of vaccine-specific antibodies to compete and decrease hIgG Fc binding by HSV-2 gE/gI protein. FIG. 44A=AS01-HSV-1 gE/gI unmutated over NaCl; FIG. 44B=AS01-HSV-1 gE_P319R/gI over NaCl; FIG. 44C=AS01-HSV-1 gE_P321D/gI over NaCl; FIG. 44D=AS01-HSV-1 gE_R322D/gI over NaCl; FIG. 44E=AS01-HSV-1 gE_N243A_R322D/gI over NaCl; FIG. 44F=AS01-HSV-1 gE_A340G_S341G_V342G/gI over NaCl.

FIG. 45—Comparison of the ability of vaccine-specific antibodies to decrease, in-vitro, hIgG Fc binding by HSV-2 gE/gI antigen 14 days after the third immunization with different versions of AS01-adjuvanted HSV-1 gE/gI protein. At days 0, 14 & 28, CB6F1 mice (n=6/gr1-6) were intramuscularly (i.m) immunized with different versions of HSV-1 gE/gI protein adjuvanted in AS01 (5 μg/each). An additional group of mice was i.m injected with saline solution (NaCl 150 mM) following the same schedule of immunization and used as negative control group (n=4/gr7). On days 42 (14PIII), serum sample was collected to evaluate the ability of vaccine-specific antibodies to compete and decrease hIgG Fc binding by HSV-2 gE/gI protein. FIG. 45A: Each dot represents ED50 value from individual mice while each bar represents GMT+95% CIs. The positivity threshold value corresponds to the 1^{st} sample dilution. Negative samples are illustrated by the 1^{st} samples dilution/2 (ED50 value=5). The number of mice by group (N) and the Geometric mean (GM) for each group is indicated below the x axis of the graph. FIG. 45B: GMR with 95% CIs of inhibition of hIgG Fc binding on HSV-2 gE/gI protein (ED50). The black error bar represents the 95% Confidence Interval (CI) of each GMR. The GMs of the two groups compared and GMRs are indicated at the right of the graph.

FIG. 46—Evaluation of the ability of HSV-2 gE/gI cross reactive antibodies collected 14 days after the third immunization with different versions of AS01-adjuvanted HSV-1 gE/gI protein to bind and activate mouse FcγRIII after incubation with HSV-2 gE/gI transfected cells. At days 0, 14 & 28, CB6F1 mice (n=6/gr1-6) were intramuscularly (i.m) immunized with different versions of HSV-1 gE/gI protein adjuvanted in AS01 (5 μg/each). An additional group of mice was i.m injected with saline solution (NaCl 150 mM) following the same schedule of immunization and used as negative control group (n=4/gr7). On days 42 (14PIII), serum samples were collected to evaluate the ability of vaccine-specific antibodies to cross-bind and activate mouse FcγRIII expressed by modified Jurkat reporter cells after incubation with HSV-2 gE/gI transfected 3T3 cells. FIG. 46A-F: Each curve illustrates data from pools of 2 mouse sera immunized with different versions of AS01-adjuvanted HSV-1 gE/gI protein or treated with a NaCl 150 mM solution.

FIG. 47—Comparison of the ability of HSV-2 gE/gI cross reactive antibodies collected 14 days after the third immunization with different versions of AS01-adjuvanted HSV-1 gE/gI protein to bind and activate mouse FcγRIII after incubation with HSV-2 gE/gI transfected cells. At days 0, 14 & 28, CB6F1 mice (n=6/gr1-6) were intramuscularly (i.m) immunized with different versions of HSV-1 gE/gI protein adjuvanted in AS01 (5 μg/each). An additional group of mice was i.m injected with saline solution (NaCl 150 mM) following the same schedule of immunization and used as negative control group (n=4/gr7). On days 42 (14PIII), serum samples were collected to evaluate the ability of vaccine-specific antibodies to cross-react and activate mouse FcγRIII expressed by modified Jurkat reporter cells after incubation with HSV-2 gE/gI transfected 3T3 cells. Each dot represents a pool of 2 individual mouse and is represented as the ratio of the area under the curve (AUC) normalized on control positive sample.

FIG. 48—Percentage of vaccine-specific or HSV-2 gE cross reactive CD4+/CD8+T cell responses induced in CB6F1 mice 14 days after the third immunization with different versions of HSV-1 gE/gI protein adjuvanted in AS01. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM), following the same schedule of immunization, and used as negative control group (n=4/gr7). On days 42 (14PIII) mice were culled to evaluate HSV-1 gE and HSV-1 gI-specific & HSV-2 gE cross-reactive CD4+/CD8+ T cell response in the spleen. Splenocytes were stimulated ex-vivo during 6 hours with pools of 15mer peptides covering the whole amino acids sequence of the gE or gI protein from HSV-1, as well as gE protein from HSV-2. Human β-actin peptides pool stimulation was used to evaluate non-specific response. The frequencies of CD4+/CD8+ T cells secreting IL-2, IFN-γ and/or TNF-α were measured by intracellular cytokine staining and results are shown in FIGS. 48A and 48B. Circle, triangle, square and diamond shapes represent individual % of CD4+/CD8+ T cell response detected for each antigen (HSV-1 or HSV-2 gE or gI antigens, or Human β-actin). Black line represents the geometric means (GM) of the response and black dotted line indicates the percentile 95th (P95) obtained with all the stimulations in the saline group. The number of animals/group with valid result (N) and the geometric mean (GM) of each group are indicated under the graph.

FIG. 49—Head to head comparison of vaccine-specific or HSV-2 gE cross reactive CD4+T cell responses between vaccinated and unvaccinated groups 14 days after the third immunization in CB6F1 mice. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 42 (14PIII) mice were culled to evaluate HSV-1 gE and HSV-1 gI-specific & HSV-2 gE cross-reactive CD4+T cell responses in the spleen. The black error bar represents the 95% Confidence Interval (CI) of each geometric mean ratio (GMR). The Geometric means (GM)s of the two groups compared and GMRs are indicated at the right of the graph. FIG. 49A: GMR with 95% CIs of HSV-1 gE-specific CD4+ T cells; FIG. 49B: GMR with 95% CIs of HSV-2 gE-specific CD4+ T cells; FIG. 49C: GMR with 95% CIs of HSV-1 gI-specific CD4+ T cells. The black error bar represents the 95% Confidence Interval (CI) of each GMR. The GMs of the two groups compared and GMRs are indicated at the right of the graph.

FIG. 50—Head to head comparison of HSV-1 gI-specific CD8+ T cell response between vaccinated and unvaccinated groups 14 days after the third immunization in CB6F1 mice. At days 0, 14 & 28, CB6F1 mice (n=6/gr1-6) were intramuscularly (i.m) immunized with different versions of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 42 (14PIII) mice were culled to evaluate HSV-1 gI-specific CD8+ T cell response in the spleen. The black error bar represents the 95% Confidence Interval (CI) of each GMR. The GMs of the two groups compared and GMRs are indicated at the right of the graph.

FIG. 51—Head to head comparison of HSV-1 gE- specific or HSV-2 gE cross reactive CD4+T cell responses between vaccinated groups 14 days after the third immunization in CB6F1 mice. At days 0, 14 & 28, CB6F1 mice (n=6/gr1-6) were intramuscularly (i.m) immunized with different versions of HSV-1 gE/gI protein adjuvanted with AS01(5 μg/each). An additional group of mice was i.m injected with a saline solution (NaCl 150 mM), following the same schedule of immunization, and used as negative control group (n=4/gr7). On days 42 (14PIII) mice were culled to evaluate HSV-1 gE (FIG. 51A)-specific and HSV-2 gE (FIG. 51B) cross-reactive CD4+T cell responses in the spleen. The black error bar represents the 95% Confidence Interval (CI) of each GMR. The GMs of the two groups compared and GMRs are indicated at the right of the graph.

FIG. 52—Head to head comparison of HSV-1 gI-specific CD4+T cell response between vaccinated groups 14 days after the third immunization in CB6F1 mice. At days 0, 14 & 28, CB6F1 mice (n=6/gr1-6) were intramuscularly (i.m) immunized with different version of HSV-1 gE/gI protein adjuvanted with AS01 (5 μg/each). An additional group of mice was i.m injected with a saline solution (NaCl 150 mM) following the same schedule of immunization and used as negative control group (n=4/gr7). On days 42 (14PIII) mice were culled to evaluate HSV-1 gI-specific CD4+T cell response in the spleen. The black error bar represents the 95% Confidence Interval (CI) of each GMR. The GMs of the two groups compared and GMRs are indicated at the right of the graph.

FIG. 53—Anti-HSV-1 gE/gI-specific IgG antibody response measured 28 days after the first or 21 days after the second immunization with different mutated versions of SAM HSV-1 gE/gI vector formulated in Lipid nanoparticles (LNP). At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=6/gr6). On days 28 & 49 (28PI & 21PII), serum sample from each animal was collected to evaluate the total anti-HSV-1 gE/gI-specific IgG antibody titer by ELISA. Each shape represents individual animal at different timepoints (dot=28PI; triangle=21PII) while the black bars represents the Geometric mean of each group. Geometric mean (GM) and number of animals (N) for each group is indicated on the x axis.

FIG. 54—Head to head comparison of anti-HSV-1 gE/gI-specific IgG antibody response between LNP/SAM HSV-1 vaccinated and unvaccinated groups 21 days after the second immunization in CB6F1mice. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM), following the same schedule of immunization, and used as negative control group (n=6/gr6). On days 28 & 49 (28PI & 21PII), serum sample from each animal was collected to evaluate the total anti-HSV-1 gE/gI-specific IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 55—Timepoint comparison of anti-HSV-1 gE/gI-specific IgG antibody response measured 28 days after the first and 21 days after the second immunization with different mutated versions of SAM HSV-1 gE/gI vector formulated in Lipid nanoparticles (LNP). At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=6/gr6). On days 28 & 49 (28PI & 21PII), serum sample from each animal was collected to evaluate the total anti-HSV-1 gE/gI-specific IgG antibody titer by ELISA. The black error bar represents the 95% Confidence Interval (CI) of each geometric mean ratio (GMR). The geometric means (GMs) of the two time points and GMRs are indicated at the right of the graph.

FIG. 56—Head to head comparison of anti-HSV-1 gE/gI-specific IgG antibody response measured in serum samples collected 21 days after the second immunization with different mutated versions of SAM HSV-1 gE/gI vector formulated in Lipid nanoparticles (LNP). At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=6/gr6). On days 28 & 49 (28PI & 21PII), serum sample from each animal was collected to evaluate the total anti-HSV-1 gE/gI-specific IgG antibody titer by ELISA. The black error bar represents the 95% Confidence Interval (CI) of each geometric mean ratio (GMR). The geometric means (GMs) of the two groups and GMRs are indicated at the right of the graph.

FIG. 57—Anti-HSV-2 gE or gI cross-reactive IgG antibody responses measured 21 days after the second immunization with different mutated versions of SAM HSV-1 gE/gI vector formulated in Lipid nanoparticles (LNP). At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=6/gr6). On day 49 (21PII), serum sample from each animal was collected to evaluate the total anti-HSV-2 gE (FIG. 57A) or gI (FIG. 57B) cross-reactive IgG antibody titers by ELISA. Each shape represents individual animal at timepoint 21PII while the black bars represents the Geometric mean of each group. Geometric mean (GM) and number of animals (N) for each group is indicated on the x axis.

FIG. 58—Head to head comparison of anti-HSV-2 gE or HSV-2 gI cross-reactive IgG antibody responses between LNP/SAM HSV-1 vaccinated and unvaccinated groups 21 days after the second immunization in CB6F1mice. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM), following the same schedule of immunization, and used as negative control group (n=6/gr6). On day 49 (21PII), serum sample from each animal was collected to evaluate the total anti-HSV-2 gE (FIG. 58A) or HSV-2 gI (FIG. 58B) cross-reactive IgG antibody titer by ELISA. The black error bar represents the 95% Confidence Interval (CI) of each geometric mean ratio (GMR). The geometric means (GMs) of the two groups compared and GMRs are indicated at the right of the graph.

FIG. 59—Head to head comparison of anti-HSV-2 gE or HSV-2 gI cross-reactive IgG antibody responses measured in serum samples collected 21 days after the second immunization with different mutated versions of SAM HSV-1 gE/gI vector formulated in Lipid nanoparticles (LNP). At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=6/gr6). On day 49 (21PII), serum sample from each animal was collected to evaluate the total anti-HSV-2 gE (FIG. 59A) or HSV-2 gI (FIG. 59B) cross-reactive IgG antibody titer by ELISA. The black error bar represents the 95% Confidence Interval (CI) of each geometric mean ratio (GMR). The geometric means (GMs) of the two groups and GMRs are indicated at the right of the graph.

FIG. 60—HSV-1-specific neutralizing antibody titers measured in serum samples collected 21 days after the second immunization with different mutated versions of LNP-formulated HSV-1 gE/gI vector. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) following the same schedule of immunization and used as negative control group (n=5/gr6). On days 49 (21PII), serum sample was collected to evaluate the titer of neutralizing antibodies towards HSV-1 VR1789 strain (viral load 400 TCID₅₀). FIG. 60A: Each dot represents individual animal titer while bar represents Geometric mean (GM)+95% confidence intervals (CIs). The positivity threshold value corresponds to the 1^{st} sample dilution. Negative samples are illustrated by the 1^{st} samples dilution/2 (neutralizing titer=5). The number of mice by group (N) and the GM for each group are indicated below the x axis of the graph. FIG. 60B: GMR with 95% of confidence interval (CI). The black error bar represents the 95% Confidence Interval (CI) of each GMR. The GMs of the two groups compared and GMRs are indicated at the right of the graph.

FIG. 61—HSV-2 cross-reactive neutralizing antibody titers measured in serum samples collected 21 days after the second immunization with different mutated versions of LNP-formulated HSV-1 gE/gI vector. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) following the same schedule of immunization and used as negative control group (n=5/gr6). On days 49 (21PII), serum sample was collected to evaluate the titer of cross-reactive neutralizing antibodies towards HSV-2 MS strain (viral load 400 TCID₅₀). Each dot represents individual animal titer while bar represents Geometric mean (GM)+95% confidence intervals (CIs). The positivity threshold value corresponds to the 1^{st} sample dilution. Negative samples are illustrated by the 1^{st} samples dilution/2 (neutralizing titer=5). The number of mice by group (N) and the GM for each group are indicated below the x axis of the graph.

FIG. 62—Evaluation of the ability of vaccine-specific antibodies to decrease, in-vitro, hIgG Fc binding by HSV-1 gE/gI protein 21 days after the second immunization with different mutated versions of LNP-formulated SAM-HSV-1 gE/gI vector. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=5/gr6). On days 49 (21PII), serum sample was collected to evaluate the ability of vaccine-specific antibodies to compete and decrease hIgG Fc binding by HSV-1 gE/gI protein. Each curve represents individual mice. LNP/SAM-HSV-1 gE_P319R/gI over NaCl (FIG. 62A); LNP/SAM-HSV-1 gE_P321D/gI over NaCl (FIG. 62B); LNP/SAM-HSV-1 gE_R322D/gI over NaCl (FIG. 62C); LNP/SAM-HSV-1 gE_N243A_R322D/gI over NaCl (FIG. 62D); LNP/SAM-HSV-1 gE_A340G_S341G_V342G/gI over NaCl (FIG. 62E).

FIG. 63—Comparison of the ability of vaccine-specific antibodies to decrease, in-vitro, human IgG Fc binding by HSV-1 gE/gI antigen 21 days after the second immunization with different mutated versions of LNP-formulated SAM HSV-1 gE/gI vector. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=5/gr6). On days 49 (21PII), serum sample was collected to evaluate the ability of vaccine-specific antibodies to compete and decrease hIgG Fc binding by HSV-1 gE/gI protein. FIG. 63A: Each dot represents ED50 titer with 95% CIs from individual mice. The positivity threshold value corresponds to the 1^{st} sample dilution. Negative samples are illustrated by the 1^{st} samples dilution/2 (ED50 titer=5). The number of mice by group (N) and the Geometric mean (GM) for each group is indicated below the x axis of the graph. FIG. 63B: GMR with 95% of confidence interval (CI). The black error bar represents the 95% Confidence Interval (CI) of each geometric mean ratio (GMR). The GMs of the two groups compared and GMRs are indicated at the right of the graph.

FIG. 64—Evaluation of the ability of vaccine-specific antibodies to decrease, in-vitro, hIgG Fc binding by HSV-2 gE/gI protein 21 days after the second immunizations with different mutated versions of LNP-formulated SAM-HSV-1 gE/gI vector. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=5/gr6). On days 49 (21PII), serum sample was collected to evaluate the ability of vaccine-specific antibodies to compete and decrease hIgG Fc binding by HSV-2 gE/gI protein. Each curve represents individual mice. LNP/SAM-HSV-1 gE_P319R/gI over NaCl (FIG. 64A); LNP/SAM-HSV-1 gE_P321D/gI over NaCl (FIG. 64B); LNP/SAM-HSV-1 gE_R322D/gI over NaCl (FIG. 64C); LNP/SAM-HSV-1 gE_N243A_R322D/gI over NaCl (FIG. 64D); LNP/SAM-HSV-1 gE_A340G_S341G_V342G/gI over NaCl (FIG. 64E).

FIG. 65—Comparison of the ability of vaccine-specific antibodies to decrease, in-vitro, human IgG Fc binding by HSV-2 gE/gI protein 21 days after the second immunization with different mutated versions of LNP-formulated SAM HSV-1 gE/gI vector. At days 0 & 28, CB6F1 mice (n=6/gr1-5) were intramuscularly (i.m) immunized with 1 μg of different mutated versions of SAM HSV-1 gE/gI vector formulated in LNP. An additional group of mice was i.m injected with a saline solution (NaCl 150 mM) and used as negative control group (n=5/gr6). On days 49 (21PII), serum sample was collected to evaluate the ability of vaccine-specific antibodies to compete and decrease hIgG Fc binding by HSV-2 gE/gI protein. FIG. 65A: Each dot represents ED50 titer with 95% CIs from individual mice. The positivity threshold value corresponds to the 1^{st} sample dilution. Negative samples are illustrated by the 1^{st} samples dilution/2 (ED50 titer=5). The number of mice by group (N) and the Geometric mean (GM) for each group is indicated below the x axis of the graph. FIG. 65B: GMR with 95% of confidence interval (CI). The black error bar represents the 95% Confidence Interval (CI) of each geometric mean ratio (GMR). The GMs of the two groups compared and GMRs are indicated at the right of the graph.

FIG. 66—Evaluation of the ability of HSV-2 gE/gI cross reactive antibodies collected 21 days after the second immunization with different mutated versions of LNP-formulated SAM HSV-1 gE/gI vector to bind and activate mouse FcγRIII after incubation with HSV-2 gE/gI transfected cells. At days 0 & 28, CB6F1 mice (n=6/gr1-5) were intramuscularly (i.m) immunized with 1 μg of different mutated versions of SAM-HSV-1 gE/gI vector formulated in LNP. An additional group of mice was i.m injected with a saline solution (NaCl 150 mM) and used as negative control group (n=5/gr6). On days 49 (21PII), serum sample was collected to evaluate the ability of HSV-2 gE/gI cross reactive antibodies to bind and activate mouse FcγRIII expressed by modified Jurkat reporter cells after incubation with HSV-2 gE/gI transfected 3T3 cells. FIG. 66A-E: Each curve illustrates data from pools of 2 sera from mice immunized with different mutated versions of LNP/SAM-HSV-1 gE/gI or with a NaCl 150 mM solution.

FIG. 67—Percentage of vaccine-specific and HSV-2 gE cross-reactive CD4+/CD8+T cell responses induced 21 days after the second immunization with different mutated versions of SAM HSV-1 gE/gI vector formulated in LNP in CB6F1 mice. At days 0 & 28, CB6F1 mice (n=6/gr1-5) were intramuscularly (i.m) immunized with lag of different SAM-HSV-1 gE/gI mutant candidates formulated in lipid nanoparticles (LNP). An additional group of mice was i.m injected with a saline solution (NaCl 150 mM) and used as negative control group (n=6/gr6). On day 49 (21PII), mice were culled to evaluate vaccine-specific and HSV_2 gE cross-reactive CD4+/CD8+T cell responses in the spleen. Splenocytes were stimulated ex-vivo during 6 hours with pools of 15mer peptides covering the whole amino acids sequence of the gE or gI protein from HSV-1, as well as gE protein from HSV-2. Human β-actin peptides pool stimulation was used to evaluate non-specific response. The frequencies of CD4+/CD8+ T cells secreting IL-2, IFN-γ and/or TNF-α were measured by intracellular cytokine staining. Circle, triangle, square and diamond shapes represent individual % of CD4+(FIG. 67A) and CD8+(FIG. 67B) T cell responses detected for each antigen (HSV-1 or HSV-2 gE or HSV-2 gI antigens, or Human β-actin). Black line represents the geometric means (GM) of the response and black dotted line indicates the percentile 95th (P95) obtained with all the stimulations in the saline group. The number of animals/group with valid result (N) and the GM of each group are indicated under the graph.

FIG. 68—Head to head comparison of vaccine-specific and HSV-2 gE cross-reactive CD4+/CD8+T cell responses between vaccinated and unvaccinated groups in CB6F1 mice 21 days after the second immunization in CB6F1 mice. At days 0 & 28, CB6F1 mice (n=6/gr1-5) were intramuscularly (i.m) immunized with 1 μg of different SAM-HSV-1 gE/gI mutant candidates formulated in LNP. An additional group of mice was i.m injected with a saline solution (NaCl 150 mM) following the same schedule of immunization and used as negative control group (n=6/gr6). On day 49 (21PII), mice were culled to evaluate vaccine-specific and HSV-2 gE cross-reactive CD4+ and CD8+ T cell responses in the spleen. The black error bar represents the 95% Confidence Interval (CI) of each the geometric mean ratio (GMR). The geometric means (GMs) of the two groups compared and GMRs are indicated at the right of the graph. FIG. 68A: GMR with 95% CI of HSV-1 gE-specific CD4+ T cells; FIG. 68B: GMR with 95% CI of HSV-2 gE-cross reactive CD4+ T cells; FIG. 68C: GMR with 95% CI of HSV-1 gI-specific CD4+ T cells; FIG. 68D: GMR with 95% CI of HSV-1 gI-specific CD8+ T cells.

FIG. 69—Head to head comparison of vaccine-specific and HSV-2 gE cross-reactive CD4+T cell responses between vaccinated groups 21 days after the second immunization in CB6F1 mice. At days 0 & 28, CB6F1 mice (n=6/gr1-5) were intramuscularly (i.m) immunized with lag of different SAM-HSV-1 gE/gI mutant candidates formulated in LNP. An additional group of mice was i.m injected with a saline solution (NaCl 150 mM) following the same schedule of immunization and used as negative control group (n=6/gr6). On day 49 (21PII), mice were culled to evaluate vaccine-specific and HSV-2 gE cross-reactive CD4+T cell responses in the spleen. The black error bar represents the 95% Confidence Interval (CI) of each geometric mean ratio (GMR). The geometric means (GMs) of the two groups compared and GMRs are indicated at the right of the graph. FIG. 69A: GMR with 95% CI of HSV-1 gE-specific CD4+ T cells; FIG. 69B: GMR with 95% CI of HSV-2 gE cross-reactive CD8+ T cells.

FIG. 70—Head to head comparison of HSV-1 gI-specific CD4+/CD8+T cell responses between vaccinated groups 21 days after the second immunization in CB6F1 mice. At days 0 & 28, CB6F1 mice (n=6/gr1-5) were intramuscularly (i.m) immunized with 1 μg of different SAM HSV-1 gE/gI mutant candidates formulated in LNP. An additional group of mice was i.m injected with saline solution (NaCl 150 mM) following the same schedule of immunization and used as negative control group (n=6/gr6). On day 49 (21PII), mice were culled to evaluate anti-HSV-1 gI-specific CD4+/CD8+ T cell responses in the spleen. The black error bar represents the 95% Confidence Interval (CI) of each geometric mean ratio (GMR). The geometric means (GMs) of the two groups compared and GMRs are indicated at the right of the graph. FIG. 70A: GMR with 95% CI of HSV-1 gI-specific CD4+ T cells; FIG. 70B: GMR with 95% CI of HSV-1 gI-specific CD8+ T cells.

FIG. 71—Evaluation of the ability of HSV-1 gE/gI cross reactive antibodies to decrease, in-vitro, human IgG Fc binding by HSV-1 gE/gI antigen 14 days after third immunizations with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant proteins adjuvanted in AS01 (5 μg/each). An additional group of mice was i.m injected with a saline solution (NaCl 150 mM) following the same schedule of immunization and used as negative control group (n=4/gr6). On days 42 (14PIII), serum samples were collected to evaluate the ability of HSV-1 cross reactive antibodies to compete and decrease hIgG Fc binding by HSV-1gE/gI protein. Each curve represents individual mice data. FIG. 71A: AS01/HSV-2 gE/gI V340W over NaCl; FIG. 71B: AS01/HSV-2 gE/gI A248T over NaCl; FIG. 71C: AS01/HSV-2 gE/gI A246W over NaCl; FIG. 71D: AS01/HSV-2 gE/gI P318I over NaCl; FIG. 71E: AS01/HSV-2 gE/gI A248T_V340W over NaCl.

FIG. 72—Evaluation of the ability of HSV-1 gE/gI cross reactive antibodies to bind and activate mFcγRIII after incubation with HSV-1 gE/gI positive cells 14 days after third immunizations with different mutated versions of AS01-adjuvanted HSV-2 gE/gI protein. At days 0, 14 & 28, CB6F1 mice (n=6/gr1-5) were intramuscularly (i.m) immunized with different HSV-2 gE/gI mutant candidates adjuvanted with AS01 (5 μg/each). An additional group of mice was i.m injected with a saline solution (NaCl 150 mM) following the same schedule of immunization and used as negative control group (n=4/gr6). On days 42 (14PIII), serum samples were collected to evaluate the ability of vaccine-specific antibodies to bind HSV-1 gE/gI positive 3T3 cells and activate the mouse F_{c}γRIII expressed by modified Jurkat reporter cells. FIG. 72A-E each curve illustrate data from pools of 2 mouse sera immunized with different AS01-HSV-2 gE/gI mutants over NaCl while FIG. 12F illustrates Geoemetric mean of each AS01-HSV-2 gE/gI vaccinated group over NaCl.

FIG. 73—Evaluation of the ability of HSV-1 gE/gI cross reactive antibodies to decrease, in-vitro, hIgG Fc binding by HSV-1 gE/gI protein 21 days after the third immunization with different LNP-formulated SAM-HSV-2 gE/gI mutants. At days 0, 21 & 42, CB6F1 mice (n=6/gr1-6) were intramuscularly (i.m) immunized with 0.8 μg of different mutated versions of SAM-HSV-2 gE/gI vector formulated in Lipid nanoparticles (LNP). An additional group of mice was i.m injected with a saline solution (NaCl 150 mM) following the same schedule of immunization and used as negative control group (n=4/gr7). On days 63 (21PIII), serum sample from each animal was collected to evaluate the ability of HSV-1 cross reactive antibodies to compete and decrease hIgG Fc binding by HSV-1 gE/gI protein. Each curve represents individual mice. LNP/SAM-HSV-2 gE/gI V340W over NaCl group (FIG. 73A); LNP/SAM-HSV-2 gE/gI A248T over NaCl group (FIG. 73B); LNP/SAM-HSV-2 gE/gI A246W over NaCl group (FIG. 73C); LNP/SAM-HSV-2 gE/gI P318I over NaCl group (FIG. 73D); LNP/SAM-HSV-2 gE/gI A248T_V340W over NaCl group (FIG. 73E); LNP/SAM-HSV-2 gE/gI insert ARAA over NaCl group; (FIG. 73F).

FIG. 74—Evaluation of the ability of HSV-1 gE/gI-specific antibodies collected 14 days after the third immunization with different versions of AS01-adjuvanted HSV-1 gE/gI protein to bind and activate mouse FcγRIII after incubation with HSV-1 gE/gI transfected cells. At days 0, 14 & 28, CB6F1 mice (n=6/gr1-6) were intramuscularly (i.m) immunized with different versions of HSV-1 gE/gI protein adjuvanted in AS01 (5 μg/each). An additional group of mice was i.m injected with saline solution (NaCl 150 mM) following the same schedule of immunization and used as negative control group (n=4/gr7). On days 42 (14PIII), serum samples were collected to evaluate the ability of vaccine-specific antibodies to bind and activate mouse FcγRIII expressed by modified Jurkat reporter cells after incubation with HSV-1 gE/gI transfected 3T3 cells. FIG. 74A-F: Each curve illustrates data from pools of 2 mouse sera immunized with different versions of AS01-adjuvanted HSV-1 gE/gI protein or treated with a NaCl 150 mM solution. FIG. 74G: illustrates Geoemetric mean of each AS01-HSV-1 gE/gI vaccinated group over NaCl.

FIG. 75—Annotated amino acid sequences for HSV2 gE (UniprotKB: A7U881) and HSV1 gE (UniprotKB: Q703E9). Sequence alignment on EBIO using GAP, Gap Weight: 8, Length Weight: 2, Similarity: 78.68%, Identity: 76.10%. Underlined: Signal peptide (SP); bold underlined: transmembrane domain; Italic underlined: Fc-binding region; bold italic: region required for heterodimer complex formation.

FIG. 76—Annotated amino acid sequences for HSV2 gI (UniprotKB: A8U5L5) and HSV1 gI (UniprotKB: P06487). Sequence alignment on EBIO using GAP; Gap Weight: 8; Length Weight: 2; Similarity: 73.37%; Identity: 70.38%. Underlined: Signal peptide (SP); Bold underlined: transmembrane domain; bold italic: region required for heterodimer complex formation.

FIG. 77—Alignment of HSV2 gE ectodomain protein sequences. Black/dark grey/light grey shading: 100%/80%/60% similarity respectively across all aligned sequences.

FIG. 78—Alignment of HSV2 gI ectodomain protein sequences. Black/dark grey/light grey shading: 100%/80%/60% similarity respectively across all aligned sequences.

FIG. 79—DNA sequence of the plasmid that expresses the RNA sequence for the SAM-gEgI constructs. Upper case: SAM backbone; Lower case: non-SAM sequence; underlined: 5′ UTR of SAM; bold underlined: 3′ UTR of SAM; grey shade: Insert encoding the gEgI heterodimer.

FIG. 80—Design of several ThHSV SAM vectors encoding for gEgI heterodimer. Cloning was performed into VEEV TC-83 SAM vector (Venezuelan Equine Encephalitis virus-attenuated strain). HSV2 gE P317R mutant (Fc binding KO) versions were also generated. A) Screening of different regulatory elements to drive gI expression. The selected regulatory elements were i) Enterovirus 71 Internal Ribosome entry site (EV71 IRES), ii) two 2A peptide sequences (GSG-P2A: Porcine teschovirus-1 2A with GSG linker, F2A: 2A peptide from the foot-and-mouth disease virus (F2A)) and iii) the promoter for 26S RNA (26S prom). Size (bp) of each regulatory element is indicated. B) Same constructs as in A), including an HA-tag in C-term of HSV2 gE and gI proteins.

### DETAILED DESCRIPTION OF THE INVENTION

The present invention relates to the use of a nucleic acid encoding a HSV Fc receptor or an immunogenic fragment or variant thereof, in particular glycoprotein gE from HSV1 or HSV2 either alone or with its binding partner gI, in a therapeutic vaccine against HSV, wherein the nucleic acid encoding a HSV Fc receptor or immunogenic fragment or variant thereof induces a cross reactive immune response against HSV1 or HSV2 when administered to a subject. The inventors have shown that immunisation of mice with a RNA encoding the ectodomain of HSV2 Fc receptor (in particular gE/gI) or variants thereof with mutations of gE/gI that reduce or abolish binding to a human antibody, results in a functional cross reactive immune response against HSV1 viruses or HSV1 antigens. It was also demonstrated that the immunisation of mice with a RNA encoding the ectodomain of HSV1 Fc receptor (in particular gE/gI) or variants thereof with mutations of gE/gI that reduce or abolish binding to a human antibody, results in a functional cross reactive immune response against HSV2 viruses or HSV2 antigens. Amino acid sequence alignments between HSV1 KOS strain and HSV2 SD90e strain revealed gE and gI proteins to respectively share 76.10%/67.4% and 78.68%/75.5% of identity/similarity. But a functional cross-reactive immune response between HSV-1 and HSV-2 gE/gI was unexpected. Even at significantly higher levels of sequence similarity between antigens, this does not imply a functional cross-reactive immune response. For example, some influenza virus HA antigens (from strains post 2009) share approximately 90% amino acid sequence similarity yet show no functional cross-reactive immune response. Hence, it was surprising that administration of a nucleic acid encoding a first HSV serotype would show cross-reactivity and elicit an immune response in a subject for a different HSV serotype.

Hence, there is provided a nucleic acid encoding a HSV2 Fc receptor or an immunogenic fragment or variant thereof for use in inducing a cross reactive immune response against HSV-1 when administered to a subject. Also provided is a nucleic acid encoding a HSV1 Fc receptor or an immunogenic fragment or variant thereof for use in inducing a cross reactive immune response against HSV2 when administered to a subject. The nucleic acid encoding a HSV1 Fc receptor or an immunogenic fragment or variant thereof may be used in treating a subject infected with HSV1 and/or HSV2, preferably for use as a pan-HSV vaccine. The nucleic acid encoding a HSV2 Fc receptor or an immunogenic fragment or variant thereof may be used in treating a subject infected with HSV1 and/or HSV2, preferably for use as a pan-HSV vaccine. The nucleic acid for use according to the invention which encodes a HSV1 or HSV2 Fc receptor or immunogenic fragment or variant thereof may be used in particular against recurrent infections with HSV1 and/or HSV2 and related clinical and sub-clinical manifestations.

Cross Reactive Immune Response

The immune response is cross reactive in that the nucleic acid encoding a HSV Fc receptor or immunogenic fragment or variant thereof can induce an antigen specific humoral and/or cellular immune response against an antigen from a serotype of HSV other than the one from which it is derived, i.e. it induces a cross serotype response. For example, in the case of a nucleic acid encoding a HSV2 Fc receptor or immunogenic fragment or variant thereof, it can induce a humoral and/or cellular immune response against HSV1, in the case of a nucleic acid encoding a HSV1 Fc receptor or immunogenic fragment or variant thereof, it can induce a humoral and/or cellular immune response against HSV2. Thus, in one embodiment, the nucleic acid described herein induces a humoral and/or cellular immune response to both homologous and heterologous serotypes of HSV. Hence, the nucleic acid encoding a HSV Fc receptor or an immunogenic fragment or variant thereof, may induce an immune response against both the serotype of HSV from which it is derived and against another serotype of HSV from which the Fc receptor is not derived. As there are two serotypes of HSV (HSV1 and HSV2), the nucleic acid encoding a HSV Fc receptor or immunogenic fragment or variant thereof may induce a humoral and/or cellular immune response against both HSV1 and HSV2. The humoral immune response may comprise the generation of antibodies that bind to an antigen from a serotype of HSV other than the one from which the HSV Fc receptor or immunogenic fragment or variant thereof is derived. Alternatively, or in addition, the cross-reactive immune response may lead to the generation of a specific T lymphocyte cell response, for example a CD4+ T cell response and/or a CD8+ T cell response.

The immune response may be functional, for example not only binding of an antibody to the encoded Fc receptor antigen or antigen associated molecule or cell, but that initiates downstream effector functions that contribute to an effective immune response. In one embodiment, a functional antibody response is induced. For example, a functional antibody response may include the activation of complement, granulocytes and/or cytotoxic T cells, neutralisation of viral particles, or other means to inhibit or immobilise HSV or inhibition of HSV's immune evasion activity. As a cross reactive functional immune response, the encoded HSV Fc receptor or immunogenic fragment or variant thereof may initiate downstream effector functions (e.g. activation of complement, granulocytes and/or cytotoxic T cells, neutralisation, or other means to inhibit or immobilise HSV or inhibition of HSV's immune evasion activity) against a serotype of HSV from which it is not derived, i.e. generating a cross serotype response. Preferably, the functional antibody response comprises inhibition of HSV's immune evasion activity. The latter may be achieved by generation of antibodies that bind HSV Fc receptor and inhibit the binding of HSV Fc receptor to human IgG. In this way, the functional immune response may include inhibition of antibody bipolar bridging, for example bipolar bridging of antibodies bound to the surface of HSV-infected cells.

The generation of cross reactive antibodies that bind for example, HSV Fc receptor, can be measured by techniques well known to those skilled in the art, for example, an ELISA, or indirect ELISA for detection of cross reactive antibodies that bind a serotype of HSV from which the Fc receptor antigen was not derived. The ability to inhibit immune evasion activity may be measured for example, by a competitive ELISA to assess the ability of vaccine specific antibodies to decrease human IgG Fc binding by HSV Fc receptor in vitro. A neutralisation assay can be used to assess the presence of cross neutralising antibodies. Neutralising antibodies may prevent the replication of HSV in permissive host cells, cells which allow the penetration and multiplication of HSV. A neutralisation assay can assess the ability of the antibodies to neutralise the virus strain, for example by assessing the cytopathic effect of virus infected host cells in the presence or absence of serum containing such antibodies.

The functional immune response may comprise the generation of a cross reactive (e.g. cross serotype) T cell response. Two main cellular effectors of the specific T cell response are the CD4+ helper T cells and CD8+T lymphocytes (or cytotoxic T lymphocytes or CTLs). The activation of CD4+ T cells causes them to release cytokines which influence the activity of many cell types such as B lymphocytes, CTLs and antigen presenting cells (APCs). CD4+ T cells can be further subdivided into Th1 and Th2 which produce Th1-type cytokines (e.g. IL-2 and IFN-γ) and Th2-type cytokines. The activation of CD4+ T cells and secretion of cytokines can stimulate both cell-mediated and antibody-mediated branches of the immune system. Preferably, the cross reactive CD4+ T cell response is primarily a Th1 cell-mediated immune response, for example inducing IL-2 and/or IFN-γ. CD8+ T cells (or cytotoxic T cells (CTLs)) may also be involved in T cell-medicated immune responses and can induce the death of cells infected with viruses. CD8+ T cells can produce cytokines (e.g. IFN-γ) upon activation following specific antigen stimulation. The release of cytokines upon activation of CD4+ helper T cells or CD8+ T cells can be measured to assess and quantify this activation. Techniques well known to those skilled in the art may be used to measure the cytokine response associated with CD4+/CD8+ T cell activation and include but are not limited to use of a specific ELISA or ELISPOT or flow cytometry designed for measurement of cytokines such as IFN-γ, IL-2 and/or others. In particular, an intracellular cytokine staining assay may be used to detect intracellular cytokines in these cells and then measured for example using flow cytometry. Cytotoxicity can be assessed using for example in vitro cytolytic assays whereby antigen-loaded target cells are lysed by specific cytotoxic T cells that are generated in vivo following vaccination or infection. Techniques for this are well known to those skilled in the art and include for example a chromium release assay or an antibody-dependent cell-mediated cytotoxicity (ADCC) bioassay. ADCC bioassays may detect killing of target cells (e.g. by PBMCs or NK cells) after cytotoxic pathways are activated when membrane surface antigens are bound by specific antibodies. Alternative assays such as the Promega ADCC Reporter Bioassay may detect FcγR mediated activation of luciferase activity in cell lines stably expressing human FcγRIIIa induced luciferase. Following engagement with the Fc region of a relevant antibody bound to a target cell, the bioassay effector cells expressing FcγRIIIa transduce intracellular signals resulting in luciferase activity that can be easily quantified. In one embodiment, is provided a method for determining a cross reactive immune response in a subject following administration of an immunologically effective amount of a nucleic acid encoding a HSV2 Fc receptor or immunogenic fragment or variant thereof, comprising taking a tissue (e.g. blood) sample from the subject and performing an in vitro assay to detect a cross reactive immune response against HSV1. In a further embodiment, is provided a method for determining a cross reactive immune response in a subject following administration of an immunologically effective amount of a nucleic acid encoding a HSV1 Fc receptor or immunogenic fragment or variant thereof, comprising taking a tissue (e.g. blood) sample from the subject and performing an in vitro assay to detect a cross reactive immune response against HSV2. In a further embodiment, is provided a method for determining a cross reactive immune response in a subject following administration of an immunologically effective amount of a nucleic acid encoding a HSV2 Fc receptor or immunogenic fragment or variant thereof, comprising taking a tissue (e.g. blood) sample from the subject and performing an in vitro assay to detect a cross reactive immune response against HSV1. In one embodiment, the in vitro assay is used to detect cross reactive antibodies in a blood sample.

In one embodiment is provided a nucleic acid encoding a HSV2 Fc receptor or an immunogenic fragment or variant thereof for the use described herein wherein the cross-reactive immune response comprises a cross serotype antibody response against HSV1, preferably for use as a pan-HSV vaccine. In a further embodiment is provided a nucleic acid encoding a HSV1 Fc receptor or an immunogenic fragment or variant thereof for the use described herein wherein the cross-reactive immune response comprises a cross serotype antibody response against HSV2, preferably for use as a pan-HSV vaccine.

In one embodiment is provided a nucleic acid encoding a HSV Fc receptor or an immunogenic fragment or variant thereof for use in inducing a cross reactive immune response against HSV of a different serotype when administered to a subject.

In one embodiment is provided a nucleic acid encoding a HSV2 Fc receptor or an immunogenic fragment or variant thereof for the use described herein wherein:
the nucleic acid is a RNA molecule and
the cross-reactive immune response comprises a cross serotype antibody response against HSV.

In a further embodiment is provided a nucleic acid encoding a HSV1 Fc receptor or an immunogenic fragment or variant thereof for the use described herein wherein:
the nucleic acid is a RNA molecule and
the cross-reactive immune response comprises a cross serotype antibody response against HSV2.

In one embodiment is provided a nucleic acid encoding a HSV2 Fc receptor or an immunogenic fragment or variant thereof for the use described herein wherein:
the nucleic acid is a RNA molecule,
the cross-reactive immune response comprises a cross serotype antibody response against HSV1 and
the HSV2 Fc receptor is part of a heterodimer with a binding partner from HSV or a fragment thereof, preferably wherein the HSV2 Fc receptor is HSV2 gE2 or an immunogenic fragment or variant thereof.

In a further embodiment is provided a nucleic acid encoding a HSV1 Fc receptor or an immunogenic fragment or variant thereof for the use described herein wherein:
the nucleic acid is a RNA molecule,
the cross-reactive immune response comprises a cross serotype antibody response against HSV2 and
the HSV1 Fc receptor is part of a heterodimer with a binding partner from HSV or a fragment thereof, preferably wherein the HSV1 Fc receptor is HSV1 gE1 or an immunogenic fragment or variant thereof.

In one embodiment is provided a nucleic acid encoding a HSV2 Fc receptor or an immunogenic fragment or variant thereof for use in inducing a cross reactive immune response against HSV1 when administered to a subject and for use as a pan-HSV vaccine, wherein:
the nucleic acid is a RNA molecule and
the HSV2 Fc receptor is part of a heterodimer with a binding partner from HSV or a fragment thereof, preferably wherein the HSV2 Fc receptor is HSV2 gE2 or an immunogenic fragment or variant thereof.

In a further embodiment is provided a nucleic acid encoding a HSV1 Fc receptor or an immunogenic fragment or variant thereof for use in inducing a cross reactive immune response against HSV2 when administered to a subject and for use as a pan-HSV vaccine, wherein:
the nucleic acid is a RNA molecule and
the HSV1 Fc receptor is part of a heterodimer with a binding partner from HSV or a fragment thereof, preferably wherein the HSV1 Fc receptor is HSV1 gE1 or an immunogenic fragment or variant thereof.

In one embodiment is provided a nucleic acid encoding a HSV2 Fc receptor or an immunogenic fragment or variant thereof for the use described herein wherein:
the nucleic acid is a RNA molecule and
said use does not comprise administration to the subject together with HSV2 gD2.

In a further embodiment is provided a nucleic acid encoding a HSV1 Fc receptor or an immunogenic fragment or variant thereof for the use described herein wherein:
the nucleic acid is a RNA molecule and
said use does not comprise administration to the subject together with HSV1 gD1.

In one embodiment is provided a nucleic acid encoding a HSV2 Fc receptor or an immunogenic fragment or variant thereof for the use described herein wherein:
the nucleic acid is a RNA molecule
the cross-reactive immune response comprises a cross serotype antibody response against HSV1 and optionally a cross serotype T cell response and
said use does not comprise administration to the subject together with HSV2 gD2.

In a further embodiment is provided a nucleic acid encoding a HSV1 Fc receptor or an immunogenic fragment or variant thereof for the use described herein wherein:
the nucleic acid is a RNA molecule
the cross-reactive immune response comprises a cross serotype antibody response against HSV2 and
said use does not comprise administration to the subject together with HSV1 gD1.

In one embodiment, there is provided a nucleic acid encoding a HSV2 Fc receptor or an immunogenic fragment or variant thereof for the use described herein, wherein the cross reactive immune response comprises a cross serotype antibody response, a cross serotype cytotoxic antibody response, a cross serotype T cell response and/or generation of antibodies that can inhibit the immune evasion activity of HSV. Preferably, the cross-reactive immune response comprises a cross serotype T cell response (e.g. a CD8+ T cell response) and cross serotype antibody response against HSV1.

In a further embodiment, there is provided a nucleic acid encoding a HSV1 Fc receptor or an immunogenic fragment or variant thereof for the use described herein, wherein the cross reactive immune response comprises a cross serotype antibody response, a cross serotype cytotoxic antibody response, a cross serotype T cell response and/or generation of antibodies that can inhibit the immune evasion activity of HSV. Preferably, the cross-reactive immune response comprises a cross serotype antibody response, a cross serotype cytotoxic antibody response and/or the generation of antibodies that can inhibit the immune evasion activity of HSV2 (e.g. by inhibiting binding of HSV2 Fc receptor to IgG).

In one embodiment is provided a nucleic acid encoding a HSV2 Fc receptor or an immunogenic fragment or variant thereof for the use described herein wherein:
the nucleic acid is a RNA molecule
the cross-reactive immune response comprises a T cell response (e.g. a CD8+ T cell response) and
said use does not comprise administration to the subject together with HSV2 gD2 or a fragment thereof comprising immunodominant epitopes.

In a further embodiment is provided a nucleic acid encoding a HSV1 Fc receptor or an immunogenic fragment or variant thereof for the use described herein wherein:
the nucleic acid is a RNA molecule
the cross-reactive immune response comprises the generation of antibodies that can inhibit the immune evasion activity of HSV (e.g. by inhibiting the binding of HSV2 Fc receptor to IgG) and
said use does not comprise administration to the subject together with HSV1 gD1 or a fragment thereof comprising immunodominant epitopes.

In one embodiment is provided a nucleic acid encoding a HSV2 Fc receptor or an immunogenic fragment or variant thereof for the use described herein wherein:
the nucleic acid is a RNA molecule (e.g. a self replicating RNA molecule) and the RNA is encapsulated in lipid nanoparticles (LNP) and
said use does not comprise administration to the subject together with HSV2 gD2 or a fragment thereof comprising immunodominant epitopes.

In a further embodiment is provided a nucleic acid encoding a HSV1 Fc receptor or an immunogenic fragment or variant thereof for the use described herein wherein:
the nucleic acid is a RNA molecule (e.g. a self-replicating RNA molecule) and the RNA is encapsulated in lipid nanoparticles (LNP) and
said use does not comprise administration to the subject together with HSV1 gD1 or a fragment thereof comprising immunodominant epitopes.

In one embodiment is provided a nucleic acid encoding a HSV2 Fc receptor or an immunogenic fragment or variant thereof for the use described herein wherein:
the nucleic acid is a RNA molecule (e.g. a self replicating RNA molecule) and the RNA is encapsulated in lipid nanoparticles (LNP)
said use does not comprise administration to the subject together with HSV2 gD2 or a fragment thereof comprising immunodominant epitopes and
said use includes use as a pan-HSV vaccine.

In a further embodiment is provided a nucleic acid encoding a HSV1 Fc receptor or an immunogenic fragment or variant thereof for the use described herein wherein:
the nucleic acid is a RNA molecule (e.g. a self-replicating RNA molecule) and the RNA is encapsulated in lipid nanoparticles (LNP)
said use does not comprise administration to the subject together with HSV1 gD1 or a fragment thereof comprising immunodominant epitopes and
said use includes use as a pan-HSV vaccine.

Herpes Simplex Virus and Immune Evasion

Alphaherpesviruses, such as herpes simplex virus (HSV), have evolved specialized mechanisms enabling virus spread in epithelial and neuronal tissues. Primary infection involves entry into mucosal epithelial cells, followed by rapid virus spread between these cells. During this phase of virus replication and spread, viruses enter sensory neurons by fusion of the virion envelope with neuronal membranes so that capsids are delivered into the cytoplasm. Capsids undergo retrograde axonal transport on microtubules toward neuronal cell bodies or nuclei in ganglia, where latency is established. Later, following stimulation of neurons, latent virus reactivates and there is production of virus particles that undergo fast axonal transport on microtubules in the anterograde direction from cell bodies to axon tips. An essential phase of the life cycle of herpes simplex virus (HSV) and other alphaherpesviruses is the capacity to reactivate from latency and then spread from infected neurons to epithelial tissues. This spread involves at least two steps: (i) anterograde transport to axon tips followed by (ii) exocytosis and extracellular spread from axons to epithelial cells. HSV gE/gI is a heterodimer formed from two viral membrane glycoproteins, gE and gI. The HSV gE/gI heterodimer has been shown to facilitate virus spread. (Howard, Paul W., et al. “Herpes simplex virus gE/gI extracellular domains promote axonal transport and spread from neurons to epithelial cells.” Journal of virology 88.19 (2014): 11178-11186.)

When HSV1 or HSV2 reactivates in an infected cell, the virus becomes more visible to the immune system and therefore more vulnerable. Typically, host IgG recognize viral antigens on the virion or at the cell surface of infected cells and the host IgG Fc domain can mediate important antibody effector activities by interacting with Fc gamma receptor on NK cells, granulocytes and macrophages to trigger antibody-dependent cellular cytotoxicity (ADCC), and by interacting with Fc gamma receptor on macrophages, monocytes, neutrophils and dendritic cells to trigger antibody-dependent cellular phagocytosis (ADCP).

HSV1 or HSV2 gE can form a noncovalent heterodimer complex with HSV1 or HSV2 (respectively) glycoprotein I (gI). The gEgI heterodimer functions as a viral Fc gamma receptor (FcγR), meaning it has the capacity to interact with the Fc portion of human IgG. Indeed, HSV1 or HSV2 gE or gE/gI heterodimer, when displayed at the cell surface of HSV infected cells, binds host IgG through their Fc portion. The interaction between gE and gI is thought to increase Fc binding affinity by a factor of about a hundred as compared to gE alone. This interaction has been linked to an immune evasion mechanism. Indeed, human IgGs which can bind HSV1 or HSV2 antigens (for example gD) on the virion or infected cell through the IgG Fab domain can also bind the Fc binding domain on the viral gE through their Fc domain, leading to endocytosis of the immune complex through a clathrin-mediated mechanism. This mechanism is referred to as antibody bipolar bridging and is postulated to be a major immune evasion strategy competing with innate immune cell activation. Through antibody Fc binding, the viral FcγR inhibits IgG Fc-mediated activities, including complement binding and antibody-dependent cellular cytotoxicity (ADCC) allowing the virus to circumvent the recognition by the immune system. (Ndjamen, Blaise, et al. “The herpes virus Fc receptor gE-gI mediates antibody bipolar bridging to clear viral antigens from the cell surface.” PLoS pathogens 10.3 (2014): el003961.; Dubin, G., et al. “Herpes simplex virus type 1 Fc receptor protects infected cells from antibody-dependent cellular cytotoxicity.” Journal of virology 65.12 (1991): 7046-7050.; Sprague, Elizabeth R., et al. “Crystal structure of the HSV1 Fc receptor bound to Fc reveals a mechanism for antibody bipolar bridging.” PLoS biology 4.6 (2006): e148.)

HSV2 prophylactic subunit gD2 vaccines did not efficiently prevent HSV-2 disease or infection in human trials (Johnston, Christine, Sami L. Gottlieb, and Anna Wald. “Status of vaccine research and development of vaccines for herpes simplex virus.” Vaccine 34.26 (2016): 2948-2952). Another HSV2 vaccine candidate based on a truncated gD2 and ICP4.2 antigens adjuvanted with Matrix-M2 reduced genital HSV2 shedding and lesion rates in a phase 2 trial (Van Wagoner, Nicholas, et al. “Effects of different doses of GEN-003, a therapeutic vaccine for genital herpes simplex virus-2, on viral shedding and lesions: results of a randomized placebo-controlled trial.” The Journal of infectious diseases 218.12 (2018): 1890-1899.) A trivalent adjuvanted vaccine including a virus entry molecule (gD2) and two antigens that block HSV2 immune evasion, gC2 which inhibits complement and gE2, showed efficacy in animal models. (Awasthi, Sita, et al. “Blocking herpes simplex virus 2 glycoprotein E immune evasion as an approach to enhance efficacy of a trivalent subunit antigen vaccine for genital herpes.” Journal of virology 88.15 (2014): 8421-8432.; Awasthi, Sita, et al. “An HSV2 trivalent vaccine is immunogenic in rhesus macaques and highly efficacious in guinea pigs.” PLoS pathogens 13.1 (2017): e1006141.; Awasthi, Sita, et al. “A trivalent subunit antigen glycoprotein vaccine as immunotherapy for genital herpes in the guinea pig genital infection model.” Human vaccines & immunotherapeutics 13.12 (2017): 2785-2793.; Hook, Lauren M., et al. “A trivalent gC2/gD2/gE2 vaccine for herpes simplex virus generates antibody responses that block immune evasion domains on gC2 better than natural infection.” Vaccine 37.4 (2019): 664-669.)

Directing an immune response against the Fc binding domain of gE may prevent or interfere with the above described immune evasion mechanism (antibody bipolar bridging), allowing natural immunity to viral proteins, in particular the immune-dominant HSV1 or HSV2 gD antigen, to become more potent. Specific targeting of gE or the gE//gI heterodimer through vaccination may enhance subdominant immune responses, in particular antibody dependent cellular cytotoxicity (ADCC) and antibody dependent cellular phagocytosis (ADCP), and redirect the immune system to protective mechanisms. Without wishing to be bound by theory, the present inventors thus believe that specifically raising a cross reactive immune response through vaccination with a nucleic acid encoding gE alone or in combination with its heterodimer binding partner gI could effectively treat subjects infected with HSV1 or HSV2 or prevent subjects from being infected with HSV1 or HSV2. In some aspects, specifically raising a cross-reactive immune response through vaccination with a nucleic acid encoding gE alone or in combination with its heterodimer binding partner gI could effectively treat subjects to reduce or abolish latent viral infection with HSV1 or HSV2 and/or treat or reduce acute infection arising from the latent viral infection with HSV1 or HSV2 or prevent subjects from being infected with HSV1 or HSV2. In some aspects, specifically raising a cross-reactive immune response through vaccination with a nucleic acid encoding gE alone or in combination with its heterodimer binding partner gI could effectively treat subjects to reduce or abolish latent viral infection with HSV1 or HSV2 and/or treat or reduce acute infection arising from the latent viral infection with HSV1 or HSV2.

For the treatment of subjects that have already been infected by a herpes virus (seropositive subjects), whether they are in a symptomatic or asymptomatic phase, it may not be necessary to include an immunodominant antigen such as HSV gD in a therapeutic composition. Indeed, gD is a dominant antigen and seropositive subjects, whether symptomatic or asymptomatic, already have high levels of naturally generated neutralising antibodies against gD (Cairns, Tina M., et al. “Patient-specific neutralizing antibody responses to herpes simplex virus are attributed to epitopes on gD, gB, or both and can be type specific.” Journal of virology 89.18 (2015): 9213-9231.). By inducing an immune response against a viral Fc receptor such as HSV gE or gE/gI, the present inventors have hypothesized the gE/gI immune evasion mechanism will be circumvented and the natural immunity will more fully play its role, in particular the natural antibody responses directed against immunodominant antigens such as gD. In addition to acting on the immune evasion mechanism, the gE or gE/gI antigen may also induce a humoral response (anti gE or anti gE and gI antibodies) that would lead to the destruction of infected cells by cytotoxic and/or phagocytic mechanisms (ADCC/ADCP). In the case of seropositive subjects, the ADCC and/or ADCP mechanisms may be more efficient than neutralising antibody mechanisms (such as the response driven by the dominant HSV antigen gD) to control at early stage viral replication. Finally, it is hypothesised that the induction of CD4+ T cells with a gE or gE/gI antigen would also be helpful against recurrent HSV infections.

Fc Receptor

In a first aspect, the invention provides a nucleic acid encoding a HSV-2 Fc receptor or an immunogenic fragment or variant thereof for use in inducing a cross reactive immune response against HSV-1 when administered to a subject. In a second aspect, is provided a nucleic acid encoding a HSV-1 Fc receptor or an immunogenic fragment or variant thereof for use in inducing a cross reactive immune response against HSV-2 when administered to a subject.

As used herein, an “Fc receptor” (or “FcR”) is a protein found at the surface of certain cells and which has the ability to bind the Fc region of an antibody. Fc receptors are classified based on the type of antibody that they recognize. Fc receptors which bind IgG, the most common class of antibody, are referred to as “Fc-gamma receptors” (or “FcγR”), those that bind IgA are called “Fc-alpha receptors” (or “FcαR”) and those that bind IgE are called “Fc-epsilon receptors” (or “FcεR”). Herein, Fc receptors displayed on the surface of cells from a given multicellular organism as a result of the expression of endogenous genes are referred to as “host Fc receptors”. Host Fc receptors are found in particular on the surface of host immune effector cells such as B lymphocytes, follicular dendritic cells, natural killer (NK) cells, macrophages, neutrophils, eosinophils, basophils, human platelets, and mast cells. The binding of host Fc receptors to the Fc region of antibodies that are bound to infected cells or invading pathogens through their Fab region triggers phagocytosis or destruction of the infected cells or invading pathogens by antibody-mediated cellular phagocytosis (ADCP) or antibody-dependent cellular cytotoxicity (ADCC).

Some viruses, in particular herpes viruses, express viral Fc receptors that bind the Fc portion of the host IgGs, thereby preventing binding of the IgG to host Fc receptors on immune effector cells and allowing the virus to evade host ADCC or ADCP immune responses.

In one embodiment, the encoded HSV Fc receptor is a FcγR. Preferably, the FcγR is selected from HSV2 gE2 and HSV1 gE1.

Herein, a “HSV2 gE2” (or “HSV2 gE”) is a HSV2 gE glycoprotein encoded by HSV2 gene US8 and displayed on the surface of infected cells and which functions as a viral FcγR. Suitably, the HSV2 gE2 is selected from the HSV2 gE glycoproteins shown in Table 1 or variants therefrom which are at least 60%, 65%, 70%, 75% 80%, 85%, 90%, 95%, 96%, 97%, 98%, 99% or 99.5% identical thereto.

TABLE 1
HSV2 gE glycoproteins and ectodomains

| Genbank accession number | SEQ ID NO | Ectodomain |
|---|---|---|
| AHG54732.1 | 1 | 1-419 |
| AKC59449.1 | 13 | 1-419 |
| AKC42830.1 | 14 | 1-419 |
| ABU45436.1 | 15 | 1-419 |
| ABU45439.1 | 16 | 1-419 |
| ABU45437.1 | 17 | 1-419 |
| ABU45438.1 | 18 | 1-419 |
| AMB66104.1 | 19 | 1-416 |
| AMB66173.1 | 20 | 1-416 |
| AMB66246.1 | 21 | 1-419 |
| AKC59520.1 | 22 | 1-419 |
| AKC59591.1 | 23 | 1-419 |
| AKC59307.1 | 24 | 1-419 |
| AMB66465.1 | 25 | 1-419 |
| AKC59378.1 | 26 | 1-419 |
| AEV91407.1 | 27 | 1-416 |
| CAB06715.1 | 28 | 1-416 |
| YP_009137220.1 | 29 | 1-416 |
| ABW83306.1 | 30 | 1-419 |
| ABW83324.1 | 31 | 1-419 |
| ABW83308.1 | 32 | 1-419 |
| ABW83310.1 | 33 | 1-419 |
| ABW83312.1 | 34 | 1-419 |
| ABW83314.1 | 35 | 1-419 |
| ABW83316.1 | 36 | 1-419 |
| ABW83318.1 | 37 | 1-419 |
| ABW83320.1 | 38 | 1-419 |
| ABW83322.1 | 39 | 1-419 |
| ABW83398.1 | 40 | 1-419 |
| ABW83380.1 | 41 | 1-419 |
| ABW83396.1 | 42 | 1-416 |
| ABW83382.1 | 43 | 1-419 |
| ABW83384.1 | 44 | 1-419 |
| ABW83394.1 | 45 | 1-416 |
| ABW83386.1 | 46 | 1-419 |
| ABW83388.1 | 47 | 1-419 |
| ABW83390.1 | 48 | 1-419 |
| ABW83392.1 | 49 | 1-419 |
| ABW83400.1 | 50 | 1-419 |
| ABW83342.1 | 51 | 1-419 |
| ABW83340.1 | 52 | 1-419 |
| ABW83346.1 | 53 | 1-419 |
| ABW83348.1 | 54 | 1-419 |
| ABW83326.1 | 55 | 1-419 |
| ABW83350.1 | 56 | 1-419 |
| ABW83352.1 | 57 | 1-419 |
| ABW83336.1 | 58 | 1-419 |
| ABW83334.1 | 59 | 1-419 |
| ABW83354.1 | 60 | 1-419 |
| ABW83338.1 | 61 | 1-419 |

In a preferred embodiment, the encoded HSV2 gE2 is the gE from HSV2 strain SD90e (Genbank accession number AHG54732.1, UniProtKB accession number: A7U881) which has the amino acid sequence shown in SEQ ID NO:1, or a variant therefrom which is at least 60%, 65%, 70%, 75% 80%, 85%, 90%, 95%, 96%, 97%, 98%, 99% or 99.5% identical thereto.

As used herein, a “Variant” is a peptide sequence that differs in sequence from a reference antigen sequence but retains at least one essential property of the reference antigen. Changes in the sequence of peptide variants may be limited or conservative, so that the sequences of the reference peptide and the variant are closely similar overall and, in many regions, identical. A variant and reference antigen can differ in amino acid sequence by one or more substitutions, additions or deletions in any combination. A variant of an antigen can be naturally occurring such as an allelic variant, or can be a variant that is not known to occur naturally. Non-naturally occurring variants of nucleic acids and polypeptides may be made by mutagenesis techniques or by direct synthesis. In a preferred embodiment, the essential property retained by the variant is the ability to induce an immune response, suitably a humoral or T cell response, which is similar to the immune response induced by the reference antigen. Suitably, the variant induces a humoral or T cell response in mice which is not more than 10-fold lower, more suitably not more than 5-fold lower, not more than 2-fold lower or not lower, than the immune response induced by the reference antigen.

Herein, a “HSV1 gE1” (or “HSV1 gE”) is a HSV1 gE glycoprotein encoded by HSV1 gene US8 and displayed on the surface of infected cells and which functions as a viral Fc7R. Suitably, the encoded HSV1 gE1 is the gE from HSV1 strain KOS321 (UniProtKB accession number: Q703E9) which has the amino acid sequence shown in SEQ ID NO:3, or a variant therefrom which is at least 60%, 65%, 70%, 75% 80%, 85%, 90%, 95%, 96%, 97%, 98%, 99% or 99.5% identical thereto.

In one embodiment, an immunogenic fragment of a HSV Fc receptor is used.

As used herein, an “immunogenic fragment” refers to a fragment of a reference antigen containing one or more epitopes (e.g., linear, conformational or both) capable of stimulating a host's immune system to make a humoral and/or cellular antigen-specific immunological response (i.e. an immune response which specifically recognizes a naturally occurring polypeptide, e.g., a viral or bacterial protein). An “epitope” is that portion of an antigen that determines its immunological specificity. T- and B-cell epitopes can be identified empirically (e.g. using PEPSCAN or similar methods). In a preferred embodiment, the immunogenic fragment induces an immune response, suitably a humoral or T cell response, which is similar to the immune response induced by the reference antigen. Suitably, the immunogenic fragment induces a humoral or T cell response in mice which is not more than 10-fold lower, more suitably not more than 5-fold lower, not more than 2-fold lower or not lower, than the immune response induced by the reference antigen.

As used herein, an “immunogenic fragment of a HSV Fc receptor” refers to a fragment of a naturally-occurring viral Fc receptor of at least 10, 15, 20, 30, 40, 50, 60, 100, 200, 300 or more amino acids, or a peptide having an amino acid sequence of at least 60%, 65%, 70%, 75%, 80%, 85%, 90%, 95%, 97%, 98%, 99%, or 99.5% sequence identity to a naturally-occurring viral Fc receptor (or to a fragment of a naturally-occurring viral Fc receptor of at least about 10, 15, 20, 30, 40, 50, 60 or more amino acids). Thus, an immunogenic fragment of an antigenic viral Fc receptor may be a fragment of a naturally occurring viral Fc receptor, of at least 10 amino acids, and may comprise one or more amino acid substitutions, deletions or additions.

Any of the encoded HSV Fc receptor immunogenic fragments may additionally comprise an initial methionine residue where required.

Suitably, the encoded HSV Fc receptor or immunogenic fragment thereof does not comprise a functional transmembrane domain. Suitably, the encoded HSV Fc receptor or immunogenic fragment thereof does not comprise a cytoplasmic domain. Preferably, the encoded HSV Fc receptor or immunogenic fragment thereof neither comprises a functional transmembrane domain, nor a cytoplasmic domain. In other words, in a preferred embodiment, the encoded HSV Fc receptor or immunogenic fragment consists of a HSV FcR ectodomain (or extracellular domain). More preferably, the encoded HSV Fc receptor or immunogenic fragment comprises or consists of a HSV2 gE2 ectodomain or a HSV1 gE1 ectodomain.

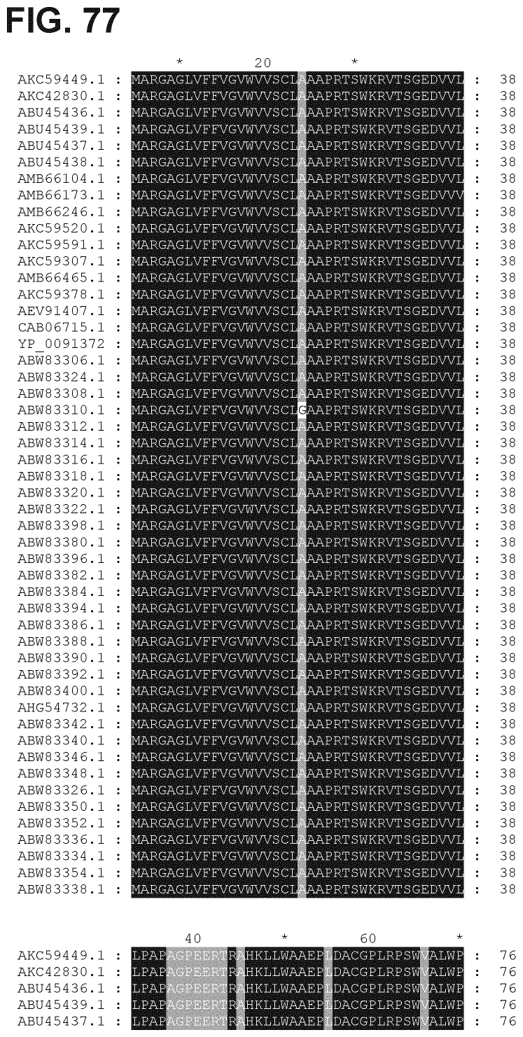

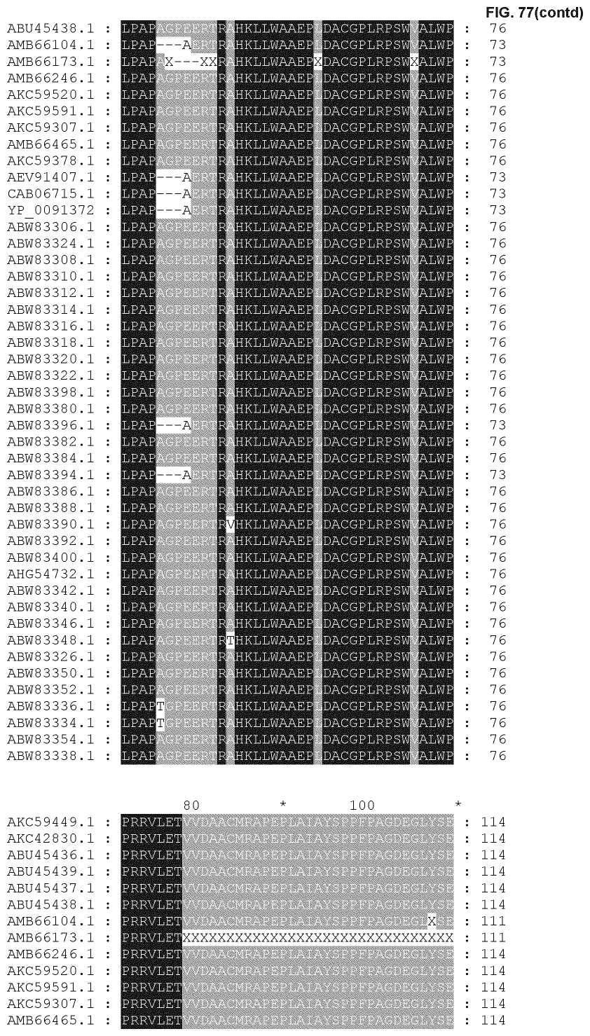

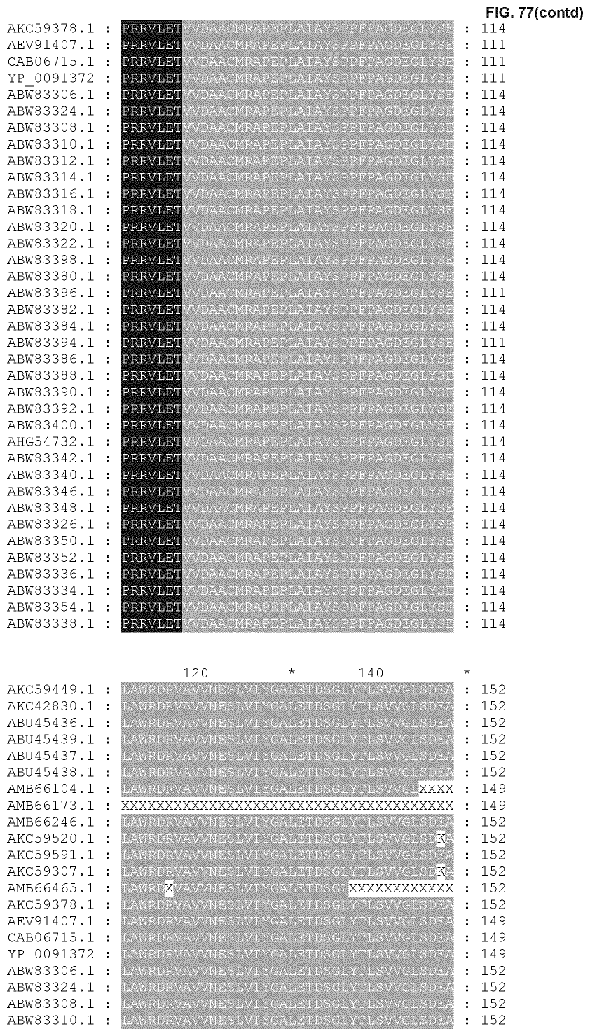

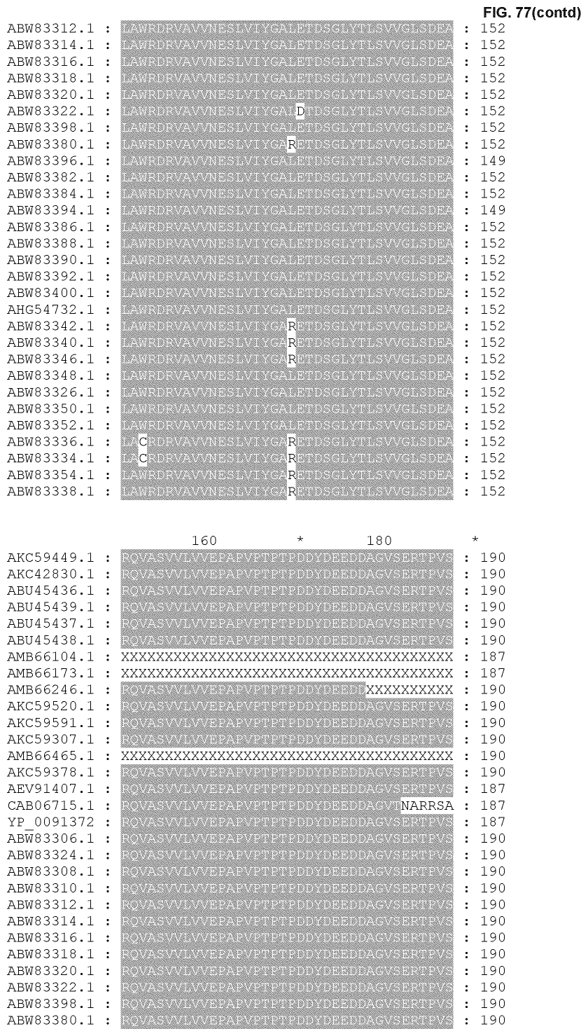

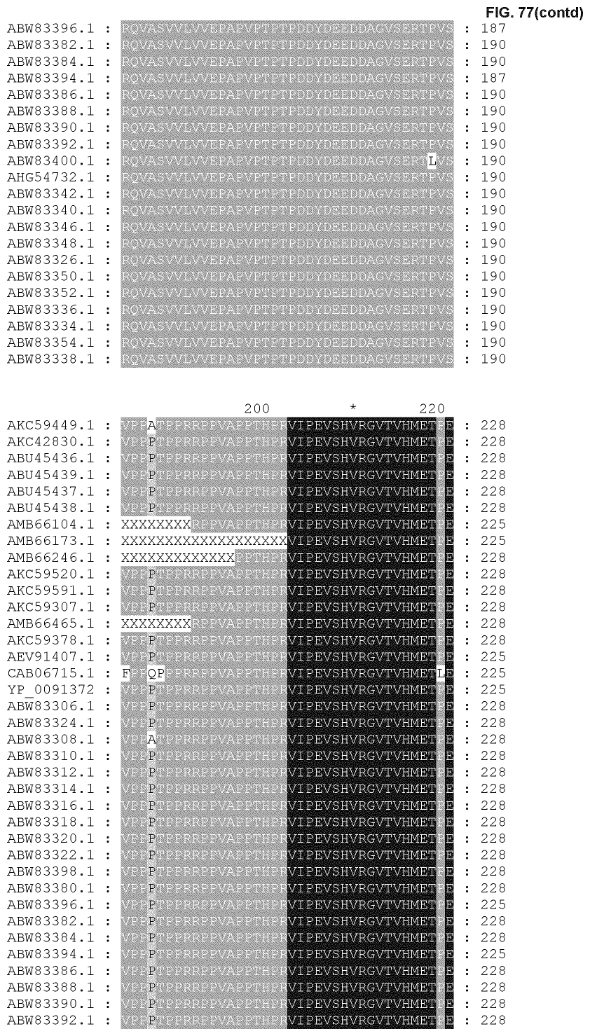

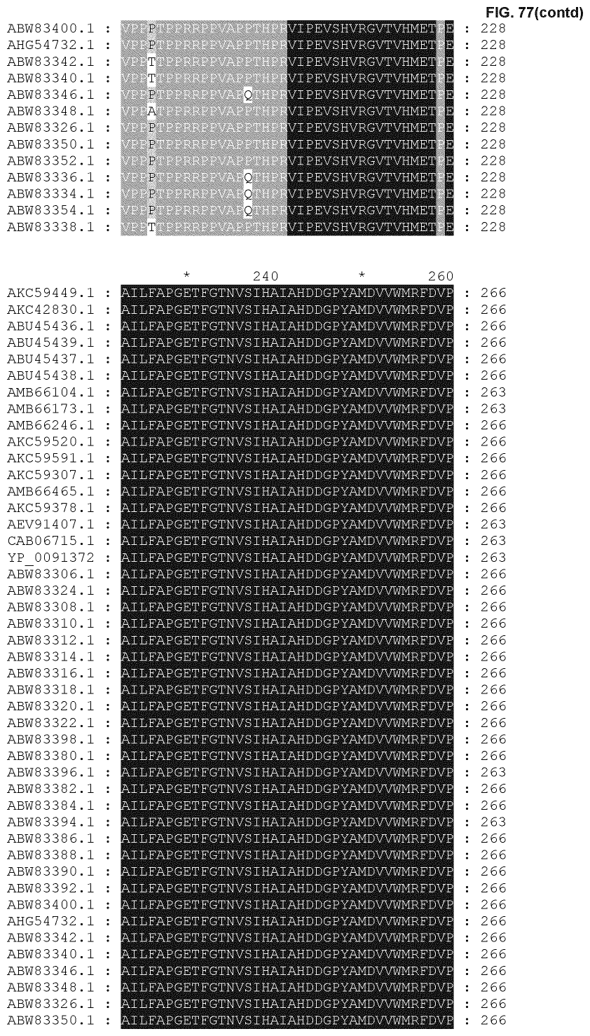

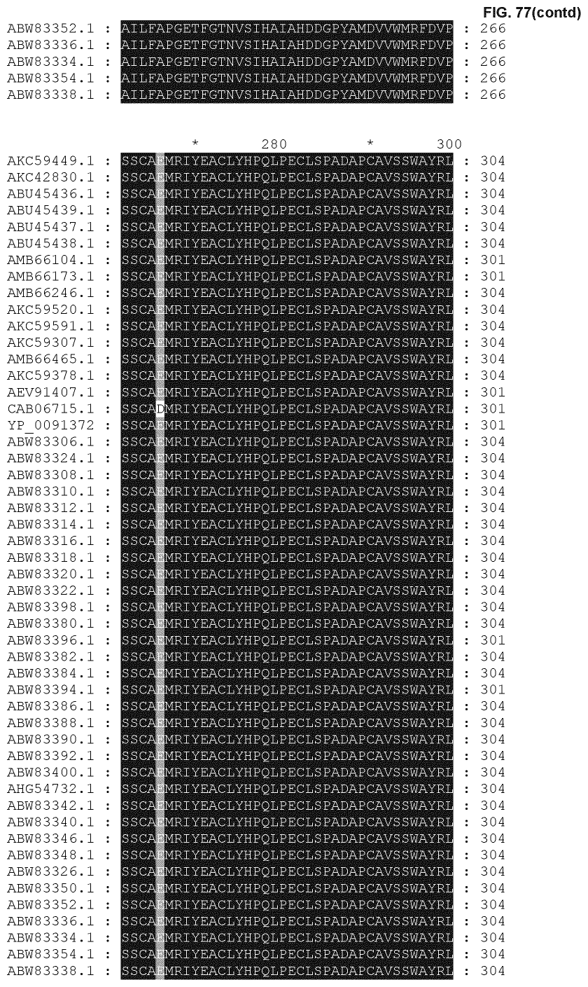

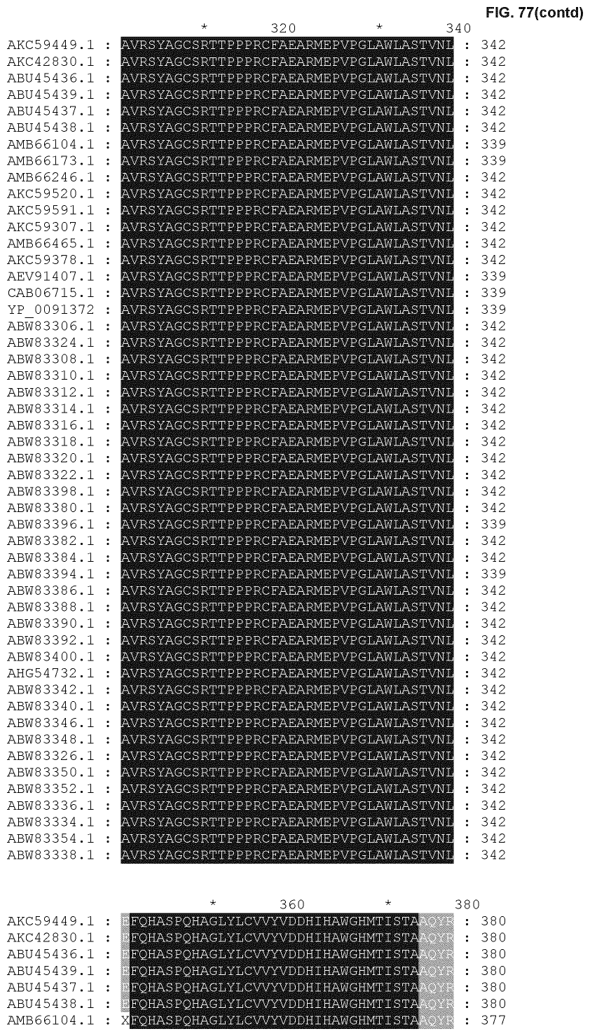

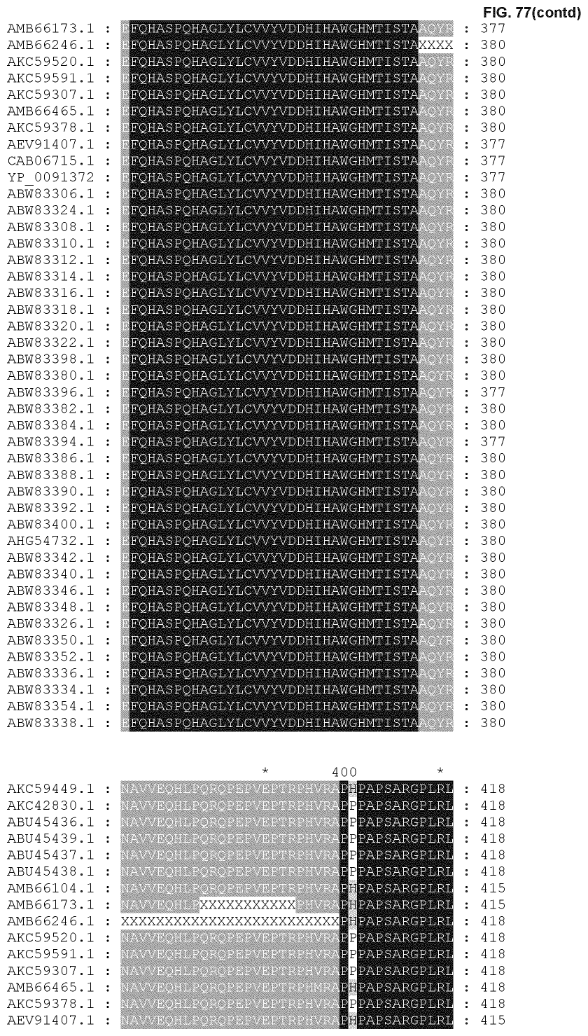

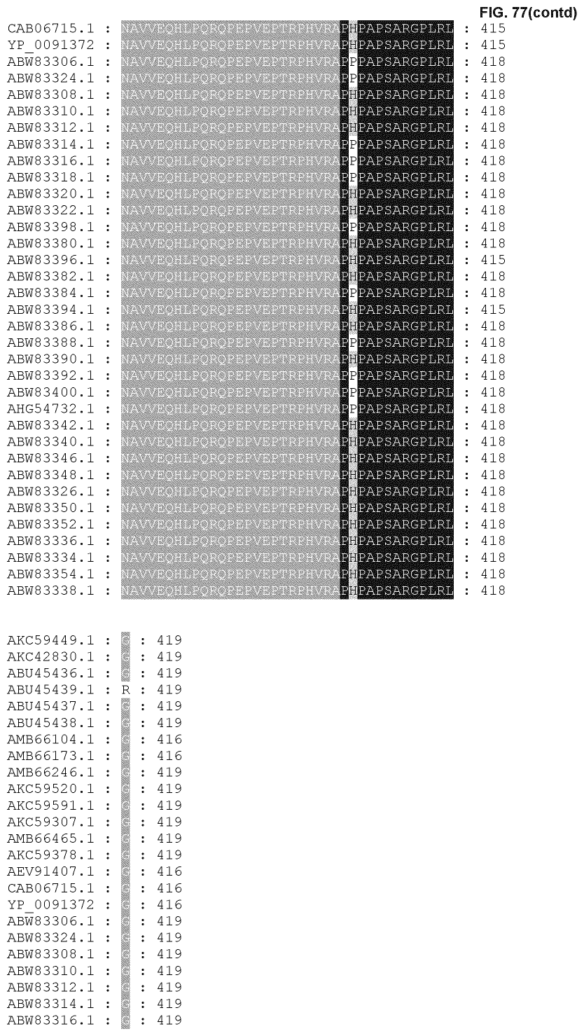

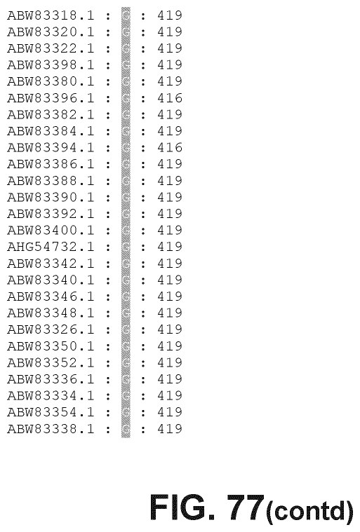

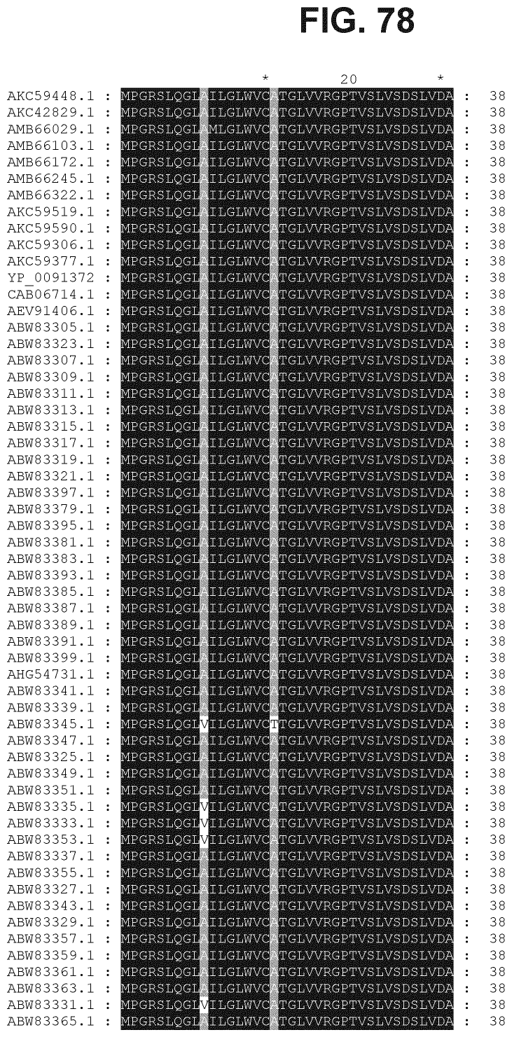

In a preferred embodiment, the encoded HSV FcR ectodomain is a HSV2 gE2 ectodomain which comprises or consists of the amino acid sequence shown on SEQ ID NO: 7 (corresponding to amino acid residues 1-419 of SEQ ID NO: 1), or a sequence which is at least 60%, 65%, 70%, 75% 80%, 85%, 90%, 95%, 96%, 97%, 98%, 99% or 99.5% identical thereto. Suitably, the encoded HSV FcR ectodomain has a sequence selected from the sequences shown in Table 1 or FIG. 77. In a preferred embodiment, the viral FcR ectodomain is a HSV2 gE2 ectodomain which is at least 90%, 95%, 96%, 97%, 98%, 99%, 99.5% or 100% identical to SEQ ID NO: 7.

Suitably, the encoded HSV2 Fc receptor ectodomain may comprise one or more amino acid residue substitution, deletion, or insertion relative to the amino acid sequence shown at SEQ ID NO: 7, for example 1, 2, 3, 4, 5, 6, 7, 8, 9 or 10 amino acid residue substitutions, deletions, or insertions.

In another embodiment, the encoded HSV2 FcR ectodomain is a HSV2 gE2 ectodomain which comprises or consists of the amino acid sequence corresponding to amino acid residues 1-417 of SEQ ID NO: 1, or a sequence which is at least 60%, 65%, 70%, 75% 80%, 85%, 90%, 95%, 96%, 97%, 98%, 99% or 99.5% identical thereto. Suitably, the encoded HSV2 Fc receptor ectodomain may comprise one or more amino acid residue substitutions, deletions, or insertions relative to the amino acid sequence corresponding to amino acid residues 1-417 of SEQ ID NO: 1, for example 1, 2, 3, 4, 5, 6, 7, 8, 9 or 10 amino acid residue substitutions, deletions, or insertions.

In another embodiment, the encoded HSV1 FcR ectodomain is a HSV1 gE1 ectodomain which comprises or consists of the amino acid sequence shown in SEQ ID NO: 9 (corresponding to amino acid residues 1-421 of SEQ ID NO: 3), or a sequence which is at least 60%, 65%, 70%, 75% 80%, 85%, 90%, 95%, 96%, 97%, 98%, 99% or 99.5% identical thereto. In a preferred embodiment, the encoded HSV1 FcR ectodomain is a HSV1 gE1 ectodomain which is at least 90%, 95%, 96%, 97%, 98%, 99%, 99.5% or 100% identical to SEQ ID NO: 9.

Suitably, the encoded HSV Fc receptor HSV1 ectodomain may comprise one or more amino acid residue substitutions, deletions, or insertions relative to the amino acid sequence shown at SEQ ID NO: 9, for example 1, 2, 3, 4, 5, 6, 7, 8, 9 or 10 amino acid residue substitutions, deletions, or insertions.

In another embodiment, the encoded HSV1 FcR HSV1 ectodomain comprises or consists of the amino acid sequence corresponding to amino acid residues 1-419 of SEQ ID NO: 3, or a sequence which is at least 60%, 65%, 70%, 75% 80%, 85%, 90%, 95%, 96%, 97%, 98%, 99% or 99.5% identical thereto. Suitably, the encoded HSV1 Fc receptor HSV1 ectodomain may comprise one or more amino acid residue substitutions, deletions, or insertions relative to the amino acid sequence corresponding to amino acid residues 1-419 of SEQ ID NO: 3, for example 1, 2, 3, 4, 5, 6, 7, 8, 9 or 10 amino acid residue substitutions, deletions, or insertions.

In another embodiment, the encoded immunogenic fragment of a HSV1 or HSV2 FcR comprises or consists of a Fc binding domain from a HSV FcR, or a variant thereof.

In one embodiment, the encoded immunogenic fragment of a HSV2 FcR comprises or consists of a Fc binding domain from a HSV2 gE, for example the amino acid sequence corresponding to amino acid residues 233-378 of SEQ ID NO: 1, or a sequence which is at least 60%, 65%, 70%, 75% 80%, 85%, 90%, 95%, 96%, 97%, 98%, 99% or 99.5% identical thereto. Suitably, the encoded HSV Fc receptor HSV2 Fc binding domain may comprise one or more amino acid residue substitutions, deletions, or insertions relative to the amino acid sequence corresponding to amino acid residues 233-378 of SEQ ID NO: 1, for example 1, 2, 3, 4, 5, 6, 7, 8, 9 or 10 amino acid residue substitutions, deletions, or insertions.

In one embodiment, the encoded immunogenic fragment of a HSV1 FcR comprises or consists of a Fc binding domain from a HSV1 gE, for example the amino acid sequence corresponding to amino acid residues 235-380 of SEQ ID NO: 3, or a sequence which is at least 60%, 65%, 70%, 75% 80%, 85%, 90%, 95%, 96%, 97%, 98%, 99% or 99.5% identical thereto. Suitably, the encoded HSV1 Fc receptor HSV1 Fc binding domain may comprise one or more amino acid residue substitutions, deletions, or insertions relative to the amino acid sequence corresponding to amino acid residues 235-380 of SEQ ID NO: 3, for example 1, 2, 3, 4, 5, 6, 7, 8, 9 or 10 amino acid residue substitutions, deletions, or insertions.

In a preferred embodiment, the ability of the HSV Fc receptor or immunogenic fragment or variant thereof to bind to a human antibody Fc domain is reduced or abolished compared to the corresponding native HSV Fc receptor. Suitably, the encoded HSV Fc receptor or immunogenic fragment thereof comprises one or more amino acid substitutions, deletions or insertions compared to the native sequence of the HSV Fc receptor or immunogenic fragment thereof, that reduce or abolish the binding affinity between the HSV FcR or immunogenic fragment or variant thereof and the antibody Fc domain compared to the native HSV Fc receptor.

The binding affinity between the encoded HSV FcR or immunogenic fragment or variant thereof and the antibody Fc domain can be determined by methods well known to those skilled in the art. For example, the association rate (kₒₙ), dissociation rate (k_{off}), equilibrium dissociation constant (K_{D}=k_{off}/kₒₙ) and equilibrium association constant (K_{A}=1/K_{D}=kₒₙ/k_{off}) can be determined by BiLayer Interferometry.

In a preferred embodiment, the kₒₙ between the HSV FcR or immunogenic fragment or variant thereof and human IgGs is lower than the kₒₙ between the corresponding native HSV FcR and human IgGs (slow binder).

In a preferred embodiment, the k_{off} between the HSV FcR or immunogenic fragment or variant thereof and human IgGs is higher than the k_{off} between the corresponding native HSV FcR and human IgGs (fast releaser).

In a more preferred embodiment, the kₒₙ between the HSV FcR or immunogenic fragment or variant thereof and human IgGs is lower than the kₒₙ between the corresponding native HSV FcR and human IgGs, and the k_{off} between the HSV FcR or immunogenic fragment or variant thereof and human IgGs is higher than the k_{off} between the corresponding native HSV FcR and human IgGs (slow binder/fast releaser).

In a preferred embodiment, the equilibrium dissociation constant (K_{D}) between the HSV FcR or immunogenic fragment or variant thereof and human IgGs is higher than the K D between the corresponding native HSV FcR and human IgGs.

The relative affinity between the encoded HSV FcR or immunogenic fragment or variant thereof and human IgGs can be determined by dividing the K_{D} determined for the native HSV FcR by the K_{D} determined for the HSV FcR or immunogenic fragment or variant thereof.

In a preferred embodiment, the relative affinity between the encoded HSV FcR or immunogenic fragment or variant thereof and human IgGs is less than 100%, for example less than 90%, 80%, 70%, 60%, 50%, 40%, 30%, 20%, 15% or 10% of the affinity between the corresponding native HSV FcR and human IgGs. In a more preferred embodiment, the relative affinity between the HSV FcR or immunogenic fragment or variant thereof and human IgGs is less than 15%, more preferably still less than 10% of the affinity between the corresponding native HSV FcR and human IgGs.

In a preferred embodiment, the equilibrium dissociation constant (K_{D}) between the encoded HSV FcR or immunogenic fragment or variant thereof and human IgGs is higher than 2×10⁻⁷ M, preferably higher than 5×10⁻⁷ M, more preferably higher than 1×10⁻⁶ M.

Alternatively, the ability of the encoded HSV Fc receptor or immunogenic fragment thereof to bind to a human antibody Fc domain can be assessed by measuring the response (expressed in nm) in a BiLayer Interferometry assay.

In a preferred embodiment, the response in a BiLayer Interferometry assay corresponding to the binding between the encoded HSV Fc receptor or immunogenic fragment or variant thereof and human IgGs is less than 80%, suitably less than 70%, 60%, 50%, 40% of the response obtained with the corresponding native HSV Fc receptor. In a preferred embodiment, the response in a BiLayer Interferometry assay corresponding to the binding between the encoded HSV Fc receptor or immunogenic fragment or variant thereof and human IgGs is lower than 0.4 nm, suitably lower than 0.3 nm, 0.2 nm or 0.1 nm.

Suitably, the encoded HSV2 gE2 or immunogenic fragment or variant thereof comprises one or more mutations (insertions, substitutions or deletions) at positions selected from N241, H245, A246, A248, R314, P317, P318, P319, F322, R320, A337, S338 or V340 of the HSV2 gE2 sequence shown in SEQ ID NO: 1.

Exemplary mutations that may be used herein to reduce or abolish the binding affinity between HSV2 gE2 or immunogenic fragment or variant thereof and an antibody Fc domain include the single point substitution mutations of the sequence shown in SEQ ID NO: 1 selected from H245A, H245K, P317R, P319A, P319R, P319G, P319K, P319T, A337G, P319D, P319S, S338D, N241A, R320D, H245E, H245V, H245R, H245D, H245Q, H245G, H2451, H245K, H245S, H245T, A246W, A248K, A248T, A248G, R314A, R314N, R314D, R314Q, R314E, R314G, R314I, R314L, R314K, R314M, R314F, R314P, R314S, R314T, R314Y, R314V, P317N, P317G, P317I, P317L, P317K, P317F, P317S, P318R, P318D, P318Q, P318I, P318S, P318T, P318Y, P319L, R320A, R320S, R320N, R320Q, R320E, R320G, R320H, R320I, R320L, R320M, R320P, R320T, R320V, F322A, F322N, F322I, F322K, F322P, F322T, S338G, S338E, S338L, S338T, V340A, V340R, V340D, V340Q, V340M, V340F, V340P and V340W.

Exemplary mutations that may be used herein to reduce or abolish the binding affinity between HSV2 gE2 or immunogenic fragment thereof and an antibody Fc domain also include the double point substitution mutations of the sequence shown in SEQ ID NO: 1 selected from H245A and P319A; H245A and P319R; H245A and P319G; H245A and P319K; H245A and P319T; N241A and R320D; N241A and P319D; A246W and P317K; A246W and P317F; A246W and P317S; A246W and R320D; A246W and R320G; A246W and R320I; A248K and V340R; A248K and V340M; A248K and V340W; A248T and V340R; A248T and V340M; A248T and V340W; A248G and V340R; A248G and V340M; A248G and V340W; A248K and F322A; A248K and F322I; A248K and F322P; A248T and F322A; A248T and F322I; A248T and F322P; A248G and F322A; A248G and F322I; A248G and F322P; H245A and R320D; H245A and R320G; H245A and R320T; H245G and R320D; H245G and R320G; H245G and R320T; H245S and R320D; H245S and R320G; H245S and R320T; H245A and P319G; H245A and P319L; H245G and P319G; H245G and P319L; H245S and P319G; H245S and P319L; R314G and P318R; R314G and P318D; R314G and P318I; R314L and P318R; R314L and P318D; R314L and P318I; R314P and P318R; R314P and P318D; R314P and P318I; R314G and F322A; R314G and F322I; R314G and F322P; R314L and F322A; R314L and F322I; R314L and F322P; R314P and F322A; R314P and F322I; R314P and F322P; R314G and V340R; R314G and V340M; R314G and V340W; R314L and V340R; R314L and V340M; R314L and V340W; R314P and V340R; R314P and V340M; R314P and V340W; P317K and V340R; P317K and V340M; P317K and V340W; P317F and V340R; P317F and V340M; P317F and V340W; P317S and V340R; P317S and V340M; P317S and V340W; P317K and S338G; P317K and S338H; P317K and S338L; P317F and S338G; P317F and S338H; P317F and S338L; P317S and S338G; P317S and S338H; P317S and S338L; P318R and S338G; P318R and S338H; P318R and S338L; P318D and S338G; P318D and S338H; P318D and S338L; P318I and S338G; P318I and S338H; P318I and S338L; P319G and V340R; P319G and V340M; P319G and V340W; P319L and V340R; P319L and V340M; P319L and V340W; P317R and P319D; P317R and R320D; P319D and R320D.

Exemplary mutations that may be used herein to reduce or abolish the binding affinity between HSV2 gE2 or immunogenic fragment or variant thereof and an antibody Fc domain also include deletion mutations at positions P319 and/or R320 of the sequence shown in SEQ ID NO: 1, alone or in combination with substitution mutations, in particular mutations selected from P319 deletion; R320 deletion; P319 deletion/R320 deletion; P319 deletion/R320 deletion/P317G/P318G; P319 deletion/R320 deletion/P318E; P319 deletion/R320 deletion/P318G; P319 deletion/R320 deletion/P318K; P319 deletion/R320 deletion/P317R/P318E; P319 deletion/R320 deletion/P317R/P318G; P319 deletion/R320 deletion/P317R/P318K; P319 deletion/R320 deletion/P317G/P318K.

Exemplary mutations that may be used herein to reduce or abolish the binding affinity between HSV2 gE2 or immunogenic fragment or variant thereof and an antibody Fc domain also include the insertion mutations selected from:
insertion of peptide sequence LDIGE between amino acid residues Y275 and E276 of SEQ ID NO: 1 (275_insert_LDIGE),
insertion of peptide sequence ADIGL between amino acid residues S289 and P290 of SEQ ID NO: 1 (289_insert_ADIGL),
insertion of peptide sequence ARAA between amino acid residues A337 and S338 of SEQ ID NO: 1 (337_insert_ARAA),
insertion of peptide sequence ARAA between amino acid residues S338 and T339 of SEQ ID NO: 1 (338_insert_ARAA), and
insertion of peptide sequence ADIT between amino acid residues H346 and A347 of SEQ ID NO: 1 (346_insert_ADIT).

In a preferred embodiment, the encoded HSV2 gE2 or immunogenic fragment or variant thereof comprises a mutation or a combination of mutations with respect to the sequence shown in SEQ ID NO: 1 selected from 289_insert ADIGL; 338_insert ARAA; H245K; P317R; P319R; P319G; P319K; H245A_P319R; H245A_P319G; H245A_P319K; H245A_P3191; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248I; P318S; P317K; P317S_V340W; H245D; R314P_V340W; R314L_318D; P319L_V340W; P317F; P318D_S338G; R314G_V340W; P317K_S338H; R314L_V340W; P318R; P318Q; P317F_S338G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R320T; R314P_P318R; A248G_F322I; R320N; P317N; R314D; R314Y; R314P_F322I; P319G_V340M; P317S_V340R; R314V; P317R_P319D; P317R_R320D; P319D_R320D; 4319_Δ320; P317G_P318G_Δ319_Δ320; P318E_Δ319_Δ320; P318G_Δ319_Δ320; P318K_Δ319_Δ320; P317R_P318E_Δ319_320; P317R_P318G_Δ319_Δ320 and P317G_P318K_Δ319_Δ320. (A means deleted residue).

In a more preferred embodiment, the encoded HSV2 gE2 or immunogenic fragment or variant thereof comprises a mutation or a combination of mutations with respect to the sequence shown in SEQ ID NO: 1 selected from 338_insert ARAA; P317R; P319D; R320D; A248T_V340W; V340W; A248T; P318I and A246W.

Corresponding mutations in other HSV2 gE2 sequences, for example the sequences listed in Table 1 and shown on the alignment presented in FIG. 77, to the exemplary substitution, selection and insertion mutations listed above are also in the scope of the present invention.

All possible combinations of the exemplary single and double substitution mutations and insertion mutations listed above are also in the scope of the present invention.

Suitably, the encoded HSV1 gE1 or immunogenic fragment or variant thereof comprises one or more mutations (insertions, substitutions or deletions) at positions selected from H247, P319 and P321 of the HSV1 gE1 sequence shown in SEQ ID NO: 3.

Exemplary mutations that may be used herein to reduce or abolish the binding affinity between HSV1 gE1 or immunogenic fragment thereof and an antibody Fc domain include the single point substitution mutations of the sequence shown in SEQ ID NO: 3 selected from H247A, H247K, P319R, P321A, P321R, P321G, P321K, P321T, A339G, P321D, P321S, A340D, N243A and R322D, and the double point substitutions mutations of the sequence shown in SEQ ID NO: 3 selected from H247A/P321A, H247A/P321R, H247A/P321G, H247A/P321K, H247A/P321T, N243A/R322D, N243A/P321D, H247G/P319G, P319G/P321G and A340G/S341G/V342G.

Exemplary mutations that may be used herein to reduce or abolish the binding affinity between HSV1 gE1 or immunogenic fragment or variant thereof and an antibody Fc domain also include the insertion mutations selected from:
insertion of peptide sequence LDIGE between amino acid residues Y277 and E278 of SEQ ID NO: 3 (277_insert_LDIGE);
insertion of peptide sequence ADIGL between amino acid residues S291 and P292 of SEQ ID NO: 3 (291_insert_ADIGL);
insertion of peptide sequence ARAA between amino acid residues A339 and A340 of SEQ ID NO: 3 (339_inset_ARAA);
insertion of peptide sequence ARAA between amino acid residues A340 and S341 of SEQ ID NO: 3 (340_inset_ARAA); and
insertion of peptide sequence ADIT between amino acid residues D348 and A349 of SEQ ID NO: 3 (348_inset_ADIT).

In a preferred embodiment, the encoded HSV1 gE1 or immunogenic fragment or variant thereof comprises a mutation or a combination of mutations with respect to the sequence shown in SEQ ID NO: 3 selected from P321K; P321D; R322D; N243A_R322D; N243A_P321D; A340G_S341G_V342G; H247G_P319G; P321R; H247A_P321K; 291_insert_ADIGL; 339 insert ARAA; P319R; P319G_P321G and H247A_P321R.

In a more preferred embodiment, the encoded HSV1 gE1 or immunogenic fragment or variant thereof comprises a mutation or a combination of mutations with respect to the sequence shown in SEQ ID NO: 3 selected from P319R, P321D, R322D, N243A_R322D or A340G_S341G_V342G.

Corresponding mutations in other HSV1 gE1 sequences to the exemplary single and double substitution mutations and insertion mutations listed above are also in the scope of the present invention.

All possible combinations of the exemplary single and double substitution mutations and insertion mutations listed above are also in the scope of the present invention.

In a preferred embodiment, when the encoded HSV Fc receptor or immunogenic fragment or variant thereof is a HSV FcR ectodomain, the ability of the ectodomain to bind to an antibody Fc domain is reduced or abolished compared to the native HSV Fc receptor. Suitably, the encoded HSV FcR ectodomain comprises one or more amino acid substitutions, deletions or insertions compared to the native sequence of the HSV FcR ectodomain, that reduce or abolish the binding affinity between the HSV FcR ectodomain and the antibody Fc domain compared to the native HSV FcR.

The binding affinity between the encoded HSV FcR ectodomain and the antibody Fc domain can be determined by methods described above.

In a preferred embodiment, the kₒₙ between the HSV FcR ectodomain and human IgGs is lower than the kₒₙ between the corresponding native HSV FcR ectodomain and human IgGs (slow binder).

In a preferred embodiment, the k_{off} between the HSV FcR ectodomain and human IgGs is higher than the k_{off} between the corresponding native HSV FcR ectodomain and human IgGs (fast releaser).

In a more preferred embodiment, the km, between the HSV FcR ectodomain and human IgGs is lower than the kₒₙ between the corresponding native HSV FcR ectodomain and human IgGs, and the k_{off} between the HSV FcR ectodomain and human IgGs is higher than the k_{off} between the corresponding native HSV FcR ectodomain and human IgGs (slow binder/fast releaser).

In a preferred embodiment, the equilibrium dissociation constant (K_{D}) between the encoded HSV FcR ectodomain and human IgGs is higher than the K_{D} between the corresponding native HSV FcR ectodomain and human IgGs.

In a preferred embodiment, the relative affinity between the encoded HSV FcR ectodomain and human IgGs is less than 100%, for example less than 90%, 80%, 70%, 60%, 50%, 40%, 30%, 20%, 15% or 10% of the affinity between the corresponding native HSV FcR ectodomain and human IgGs. In a more preferred embodiment, the relative affinity between the HSV FcR ectodomain and human IgGs is less than 15%, more preferably still less than 10% of the affinity between the corresponding native HSV FcR ectodomain and human IgGs.

In a preferred embodiment, the equilibrium dissociation constant (K_{D}) between the encoded HSV FcR ectodomain and human IgGs is higher than 2×10⁻⁷ M, preferably higher than 5×10⁻⁷ M, more preferably higher than 1×10⁻⁶ M.

Alternatively, the ability of the encoded HSV FcR ectodomain to bind to a human antibody Fc domain can be assessed by measuring the response (expressed in nm) in a BiLayer Interferometry assay. In a preferred embodiment, the response in a BiLayer Interferometry assay corresponding to the binding between the HSV FcR ectodomain and human IgGs is less than 80%, suitably less than 70%, 60%, 50%, 40% of the response obtained with the corresponding native HSV FcR ectodomain.

In a preferred embodiment, the response in a BiLayer Interferometry assay corresponding to the binding between the viral FcR ectodomain and human IgGs is lower than 0.6 nm, suitably lower than 0.5 nm, 0.4 nm, 0.3 nm or 0.2 nm.

Suitably, the encoded HSV2 gE2 ectodomain comprises one or more mutations (insertions, substitutions or deletions) at positions selected from N241, H245, A246, A248, R314, P317, P318, P319, F322, R320, A337, S338 or V340 of the HSV2 gE2 ectodomain sequence shown in SEQ ID NO: 7.

Exemplary mutations that may be used herein to reduce or abolish the binding affinity between HSV2 gE2 ectodomain and an antibody Fc domain include the single point substitution mutations of the sequence shown in SEQ ID NO: 7 selected from H245A, H245K, P317R, P319A, P319R, P319G, P319K, P319T, A337G, P319D, P319S, S338D, N241A, R320D, H245E, H245V, H245R, H245D, H245Q, H245G, H2451, H245K, H245S, H245T, A246W, A248K, A248T, A248G, R314A, R314N, R314D, R314Q, R314E, R314G, R314I, R314L, R314K, R314M, R314F, R314P, R314S, R314T, R314Y, R314V, P317N, P317G, P317I, P317L, P317K, P317F, P317S, P318R, P318D, P318Q, P318I, P318S, P318T, P318Y, P319L, R320A, R320S, R320N, R320Q, R320E, R320G, R320H, R320I, R320L, R320M, R320P, R320T, R320V, F322A, F322N, F322I, F322K, F322P, F322T, S338G, S338E, S338L, S338T, V340A, V340R, V340D, V340Q, V340M, V340F, V340P and V340W.

Exemplary mutations that may be used herein to reduce or abolish the binding affinity between HSV2 gE2 ectodomain and an antibody Fc domain also include the double point substitution mutations of the sequence shown in SEQ ID NO: 7 selected from H245A and P319A; H245A and P319R; H245A and P319G; H245A and P319K; H245A and P319T; N241A and R320D; N241A and P319D; A246W and P317K; A246W and P317F; A246W and P317S; A246W and R320D; A246W and R320G; A246W and R320T; A248K and V340R; A248K and V340M; A248K and V340W; A248T and V340R; A248T and V340M; A248T and V340W; A248G and V340R; A248G and V340M; A248G and V340W; A248K and F322A; A248K and F322I; A248K and F322P; A248T and F322A; A248T and F322I; A248T and F322P; A248G and F322A; A248G and F322I; A248G and F322P; H245A and R320D; H245A and R320G; H245A and R320T; H245G and R320D; H245G and R320G; H245G and R320T; H245S and R320D; H245S and R320G; H245S and R320T; H245A and P319G; H245A and P319L; H245G and P319G; H245G and P319L; H245S and P319G; H245S and P319L; R314G and P318R; R314G and P318D; R314G and P318I; R314L and P318R; R314L and P318D; R314L and P318I; R314P and P318R; R314P and P318D; R314P and P318I; R314G and F322A; R314G and F322I; R314G and F322P; R314L and F322A; R314L and F322I; R314L and F322P; R314P and F322A; R314P and F322I; R314P and F322P; R314G and V340R; R314G and V340M; R314G and V340W; R314L and V340R; R314L and V340M; R314L and V340W; R314P and V340R; R314P and V340M; R314P and V340W; P317K and V340R; P317K and V340M; P317K and V340W; P317F and V340R; P317F and V340M; P317F and V340W; P317S and V340R; P317S and V340M; P317S and V340W; P317K and 5338G; P317K and S338H; P317K and S338L; P317F and S338G; P317F and S338H; P317F and S338L; P317S and S338G; P317S and S338H; P317S and S338L; P318R and S338G; P318R and S338H; P318R and S338L; P318D and S338G; P318D and S338H; P318D and S338L; P318I and S338G; P318I and S338H; P318I and S338L; P319G and V340R; P319G and V340M; P319G and V340W; P319L and V340R; P319L and V340M; and P319L; V340W; P317R and P319D; P317R and R320D; P319D and R320D.

Exemplary mutations that may be used herein to reduce or abolish the binding affinity between HSV2 gE2 ectodomain and an antibody Fc domain also include deletion mutations at positions P319 and/or R320 of the sequence shown in SEQ ID NO: 7 alone or in combination with substitution mutations, in particular mutations selected from P319 deletion; R320 deletion; P319 deletion/R320 deletion; P319 deletion/R320 deletion/P317G/P318G; P319 deletion/R320 deletion/P318E; P319 deletion/R320 deletion/P318G; P319 deletion/R320 deletion/P318K; P319 deletion/R320 deletion/P317R/P318E; P319 deletion/R320 deletion/P317R/P318G; P319 deletion/R320 deletion/P317R/P318K; P319 deletion/R320 deletion/P317G/P318K.

Exemplary mutations that may be used herein to reduce or abolish the binding affinity between HSV2 gE2 ectodomain and an antibody Fc domain also include the insertion mutations selected from:
insertion of peptide sequence LDIGE between amino acid residues Y275 and E276 of SEQ ID NO: 7 (275_insert_LDIGE),
insertion of peptide sequence ADIGL between amino acid residues S289 and P290 of SEQ ID NO: 7 (289_insert_ADIGL),
insertion of peptide sequence ARAA between amino acid residues A337 and S338 of SEQ ID NO: 7 (337_insert_ARAA),
insertion peptide sequence ARAA between amino acid residues S338 and T339 of SEQ ID NO: 7 (338_insert_ARAA), and
insertion peptide sequence ADIT between amino acid residues H346 and A347 of SEQ ID NO: 7 (346_insert_ADIT).

In a preferred embodiment, the encoded HSV2 gE2 ectodomain comprises a mutation or a combination of mutations with respect to the sequence shown in SEQ ID NO: 7 selected from 289_insert_ADIGL; 338_insert ARAA; H245K; P317R; P319R; P319G; P319K; H245AP319R; H245A_P319G; H245A_P319K; H245A_P319T; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248T; P318S; P317K; P317S_V340W; H245D; R314P_V340W; R314L_318 D; P319L_V340W; P317F; P318D_S338G; R314G_V340W; P317K_S338H; R314L_V340W; P318R; P318Q; P317F_S338G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R320T; R314P_P318R; A248G_F322I; R320N; P317N; R314D; R314Y; R314P_F322I; P319G_V340M; P317S_V340R; R314V; P317R_P319D; P317R_R320D; P319D_R320D; Δ319_Δ320; P317G_P318G_Δ319_Δ320; P318E_Δ319_Δ320; P318G_Δ319_Δ320; P318K_Δ319_Δ320; P317R_P318E_Δ319_320; P317R_P318G_Δ319_Δ320 and P317G_P318K_Δ319_Δ320.

In a more preferred embodiment, the encoded HSV2 gE2 ectodomain comprises a mutation or a combination of mutations with respect to the sequence shown in SEQ ID NO: 7 selected from 338_insert ARAA; P317R; P319D; R320D; A248T_V340W; V340W; A248T; P318I and A246W.

Corresponding mutations in other HSV2 gE2 ectodomain sequences, for example the sequences listed in Table 1 and shown on the alignment presented in FIG. 77, to the exemplary substitution, deletion and insertion mutations listed above are also in the scope of the present invention.

All possible combinations of the exemplary single and double substitution mutations and insertion mutations listed above are also in the scope of the present invention.

Suitably, the encoded HSV1 gE1 ectodomain comprises one or more mutations (insertions, substitutions or deletions) at positions selected from H247, P319 and P321 of the HSV1 gE1 ectodomain sequence shown in SEQ ID NO: 9.

Exemplary mutations that may be used herein to reduce or abolish the binding affinity between HSV1 gE1 ectodomain and an antibody Fc domain include the single point substitution mutations of the sequence shown in SEQ ID NO: 9 selected from H247A, H247K, P319R, P321A, P321R, P321G, P321K, P321T, A339G, P321D, P321S, A340D, N243A and R322D, and the double point substitutions mutations of the sequence shown in SEQ ID NO: 9 selected from H247A/P321A, H247A/P321R, H247A/P321G, H247A/P321K, H247A/P321T, N243A/R322D, N243A/P321D, H247G/P319G, P319G/P321G and A340G/S341G/V342G.

Exemplary mutations that may be used herein to reduce or abolish the binding affinity between HSV1 gE1 ectodomain and an antibody Fc domain also include the insertion mutations selected from:
insertion of peptide sequence LDIGE between amino acid residues Y277 and E278 of SEQ ID NO: 9 (277_insert_LDIGE);
insertion of peptide sequence ADIGL between amino acid residues S291 and P292 of SEQ ID NO: 9 (291_inset_ADIGL);
insertion of peptide sequence ARAA between amino acid residues A339 and A340 of SEQ ID NO: 9 (339_inset_ARAA);
insertion of peptide sequence ARAA between amino acid residues A340 and S341 of SEQ ID NO: 9 (340_inset_ARAA); and
insertion of peptide sequence ADIT between amino acid residues D348 and A349 of SEQ ID NO: 9 (348 insert_ADIT).

In a preferred embodiment, the encoded HSV1 gE1 ectodomain comprises a mutation or a combination of mutations with respect to the sequence shown in SEQ ID NO: 9 selected from P321K; P321D; R322D; N243A_R322D; N243A_P321D; A340G_S341G_V342G; H247G_P319G; P321R; H247A_P321K; 291_insert ADIGL; 339_insert ARAA; P319R; P319G_P321G and H247A_P321R.

In a more preferred embodiment, the encoded HSV1 gE ectodomain comprises a mutation or a combination of mutations with respect to the sequence shown in SEQ ID NO: 9 selected from P321D; R322D; A340G_S341G_V342G and P319R.

Corresponding mutations in other HSV1 gE1 ectodomain sequences to the exemplary single and double substitution mutations and insertion mutations listed above are also in the scope of the present invention.

All possible combinations of the exemplary single and double substitution mutations and insertion mutations listed above are also in the scope of the present invention.

In a preferred embodiment, the encoded HSV Fc receptor or immunogenic fragment or variant thereof is part of a heterodimer with a binding partner from said virus or a fragment thereof.

As used herein, a “binding partner” is a viral protein (or glycoprotein) or fragment thereof which forms a noncovalent heterodimer complex with the Fc receptor or immunogenic fragment or variant thereof.

In a preferred embodiment, the encoded HSV Fc receptor is HSV2 gE2 or an immunogenic fragment or variant thereof and the binding partner is HSV2 gI2 or a fragment thereof.

Herein, a “HSV2 gI2” (or “HSV2 gI”) is a HSV2 gI glycoprotein encoded by HSV2 gene US7 and displayed on the surface of infected cells where it associates with HSV2 gE2 to form a heterodimer. Suitably, the HSV2 gI2 is selected from the HSV2 gI glycoproteins shown in Table 2 or variants therefrom which are at least 60%, 65%, 70%, 75% 80%, 85%, 90%, 95%, 96%, 97%, 98%, 99% or 99.5% identical thereto.

TABLE 2
HSV2 gI glycoproteins and ectodomains

| Genbank accession numbers | SEQ ID NO | Ectodomain |
|---|---|---|
| AHG54731.1 | 2 | 1-256 |
| AKC42829.1 | 62 | 1-256 |
| AKC59519.1 | 63 | 1-256 |
| AKC59590.1 | 64 | 1-256 |
| AKC59306.1 | 65 | 1-256 |
| AKC59377.1 | 66 | 1-256 |
| ABW83313.1 | 67 | 1-256 |
| ABW83397.1 | 68 | 1-256 |
| ABW83385.1 | 69 | 1-256 |
| ABW83327.1 | 70 | 1-256 |
| ABW83341.1 | 71 | 1-256 |
| ABW83339.1 | 72 | 1-256 |
| ABW83325.1 | 73 | 1-256 |
| ABW83351.1 | 74 | 1-256 |
| ABW83337.1 | 75 | 1-256 |
| ABW83355.1 | 76 | 1-256 |
| ABW83343.1 | 77 | 1-256 |
| ABW83329.1 | 78 | 1-256 |
| ABW83357.1 | 79 | 1-256 |
| ABW83365.1 | 80 | 1-256 |
| ABW83367.1 | 81 | 1-256 |
| ABW83371.1 | 82 | 1-256 |
| ABW83377.1 | 83 | 1-256 |
| AKC59448.1 | 84 | 1-256 |
| ABW83319.1 | 85 | 1-256 |
| ABW83379.1 | 86 | 1-256 |
| ABW83381.1 | 87 | 1-256 |
| ABW83383.1 | 88 | 1-256 |
| ABW83389.1 | 89 | 1-256 |
| ABW83347.1 | 90 | 1-256 |
| ABW83349.1 | 91 | 1-256 |
| ABW83335.1 | 92 | 1-256 |
| ABW83333.1 | 93 | 1-256 |
| ABW83353.1 | 94 | 1-256 |
| ABW83359.1 | 95 | 1-256 |
| ABW83363.1 | 96 | 1-256 |
| ABW83331.1 | 97 | 1-256 |
| ABW83369.1 | 98 | 1-256 |
| ABW83375.1 | 99 | 1-256 |
| AMB66172.1 | 100 | 1-256 |
| YP_009137219.1 | 101 | 1-256 |
| CAB06714.1 | 102 | 1-256 |
| AEV91406.1 | 103 | 1-256 |
| ABW83305.1 | 104 | 1-256 |
| ABW83323.1 | 105 | 1-256 |
| ABW83307.1 | 106 | 1-256 |
| ABW83311.1 | 107 | 1-256 |
| ABW83315.1 | 108 | 1-256 |
| ABW83317.1 | 109 | 1-256 |
| ABW83321.1 | 110 | 1-256 |
| ABW83395.1 | 111 | 1-256 |
| ABW83393.1 | 112 | 1-256 |
| ABW83387.1 | 113 | 1-256 |
| ABW83391.1 | 114 | 1-256 |
| ABW83399.1 | 115 | 1-256 |
| ABW83345.1 | 116 | 1-256 |
| ABW83361.1 | 117 | 1-256 |
| ABW83309.1 | 118 | 1-256 |
| ABW83373.1 | 119 | 1-256 |
| AMB66029.1 | 120 | 1-256 |
| AMB66103.1 | 121 | 1-256 |
| AMB66322.1 | 122 | 1-256 |
| AMB66245.1 | 123 | 1-256 |

In a preferred embodiment, the encoded HSV2 gI2 is the gI from HSV2 strain SD90e (Genbank accession numbers AHG54731.1, UniProtKB accession number: A8U5L5, SEQ ID NO: 2) which has the amino acid sequence shown in SEQ ID NO:2, or a variant therefrom which is at least 60%, 65%, 70%, 75% 80%, 85%, 90%, 95%, 96%, 97%, 98%, 99% or 99.5% identical thereto.

In another preferred embodiment, the encoded HSV Fc receptor is HSV1 gE1 or an immunogenic fragment or variant thereof and the binding partner is HSV1 gI1 or a fragment thereof.

Herein, a “HSV1 gI1” (or “HSV1 gI”) is a HSV1 gI glycoprotein encoded by HSV1 gene US7 and displayed on the surface of infected cells where it associates with HSV1 gE1 to form a heterodimer. Suitably, the HSV1 gI1 is the gI from HSV1 strain 17 (UniProtKB accession number: P06487, SEQ ID NO: 4) which has the amino acid sequence shown in SEQ ID NO: 4, or a variant therefrom which is at least 60%, 65%, 70%, 75% 80%, 85%, 90%, 95%, 96%, 97%, 98%, 99% or 99.5% identical thereto.

In one embodiment, a fragment of the HSV FcR binding partner is used.

As used herein, the term “fragment” as applied to a protein or peptide refers to a subsequence of a larger protein or peptide. A “fragment” of a protein or peptide is at least about 10 amino acids in length (amino acids naturally occurring as consecutive amino acids; e.g., as for a single linear epitope); for example at least about 15, 20, 30, 40, 50, 60, 100, 200, 300 or more amino acids in length (and any integer value in between).

In a preferred embodiment, the encoded HSV FcR binding partner fragment is an immunogenic fragment.

As used herein, an “fragment of a HSV FcR binding partner” refers to a fragment of a naturally-occurring HSV FcR binding partner of at least 10, 15, 20, 30, 40, 50, 60, 100, 200, 300 or more amino acids, or a peptide having an amino acid sequence of at least 60%, 65%, 70%, 75%, 80%, 85%, 90%, 95%, 97%, 98%, 99%, or 99.5% sequence identity to a naturally-occurring HSV FcR binding partner (or to a fragment of a naturally-occurring HSV FcR binding partner of at least about 10, 15, 20, 30, 40, 50, 60 or more amino acids). Thus, a fragment of a HSV FcR binding partner may be a fragment of a naturally occurring HSV FcR binding partner, of at least 10 amino acids, and may comprise one or more amino acid substitutions, deletions or additions.

Any of the encoded HSV FcR binding partner fragments may additionally comprise an initial methionine residue where required.

A transmembrane protein is a type of integral membrane protein that has the ability to span across a cell membrane under normal culture conditions. Herein a transmembrane domain is the section of a transmembrane protein that finds itself within the cell membrane under normal culture conditions. Herein, a cytoplasmic domain is the section of a transmembrane protein that finds itself on the cytosolic side of the cell membrane under normal culture conditions. Herein, an ectodomain is the section of a transmembrane protein that finds itself on the external side of the cell membrane under normal culture conditions. Suitably, the encoded HSV FcR binding partner or fragment thereof does not comprise a transmembrane domain. Suitably, the encoded HSV FcR binding partner or immunogenic fragment thereof does not comprise a cytoplasmic domain. Preferably, the encoded HSV FcR binding partner or immunogenic fragment thereof neither comprises a transmembrane domain, nor a cytoplasmic domain. In other words, in a preferred embodiment, the encoded HSV FcR binding partner or immunogenic fragment consists of a HSV FcR binding partner ectodomain (or extracellular domain).

More preferably, the encoded HSV FcR binding partner or immunogenic fragment is selected from a HSV2 gI2 ectodomain and a HSV1 gI1 ectodomain.

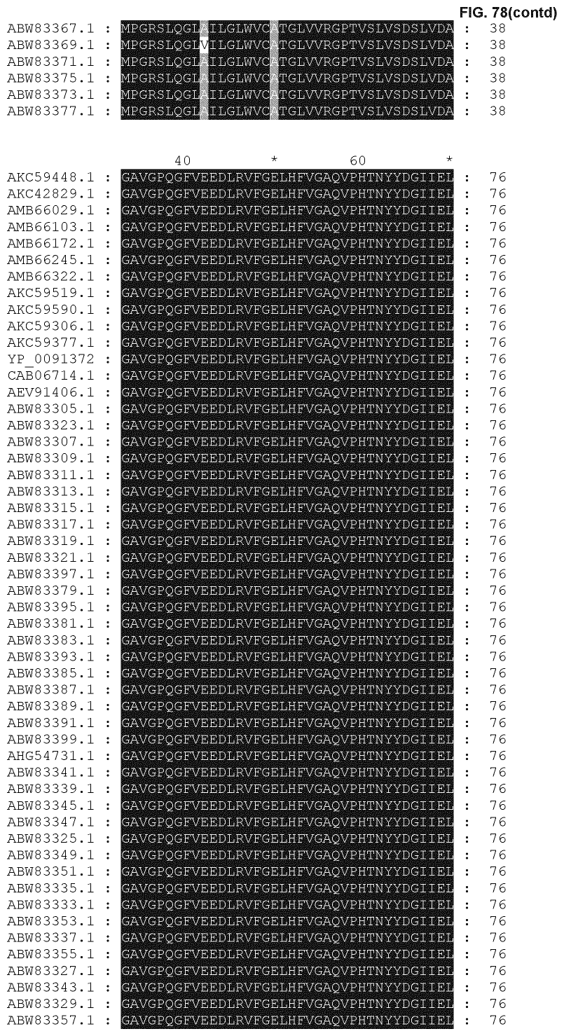

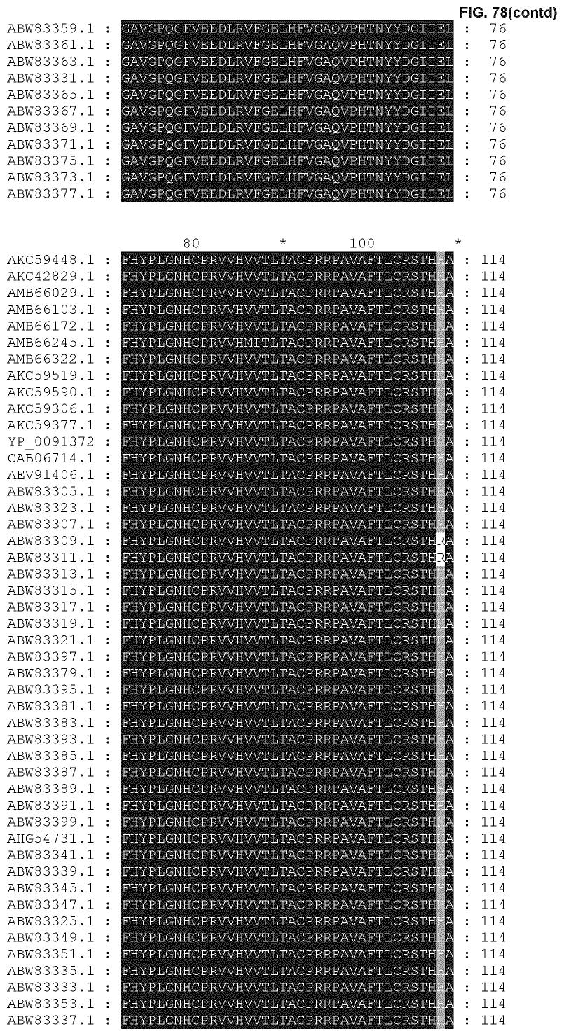

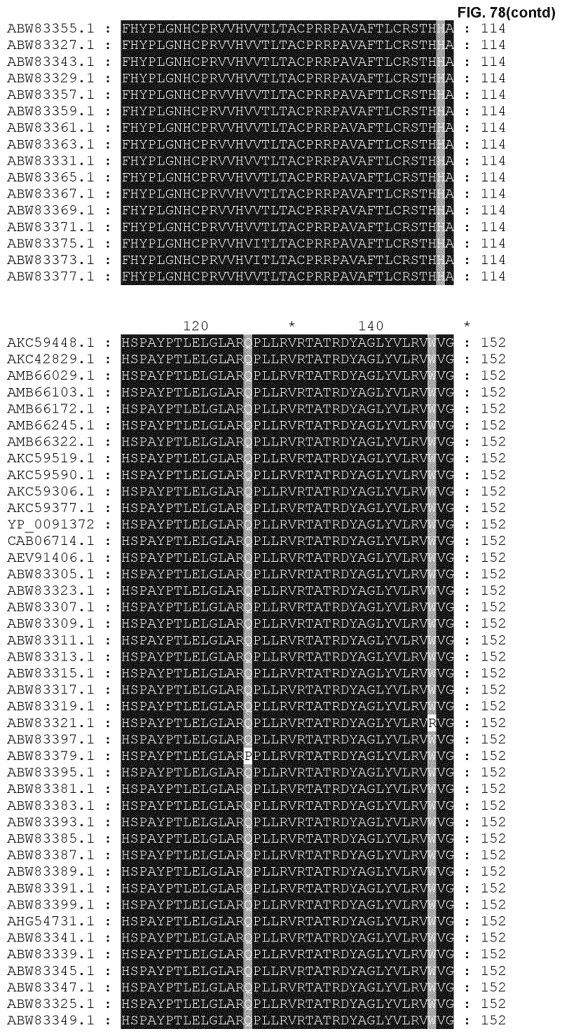

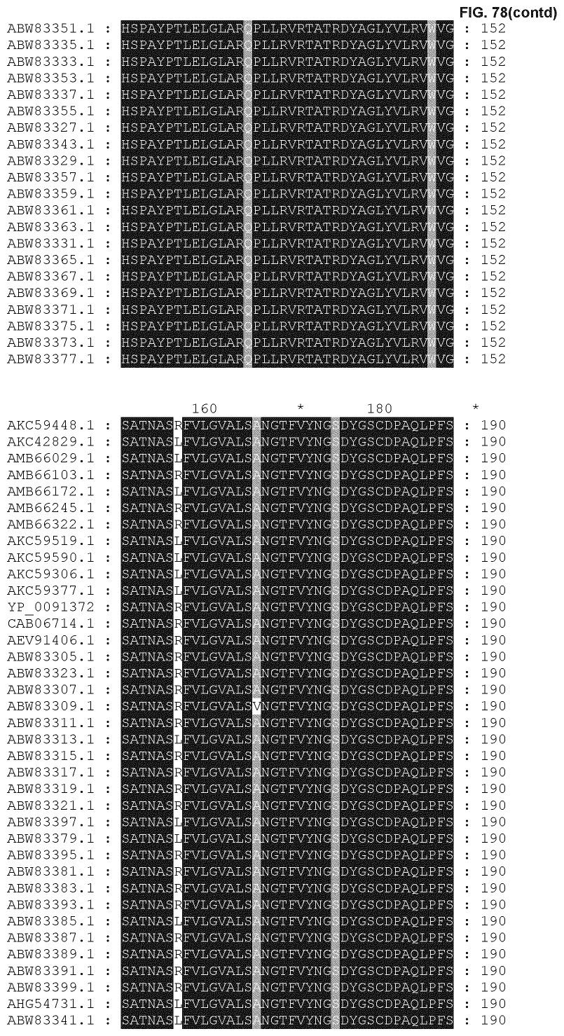

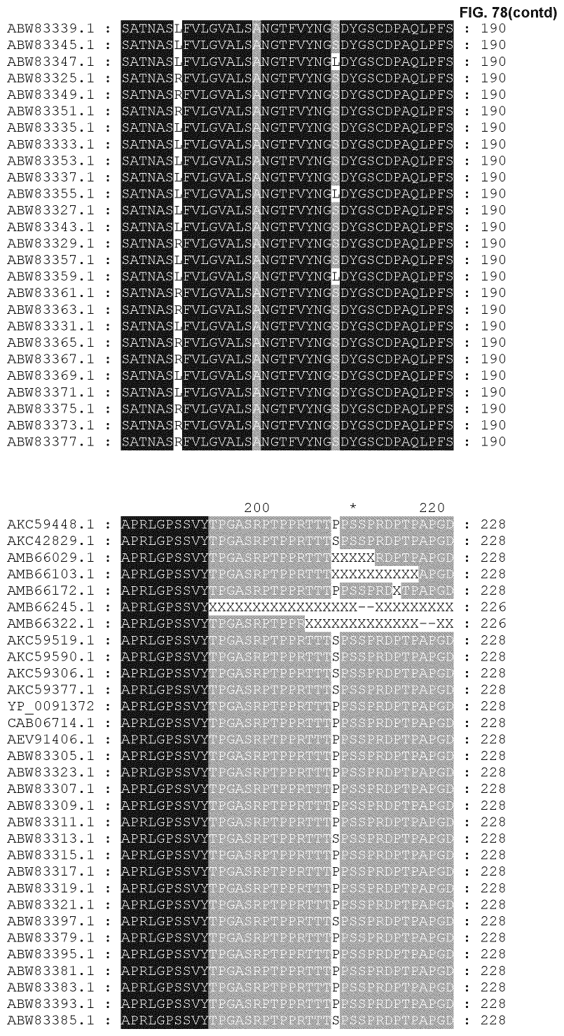

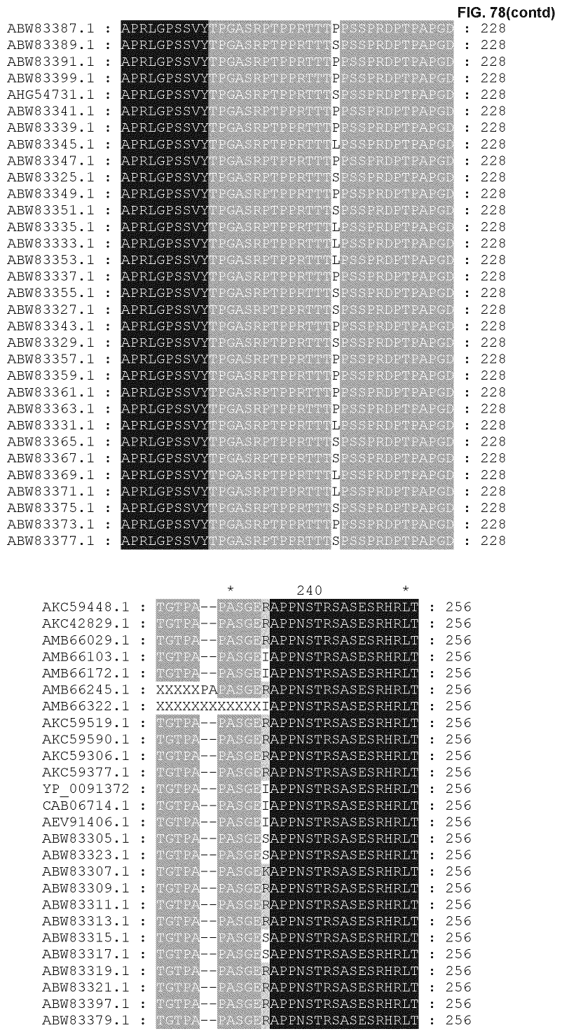

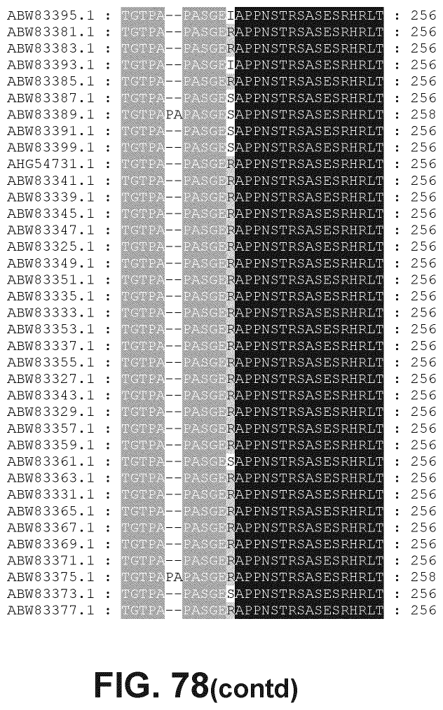

In a preferred embodiment, the encoded HSV FcR binding partner ectodomain is a HSV2 gI2 ectodomain which comprises or consists of the amino acid sequence shown on SEQ ID NO: 8 (corresponding to amino acid residues 1-256 of SEQ ID NO: 2), or a sequence which is at least 60%, 65%, 70%, 75% 80%, 85%, 90%, 95%, 96%, 97%, 98%, 99% or 99.5% identical thereto. Suitably, the HSV FcR ectodomain has a sequence selected from the sequences shown in Table 2 or FIG. 78. In a preferred embodiment, the encoded HSV FcR ectodomain is a HSV2 gE2 ectodomain which is at least 90%, 95%, 96%, 97%, 98%, 99%, 99.5% or 100% identical to SEQ ID NO: 8.

Suitably, the encoded HSV FcR binding partner HSV2 gI2 ectodomain may comprise one or more amino acid residue substitutions, deletions, or insertions relative to the amino acid sequence shown at SEQ ID NO: 8, for example 1, 2, 3, 4, 5, 6, 7, 8, 9 or 10 amino acid residue substitutions, deletions, or insertions.

In another embodiment, the encoded HSV FcR binding partner is a HSV2 gI2 ectodomain which comprises or consists of the amino acid sequence corresponding to amino acid residues 1-262 of SEQ ID NO: 2, or a sequence which is at least 60%, 65%, 70%, 75% 80%, 85%, 90%, 95%, 96%, 97%, 98%, 99% or 99.5% identical thereto. Suitably, the encoded HSV FcR binding partner HSV2 gI2 ectodomain may comprise one or more amino acid residue substitution, deletion, or insertion relative to the amino acid sequence corresponding to amino acid residues 1-262 of SEQ ID NO: 2, for example 1, 2, 3, 4, 5, 6, 7, 8, 9 or 10 amino acid residue substitutions, deletions, or insertions.

In another embodiment, the encoded HSV FcR binding partner ectodomain is a HSV1 gI1 ectodomain which comprises or consists of the amino acid sequence shown on SEQ ID NO: 10 (corresponding to amino acid residues 1-270 of SEQ ID NO: 4), or a sequence which is at least 60%, 65%, 70%, 75% 80%, 85%, 90%, 95%, 96%, 97%, 98%, 99% or 99.5% identical thereto. In a preferred embodiment, the HSV FcR ectodomain is a HSV1 gE1 ectodomain which is at least 90%, 95%, 96%, 97%, 98%, 99%, 99.5% or 100% identical to SEQ ID NO: 10.

Suitably, the encoded HSV FcR binding partner HSV1 gI1 ectodomain may comprise one or more amino acid residue substitution, deletion, or insertion relative to the amino acid sequence shown at SEQ ID NO: 10, for example 1, 2, 3, 4, 5, 6, 7, 8, 9 or 10 amino acid residue substitution, deletion, or insertions.

In another embodiment, the encoded HSV FcR binding partner is a HSV1 gI1 ectodomain which comprises or consists of the amino acid sequence corresponding to amino acid residues 1-276 of SEQ ID NO: 4, or a sequence which is at least 60%, 65%, 70%, 75% 80%, 85%, 90%, 95%, 96%, 97%, 98%, 99% or 99.5% identical thereto. Suitably, the encoded HSV FcR binding partner HSV1 gI1 ectodomain may comprise one or more amino acid residue substitution, deletion, or insertion relative to the amino acid sequence corresponding to amino acid residues 1-276 of SEQ ID NO: 4, for example 1, 2, 3, 4, 5, 6, 7, 8, 9 or 10 amino acid residue substitution, deletion, or insertions.

Antibodies against gD, gB and gC are detected in subjects infected with HSV, and gH/gL to a lesser extent. The dominant neutralising response was to gD (Cairns, Tina M., et al. “Dissection of the antibody response against herpes simplex virus glycoproteins in naturally infected humans.” Journal of virology 88.21 (2014): 12612-12622.).

In one embodiment, the encoded HSV Fc receptor or immunogenic fragment or variant thereof is not administered to the subject in combination with an immunodominant HSV antigen (e.g. gD1 or gD2).

Immunodominance is the immunological phenomenon in which immune responses are mounted against only a subset of the antigenic peptides produced by a pathogen. Immunodominance has been evidenced for antibody-mediated and cell-mediated immunity. As used herein, an “immunodominant antigen” is an antigen which comprises immunodominant epitopes. An immunodominant viral antigen is an immunodominant antigen of viral origin. In contrast, a “subdominant antigen” is an antigen which does not comprise immunodominant epitopes, or in other terms, only comprises subdominant epitopes. As used herein, an “immunodominant epitope” is an epitope that is dominantly targeted, or targeted to a higher degree, by neutralising antibodies during an immune response to a pathogen as compared to other epitopes from the same pathogen. As used herein, a “subdominant epitope” is an epitope that is not targeted, or targeted to a lower degree, by neutralising antibodies during an immune response to a pathogen as compared to other epitopes from the same pathogen. For example, gD2 is an immunodominant antigen for HSV2 and gD1 is an immunodominant antigen for HSV1. In contrast, gB2, gC2, gE2/gI2 and gH2/gL2 are subdominant antigens of HSV2 and gB1, gC1, gE1/gI1 and gH1/gL1 heterodimer are subdominant antigens of HSV1.

Suitably, where the encoded HSV Fc receptor is HSV2 gE2 or HSV1 gE1, the Fc receptor or immunogenic fragment or variant thereof is not administered to the subject together with HSV2 gD2 or HSV1 gD1, or a fragment thereof comprising immunodominant epitopes. In a particular embodiment where the encoded HSV Fc receptor is HSV2 gE2, the HSV Fc receptor or immunogenic fragment or variant thereof is not administered to the subject together with HSV2 gD2 or a fragment thereof comprising immunodominant epitopes. In another particular embodiment where the encoded HSV Fc receptor is HSV1 gE1, the HSV Fc receptor or immunogenic fragment or variant thereof is not administered to the subject together with HSV1 gD1 or a fragment thereof comprising immunodominant epitopes.

Glycoprotein gC from HSV1 and HSV2 is also involved in an immune escape mechanism by inhibiting complement (Awasthi, Sita, et al. “Blocking herpes simplex virus 2 glycoprotein E immune evasion as an approach to enhance efficacy of a trivalent subunit antigen vaccine for genital herpes.” Journal of virology 88.15 (2014): 8421-8432.).

In one embodiment, the encoded HSV Fc receptor is HSV2 gE2 and is administered to the subject together with HSV2 gC2, or an immunogenic fragment or variant thereof.

In one embodiment, the encoded HSV Fc receptor is HSV1 gE1 and is administered to the subject together with HSV1 gC1, or an immunogenic fragment or variant thereof.

In one embodiment, the ability of the encoded HSV FcR or immunogenic fragment or variant thereof to bind to a human antibody Fc domain is reduced or abolished compared to the corresponding native HSV Fc receptor.

Suitably, the encoded HSV Fc receptor or immunogenic fragment or variant thereof comprises one or more amino acid substitutions, deletions or insertions compared to the native sequence of the HSV Fc receptor or immunogenic fragment thereof, that reduce or abolish the binding affinity between the HSV FcR or immunogenic fragment or variant thereof and the antibody Fc domain compared to the native HSV Fc receptor.

In a preferred embodiment, the kₒₙ between the encoded HSV FcR or immunogenic fragment or variant thereof and human IgGs is lower than the kₒₙ between the corresponding native HSV FcR and human IgGs (slow binder). In a preferred embodiment, the k_{off} between the encoded HSV FcR or immunogenic fragment or variant thereof and human IgGs is higher than the k_{off} between the corresponding native HSV FcR and human IgGs (fast releaser). In a more preferred embodiment, the kₒₙ between the encoded HSV FcR or immunogenic fragment or variant thereof and human IgGs is lower than the kₒₙ between the corresponding native HSV FcR and human IgGs, and the k_{off} between the encoded HSV FcR or immunogenic fragment or variant thereof and human IgGs is higher than the k_{off} between the corresponding native HSV FcR and human IgGs (slow binder/fast releaser).

In a preferred embodiment, the equilibrium dissociation constant (K_{D}) between the encoded HSV FcR or immunogenic fragment or variant thereof and human IgGs is higher than the K D between the corresponding native HSV FcR and human IgGs.

In a preferred embodiment, the relative affinity between the encoded HSV FcR or immunogenic fragment or variant thereof and human IgGs is less than 100%, for example less than 90%, 80%, 70%, 60%, 50%, 40%, 30%, 20%, 15% or 10% of the affinity between the corresponding native viral FcR and human IgGs. In a more preferred embodiment, the relative affinity between the encoded HSV FcR or immunogenic fragment or variant thereof and human IgGs is less than 15%, more preferably still less than 10% of the affinity between the corresponding native HSV FcR and human IgGs.

In a preferred embodiment, the equilibrium dissociation constant (K D) between the encoded HSV FcR or immunogenic fragment or variant thereof and human IgGs is higher than 2×10⁻⁷ M, preferably higher than 5×10⁻⁷ M, more preferably higher than 1×10⁻⁶ M.

Alternatively, the ability of the HSV FcR or immunogenic fragment or variant thereof to bind to a human antibody Fc domain can be assessed by measuring the response (expressed in nm) in a BiLayer Interferometry assay.

In a preferred embodiment, the response in a BiLayer Interferometry assay corresponding to the binding between the HSV FcR or immunogenic fragment or variant thereof and human IgGs is less than 80%, suitably less than 70%, 60%, 50%, 40% of the response obtained with the corresponding native HSV FcR. In a preferred embodiment, the response in a BiLayer Interferometry assay corresponding to the binding between the HSV FcR or immunogenic fragment or variant thereof and human IgGs is lower than 0.4 nm, suitably lower than 0.3 nm, 0.2 nm or 0.1 nm.

In a preferred embodiment, the encoded HSV FcR or immunogenic fragment or variant thereof is HSV2 gE2 or an immunogenic fragment or variant thereof. Suitably, the encoded HSV2 gE2 or immunogenic fragment or variant thereof comprises one or more mutations (insertions, substitutions or deletions) at positions selected from N241, H245, A246, A248, R314, P317, P318, P319, F322, R320, A337, S338 or V340 of the HSV2 gE2 sequence shown in SEQ ID NO: 1.

Exemplary mutations that may be used herein to reduce or abolish the binding affinity between the encoded HSV2 gE2 or immunogenic fragment or variant thereof and an antibody Fc domain include the single point substitution mutations of the sequence shown in SEQ ID NO: 1 selected from H245A, H245K, P317R, P319A, P319R, P319G, P319K, P319T, A337G, P319D, P319S, S338D, N241A, R320D, H245E, H245V, H245R, H245D, H245Q, H245G, H2451, H245K, H245S, H245T, A246W, A248K, A248T, A248G, R314A, R314N, R314D, R314Q, R314E, R314G, R314I, R314L, R314K, R314M, R314F, R314P, R314S, R314T, R314Y, R314V, P317N, P317G, P317I, P317L, P317K, P317F, P317S, P318R, P318D, P318Q, P318I, P318S, P318T, P318Y, P319L, R320A, R320S, R320N, R320Q, R320E, R320G, R320H, R320I, R320L, R320M, R320P, R320T, R320V, F322A, F322N, F322I, F322K, F322P, F322T, S338G, S338E, S338L, S338T, V340A, V340R, V340D, V340Q, V340M, V340F, V340P and V340W.

Exemplary mutations that may be used herein to reduce or abolish the binding affinity between the encoded HSV2 gE2 or immunogenic fragment or variant thereof and an antibody Fc domain also include the double point substitution mutations of the sequence shown in SEQ ID NO: 1 selected from H245A and P319A; H245A and P319R; H245A and P319G; H245A and P319K; H245A and P319T; N241A and R320D; N241A and P319D; A246W and P317K; A246W and P317F; A246W and P317S; A246W and R320D; A246W and R320G; A246W and R320T; A248K and V340R; A248K and V340M; A248K and V340W; A248T and V340R; A248T and V340M; A248T and V340W; A248G and V340R; A248G and V340M; A248G and V340W; A248K and F322A; A248K and F322I; A248K and F322P; A248T and F322A; A248T and F322I; A248T and F322P; A248G and F322A; A248G and F322I; A248G and F322P; H245A and R320D; H245A and R320G; H245A and R320T; H245G and R320D; H245G and R320G; H245G and R320T; H245S and R320D; H245S and R320G; H245S and R320T; H245A and P319G; H245A and P319L; H245G and P319G; H245G and P319L; H245S and P319G; H245S and P319L; R314G and P318R; R314G and P318D; R314G and P318I; R314L and P318R; R314L and P318D; R314L and P318I; R314P and P318R; R314P and P318D; R314P and P318I; R314G and F322A; R314G and F322I; R314G and F322P; R314L and F322A; R314L and F322I; R314L and F322P; R314P and F322A; R314P and F322I; R314P and F322P; R314G and V340R; R314G and V340M; R314G and V340W; R314L and V340R; R314L and V340M; R314L and V340W; R314P and V340R; R314P and V340M; R314P and V340W; P317K and V340R; P317K and V340M; P317K and V340W; P317F and V340R; P317F and V340M; P317F and V340W; P317S and V340R; P317S and V340M; P317S and V340W; P317K and S338G; P317K and S338H; P317K and S338L; P317F and S338G; P317F and S338H; P317F and S338L; P317S and S338G; P317S and S338H; P317S and S338L; P318R and S338G; P318R and S338H; P318R and S338L; P318D and S338G; P318D and S338H; P318D and S338L; P318I and S338G; P318I and S338H; P318I and S338L; P319G and V340R; P319G and V340M; P319G and V340W; P319L and V340R; P319L and V340M; P319L and V340W; P317R and P319D; P317R and R320D; P319D and R320D.

Exemplary mutations that may be used herein to reduce or abolish the binding affinity between the encoded HSV2 gE2 or immunogenic fragment or variant thereof and an antibody Fc domain also include deletion mutations at positions P319 and/or R320 of the sequence shown in SEQ ID NO: 1, alone or in combination with substitution mutations, in particular mutations selected from P319 deletion; R320 deletion; P319 deletion/R320 deletion; P319 deletion/R320 deletion/P317G/P318G; P319 deletion/R320 deletion/P318E; P319 deletion/R320 deletion/P318G; P319 deletion/R320 deletion/P318K; P319 deletion/R320 deletion/P317R/P318E; P319 deletion/R320 deletion/P317R/P318G; P319 deletion/R320 deletion/P317R/P318K; P319 deletion/R320 deletion/P317G/P318K.

Exemplary mutations that may be used herein to reduce or abolish the binding affinity between the encoded HSV2 gE2 or immunogenic fragment or variant thereof and an antibody Fc domain also include the insertion mutations selected from:
insertion of peptide sequence LDIGE between amino acid residues Y275 and E276 of SEQ ID NO: 1 (275_insert_LDIGE),
insertion of peptide sequence ADIGL between amino acid residues S289 and P290 of SEQ ID NO: 1 (289_insert_ADIGL),
insertion of peptide sequence ARAA between amino acid residues A337 and S338 of SEQ ID NO: 1 (337_insert ARAA),
insertion of peptide sequence ARAA between amino acid residues S338 and T339 of SEQ ID NO: 1 (338_insert_ARAA), and
insertion of peptide sequence ADIT between amino acid residues H346 and A347 of SEQ ID NO: 1 (346_insert_ADIT).

In a preferred embodiment, the encoded HSV2 gE2 or immunogenic fragment or variant thereof comprises a mutation or a combination of mutations with respect to the sequence shown in SEQ ID NO: 1 selected from 289_insert_ADIGL; 338_insert ARAA; H245K; P317R; P319R; P319G; P319K; H245A_P319R; H245A_P319G; H245A_P319K; H245A_P319T; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248I; P318S; P317K; P317S_V340W; H245D; R314P_V340W; R314L_318D; P319L_V340W; P317F; P318D_S338G; R314G_V340W; P317K_S338H; R314L_V340W; P318R; P318Q; P317F_5338 G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R320T; R314P_P318R; A248G_F322I; R320N; P317N; R314D; R314Y; R314P_F322I; P319G_V340M; P317S_V340R; R314V; P317R_P319D; P317R_R320D; P319D_R320D; Δ319_Δ320; P317G_P318G_Δ319_Δ320; P318E_Δ319_Δ320; P318G_Δ319_Δ320; P318K_Δ319_Δ320; P317R_P318E_Δ319_320; P317R_P318G_Δ319_Δ320 and P317G_P318K_Δ319_Δ320.

In a more preferred embodiment, the HSV2 gE2 or immunogenic fragment or variant thereof comprises a mutation or a combination of mutations with respect to the sequence shown in SEQ ID NO: 1 selected from 338_insert ARAA; P317R; P319D; R320D; A248T_V340W; V340W; A248T; P318I and A246W.

Corresponding mutations in other HSV2 gE2 sequences, for example the sequences listed in Table 1 and shown on the alignment presented in FIG. 77, to the exemplary single and double substitution, selection and mutations and insertion mutations listed above are also in the scope of the present invention.

All possible combinations of the exemplary single and double substitution mutations and insertion mutations listed above are also in the scope of the present invention.

In a preferred embodiment, the encoded HSV2 gE2 or immunogenic fragment or variant thereof is a encoded HSV2 gE2 ectodomain as described herein.

In another embodiment, the encoded HSV FcR or immunogenic fragment or variant thereof is a encoded HSV1 gE1 or an immunogenic fragment or variant thereof. Suitably, the encoded HSV1 gE1 or immunogenic fragment or variant thereof comprises one or more mutations (insertions, substitutions or deletions) at positions selected from H247, P319 and P321 of the HSV1 gE1 sequence shown in SEQ ID NO: 3.

Exemplary mutations that may be used herein to reduce or abolish the binding affinity between the encoded HSV1 gE1 or immunogenic fragment or variant thereof and an antibody Fc domain include the single point substitution mutations of the sequence shown in SEQ ID NO: 3 selected from H247A, H247K, P319R, P321A, P321R, P321G, P321K, P321T, A339G, P321D, P321S, A340D, N243A and R322D, and the double point substitutions mutations of the sequence shown in SEQ ID NO: 3 selected from H247A/P321A, H247A/P321R, H247A/P321G, H247A/P321K, H247A/P321T, N243A/R322D, N243A/P321D, H247G/P319G, P319G/P321G and A340G/S341G/V342G.

Exemplary mutations that may be used herein to reduce or abolish the binding affinity between the encoded HSV1 gE1 or immunogenic fragment or variant thereof and an antibody Fc domain also include the insertion mutations selected from:
insertion of peptide sequence LDIGE between amino acid residues Y277 and E278 of SEQ ID NO: 3 (277_insert_LDIGE);
insertion of peptide sequence ADIGL between amino acid residues S291 and P292 of SEQ ID NO: 3 (291_insert_ADIGL);
insertion of peptide sequence ARAA between amino acid residues A339 and A340 of SEQ ID NO: 3 (339_inset_ARAA);
insertion of peptide sequence ARAA between amino acid residues A340 and S341 of SEQ ID NO: 3 (340_inset_ARAA); and
insertion of peptide sequence ADIT between amino acid residues D348 and A349 of SEQ ID NO: 3 (348 insert_ADIT).

In a preferred embodiment, the HSV1 gE1 or immunogenic fragment or variant thereof comprises a mutation or a combination of mutations with respect to the sequence shown in SEQ ID NO: 3 selected from P321K; P321D; R322D; N243A_R322D; N243A_P321D; A340G_S341G_V342G; H247G_P319G; P321R; H247A_P321K; 291_insert_ADIGL; 339_insert ARAA; P319R; P319G_P321G and H247A_P321R.

In a more preferred embodiment, the HSV1 gE1 or immunogenic fragment or variant thereof comprises a mutation or a combination of mutations with respect to the sequence shown in SEQ ID NO: 3 selected from P321D; R322D; A340G S341G_V342G and P319R.

In one embodiment is provided a nucleic acid encoding a HSV2 Fc receptor or an immunogenic fragment or variant thereof for the use described herein wherein:
the nucleic acid is a RNA molecule (e.g. a self replicating RNA molecule) and the RNA is encapsulated in lipid nanoparticles (LNP) and
the ability of said variant thereof to bind to a human antibody Fc domain is reduced or abolished compared to the corresponding native HSV Fc receptor.

Preferably, the encoded Fc receptor is gE2 (optionally in combination with its binding partner gI2) and the variant thereof comprises a mutation or a combination of mutations with respect to the sequence shown in SEQ ID NO: 1 selected from 338_insert ARAA; P317R; P319D; R320D; A248T_V340W; V340W; A248T; P318I and A246W.

In a further embodiment is provided a nucleic acid encoding a HSV1 Fc receptor or an immunogenic fragment or variant thereof for the use described herein wherein:
the nucleic acid is a RNA molecule (e.g. a self-replicating RNA molecule) and the RNA is encapsulated in lipid nanoparticle (LNP) and
the ability of said variant thereof to bind to a human antibody Fc domain is reduced or abolished compared to the corresponding native HSV Fc receptor.

Preferably, the encoded Fc receptor is gE1 (optionally in combination with its binding partner gI1) and the variant thereof comprises a mutation or a combination of mutations with respect to the sequence shown in SEQ ID NO: 3 selected from H247A, H247K, P319R, P321A, P321R, P321G, P321K, P321T, A339G, P321D, P321S, A340D, N243A and R322D, H247A/P321A, H247A/P321R, H247A/P321G, H247A/P321K, H247A/P321T, N243A/R322D, N243A/P321D, H247G/P319G, P319G/P321G and A340G/S341G/V342G.

Corresponding mutations in other HSV1 gE1 sequences to the exemplary single and double substitution mutations and insertion mutations listed above are also in the scope of the present invention.

All possible combinations of the exemplary single and double substitution mutations and insertion mutations listed above are also in the scope of the present invention.

In a preferred embodiment, the encoded HSV1 gE1 or immunogenic fragment or variant thereof is a HSV1 gE1 ectodomain as described herein.

In a preferred embodiment, the encoded HSV FcR or immunogenic fragment or variant thereof is part of a heterodimer with a binding partner from said virus or a fragment thereof.

In a preferred embodiment, the encoded HSV Fc receptor is recombinant HSV2 gE2 or an immunogenic fragment or variant thereof and the binding partner is HSV2 gI2 or a fragment thereof as described herein.

In another preferred embodiment, the encoded HSV Fc receptor is HSV1 gE1 or an immunogenic fragment or variant thereof and the binding partner is HSV1 gI1 or a fragment thereof or a fragment thereof as described herein.

In another embodiment, the encoded HSV FcR or immunogenic fragment or variant thereof is part of a heterodimer with a binding partner from said HSV virus or a fragment thereof. In a further embodiment, the HSV Fc receptor is HSV2 gE2 and the binding partner is HSV2 gI2. In another embodiment, the HSV Fc receptor is HSV1 gE1 and the binding partner is HSV1 gI1.

### Therapeutic Uses and Compositions

In another aspect, the invention provides a pharmaceutical composition comprising a nucleic acid encoding a HSV2 Fc receptor or an immunogenic fragment or variant thereof and a pharmaceutically acceptable carrier for use in inducing a cross reactive immune response against HSV1 when administered to a subject. In one embodiment of the pharmaceutical composition, the HSV2 Fc receptor is HSV2 gE2 and the binding partner is HSV2 gI2. Preferably, the pharmaceutical composition is for use as a pan-HSV vaccine.

In a further aspect of the invention, is provided a pharmaceutical composition comprising a nucleic acid encoding a HSV1 Fc receptor or an immunogenic fragment or variant thereof and a pharmaceutically acceptable carrier for use in inducing a cross reactive immune response against HSV2 when administered to a subject. In one embodiment of the pharmaceutical composition, the HSV1 Fc receptor is HSV1 gE1 and the binding partner is HSV1 gI1. Preferably, the pharmaceutical composition is for use as a pan-HSV vaccine.

In another aspect, the invention provides an immunogenic composition comprising a nucleic acid encoding a HSV2 Fc receptor or an immunogenic fragment or variant thereof and a pharmaceutically acceptable carrier for use in inducing a cross reactive immune response against HSV1 when administered to a subject. In a further aspect, is provided an immunogenic composition comprising nucleic acid encoding a HSV1 Fc receptor or an immunogenic fragment or variant thereof and a pharmaceutically acceptable carrier for use in inducing a cross reactive immune response against HSV2 when administered to a subject. Suitably, the immunogenic composition may be prepared for administration by being suspended or dissolved in a pharmaceutically or physiologically acceptable carrier. Preferably, the immunogenic compositions of the invention are suitable for use as therapeutic vaccines. Most preferably, the immunogenic compositions are for use as pan-HSV vaccines.

A “pharmaceutically acceptable carrier” includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The compositions may also contain a pharmaceutically acceptable diluent, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. The appropriate carrier may depend in large part upon the route of administration.

Suitably, the nucleic acid encoding a HSV Fc receptor or immunogenic fragment or variant thereof is to be administered to a subject by any route as is known in the art, including intramuscular, intravaginal, intravenous, intraperitoneal, subcutaneous, epicutaneous, intradermal, nasal, intratumoral or oral administration.

In one embodiment, the subject is a vertebrate, such as a mammal, e.g. a human, a non-human primate, or a veterinary mammal (livestock or companion animals). In a preferred embodiment, the subject is a human.

In a preferred embodiment, the subject has been infected by the virus (i.e. is seropositive), for example HSV2 and/or HSV1 prior to being treated with the nucleic acid encoding a HSV (HSV1 or HSV2) FcR or immunogenic fragment or variant thereof. The subject which has been infected with the virus prior to being treated with the nucleic acid encoding a HSV FcR or immunogenic fragment or variant thereof may have shown clinical signs of the infection (symptomatic subject) or may not have shown clinical sings of the viral infection (asymptomatic subject). In one embodiment, the symptomatic subject has shown several episodes with clinical symptoms of infections over time (recurrences) separated by periods without clinical symptoms.

In one aspect, the invention provides a nucleic acid encoding a HSV2 Fc receptor or immunogenic fragment or variant thereof, for use in i) inducing a cross reactive immune response against HSV-1 when administered to a subject and ii) in the treatment of recurrent herpes infection, or in a method for prevention or reduction of the frequency of recurrent herpes virus infection in a subject, preferably a human subject. In one embodiment, the encoded HSV2 Fc receptor is HSV2 gE2 or an immunogenic fragment or variant thereof. In another embodiment, the encoded HSV Fc receptor is a HSV2 gE2/gI2 heterodimer or immunogenic fragment or variant thereof. In a further embodiment, the encoded HSV2 Fc receptor or immunogenic fragment or variant thereof for the use described herein and for use as a pan-HSV vaccine.

In a further aspect, the invention provides a nucleic acid encoding a HSV1 Fc receptor or immunogenic fragment or variant thereof, for use in i) inducing a cross reactive immune response against HSV-2 when administered to a subject and ii) in the treatment of recurrent herpes infection, or in a method for prevention or reduction of the frequency of recurrent herpes virus infection in a subject, preferably a human subject. In one embodiment, the encoded HSV1 Fc receptor is HSV1 gE1 or an immunogenic fragment or variant thereof. In another embodiment, the encoded HSV Fc receptor is HSV1 gE1/gI1 heterodimer or immunogenic fragment or variant thereof. In a further embodiment, the encoded HSV1 Fc receptor or immunogenic fragment or variant thereof for the use described herein is for use as a pan-HSV vaccine.

In one aspect, the invention provides a nucleic acid encoding a HSV2 FcR or immunogenic fragment or variant thereof, as described herein for use in the manufacture of an immunogenic composition which when administered to a subject induces a cross reactive immune response against HSV1.

In a further aspect, the invention provides a nucleic acid encoding a HSV1 FcR or immunogenic fragment or variant thereof, as described herein for use in the manufacture of an immunogenic composition which when administered to a subject induces a cross reactive immune response against HSV2.

In one aspect, the invention provides the use of a nucleic acid encoding a HSV2 FcR or immunogenic fragment or variant thereof, as described herein in the manufacture of a medicament for the prevention or treatment of herpes infection or herpes-related disease wherein the HSV2 Fc receptor or immunogenic fragment or variant thereof can induce a cross reactive immune response against HSV1 when administered to a subject.

In a further aspect, the invention provides the use of a nucleic acid encoding said HSV1 FcR or immunogenic fragment or variant thereof, as described herein in the manufacture of a medicament for the prevention or treatment of herpes infection or herpes-related disease wherein the HSV1 Fc receptor or immunogenic fragment or variant thereof can induce a cross reactive immune response against HSV2 when administered to a subject.

In one aspect, the invention provides a nucleic acid encoding a HSV2 gE2 or gE2/gI2 heterodimer, an immunogenic fragment or variant thereof, as described herein for use in the manufacture of an immunogenic composition wherein the immunogenic composition can induce a cross reactive immune response against HSV1 when administered to a subject.

In a further aspect, the invention provides a nucleic acid encoding a HSV1 gE1 or gE1/gI1 heterodimer, an immunogenic fragment or variant thereof, as described herein for use in the manufacture of an immunogenic composition wherein the immunogenic composition can induce a cross reactive immune response against HSV2 when administered to a subject.

In one aspect, the invention provides the use of a nucleic acid encoding a HSV2 gE2 or gE2/gI2 heterodimer, an immunogenic fragment or variant thereof, as described herein in the manufacture of a medicament for the prevention or treatment of i) HSV2 infection or HSV2-related disease and ii) HSV1 infection or HSV1-related disease, wherein the nucleic acid encoding a HSV2 gE2 or gE2/gI2 heterodimer or an immunogenic fragment or variant thereof can induce a cross reactive immune response against HSV1 when administered to a subject.

In a further aspect, the invention provides the use of a nucleic acid encoding a HSV1 gE1 or gE1/gI1 heterodimer, an immunogenic fragment or variant thereof, as described herein in the manufacture of a medicament for the prevention or treatment of i) HSV1 infection or HSV1-related disease and ii) HSV2 infection or HSV2-related disease, wherein the HSV1 gE1 or gE1/gI1 heterodimer or an immunogenic fragment or variant thereof can induce a cross reactive immune response against HSV2 when administered to a subject.

In one aspect, the invention provides a method of preventing or treating a herpes virus infection or herpes virus related disease in a subject in need thereof comprising administering an immunologically effective amount of a nucleic acid encoding a HSV2 Fc receptor or immunogenic fragment or variant thereof, to the subject wherein the HSV2 Fc receptor or immunogenic fragment or variant thereof can induce a cross reactive immune response against HSV1 when administered to a subject. In one embodiment the herpes virus infection is a HSV1 infection and the herpes virus related disease is a HSV1 related disease. In a further embodiment, the encoded HSV2 Fc receptor is HSV2 gE2 or gE2/gI2 heterodimer, or an immunogenic fragment or variant thereof.

In a further aspect, the invention provides a method of preventing or treating a herpes virus infection or herpes virus related disease in a subject in need thereof comprising administering an immunologically effective amount of a nucleic acid encoding a HSV1 Fc receptor or immunogenic fragment or variant thereof, to the subject wherein the HSV1 Fc receptor or immunogenic fragment or variant thereof can induce a cross reactive immune response against HSV2 when administered to a subject. In one embodiment the herpes virus infection is a HSV2 infection and the herpes virus related disease is a HSV2 related disease. In a further embodiment, the encoded HSV1 Fc receptor is HSV1 gE1 or gE1/gI1 heterodimer, or an immunogenic fragment or variant thereof.

A further aspect of the invention provides a method of treating a subject infected with HSV1 or preventing HSV1 infection in a subject comprising administering an immunologically effective amount of a nucleic acid encoding HSV2 Fc receptor or an immunogenic fragment or variant thereof to the subject and inducing a cross reactive immune response against HSV1.

A yet further aspect of the invention provides a method of treating a subject infected with HSV2 or preventing HSV2 infection in a subject comprising administering an immunologically effective amount of a nucleic acid encoding HSV1 Fc receptor or an immunogenic fragment or variant thereof to the subject and inducing a cross reactive immune response against HSV2.

Also provided is a method comprising administering to the subject a nucleic acid encoding a Fc receptor that is HSV2 gE2 or HSV1 gE1, and not administering together with a nucleic acid encoding an immunodominant viral antigen (e.g., HSV2 gD2 or HVS1 gD1).

Also provided is a method comprising administering, in an effective immunological amount to a subject, a nucleic acid encoding a HSV Fc receptor or an immunogenic fragment or variant thereof together with a nucleic acid encoding HSV2 gC2 or an immunogenic fragment thereof or a nucleic acid encoding HSV1 gC1 or an immunogenic fragment thereof.

Also provided is a method comprising administering, in an effective immunological amount to a subject, a pharmaceutical composition comprising a nucleic acid encoding HSV2 Fc receptor or an immunogenic fragment or variant thereof and a pharmaceutically acceptable carrier, and inducing a cross reactive immune response against HSV1 when administered to the subject.

Also provided is a method comprising administering, in an effective immunological amount to a subject, a pharmaceutical composition comprising a nucleic acid encoding HSV1 Fc receptor or an immunogenic fragment or variant thereof and a pharmaceutically acceptable carrier, and inducing a cross reactive immune response against HSV2 when administered to the subject.

As used herein, the terms “treat” and “treatment” as well as words stemming therefrom, are not meant to imply a “cure” of the condition being treated in all individuals, or 100% effective treatment in any given population. Rather, there are varying degrees of treatment which one of ordinary skill in the art recognizes as having beneficial therapeutic effect(s). In this respect, the inventive methods and uses can provide any level of treatment of herpes virus infection and in particular HSV2 and/or HSV1 related disease in a subject in need of such treatment, and may comprise reduction in the severity, duration, or number of recurrences over time, of one or more conditions or symptoms of herpes virus infection, and in particular HSV2 and/or HSV1 related disease.

As used herein, “therapeutic immunization” or “therapeutic vaccination” refers to administration of the immunogenic compositions of the invention to a subject, preferably a human subject, who is known to be infected with a virus such as a herpes virus and in particular HSV2 and/or HSV1 at the time of administration, to treat the viral infection or virus-related disease. As used herein, “prophylactic immunization” or “prophylactic vaccination” refers to administration of the immunogenic compositions of the invention to a subject, preferably a human subject, who has not been infected with a virus such as a herpes virus and in particular HSV2 and/or HSV1 at the time of administration, to prevent the viral infection or virus-related disease.

For the purpose of the present invention, treatment of HSV infection aims at preventing reactivation events from the latent HSV infection state or at controlling at early stage viral replication to reduce viral shedding and clinical manifestations that occur subsequent to primary HSV infection, i.e. recurrent HSV infection. Treatment thus prevents either or both of HSV symptomatic and asymptomatic reactivation (also referred to as recurrent HSV infection), including asymptomatic viral shedding. Treatment may thus reduce the severity, duration, and/or number of episodes of recurrent HSV infections following reactivation in symptomatic individuals. Preventing asymptomatic reactivation and shedding from mucosal sites may also reduce or prevent transmission of the infection to those individuals naïve to the HSV virus (i.e. HSV2, HSV1, or both). This includes prevention of transmission of HSV through sexual intercourse, in particular transmission of HSV2 but also potential transmission of HSV1 through sexual intercourse. Thus the immunogenic construct of the present invention may achieve any of the following useful goals: preventing or reducing asymptomatic viral shedding, reducing or preventing symptomatic disease recurrences, reducing duration or severity of symptomatic disease, reducing frequency of recurrences, prolonging the time to recurrences, increasing the proportion of subjects that are recurrence-free at a given point in time, reducing the use of antivirals, and preventing transmission between sexual partners.

In particular, the nucleic acid encoding a HSV2 Fc receptor or an immunogenic fragment or variant thereof and immunogenic compositions described herein for use in inducing a cross reactive immune response against HSV-1 when administered to a subject, are useful as therapeutic vaccines, to treat or prevent recurrent viral infections in a subject in need of such treatment. Preferably, the therapeutic vaccine is a pan-HSV therapeutic vaccine. Preferably, the subject is a human.

Also, the nucleic acid encoding a HSV1 Fc receptor or an immunogenic fragment or variant thereof and immunogenic compositions described herein for use in inducing a cross reactive immune response against HSV2 when administered to a subject, are useful as therapeutic vaccines, to treat or prevent recurrent viral infections in a subject in need of such treatment. Preferably, the therapeutic vaccine is a pan-HSV therapeutic vaccine. Preferably, the subject is a human.

A pan-HSV vaccine shows a humoral and/or cellular immune response against both HSV1 and HSV2 i.e. both serotypes of HSV. Hence, a HSV1 or HSV2 Fc receptor or immunogenic fragment or variant thereof as described herein can be used as a pan-HSV vaccine for use against both types of HSV, HSV1 and HSV2. In this way, the manufacture of an antigen prepared from just one type of HSV is sufficient to make a vaccine against all serotypes of HSV, without the additional manufacture requirements and complexity of making a vaccine with antigens from both serotypes of HSV.

Suitably, the nucleic acid encoding a HSV1 or HSV2 Fc receptor or an immunogenic fragment or variant thereof and immunogenic compositions for the uses described herein are not part of a prophylactic vaccine.

Methods of use as provided herewith may be directed at both HSV2 and HSV1 infections (and thus at both HSV2 and HSV1 related disease, i.e., genital herpes and herpes labialis, respectively), or at HSV2 infections (thus primarily aiming at treatment of genital herpes), or at HSV1 infections (thus primarily aiming at treatment of herpes labialis).

By “immunologically effective amount” is intended that the administration of that amount of nucleic acid (or immunogenic composition containing the nucleic acid) to a subject, either in a single dose or as part of a series, is effective for inducing a measurable immune response against the administered antigen in the subject. This amount varies depending upon the health and physical condition of the individual to be treated, age, the taxonomic group of individual to be treated (e.g. human, non-human primate, etc.), the capacity of the individual's immune system to synthesize antibodies, the degree of protection desired, the formulation of the composition or vaccine, the treating doctor's assessment of the medical situation, the severity of the disease, the potency of the compound administered, the mode of administration, and other relevant factors. Vaccines as disclosed herein are typically therapeutic. In some embodiments, the immunogenic compositions disclosed herein may induce an effective immune response against a herpes virus infection, i.e., a response sufficient for treatment or prevention of herpes virus infection, such as recurrent HSV infection. Further uses of immunogenic compositions or vaccines comprising the nucleic acid constructs as described herein are provided herein below. It will be readily understood that the nucleic acid encoding a HSV Fc receptor or an immunogenic fragment or variant thereof and immunogenic compositions described herein are suited for use in regimens involving repeated delivery of the HSV Fc receptor or immunogenic fragment or variant thereof over time for therapeutic purposes. Suitably, a prime-boost regimen may be used. Prime-boost refers to eliciting two separate immune responses in the same individual: (i) an initial priming of the immune system followed by (ii) a secondary or boosting of the immune system weeks or months after the primary immune response has been established. Preferably, a boosting composition is administered about two to about 12 weeks after administering the priming composition to the subject, for example about 2, 3, 4, 5 or 6 weeks after administering the priming composition. In one embodiment, a boosting composition is administered one or two months after the priming composition. In one embodiment, a first boosting composition is administered one or two months after the priming composition and a second boosting composition is administered one or two months after the first boosting composition.

Dosages will depend primarily on factors such as the route of administration, the condition being treated, the age, weight and health of the subject, and may thus vary among subjects.

Exemplary effective amounts of a nucleic acid component can be between 1 ng and 100 μg, such as between 1 ng and 1 μg (e.g., 100 ng-1 μg), or between μg and 100 μg, such as 10 ng, 50 ng, 100 ng, 150 ng, 200 ng, 250 ng, 500 ng, 750 ng, or 1 μg. Effective amounts of a nucleic acid can also include from 1 μg to 500 μg, such as between 1 μg and 200 μg, such as between 10 and 100 μg, for example 1 μg, 2 μg, 5 μg, 10 μg, 20 μg, 50 μg, 75 μg, 100 μg, 150 μg, or 200 μg. Alternatively, an exemplary effective amount of a nucleic acid can be between 100 μg and 1 mg, such as from 100 μg to 500 μg, for example, 100 μg, 150 μg, 200 μg, 250 μg, 300 μg, 400 μg, 500 μg, 600 μg, 700 μg, 800 μg, 900 μg or 1 mg.

Generally, a human dose will be in a volume of between 0.1 ml and 2 ml. Thus, the composition described herein can be formulated in a volume of, for example, about 0.1, 0.15, 0.2, 0.5, 1.0, 1.5 or 2.0 ml human dose per individual or combined immunogenic components.

One of skill in the art may adjust these doses, depending on the route of administration and the subject being treated.

The therapeutic immune response against the encoded HSV Fc receptor or an immunogenic fragment or variant thereof can be monitored to determine the need, if any, for boosters. Following an assessment of the immune response (e.g., of CD4+ T cell response, CD8+ T cell response, antibody titers in the serum), optional booster immunizations may be administered.

In vitro or in vivo testing methods suitable for assessing the immune response against the encoded HSV Fc receptor or fragment thereof according to the invention are known to those of skill in the art. For example, a HSV Fc receptor or fragment or variant thereof can be tested for its effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a HSV Fc receptor or fragment thereof according to the invention can be assessed. In addition, T helper cell differentiation can be analyzed by measuring proliferation or production of TH1 (IL-2, TNF-α and IFN-γ) cytokines in CD4+ T cells by cytoplasmic cytokine staining and flow cytometry analysis. The HSV Fc receptor or fragment or variant thereof according to the invention can also be tested for ability to induce humoral immune responses, as evidenced, for example, by investigating the activation of B cells in the draining lymph node, by measuring B cell production of antibodies specific for an HSV antigen of interest in the serum. These assays can be conducted using, for example, peripheral B lymphocytes from immunized individuals.

Nucleic Acid

In a further aspect, the invention provides a nucleic acid encoding a HSV1 or HSV2 Fc receptor or immunogenic fragment or variant thereof for use in inducing a cross reactive immune response against HSV when administered to a subject.

The term “nucleic acid” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus, the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap. Nucleic acid molecules as disclosed herein can take various forms (e.g. single-stranded, double-stranded). Nucleic acid molecules may be circular or branched, but will generally be linear. Preferably, the nucleic acid is a RNA molecule. Most preferably, the RNA is a self-amplifying RNA molecule.

The nucleic acids used herein are preferably provided in purified or substantially purified form i.e. substantially free from other nucleic acids (e.g. free from naturally-occurring nucleic acids), generally being at least about 50% pure (by weight), and usually at least about 90% pure.

The nucleic acid molecules of the invention may be produced by any suitable means, including recombinant production, chemical synthesis, or other synthetic means. Suitable production techniques are well known to those of skill in the art. Typically, the nucleic acids of the invention will be in recombinant form, i.e. a form which does not occur in nature. For example, the nucleic acid may comprise one or more heterologous nucleic acid sequences (e.g. a sequence encoding another antigen and/or a control sequence such as a promoter or an internal ribosome entry site) in addition to the nucleic acid sequences encoding the HSV Fc receptor or fragment thereof or heterodimer. The sequence or chemical structure of the nucleic acid may be modified compared to naturally-occurring sequences which encode the HSV Fc receptor or fragment thereof or heterodimer. The sequence of the nucleic acid molecule may be modified, e.g. to increase the efficacy of expression or replication of the nucleic acid, or to provide additional stability or resistance to degradation.

The nucleic acid molecule encoding the HSV Fc receptor or fragment or variant thereof may be codon optimized. Herein, “codon optimized” is intended to refer to modification with respect to codon usage that may increase translation efficacy and/or half-life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O] N), which may further increase translation efficacy.

The nucleic acids may comprise one or more nucleotide analogues or modified nucleotides. As used herein, “nucleotide analogue” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)). A nucleotide analogue can contain further chemical modifications in or on the sugar moiety of the nucleoside (e.g., ribose, deoxyribose, modified ribose, modified deoxyribose, six-membered sugar analogue, or open-chain sugar analogue), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

Modified nucleobases which can be incorporated into modified nucleosides and nucleotides and be present in RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′lm (1,2′-O-dimethylinosine); m3C (3-methylcytidine); Cm (2T-0-methylcytidine); s2C (2-thiocytidine); ac4C (N4-acetylcytidine); £5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5 s2U (5-methylaminomethyl-2-thiouridine); mnm5 se 2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyl uridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); m62Am (N6,N6,0-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); £5 Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-Ce)-alkyluracil, 5-methyluracil, 5-(C2-C6)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

Exemplary effective amounts of a nucleic acid component can be between 1 ng and 100 μg, such as between 1 ng and 1 μg (e.g., 100 ng-1 μg), or between 1 μs and 100 μg, such as 10 ng, 50 ng, 100 ng, 150 ng, 200 ng, 250 ng, 500 ng, 750 ng, or 1 μg. Effective amounts of a nucleic acid can also include from 1 μg to 500 μg, such as between 1 μg and 200 μg, such as between 10 and 100 μg, for example 1 μg, 2 μg, 5 μg, 10 μg, 20 μg, 50 μg, 75 μg, 100 μg, 150 μg, or 200 μg. Alternatively, an exemplary effective amount of a nucleic acid can be between 100 μg and 1 mg, such as from 100 μg to 500 μg, for example, 100 μg, 150 μg, 200 μg, 250 μg, 300 μg, 400 μg, 500 μg, 600 μg, 700 μg, 800 μg, 900 lag or 1 mg.

In a preferred embodiment, the nucleic acid encodes a HSV2 or HSV1 Fc receptor or immunogenic fragment or variant thereof that is a heterodimer with a binding partner from HSV or a fragment thereof wherein the expression of the HSV2 or HSV1 FcR or immunogenic fragment or variant thereof is under the control of a subgenomic promoter, suitably the 26S subgenomic promoter shown in SEQ ID NO: 126, or a variant therefrom which is at least 60%, 65%, 70%, 75% 80%, 85%, 90%, 95%, 96%, 97%, 98%, 99% or 99.5% identical thereto.

In a preferred embodiment, the encoded HSV1 or HSV2 FcR or immunogenic fragment or variant thereof and its binding partner or fragment thereof are separated by an internal ribosomal entry site (IRES) sequence. In a preferred embodiment, the IRES sequence is an IRES EV71 sequence. In a preferred embodiment, the IRES sequence comprises or consists of the sequence shown in SEQ ID NO: 127, or a variant therefrom which is at least 60%, 65%, 70%, 75% 80%, 85%, 90%, 95%, 96%, 97%, 98%, 99% or 99.5% identical thereto.

In another embodiment, the sequences encoding the HSV2 or HSV1 FcR or immunogenic fragment or variant thereof and its binding partner or fragment thereof are separated by a 2A “self-cleaving” peptide sequence. In one embodiment, the 2A “self-cleaving” peptide sequence is a GSG-P2A sequence, suitably comprising or consisting of the sequence shown in SEQ ID NO: 124, or a variant therefrom which is at least 60%, 65%, 70%, 75% 80%, 85%, 90%, 95%, 96%, 97%, 98%, 99% or 99.5% identical thereto. In one embodiment, the 2A “self-cleaving” peptide sequence is a F2A sequence, suitably comprising or consisting of the sequence shown in SEQ ID NO: 125, or a variant therefrom which is at least 60%, 65%, 70%, 75% 80%, 85%, 90%, 95%, 96%, 97%, 98%, 99% or 99.5% identical thereto.

In yet another embodiment, the sequences encoding the HSV2 or HSV1 FcR or immunogenic fragment or variant thereof and its binding partner or fragment thereof are separated by a subgenomic promoter. In one embodiment, the subgenomic promoter is a 26S subgenomic promoter, suitably comprising or consisting of the sequence shown in SEQ ID NO: 126, or a variant therefrom which is at least 60%, 65%, 70%, 75% 80%, 85%, 90%, 95%, 96%, 97%, 98%, 99% or 99.5% identical thereto.

The nucleic acid molecule for the use of the invention may, for example, be RNA or DNA, such as a plasmid DNA. In a preferred embodiment, the nucleic acid molecule is an RNA molecule. In a more preferred embodiment, the RNA molecule is a self-amplifying RNA molecule (“SAM”).

Self-amplifying (or self-replicating) RNA molecules are well known in the art and can be produced by using replication elements derived from, e.g., alphaviruses, and substituting the structural viral proteins with a nucleotide sequence encoding a protein of interest. A self-amplifying RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells. One suitable system for achieving self-replication in this manner is to use an alphavirus-based replicon. These replicons are +-stranded RNAs which lead to translation of a replicase (or replicase-transcriptase) after delivery to a cell. The replicase is translated as a polyprotein which auto-cleaves to provide a replication complex which creates genomic-strand copies of the +-strand delivered RNA. These −-strand transcripts can themselves be transcribed to give further copies of the +-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see WO2005/113782.

In one embodiment, the self-amplifying RNA molecule described herein encodes a RNA-dependent RNA polymerase which can transcribe RNA from the self-amplifying RNA molecule and the HSV Fc receptor or fragment or variant thereof or heterodimer. The polymerase can be an alphavirus replicase e.g. comprising one or more of alphavirus proteins nsP1, nsP2, nsP3 and nsP4.

In a preferred embodiment, the self-amplifying RNA molecule is an alphavirus-derived RNA replicon.

Whereas natural alphavirus genomes encode structural virion proteins in addition to the non-structural replicase polyprotein, in certain embodiments, the self-amplifying RNA molecules do not encode alphavirus structural proteins. Thus, the self-amplifying RNA can lead to the production of genomic RNA copies of itself in a cell, but not to the production of RNA-containing virions. In some aspects, the self-amplifying RNA is not virion-encapsulated. The inability to produce these virions means that, unlike a wild-type alphavirus, the self-amplifying RNA molecule cannot perpetuate itself in infectious form. The alphavirus structural proteins which are necessary for perpetuation in wild-type viruses are absent from self-amplifying RNAs of the present disclosure and their place is taken by gene(s) encoding the immunogen of interest, such that the subgenomic transcript encodes the immunogen rather than the structural alphavirus virion proteins. Thus, a self-amplifying RNA molecule useful with the invention may have two open reading frames. The first (5′) open reading frame encodes a replicase; the second (3′) open reading frame encodes an antigen. In some embodiments the RNA may have additional (e.g. downstream) open reading frames e.g. to encode further antigens or to encode accessory polypeptides.

Suitably, the self-amplifying RNA molecule disclosed herein has a 5′ cap (e.g. a 7-methylguanosine) which can enhance in vivo translation of the RNA. A self-amplifying RNA molecule may have a 3′ poly-A tail. It may also include a poly-A polymerase recognition sequence (e.g. AAUAAA) near its 3′ end. Self-amplifying RNA molecules can have various lengths but they are typically 5000-25000 nucleotides long. Self-amplifying RNA molecules will typically be single-stranded.

Suitably, the self-replicating RNA comprises or consists of a VEEV TC-83 replicon encoding from 5′ to 3′ viral nonstructural proteins 1-4 (nsP1-4), followed by a subgenomic promoter, and a construct (or insert) encoding the gEgI heterodimer. In a preferred embodiment, the insert comprises or consists of a gE ectodomain sequence under the control of the subgenomic promoter mentioned above, followed by an IRES regulatory sequence, followed by a gI ectodomain sequence. In a preferred embodiment, the IRES sequence is an IRES EV71 sequence. In a preferred embodiment, the IRES sequence comprises or consists of the sequence shown in SEQ ID NO: 127, or a variant therefrom which is at least 60%, 65%, 70%, 75% 80%, 85%, 90%, 95%, 96%, 97%, 98%, 99% or 99.5% identical thereto.

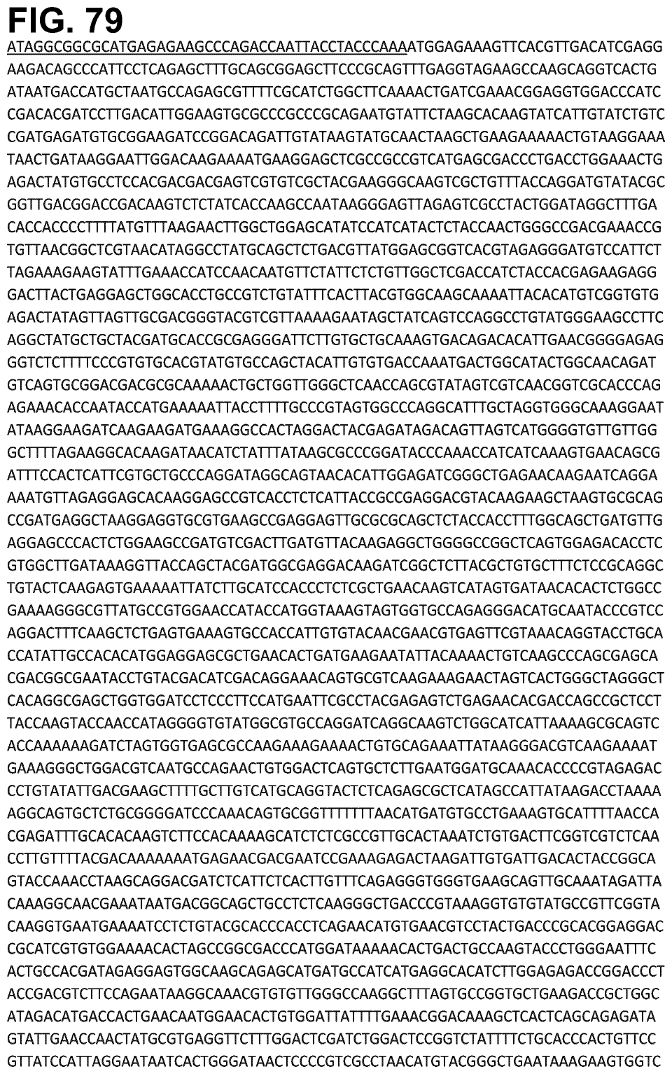

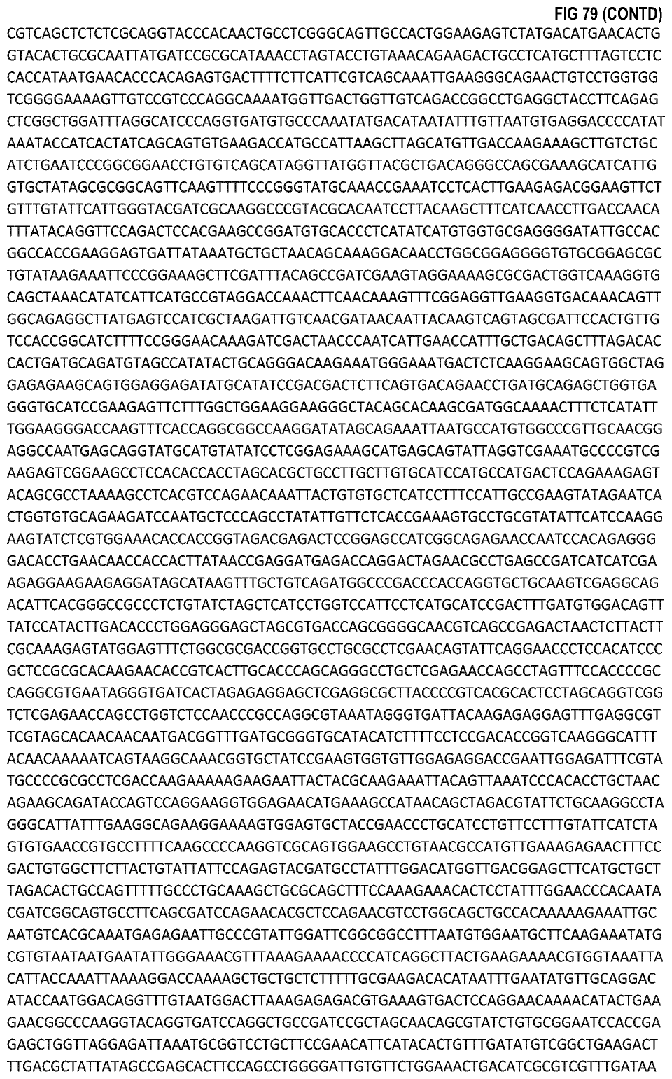

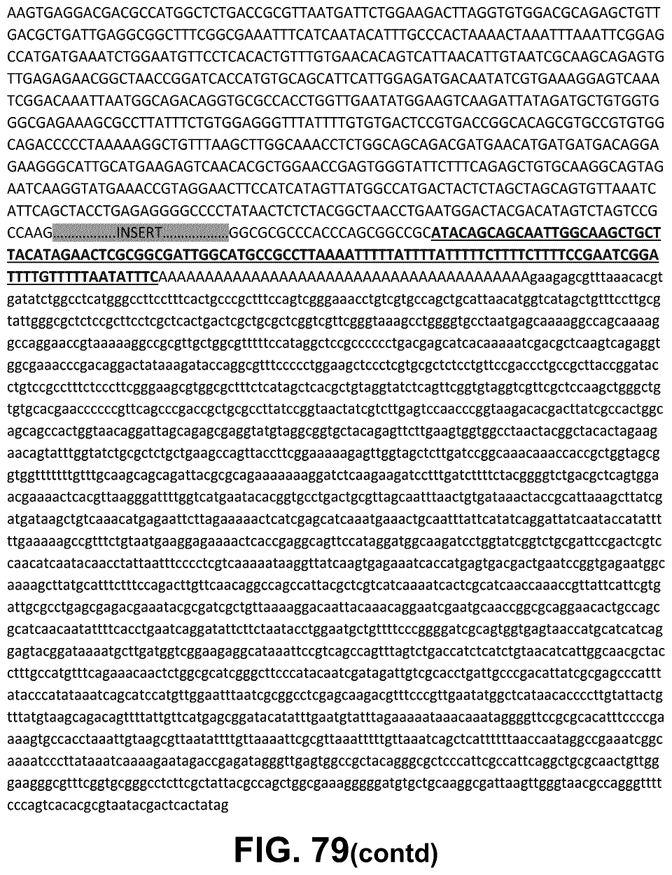

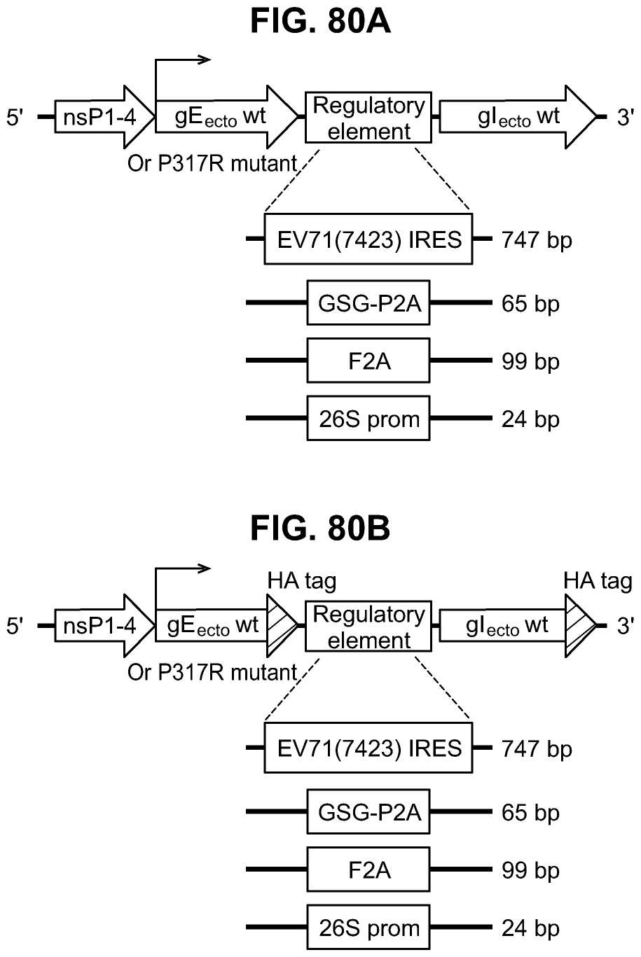

A DNA encoding an empty SAM is shown in FIG. 79 and SEQ ID NO:130; the corresponding empty SAM is shown in SEQ ID NO:133. A construct would be inserted immediately after nucleotide 7561. Thus, a SAM may comprise three regions from 5′ to 3′, the first region comprising the sequence up to the insertion point (for instance nucleotides 1-7561 of SEQ ID NO:133, herein SEQ ID NO:134), the second region comprising an insert encoding a gEgI heterodimer, and the third region comprising the sequence after the insertion point (for instance nucleotides 7562-7747 of SEQ ID NO:133, herein SEQ ID NO:135). Thus, a DNA encoding a SAM may comprise three regions from 5′ to 3′, the first region comprising the sequence up to the insertion point (for instance nucleotides 1-7561 of SEQ ID NO:130, herein SEQ ID NO:131), the second region comprising an insert encoding a gEgI hetrerodimer, and the third region comprising the sequence after the insertion point (for instance nucleotides 7562-9993 of SEQ ID NO:130, herein SEQ ID NO:132).

In one embodiment, the VEEV TC-83 replicon has the DNA sequence shown in FIG. 79 and SEQ ID NO:130, and the construct encoding the gEgI heterodimer antigen is inserted immediately after residue 7561. In one embodiment, the VEE TC-83 replicon has the RNA sequence shown in SEQ ID NO:133, and the construct encoding the antigen is inserted immediately after residue 7561.

The self-amplifying RNA can conveniently be prepared by in vitro transcription (IVT). IVT can use a (cDNA) template created and propagated in plasmid form in bacteria or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-amplifying RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-amplifying RNA can include (in addition to any 5′ cap structure) one or more nucleotides having a modified nucleobase. An RNA used with the invention ideally includes only phosphodiester linkages between nucleosides, but in some embodiments, it can contain phosphoramidate, phosphorothioate, and/or methylphosphonate linkages.

The nucleic acid molecule of the invention may be associated with a viral or a non-viral delivery system. The delivery system (also referred to herein as a delivery vehicle) may have an adjuvant effects which enhance the immunogenicity of the encoded viral Fc receptor or fragment thereof or heterodimer. For example, the nucleic acid molecule may be encapsulated in liposomes, non-toxic biodegradable polymeric microparticles or viral replicon particles (VRPs), or complexed with particles of a cationic oil-in-water emulsion. In some embodiments, the nucleic acid molecule is associated with a non-viral delivery material such as to form a cationic nano-emulsion (CNE) delivery system or a lipid nanoparticle (LNP) delivery system. In some embodiments, the nucleic acid molecule is associated with a non-viral delivery system, i.e., the nucleic acid molecule is substantially free of viral capsid. Alternatively, the nucleic acid molecule may be associated with viral replicon particles. In other embodiments, the nucleic acid molecule may comprise a naked nucleic acid, such as naked RNA (e.g. mRNA).

In a preferred embodiment, the RNA molecule or self-amplifying RNA molecule is associated with a non-viral delivery material, such as to form a cationic nanoemulsion (CNE) or a lipid nanoparticle (LNP). Most preferably, the RNA molecule or self-amplifying RNA molecule is encapsulated in lipid nanoparticles.

CNE delivery systems and methods for their preparation are described in WO2012/006380. In a CNE delivery system, the nucleic acid molecule (e.g. RNA) which encodes the antigen is complexed with a particle of a cationic oil-in-water emulsion. Cationic oil-in-water emulsions can be used to deliver negatively charged molecules, such as an RNA molecule to cells. The emulsion particles comprise an oil core and a cationic lipid. The cationic lipid can interact with the negatively charged molecule thereby anchoring the molecule to the emulsion particles. Further details of useful CNEs can be found in WO2012/006380; WO2013/006834; and WO2013/006837 (the contents of each of which are incorporated herein in their entirety).

Thus, in one embodiment, an RNA molecule, such as a self-amplifying RNA molecule, encoding the HSV Fc receptor or fragment or variant thereof or heterodimer may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. In some embodiments, the CNE comprises squalene and a cationic lipid, such as 1,2-dioleoyloxy-3-(trimethylammonio)propane (DOTAP). In some preferred embodiments, the delivery system is a non-viral delivery system, such as CNE, and the nucleic acid molecule comprises a self-amplifying RNA (mRNA). This may be particularly effective in eliciting humoral and cellular immune responses.

LNP delivery systems and non-toxic biodegradable polymeric microparticles, and methods for their preparation are described in WO2012/006376 (LNP and microparticle delivery systems); Geall et al. (2012) PNAS USA. September 4; 109(36): 14604-9 (LNP delivery system); and WO2012/006359 (microparticle delivery systems). LNPs are non-virion liposome particles in which a nucleic acid molecule (e.g. RNA) can be encapsulated. The particles can include some external RNA (e.g. on the surface of the particles), but at least half of the RNA (and ideally all of it) is encapsulated. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

Dosages will depend primarily on factors such as the route of administration, the condition being treated, the age, weight and health of the subject, and may thus vary among subjects. For example, a therapeutically effective adult human dosage of the nucleic acid of the invention may contain 0.5 to 50 μg, for example 1 to 30 μg, e.g. about 1, 3, 5, 10, 15, 20, 25 or 30 μg of the nucleic acid.

Vectors, Host Cells

Described herein is a vector comprising a nucleic acid for use according to the invention.

A vector may be any suitable nucleic acid molecule including naked DNA or RNA, a plasmid, a virus, a cosmid, phage vector such as lambda vector, an artificial chromosome such as a BAC (bacterial artificial chromosome), or an episome. Alternatively, a vector may be a transcription and/or expression unit for cell-free in vitro transcription or expression, such as a T7-compatible system. The vectors may be used alone or in combination with other vectors such as adenovirus sequences or fragments, or in combination with elements from non-adenovirus sequences. Suitably, the vector has been substantially altered (e.g., having a gene or functional region deleted and/or inactivated) relative to a wild type sequence, and replicates and expresses the inserted polynucleotide sequence, when introduced into a host cell.

Also described herein is a cell comprising a nucleic acid encoding a HSV2 or HSV1 Fc receptor or fragment or variant thereof.

The encoded HSV Fc receptor or immunogenic fragment or variant thereof, or the encoded HSV FcR binding partner or fragment thereof for use according to the invention are suitably produced by recombinant technology. “Recombinant” means that the polynucleotide is the product of at least one of cloning, restriction or ligation steps, or other procedures that result in a polynucleotide that is distinct from a polynucleotide found in nature. A recombinant vector is a vector comprising a recombinant polynucleotide.

In one embodiment, a heterodimer as described herein is expressed from a multicistronic vector. Suitably, the heterodimer is expressed from a single vector in which the nucleic sequences encoding the HSV FcR or immunogenic fragment or variant thereof and its binding partner or fragment thereof are separated by an internal ribosomal entry site (IRES) sequence (Mokrejš, Martin, et al. “IRESite: the database of experimentally verified IRES structures (www.iresite.org).” Nucleic acids research 34.suppl_1 (2006): D125-D130.). In a preferred embodiment, the IRES is an IRES EV71 sequence.

In a preferred embodiment, the IRES comprises or consists of the sequence shown in SEQ ID NO: 127, or a variant therefrom which is at least 60%, 65%, 70%, 75% 80%, 85%, 90%, 95%, 96%, 97%, 98%, 99% or 99.5% identical thereto.

Alternatively, the two nucleic sequences can be separated by a viral 2A or ‘2A-like’ sequence, which results in production of two separate polypeptides. 2A sequences are known from various viruses, including foot-and-mouth disease virus, equine rhinitis A virus, Thosea asigna virus, and porcine theschovirus-1. See e.g., Szymczak et al., Nature Biotechnology 22:589-594 (2004), Donnelly et al., J Gen Virol.; 82(Pt 5): 1013-25 (2001).

Optionally, to facilitate expression and recovery, the encoded HSV2 or HSV1 Fc receptor or immunogenic fragment or variant thereof and/or the HSV FcR binding partner or fragment thereof may include a signal peptide at the N-terminus. A signal peptide can be selected from among numerous signal peptides known in the art, and is typically chosen to facilitate production and processing in a system selected for recombinant expression. In one embodiment, the signal peptide is the one naturally present in the native HSV Fc protein or binding partner. The signal peptide of the HSV2 gE from strain SD90e is located at residues 1-20 of SEQ ID NO:1. Signal peptide for gE proteins from other HSV strains can be identified by sequence alignment. The signal peptide of the HSV2 gI from strain SD90e is located at residues 1-20 of SEQ ID NO:2. Signal peptide for gI proteins from other HSV strains can be identified by sequence alignment.

The nucleic acids described herein may be purified, for example purified following an in vitro transcription procedure or following production in bacterial cells. The term “purification” or “purifying” refers to the process of removing components from a composition or host cell or culture, the presence of which is not desired. Purification is a relative term, and does not require that all traces of the undesirable component be removed from the composition. In the context of vaccine production, purification includes such processes as centrifugation, dialyzation, ion-exchange chromatography, and size-exclusion chromatography, affinity-purification or precipitation. Thus, the term “purified” does not require absolute purity; rather, it is intended as a relative term. A preparation of substantially pure nucleic acid or protein can be purified such that the desired nucleic acid, or protein, represents at least 50% of the total nucleic acid content of the preparation. In certain embodiments, a substantially pure nucleic acid, or protein, will represent at least 60%, at least 70%, at least 80%, at least 85%, at least 90%, or at least 95% or more of the total nucleic acid or protein content of the preparation. Immunogenic molecules or antigens or antibodies which have not been subjected to any purification steps (i.e., the molecule as it is found in nature) are not suitable for pharmaceutical (e.g., vaccine) use.

Sequence Comparison

For the purposes of comparing two closely-related polynucleotide or polypeptide sequences, the “sequence identity” or “% identity” between a first sequence and a second sequence may be calculated using an alignment program, such as BLAST® (available at blast.ncbi.nlm.nih.gov, last accessed 12 Sep. 2016) using standard settings. The percentage identity is the number of identical residues divided by the length of the alignment, multiplied by 100. An alternative definition of identity is the number of identical residues divided by the number of aligned residues, multiplied by 100. Alternative methods include using a gapped method in which gaps in the alignment, for example deletions in one sequence relative to the other sequence, are considered. Polypeptide or polynucleotide sequences are said to be identical to other polypeptide or polynucleotide sequences, if they share 100% sequence identity over their entire length.

A “difference” between two sequences refers to an insertion, deletion or substitution, e.g., of a single amino acid residue in a position of one sequence, compared to the other sequence.

For the purposes of comparing a first, reference polypeptide sequence to a second, comparison polypeptide sequence, the number of additions, substitutions and/or deletions made to the first sequence to produce the second sequence may be ascertained. An addition is the addition of one amino acid residue into the sequence of the first polypeptide (including addition at either terminus of the first polypeptide). A substitution is the substitution of one amino acid residue in the sequence of the first polypeptide with one different amino acid residue. A deletion is the deletion of one amino acid residue from the sequence of the first polypeptide (including deletion at either terminus of the first polypeptide).

Suitably substitutions in the sequences of the present invention may be conservative substitutions. A conservative substitution comprises the substitution of an amino acid with another amino acid having a physico-chemical property similar to the amino acid that is substituted (see, for example, Stryer et al, Biochemistry, 5th Edition 2002, pages 44-49). Preferably, the conservative substitution is a substitution selected from the group consisting of: (i) a substitution of a basic amino acid with another, different basic amino acid; (ii) a substitution of an acidic amino acid with another, different acidic amino acid; (iii) a substitution of an aromatic amino acid with another, different aromatic amino acid; (iv) a substitution of a non-polar, aliphatic amino acid with another, different non-polar, aliphatic amino acid; and (v) a substitution of a polar, uncharged amino acid with another, different polar, uncharged amino acid. A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of alanine, valine, leucine, methionine and isoleucine. A polar, uncharged amino acid is preferably selected from the group consisting of serine, threonine, cysteine, proline, asparagine and glutamine.

In contrast to a conservative amino acid substitution, a non-conservative amino acid substitution is the exchange of one amino acid with any amino acid that does not fall under the above-outlined conservative substitutions (i) through (v).

Terms

“Encoding” refers to the inherent property of specific sequences of nucleotides in a polynucleotide, to act as a template for synthesis of other polymers and macromolecules in biological processes, e.g., synthesis of peptides or proteins. Both the coding strand of a double-stranded nucleotide molecule (the sequence of which is usually provided in sequence listings), and the non-coding strand (used as the template for transcription of a gene or cDNA), can be referred to as encoding the peptide or protein. Unless otherwise specified, as used herein a “nucleotide sequence encoding an amino acid sequence” includes all nucleotide sequences that are degenerate versions of each other and that encode the same amino acid sequence.

The term “expression” or “expressing” as used herein is defined as the transcription and/or translation of a particular nucleotide sequence driven by its operably linked promoter.

Unless otherwise explained in the context of this disclosure, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof.

Amino acid sequences provided herein are designated by either single-letter or three-letter nomenclature, as is known in the art (see, e.g., Eur. J. Biochem. 138:9-37(1984)).

Although methods and materials similar or equivalent to those described herein can be used in the practice or testing of this disclosure, suitable methods and materials are described below.

The present invention will now be further described by means of the following non-limiting examples.

### EXAMPLES

### Example 1

The general objective of this study was to generate immunogenicity data with five new recombinant HSV-2 gE/gI proteins containing mutations in gE Fc binding domain to drop the avidity to human Immunoglobulin G Fc.

The primary objective was to compare to NaCl control group, the anti-HSV-2 gE-specific polyclonal antibody (pAb) response induced by several AS01-adjuvanted recombinant HSV-2 gE/gI proteins containing mutations in the amino acid sequence14 days post third immunization.

As secondary objectives, 14 days post third immunization, the anti-HSV-2 gI-specific polyclonal antibody (pAb) response and the anti-HSV-2 gE/gI-specific CD4+T cell responses induced by different mutants of HSV-2 gE/gI protein were compared to NaCl control group. In addition, head-to-head comparison of all vaccine candidates in term of anti-HSV-2 gE and anti-HSV-2 gI-specific IgG antibody responses and CD4+ T cell responses were performed 14 days post third immunization.

Finally, the exploratory objectives were to evaluate, in different groups of mice immunized with HSV-2 gE/gI mutants, the anti-HSV-2 gE and the anti-HSV-2 gI-specific antibody responses after one and two immunizations, the anti-HSV-1 gE/gI cross-reactive antibody response after the three immunizations, the functions of vaccine-specific and HSV-1 gE/gI cross reactive polyclonal antibodies after three immunizations and the levels of anti-HSV-2 gE- and anti-HSV-2 gI-specific CD8+T cell responses after the third immunization.

Introduction

Herpesviruses have evolved diverse immune evasion strategies to survive in their natural hosts. Human Simplex Viruses (HSVs) encode namely the glycoprotein E (gE), that targets the humoral immune system. gE can form a noncovalent heterodimer complex with glycoprotein I (gI) that functions as an immunoglobulin G (IgG) Fc receptor (FcγR). Interactions between gE and gI increase human Fc binding affinity by about 100 times. Through antibody binding, the FcγR inhibits IgG Fc-mediated activities, including complement binding and antibody-dependent cellular cytotoxicity.

Previous experiments performed in mice showed that human IgG could bind to the gE Fc binding domain of the unmutated recombinant HSV-2 gE/gI protein and mask protective epitopes implicated in HSV-2 gE-mediated immune evasion mechanism. These results suggested that a similar situation could happen in clinic and negatively impact the HSV-2 vaccine therapeutic efficacy. To avoid negative immunological interference in humans, point mutations have been performed within the Fc binding domain of HSV-2 gE/gI protein to reduce the affinity to hIgG Fc domain and the HSV-2 gE-P317R/gI mutant construct was generated. Further constructs containing mutations in the gE Fc binding domain have been generated and their immunogenicity assessed. In particular, the immunogenicity in mice of five different HSV-2 gE/gI mutant proteins formulated in AS01 has been assessed.

Study Design

Female CB6F1 inbred mice aged 6-8 weeks old from Harlan laboratory (OlaHsd) were randomly assigned to the study groups (n=6 gr1-5 & n=4/gr6) and kept at the institutional animal facility under specified pathogen-free conditions. CB6F1 mice (gr1-5) were intramuscularly (i.m) immunized at days 0, 14 & 28 with 5 μg of different HSV-2 gE/gI mutant proteins formulated in AS01 (5 μg). An additional group of mice was i.m injected with a saline solution (NaCl 150 mM), following the same schedule of immunization, and used as negative control group (gr6).

Serum samples were collected at days 14, 28 & 42 post prime immunization (14PI, 14PII, 14PIII) to measure anti-HSV-2 gE- and anti-HSV-2 gI-specific and anti-HSV-1 gE/gI cross-reactive IgG antibody responses and to characterize the function of vaccine-specific and cross reactive polyclonal antibodies. Spleens were collected 14 days post third immunization (14PIII) to evaluate ex-vivo systemic CD4+/CD8+ T cell responses towards HSV-2 gE and gI antigens. Details for each group is described in the Table 3.

TABLE 3
summary of study design and formulation tested

| | | Treatment | | | | | | |
|---|---|---|---|---|---|---|---|---|
| | Number | | | | | Volume and | Immunization | Sample collection |
| | of | Vaccine name | Lot | Adjuvant dose | Adjuvant | route of | schedule | (organs, blood |
| Group | animals | and dose | numbers | (if applicable) | lot number | injection | (Days) | volume) and days |
| 1 | 6 | HSV-2 | BMP1271 | AS01 | LIQ- | 50 μL | Days 0, 14 & 28 | Days 14 & |
| | | gE/gI V340W | | | 2019/2014 | i.m | | 28: Serum - |
| | | (5 μg) | | | | | | partial bleeding |
| 2 | 6 | HSV-2 | BMP1272 | | | | | Day 42: Serum - |
| | | gE/gI A248T | | | | | | final bleeding + |
| | | (5 μg) | | | | | | Spleen |
| 3 | 6 | HSV-2 | BMP1278 | | | | | |
| | | gE/gI A246W | | | | | | |
| | | (5 μg) | | | | | | |
| 4 | 6 | HSV-2 | BMP1279 | | | | | |
| | | gE/gI P318I | | | | | | |
| | | (5 μg) | | | | | | |
| 5 | 6 | HSV-2 | BMP1273 | | | | | |
| | | gE/gI A248T_V340W | | | | | | |
| | | (5 μg) | | | | | | |
| 6 | 4 | NaCl150 mM | N/A | N/A | | | | |

Materials and Methods

The antigens used in this study were the ectodomain of the purified recombinant gE/gI mutant proteins. The material was produced by using ExpiCHO™ expression system and was used as a research lot only.

AS01 is a liposome based adjuvant system (AS) containing QS-21 (a triterpene glycoside purified from the bark of Quillaja saponaria) and MPL (3-D Monophosphoryl lipid A), with liposomes as vehicles for these immunoenhancers and a buffer including NaCl as isotonic agent (variant 3).

A single human dose of the AS01b Adjuvant System (0.5 mL) contains 50 μg of QS-21 and 50 μg of MPL. In this study, the volume injected in mice is 1/10^{th} of a human dose corresponding to a 5 μg QS-21 and 5 μg MPL per dose.

Animal Model

It is well accepted that small animal models are useful tools to study the immunogenicity profile of new vaccine candidates. In order to get a general overview of the capacity of our vaccine candidates to induce T cell immune responses, CB6F1 mice (hybrid of C57B1/6 and Balb/C mice) have been used in this study.

CB6F1 mice have been shown to generate potent CD4+/CD8+ T cell and humoral immune responses following vaccination with various types of immunogens, including adjuvanted proteins and viral vectors. The profile of the vaccine-induced immune responses generated in these mice compared to expected responses in humans may nevertheless be impacted by some differences pertaining to TLR expression, HLA background and antigen presentation. However, the capacity for inducing CD4+/CD8+ T immune responses has shown comparable trends between these mice and humans.

Detection of Total Anti-HSV-2 gE & gI Specific IgG Antibodies by ELISA

Quantification of the total anti-gE or gI specific IgG antibodies was performed using indirect ELISA. Recombinant gE (˜51 kDa) (BMP1049 or BMP1291) or gI protein (˜46 kDa) (BMP1052b or BMP1292) from HSV-2 were used as coating antigens. These proteins were produced using the ExpiHEK293F™ expression system.

Polystyrene 96-well ELISA plate (Nunc F96 Maxisorp cat 439454) were coated with 100 μL/well of antigen diluted at a concentration of 2 μg/mL (gE) and 1 μg/mL (gI) in carbonate/bicarbonate 50 mM pH 9.5 buffer and incubated overnight at 4° C. After incubation, the coating solution was removed and the plates were blocked with 200 μL/well of Difkomilk 10% diluted in PBS (blocking buffer) (ref 232100, Becton Dickinson, USA) for 1 h at 37° C. After incubation, the blocking solution was removed and a three-fold serial dilution (in PBS+0.1% Tween20+1% BSA buffer, of each serum sample was performed and added to the coated plates and incubated for 1 h at 37° C. The plates were then washed four times with PBS 0.1% Tween20 (washing buffer) and Horseradish Peroxydase conjugated AffiniPure Goat anti-mouse IgG (H+L) (ref 115-035-003, Jackson, USA) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 15 min at RT (room temperature) with 100 μL/well of a solution of 75% single-component TMB Peroxidase ELISA Substrate (ref 172-1072, Bio-Rad, USA) diluted in sodium Citrate 0.1M pH5.5 buffer. Enzymatic color development was stopped with 100 μL of 0.4N Sulfuric Acid 1M (H2SO4) per well and the plates were read at an absorbance of 450/620 nm using the Versamax ELISA reader.

Optical densities (OD) were captured and analysed using the SoftMaxPro GxP v5.3 software. A standard curve was generated by applying a 4-parameter logistic regression fit to the reference standard results (reference standard anti-gE=20180011 14PIII—Pool of mice 1.1 to 1.20 immunized with AS01/gE (5 μg of each/dose); reference standard anti-gI=20190021 14PII—Pool of mice 2.1 to 2.5 immunized with AS01/gI (5 μg of each/dose). Antibody titer in the samples was calculated by interpolation of the standard curve. The antibody titer of the samples was obtained by averaging the values from dilutions that fell within the 20-80% dynamic range of the standard curve. Titers were expressed in EU/mL (ELISA Units per mL).

Detection of Total Anti-HSV-1 gE/gI Cross-Reactive IgG Antibodies by ELISA

Quantification of the total HSV-1 gE/gI cross-reactive IgG antibodies was performed using indirect ELISA. Recombinant gE/gI heterodimer protein (BMP1299) from HSV-1 were used as coating antigen. This protein was produced using the ExpiCHO™ expression system.

Polystyrene 96-well ELISA plate (Nunc F96 Maxisorp cat 439454) were coated with 100 μL/well of recombinant HSV-1 gE/gI heterodimer protein diluted at a concentration of 2 μg/mL in carbonate/bicarbonate 50 mM pH 9.5 buffer and incubated overnight at 4° C. After incubation, the coating solution was removed and the plates were blocked with 200 μg/well of Difkomilk 10% diluted in PBS (blocking buffer) (ref 232100, Becton Dickinson, USA) for 1 h at 37° C. After incubation, the blocking solution was removed and a three-fold serial dilution (in PBS+0.1% Tween20+1% BSA buffer) of each serum sample was performed and added to the coated plates and incubated for 1 h at 37° C. The plates were then washed four times with PBS 0.1% Tween20 (washing buffer) and Horseradish Peroxidase conjugated AffiniPure Goat anti-mouse IgG (H+L) (ref 115-035-003, Jackson, USA) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 15 min at RT (room temperature) with 100 μL/well of a solution of 75% single-component TMB Peroxidase ELISA Substrate (ref 172-1072, Bio-Rad, USA) diluted in sodium Citrate 0.1M pH5.5 buffer. Enzymatic color development was stopped with 100 μL of 0.4N Sulfuric Acid 1M (H₂SO₄) per well and the plates were read at an absorbance of 450/620 nm using the Versamax ELISA reader.

Optical densities (OD) were captured and analysed using the SoftMaxPro GxP v5.3 software. A standard curve was generated by applying a 4-parameter logistic regression fit to the reference standard results (reference standard=20200023 14PIII—Pool of mice 1.1 to 1.20 immunized with AS01-HSV-1 gE/gI HEK (5 μg of each/dose). Antibody titer in the samples was calculated by interpolation of the standard curve. The antibody titer of the samples was obtained by averaging the values from dilutions that fell within the 20-80% dynamic range of the standard curve. Titers were expressed in EU/mL (ELISA Units per mL).

Anti-HSV-2 gE and gI-Specific CD4+/CD8+ T Cell Responses Measured by ICS Assay

The frequencies of anti-HSV-2 gE & anti-HSV-2 gI-specific CD4+ and CD8+ T-cells producing IL-2 and/or IFN-γ and/or TNF-α were evaluated in splenocytes collected 14 days post third immunization after ex-vivo stimulation with HSV-2 gE or gI peptides pools.

Isolation of Splenocytes

Spleens were collected from individual mouse 14 days after third immunization and placed in RPMI 1640 medium supplemented with RPMI additives (Glutamine, Penicillin/streptomycin, Sodium Pyruvate, non-essential amino-acids & 2-mercaptoethanol) (=RPMI/additives). Cell suspensions were prepared from each spleen using a tissue grinder. The splenic cell suspensions were filtered (cell stainer100 μm) and then the filter was rinsed with 40 mL of cold PBS-EDTA 2 mM. After centrifugation (335 g, 10 min at 4° C.), cells were resuspended in 7 mL of cold PBS-EDTA 2 mM. A second washing step was performed as previously described and the cells were finally resuspended in 1 mL of RPMI/additives supplemented with 5% FCS (Capricorn scientific, FBS-HI-12A). Cell suspensions were then diluted 20× (10 μL) in PBS buffer (190 μL) for cell counting (using MACSQuant Analyzer). After counting, cells were centrifuged (335 g, 10 min at RT) and resuspended at 10⁷ cells/mL in RPMI/additives supplemented with 5% FCS.

Cell Preparation

Fresh splenocytes were seeded in round bottom 96-well plates at 10⁶ cells/well (100 μL). The cells were then stimulated for 6 hours (37° C., 5% CO2) with anti-CD28 (BD Biosciences, clone 37.51) and anti-CD49d antibodies (BD Biosciences, clone 9C10 (MFR4.B)) at 1 μg/mL per well, containing 100 μL of either:—
15 mers overlapping peptide pool covering the sequence of gE protein from HSV-2 (1 μg/mL per peptide per well).
15 mers overlapping peptide pool covering the sequence of gI protein from HSV-2 (1 μg/mL per peptide per well).
15 mers overlapping peptide pool covering the sequence of Human β-actin protein (1 μg/mL per peptide per well) (irrelevant stimulation).
RPMI/additives medium (as negative control of the assay).
PMA—ionomycin solution (Sigma, P8139) at working concentrations of 0.25 μg/mL and 2.5 μg/mL respectively (as positive control of the assay).

After 2 hours of ex vivo stimulation, Brefeldin A (Golgi plug ref 555029, BD Bioscience) diluted 1/200 in RPMI/additives supplemented with 5% FCS was added for 4 additional hours to inhibit cytokine secretion. Plates were then transferred at 4° C. for overnight incubation.

Intracellular Cytokine Staining

After overnight incubation at 4° C., cells were transferred to V-bottom 96-well plates, centrifuged (189 g, 5 min at 4° C.) and washed in 250 μL of cold PBS+1% FCS (Flow buffer). After a second centrifugation (189 g, 5 min at 4° C.), cells were resuspended to block unspecific antibody binding (10 min at 4° C.) in 50 μL of Flow buffer containing anti-CD16/32 antibodies (BD Biosciences, clone 2.4G2) diluted 1/50. Then, 50 μL Flow Buffer containing mouse anti-CD4-V450 antibodies (BD Biosciences, clone RM4-5, diluted at 1/100), anti-CD8-PerCp-Cy5.5 antibodies (BD Biosciences, clone 53-6.7, diluted at 1/50) and Live/Dead™ Fixable Yellow dead cell stain (Molecular probes, L34959, diluted at 1/500) was added for 30 min in obscurity at 4° C. After incubation, 100 μL of Flow buffer was added into each well and cells were then centrifuged (189g for 5 min at 4° C.). A second washing step was performed with 200 μL of Flow buffer and after centrifugation, cells were fixed and permeabilized by adding 200 μL, of Cytofix-Cytoperm solution (BD Biosciences, 554722) for 20 min at 4° C. in the obscurity. After plates centrifugation (500 g for 5 min at 4° C.), cells were washed with 200 μL of Perm/Wash buffer (BD Biosciences, 554723), centrifuged (500 g for 5 min 4° C.) and resuspended in 50 μL of Perm/Wash buffer containing mouse anti-IL2-FITC (BD Biosciences, clone JES6-5H4, diluted 1/400), anti-IFN-γ-APC (BD Biosciences, clone XMG1.2, diluted 1/200) and anti-TNF-α-PE (BD Biosciences, clone MP6-XT22, diluted 1/700) antibodies, for 1 hour at 4° C. in the obscurity. After incubation, 100 μL of Flow buffer was added into each well and cells were then finally washed with 200 μL of Perm/Wash buffer (centrifugation 500 g for 5 min at 4° C.) and resuspended in 220 μL PBS.

Cell Acquisition and Analysis

Stained cells were analyzed by flow cytometry using a LSRII flow cytometer and the FlowJo software. Live cells were identified with the Live/Dead staining and then lymphocytes were isolated based on Forward/Side Scatter lights (FSC/SSC) gating. The acquisition was performed on ˜20.000 CD4+/CD8+ T-cell events. The percentages of IFN-γ+/−IL-2+/−and TNF-α+/−producing cells were calculated on CD4+ and CD8+ T cell populations. For each sample, unspecific signal detected after medium stimulation was removed from the specific signal detected after peptide pool stimulation.

Evaluation of the Ability of Polyclonal Sera to Bind and Activate Mouse FcγRIII after Incubation with HSV-2 or HSV-1 gE/gI Transfected Cells (ADCC Bioassay—Promega)

The mouse FcγRIII Antibody Dependent Cell Cytotoxicity (ADCC) Reporter Bioassay (Cat. #M1201), developed by Promega laboratory, is a bioluminescent cell-based assay which can be used to measure the ability of antibodies to specifically bind and activate the mouse FcγRIII expressed by modified Jurkat reporter cells.

Briefly, 3T3 cells, initially purchased from ATCC laboratories (clone A31, ATCC ref CCL-163), were grown in DMEM+10% FBS decomplemented+1% L-glutamine 2 mM+1% Penicillin/streptomycin media and seeded at 2×10⁶ cells in T175 flasks on Day 0 of experiment to ensure cells were in optimal growth phase for the next day.

On day 1, 6-well plates were prepared by adding 2 mL of growth media (DMEM (Prep Mil, Log377BA), 1% L-Glutamine 2 mM (Prep Mil, Log010D), 10% of Ultra low IgG FBS (Gibco, A33819-01)) to each well (one well per electroporation). Plates were kept warm in a 37° C. incubator (5% CO2). The electroporator (Gene Pulser, BIO-RAD) was prepared to deliver 325V, 350 μF capacitance, infinite resistance, 1 pulse for a 4 mm cuvette. 3T3 cells in growth phase were harvested into growth media and counted using a cell counter (TC20, BIO-RAD). For each electroporation, 5×10⁵ 3T3 cells (500 μL of cells at 1×10⁶ cells/mL) and 20 μg of HSV-2 gE/gI or HSV-1 gE/gI plasmid DNA (20 μL at 1 μg/μL) was used. For negative control, 10 μL of water was used. Cells and HSV-2 or HSV-1 gE/gI plasmid DNA mixture were transferred to 4 mm cuvette (Gene Pulser Electroporation Cuvettes, BIO-RAD) and immediately subjected to one pulse of electroporation using the parameters described above. After electroporation, all cuvettes were pooled to homogenise cell suspension and 500 μL of cell suspension/well were transferred into 6-well plates in 2 mL of pre-warmed media. Before incubation, the 6-well plates were slid back and forth and side-to-side several times to distribute cells evenly and then incubated at 37° C., 5% CO₂ during 48 h.

After 48 h incubation, HSV-2 or HSV-1 gE/gI transfected 3T3 cells (target cells (T)) were collected and pooled from the different 6-well plates. Cell suspension was centrifuged (10 min, 340 g, at RT) and resuspended in Promega assay buffer (96% RPMI (G7080)+4% of low IgG serum (G7110); Promega) for cell counting (TC20, BIO-RAD). Then a solution at 96.000 3T3 cells/mL was prepared in Promega assay buffer and 254 of this suspension (24.000 cells/25 μL/well) was added in 96-well plates. In a round-bottom 96-well plates (Nunc, ref 168136), a 3-fold serial dilution of each mouse serum sample (starting dilution 1/500 for HSV-2 and 1/200 for HSV-1) in 200 μL was performed in Promega assay buffer and 254 of each dilution was transferred to the corresponding well containing already the HSV-2 or HSV-1 gE/gI-transfected 3T3 cells. Finally, 254 of genetically engineered Jurkat cells expressing mouse FcγRIII (Effector Cells (E)) at a concentration of 240.0000 cells/mL (60.000 cells/25 μL/well) were added in each well (˜E/T 2.5/1) and plates were incubated for 6h at 37° C.—5% CO₂.

After incubation, plates were put at RT for 15 min and 754 of Bio-Glow reagent were added in each well. The plates were finally incubated for 20 min at RT and read using luminometer (BioTek Synergy H1).

Competitive ELISA to Evaluate the Ability of Vaccine-Specific Antibodies to Decrease Human IgG Fc Binding by HSV-2 gE/gI Protein

The ability of polyclonal sera collected in different groups of mice to decrease in-vitro hIgG antibodies binding by recombinant HSV-2 gE/gI protein was investigated by competitive ELISA. Recombinant HSV-2 gE/gI protein (BMP1063), produced using the ExpiHEK293F™ expression system was used as coating antigen.

Polystyrene 96-well ELISA plate (Nunc F96 Maxisorp cat 439454) were coated with 50 μL/well of HSV-2 gE/gI protein diluted at a concentration of 4 μg/mL in free Calcium/Magnesium PBS buffer and incubated overnight at 4° C. After incubation, the coating solution was removed and the plates were blocked with 100 μL/well of PBS supplemented with 0.1% Tween-20+1% BSA (blocking buffer) for 1 h at 37° C.

In a 96-well Clear V-Bottom Polypropylene microplate (Falcon, ref 353263) a two-fold serial dilution (starting dilution 1/10) in blocking buffer for each individual serum was prepared in 60 μl/well and mixed with 60W/well of biotinylated-hIgG antibodies (Invitrogen, ref 12000C) pre-diluted at 0.7 μg/mL in blocking buffer.

Then, after 1 h of incubation with blocking buffer, the blocking solution was removed from the coated plates and 100 μL of the mixture containing both hIgG and mice sera was transferred in the corresponding HSV-2 gE/gI coated wells and incubated 24h at 37° C. Positive control of the assay was a pool of anti-HSV-2 gE/gI serum samples from previous studies. Negative control of the assay was a pool of irrelevant HPV serum samples diluted 1/1000 and mixed with hIgG too.

After 24h of incubation, the plates were washed four times with PBS 0.1%+Tween20 (washing buffer) and 50 μL/well of Streptavidin-horsedish Peroxydase AMDEX (Amersham, ref RPN4401V) diluted 2000× were added on the wells and plates were incubated for 30 min at 37° C. Plates were then washed four times with washing buffer and 50 μL/well of a solution containing 75% single-component TMB Peroxidase ELISA Substrate (ref 172-1072, Bio-Rad, USA) diluted in sodium Citrate 0.1M pH5.5 buffer were added for 10 min at room temperature. Enzymatic color development was stopped with 50 μL/well of 0.4N Sulfuric Acid 1M (H₂SO₄) and the plates were read at an absorbance of 450/620 nm using the Versamax ELISA reader. Optical densities (OD) were captured and fitted in curve in excel program.

Titers were expressed as the effective dilution at which 50% (i.e. ED₅₀) of the signal was achieved by sample dilution.

For each plate and using a reference sample (i.e. irrelevant serum), the reference ED₅₀ value was estimated using the following formula:

ED₅₀=OD_{0%}+0.5*(OD_{100%}-OD_{0%})

where OD_{100%} is the highest OD obtained with similar samples and OD_{0%} is the lowest achievable signal. For each plate, the former was obtained by averaging (mean) 6 replicates while the latter was set at zero.

Samples ED₅₀ titers were computed by way of linear interpolation between the left and right measurements closest to the ED₅₀ estimate within the plate. The approximation was obtained, on the untransformed OD and the logarithm base 10 transformed dilutions, with the approx function of the stats R base package.

Sample were not assigned a titer in the following cases:
no measurement was available above or below the ED₅₀,
curve crossed at least twice the ED₅₀ and
one of the dilution steps (left or right) closest to the ED₅₀ was missing

Competitive ELISA to Evaluate the Ability of HSV-1 Cross-Reactive Antibodies to Decrease Human IgG Fc Binding by HSV-1 gE/gI Protein

The ability of polyclonal sera collected in different groups of mice to decrease in-vitro hIgG antibodies binding by recombinant HSV-1 gE/gI protein was investigated by competitive ELISA. Recombinant HSV-1 gE/gI heterodimer protein (BMP1299), produced using the ExpiCHO™ expression system was used as coating antigen.

Polystyrene 96-well ELISA plate (Nunc F96 Maxisorp cat 439454) were coated with 50 μL/well of HSV-1 gE/gI protein diluted at a concentration of 2 μg/mL in free Calcium/Magnesium PBS buffer and incubated overnight at 4° C. After incubation, the coating solution was removed and the plates were blocked with 100 μL/well of PBS supplemented with 0.1% Tween-20+1% BSA (blocking buffer) for 1 h at 37° C.

In another 96-well Clear V-Bottom Polypropylene microplate (Falcon, ref 353263) a two-fold serial dilution (starting dilution 1/10) in blocking buffer for each serum were prepared in 60 μl/well and mixed with 601.11/well of biotinylated-hIgG antibodies (invitrogen, ref 12000C) pre-diluted at 0.7 μg/mL in blocking buffer.

Then, after 1 h of incubation with blocking buffer, the blocking solution was removed from the coated plates and 100 μL of the mixture containing both hIgG and mice sera was transferred in the corresponding wells and incubated 24h at 37° C. Positive control of the assay was a pool of anti-HSV-1 gE/gI serum samples. Negative control of the assay was a pool of irrelevant HPV serum samples diluted 1/1000 and mix with same concentration hIgG than the one used with the samples.

After 1 h of incubation, the plates were washed four times with PBS 0.1% Tween20 (washing buffer) and 50 μL/well of Steptavidin-horsedish Peroxydase AMDEX (Amersham, ref RPN4401V) diluted 2000× were added on the wells and plates were incubated for 30 min at 37° C. Plates were then washed four times with washing buffer and 50 μL/well of a solution containing 75% single-component TMB Peroxidase ELISA Substrate (ref 172-1072, Bio-Rad, USA) diluted in sodium Citrate 0.1M pH5.5 buffer were added for 10 min at room temperature. Enzymatic color development was stopped with 50 μL/well of 0.4N Sulfuric Acid 1M (H₂SO₄) and the plates were read at an absorbance of 450/620 nm using the Versamax ELISA reader. Optical densities (OD) were captured and fitted in curve in excel program.

Titers were expressed as the effective dilution at which 50% (i.e. ED₅₀) of the signal was achieved by sample dilution.

For each plate and using a reference sample (i.e. irrelevant serum), the reference ED₅₀ value was estimated using the following formula:

ED₅₀=OD_{0%}+0.5*(OD_{100%}−OD_{0%})

where OD_{100%} is the highest OD obtained with similar samples and OD_{0%} is the lowest achievable signal. For each plate, the former was obtained by averaging (mean) 6 replicates while the latter was set at zero.

Samples ED₅₀ titers were computed by way of linear interpolation between the left and right measurements closest to the ED₅₀ estimate within the plate. The approximation was obtained, on the untransformed OD and the logarithm base 10 transformed dilutions, with the approx function of the stats R base package.

Sample were not assigned a titer in the following cases:
no measurement was available above or below the ED₅₀,
curve crossed at least twice the ED₅₀ and
one of the dilution step (left or right) closest to the ED₅₀ was missing

In-Vitro HSV-2 MS Neutralization Assay

An in-vitro neutralization assay was developed to detect and quantify HSV-2 MS neutralizing antibody titers in serum samples from different animal species. Sera (50 μL/well at starting dilution 1/10) were diluted by performing a 2-fold serial dilution in HSV medium (DMEM supplemented with 1% Neomycin and 1% gentamycin) in flat-bottom 96-well plates (Nunclon Delta Surface, Nunc, Denmark, ref 167008). Sera were then incubated for 2 h at 37° C. (5% CO₂) with 400 TCID50/50 μL/well of HSV-2 MS strain (ref ATCC VR-540) pre-diluted in HSV medium supplemented with 2% of guinea pig serum complement (Harlan, ref C-0006E). Edges of the plates were not used and one column of each plate was left without virus & sera (TC) or with virus but w/o serum (TV) and used as the negative or positive control of infection respectively. Positive control sera of the assay are pooled serum samples from mice immunized with different doses (0.22; 0.66; 2; 6 μg/dose) of HSV-2 gD/AS01(2.5 μg) and collected at 14 days post second (14PII) or third (14PIII) immunization. After the incubation of antibody-virus mixture, 10.000 Vero cells/100 μL were added to each well of each plate and plates were incubated for 4 days at 37° C. under 5% CO₂. Four days post-infection, supernatant was removed from the plates and cells were incubated for 5h at 37° C. (5% CO₂) with a WST-1 solution (reagent for measuring cell viability, Roche, ref 11644807001) diluted 15× in HSV revelation medium (DMEM supplemented with 1% Neomycin and 1% gentamycin+2% FBS).

To calculate neutralizing antibody titers, sets of data were normalized based on the mean of WST-1 optical density (O.D.) in “cells w/o virus” wells and “cells w/o serum” wells to 0 and 100% cytopathic effect (CPE) respectively. Percentage of inhibition of CPE at a dilution i was then given by:

% inhibition=(O.D.ᵢ−Mean O.D._{cells w/o serum})/(Mean O.D._{cells w/o virus}−Mean O.D._{cells w/o serum})

The reciprocal of the dilution giving a 50% reduction of CPE was then extrapolated using non-linear regression with the Softmaxpro Software.

In-Vitro HSV-1 Neutralization Assay

An in-vitro neutralization assay was developed to detect and quantify HSV-1 cross-reactive neutralizing antibody titers in serum samples from different animal species. A 2-fold serial dilution of each serum (50 μL/well at starting dilution 1/10) in HSV medium (DMEM supplemented with 1% Neomycin and 1% gentamycin) were performed in flat-bottom 96-well plates (Nunclon Delta Surface, Nunc, Denmark, ref 167008). Sera were then incubated for 2 h at 37° C. (5% CO₂) with 400 TCID50/50 μL/well of HSV-1 strain (ref ATCC VR-1789) pre-diluted in HSV medium supplemented with 2% of guinea pig serum complement (Harlan, ref C-0006E). Edges of the plates were not used and one column of each plate was left without virus & sera (TC) or with virus but w/o serum (TV) and used as the negative or positive control of infection respectively. Positive control sera of the assay are pooled serum samples from mice immunized with different doses (0.22; 0.66; 2; 6 μg/dose) of HSV-2 gD/AS01(2.5 μg) and collected at 14 days post second (14PII) or third (14PIII) immunization. After the incubation of antibody-virus mixture, 10.000 Vero cells/100 μL were added to each well of each plate and plates were incubated for 4 days at 37° C. under 5% CO₂. Four days post-infection, supernatant was removed from the plates and cells were incubated for 5h at 37° C. (5% CO₂) with a WST-1 solution (reagent for measuring cell viability, Roche, ref 11644807001) diluted 15× in HSV revelation medium (DMEM supplemented with 1% Neomycin and 1% gentamycin+25% FBS).

To calculate neutralizing antibody titers, sets of data were normalized based on the mean of WST-1 optical density (O.D.) in “cells w/o virus” wells and “cells w/o serum” wells to 0 and 100% cytopathic effect (CPE) respectively. Percentage of inhibition of CPE at a dilution i was then given by:

% inhibition=(O.D.ᵢ−Mean O.D._{cells w/o serum})/(Mean O.D._{cells w/o virus}−Mean O.D._{cells w/o serum})

The reciprocal of the dilution giving a 50% reduction of CPE was then extrapolated using non-linear regression with the Softmaxpro Software.

Statistical Methods

The distribution of each response was assumed to be lognormal.

For each vaccine-specific IgG antibody response (gE or gI), a two-way analysis of variance (ANOVA) model was fitted on log 10 titers by including groups (all except the NaCl one), time points (Day14 (14PI), Day28 (14PII) and Day42 (14PIII)) and their interactions as fixed effects. The NaCl group was not included as (almost) no response and variability was observed. Variance-covariance model selection was based on AICC criterion and individual data plot examination.

gE-specific variance-covariance for time points was modelled via an Heterogenous Compound Symmetry:

$$\begin{matrix}[ \begin{matrix}{σ}_{1}^{2} + {σ}_{4} & {σ}_{4} & {σ}_{4} \\ {σ}_{4} & {σ}_{2}^{2} + {σ}_{4} & {σ}_{4} \\ {σ}_{4} & {σ}_{4} & {σ}_{3}^{2} + {σ}_{4}\end{matrix} ] \\ CSH - Heterogeneous CS\end{matrix}$$

The compound symmetry considers same correlation between timepoints, heterogenous refers to the fact that different variances were assumed for each timepoint. A different variance-covariance matrix was modelled for each vaccine group, indicating different variances and different timepoint correlations between groups.

gI-specific variance-covariance for time points was modelled via an Heterogenous First Order Autoregressive ARH (1) structure:

$$\begin{matrix}[ \begin{matrix}{σ}_{1}^{2} & {σ}_{1} {σ}_{2} ρ & {σ}_{1} {σ}_{3} {ρ}^{2} \\ {σ}_{1} {σ}_{2} ρ & {σ}_{2}^{2} & {σ}_{1} {σ}_{2} ρ \\ {σ}_{1} {σ}_{3} {ρ}^{2} & {σ}_{2} {σ}_{3} ρ & {σ}_{3}^{2}\end{matrix} ] \\ ARH ( 1 ) - Heterogeneous AR ( 1 )\end{matrix}$$

The autoregressive structure considers correlations to be highest for adjacent times, and a systematically decreasing correlation with increasing distance between time points. Heterogenous refers to the fact that different variances were assumed for each timepoint. The same variance-covariance matrix was modelled for each vaccine group, indicating same variance between groups.

Geometric means and their 95% CIs are derived from these models.

Despite the fact that the NaCl group was not included in the ANOVA models, the comparisons associated to the primary objective were computed as follows: geometric means and 95% CI of vaccinated groups derived from the above models were divided by titer given to all the NaCl recipients for gE, or the geometric mean titer of NaCl group for gI, at the last timepoint. The resulting ratios should be understood as geometric mean ratios with their corresponding 95% CI.

For head to head comparison of vaccinated groups (at the last time point) and time point comparisons (PII/PI, PIII/PII, and PIII/PI) within each group, all the geometric mean ratios and their 95% CIs were derived from the models.

For HSV-1 gE/gI cross-reactive IgG antibody response, a one-way analysis of variance (ANOVA) model was fitted on log 10 titers by including groups (all except the NaCl one) as fixed effect. The NaCl group was not included as no response and variability was observed. No clear heterogeneity of variance was detected and therefore identical variances were assumed for the different groups. Geometric means and their 95% CIs are derived from these models. For comparison of vaccinated to NaCl groups, geometric means and 95% CI of vaccinated groups derived from the above model were divided by titer given to all the NaCl recipients. The resulting ratios should be understood as geometric mean ratios with their corresponding 95% CI. For head to head comparison of vaccinated groups, all the geometric mean ratios and their 95% CIs were derived from the model.

For % of gE or gI-specific CD4+ or CD8+ T-cell responses, a one-way analysis of variance (ANOVA) model was fitted on log 10 frequencies by including groups (all groups including the NaCl group) as fixed effect. No clear heterogeneity of variance was detected and therefore identical variances were assumed for the different groups. For both % of CD4+ and CD8+ T cell responses, the NaCl threshold was based on P95 of data across stimulation in NaCl negative control group. No modelling was performed on gI-specific CD8+ T cells since response was below the P95 NaCl threshold for all vaccine groups. Geometric means and geometric mean ratios (with their corresponding 95% Cis) were derived from these models.

For the evaluation of the dissociation of the human IgG Fc binding by the pAbs, ED50 response was calculated for each sample. On this response, a one-way analysis of variance (ANOVA) model was fitted on log 10 values by including groups (all groups excluding the NaCl group) as fixed effect. Different variances for each group were modeled. Geometric means and geometric mean ratios (their corresponding 95% Cis) were derived from these models.

For HSV-2 MS-specific neutralizing antibody titers, a one-way analysis of variance (ANOVA) model was fitted on log 10 values by including groups (all groups excluding the NaCl group) as fixed effect. Different variances for each group were modeled. Geometric means and geometric mean ratios (their corresponding 95% Cis) were derived from these models. For HSV-1 cross-reactive neutralizing antibody titers, the same method was used except that identical variances were assumed for the different groups (no clear heterogeneity of variance was detected).

Results

Anti-HSV-2 gE & gI-Specific and Anti-HSV-1 gE/gI Cross-Reactive IgG Antibodies were Induced with Different Mutated Versions of HSV-2 gE/gI Protein

The anti-HSV-2 gE & gI-vaccine-specific and anti-HSV-1 gE/gI cross-reactive IgG antibody responses were investigated by ELISA.

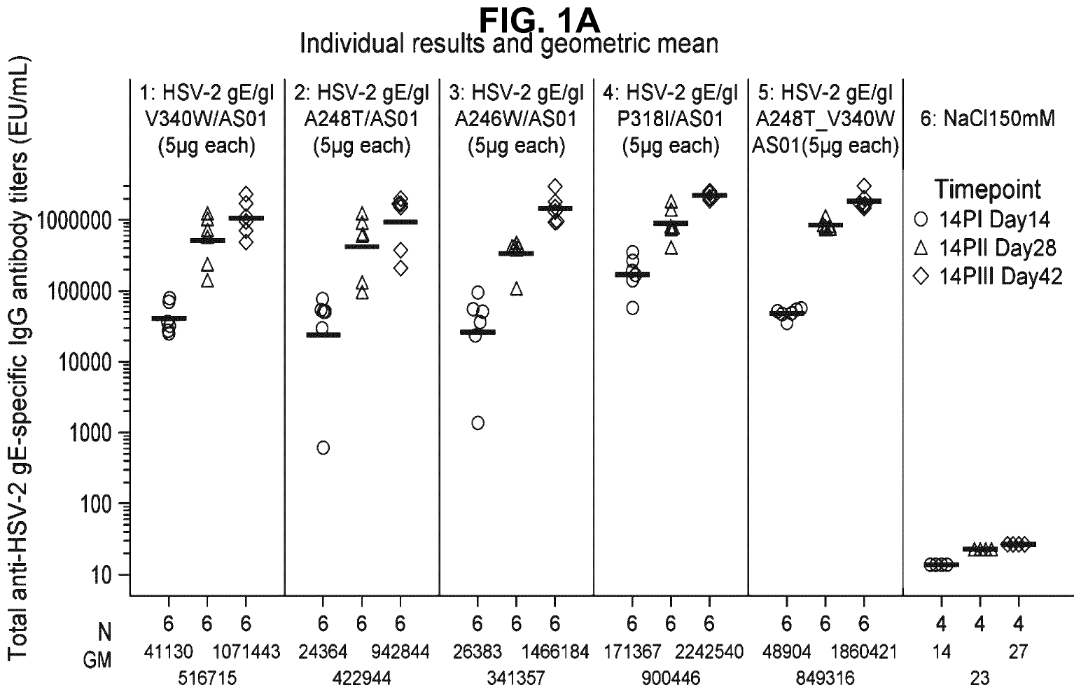

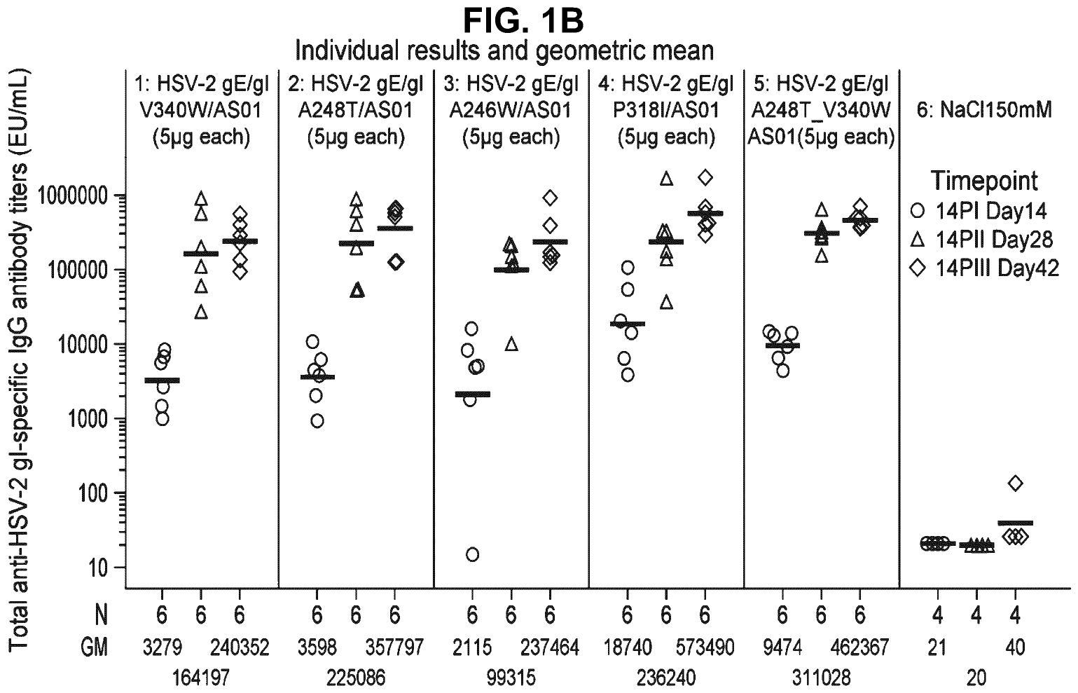

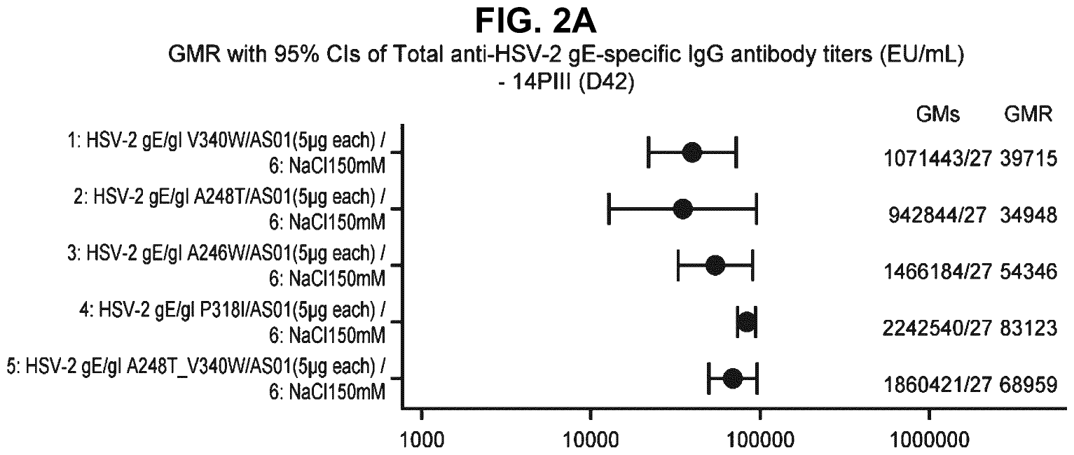

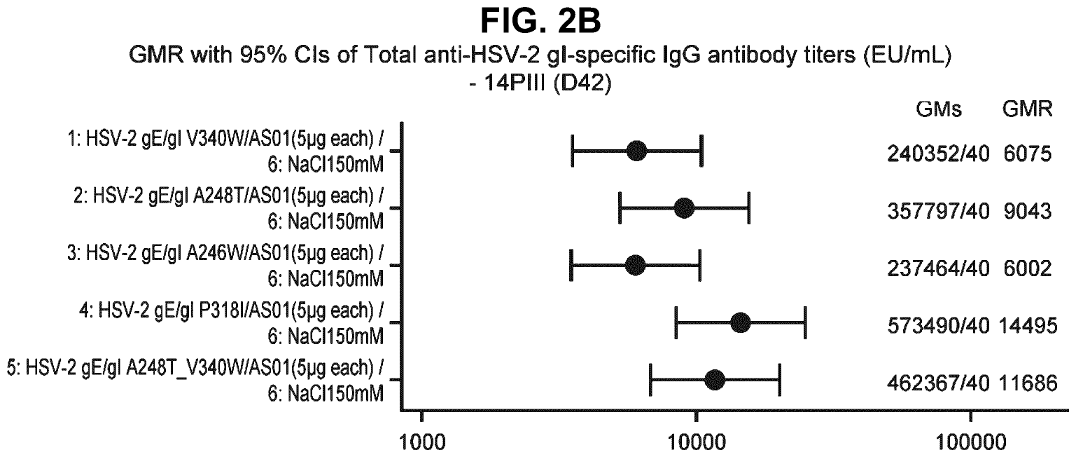

Compared to NaCl control group, high anti-HSV-2 gE or gI- vaccine-specific IgG antibody responses were induced by all mutated versions of AS01-adjuvanted HSV-2 gE/gI protein 14 days post third immunization (all GMRs>34.000 for gE and all GMRs>6000 for gI) (FIG. 1A, FIG. 1B, FIG. 2A, FIG. 2B). As expected, no anti-HSV-2 gE or gI response was observed in NaCl control group (<30 EU/ml for all mice except for one with a value of 135 EU/mL in HSV-2 gI response).

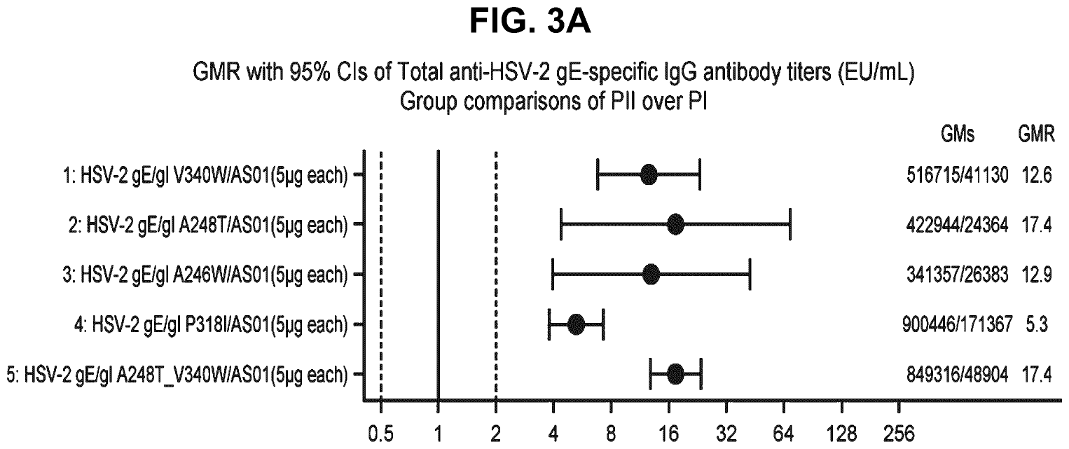

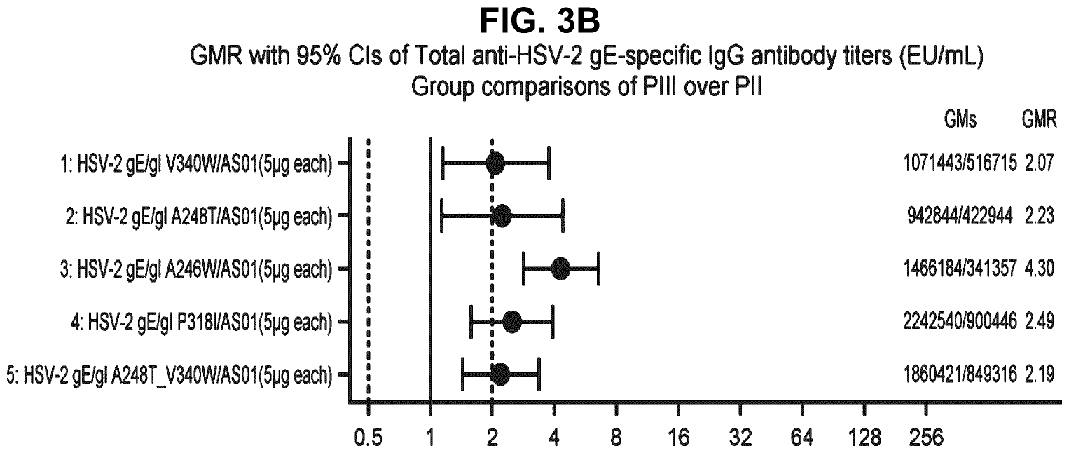

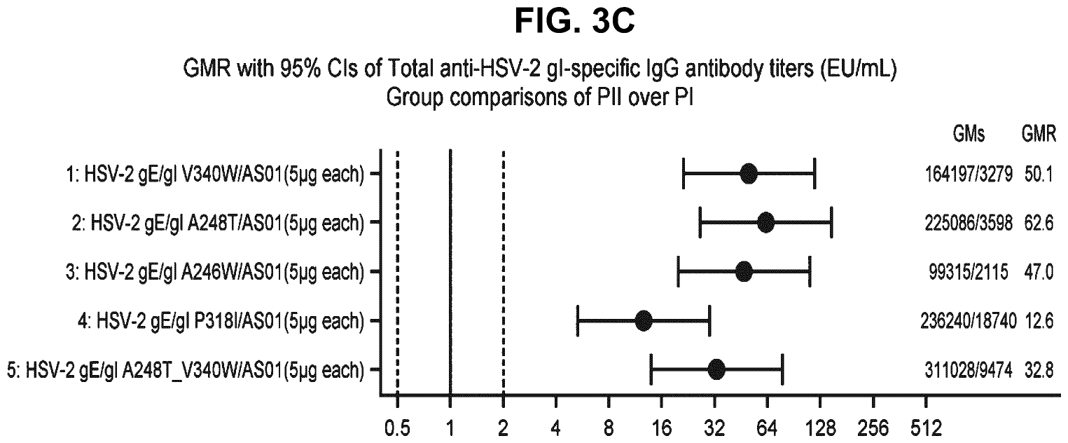

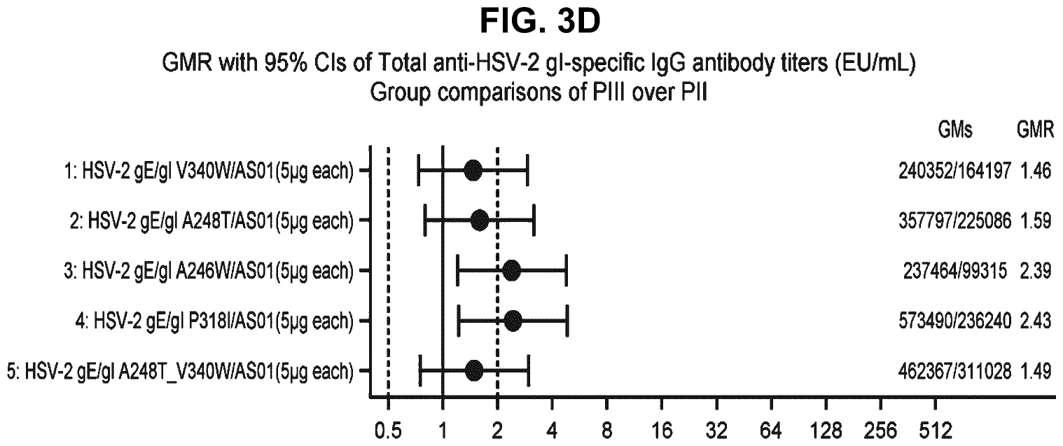

In all groups of mice immunized with AS01-adjuvanted HSV-2 gE/gI mutant proteins, levels of anti-HSV-2 gE and gI-specific IgG antibody responses increased after the second immunization (day28 (14PII)) compared to the first one (day14 (14PI)), with a fold increase ranging from 5 to 63 (FIG. 3A, FIG. 3C).

For all HSV-2 gE/gI mutant proteins, a booster effect on anti-HSV-2 gE-specific antibody response was also observed after third immunization (day42(14PIII)) compared to the second one (day28 (14PII)), with a fold increase ranging from 2 to 4 (with CI not containing 1) (FIG. 3B).

A boosted effect on anti-HSV-2 gI-specific antibody response after third immunization compared to the second one was only detected in the groups of mice immunized with AS01-adjuvanted HSV-2 gE/gI A246W (group 3) and P318I (group 4) mutant proteins. Note that in A248T (group 3), one animal had a very low anti-HSV-2 gI-response compared to the others after two immunisations but was very similar to the others after the three immunizations. This might have amplified the booster effect observed in that group. No booster effect was observed in the three other groups (<2-fold change and CI containing 1) (FIG. 3D).

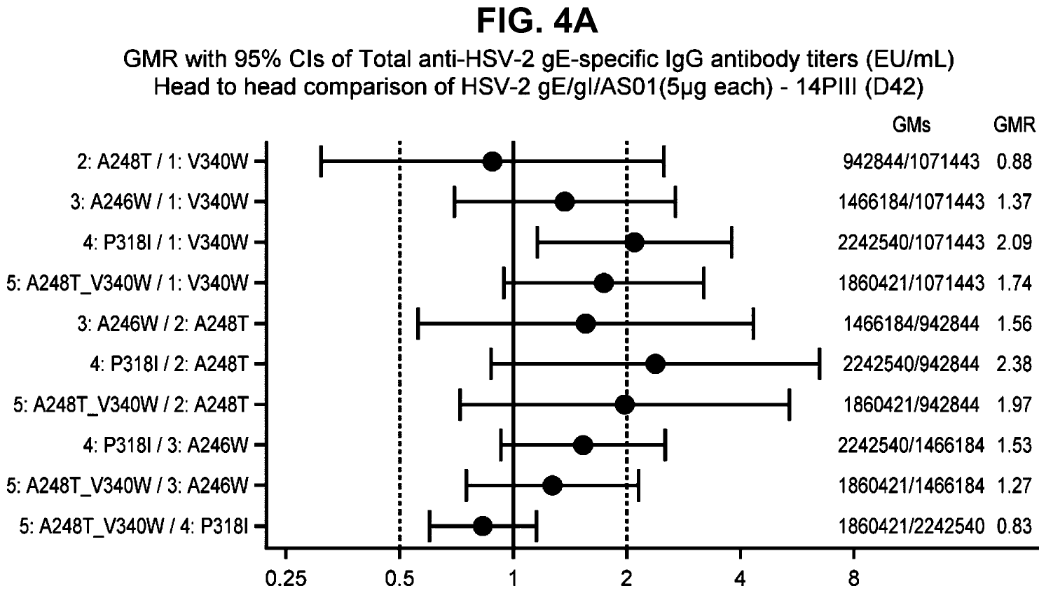

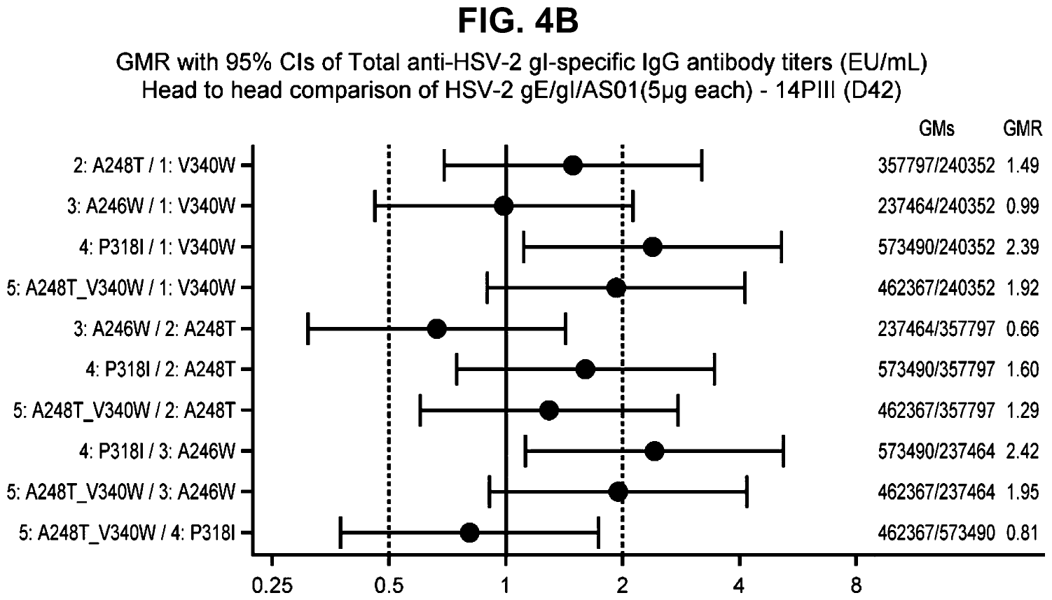

Overall, these results suggest similar anti-HSV-2 gE- or anti-HSV-2 gI-specific antibody responses between the different mutated versions of HSV-2 gE/gI protein at 14 days post third immunization. Only P318I mutant (group4) seemed to induce higher anti-HSV-2 gE- and anti-HSV-2 gI-specific antibody responses compared to V340W mutant (group 1) with a fold increase close to 2 (GMRs of 2.09 and 2.39, respectively with CIs not containing 1) (FIG. 4A & FIG. 4B). P318I mutant (group4) seemed also to induce higher anti-HSV-2 gI-specific antibody response compared to A246W mutant (group3) with a fold increase of 2.42 (with CI not containing 1) (FIG. 4B). No other differences between mutants were observed (with GMRs<2-fold change and/or CIs containing 1).

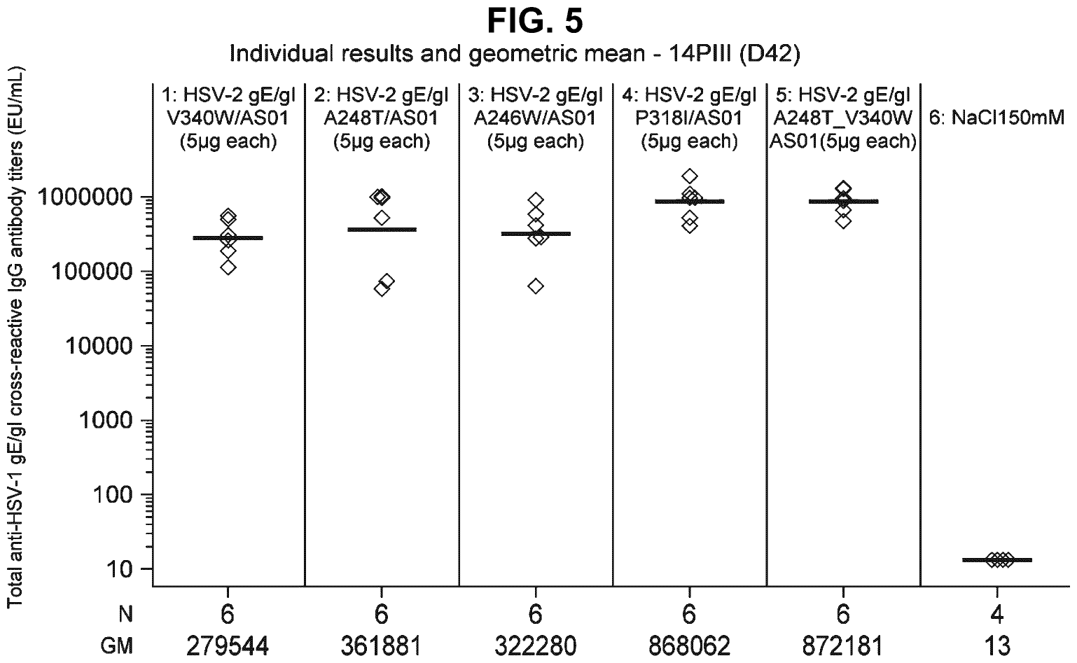

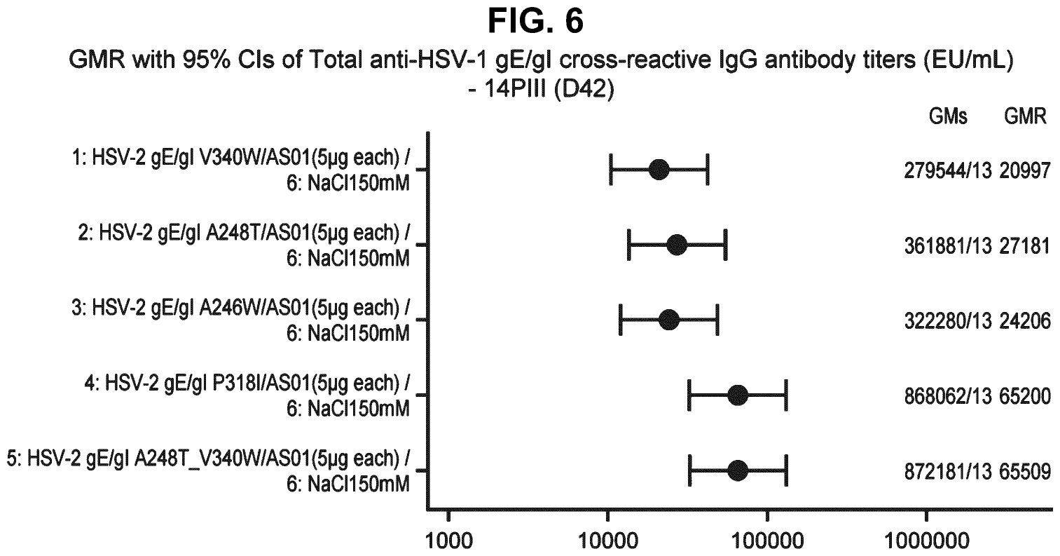

Anti-HSV-1 gE/gI cross-reactive response was not observed in NaCl control group (<20 EU/mL for all mice). At 14 days post third immunization and compared to NaCl control group, high anti-HSV-1 gE/gI cross-reactive IgG antibody response was induced in all groups of mice immunized by different mutated versions of AS01-adjuvanted HSV-2 gE/gI protein (all GMRs>20.000) (FIG. 5, FIG. 6).

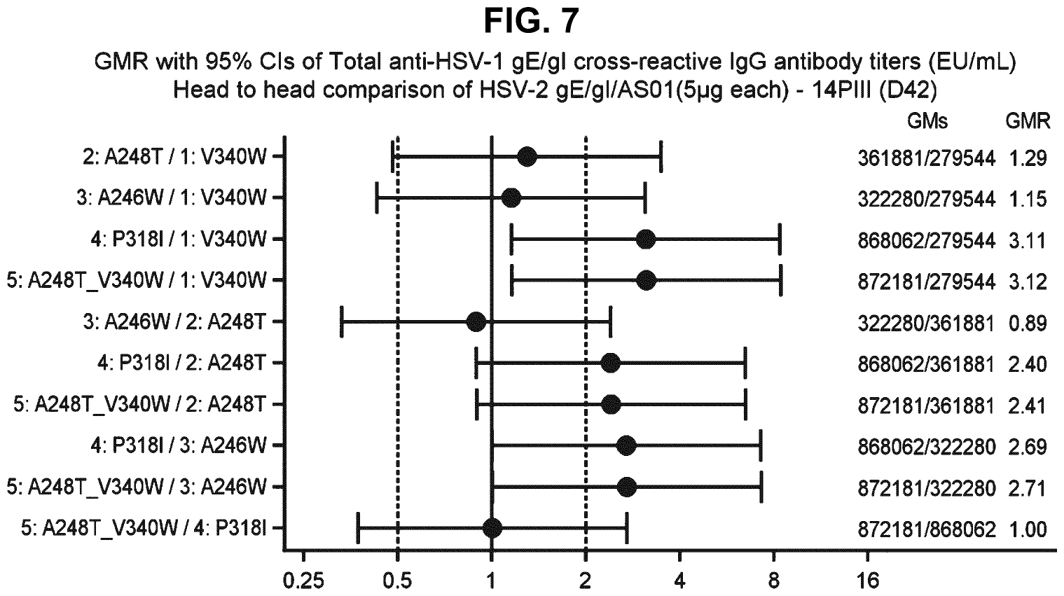

Several differences between HSV-2 gE/gI mutant candidates seemed to be observed at 14 days post third immunization. Indeed, cross-reactive response induced by both P318I (group 4) and A248T_V340W (group 5) mutants seemed to be about three times higher than the one induced by V340W (group 1) and A248T (group 3) mutants (GMRs of 3.1 with CIs not containing 1 for groups 4 or 5 versus 1, and 2.7 with lower limits of CI equal to 1 for groups 4 or 5 versus 3). No other differences between mutants seemed to be observed (with GMRs<2-fold change and/or CIs containing 1) (FIG. 7).

All Recombinant HSV-2 gE/gI Mutant Proteins can Induce Anti-HSV-2 Functional gE/gI Specific and Anti-HSV-1 gE/gI Cross-Reactive Antibodies

In this study, anti-HSV-2-specific & anti-HSV-1 cross-reactive antibody functions were only investigated in the sera collected at 14 days post third immunization. In this regard, the ability of pAbs to neutralize HSV-2 MS or HSV-1 VR1789 viruses, to bind and activate mouse FcγRIII expressed by Jurkat reporter cell line after binding of HSV-2 or HSV-1 gE/gI transfected cells and to decrease in-vitro binding of human IgG Fc by HSV-2 or HSV-1 gE/gI proteins was investigated.

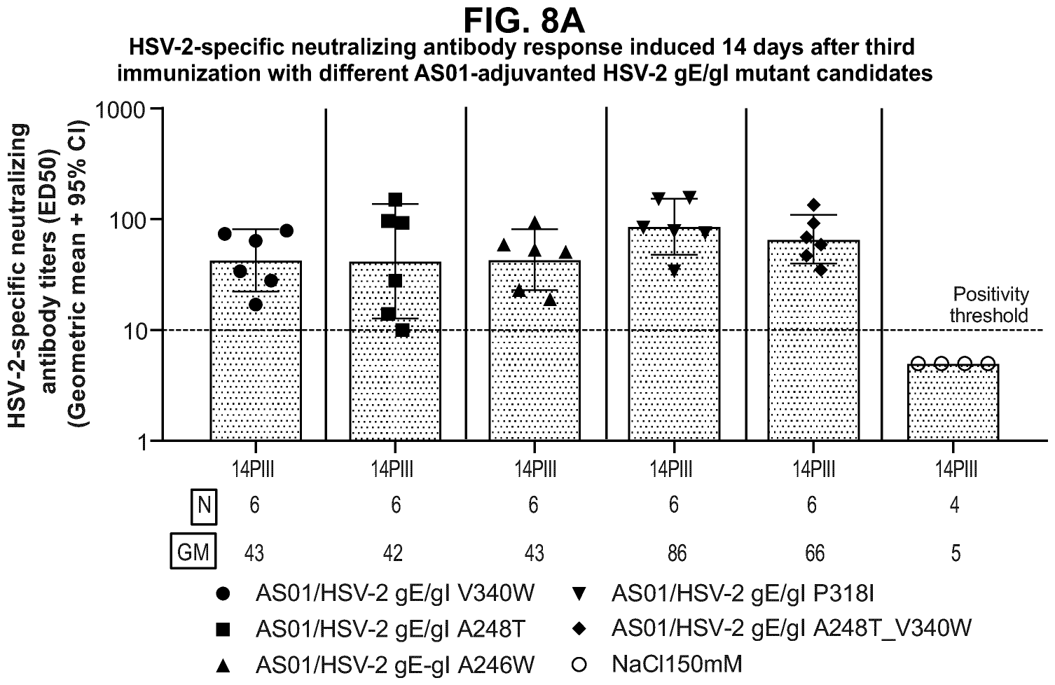

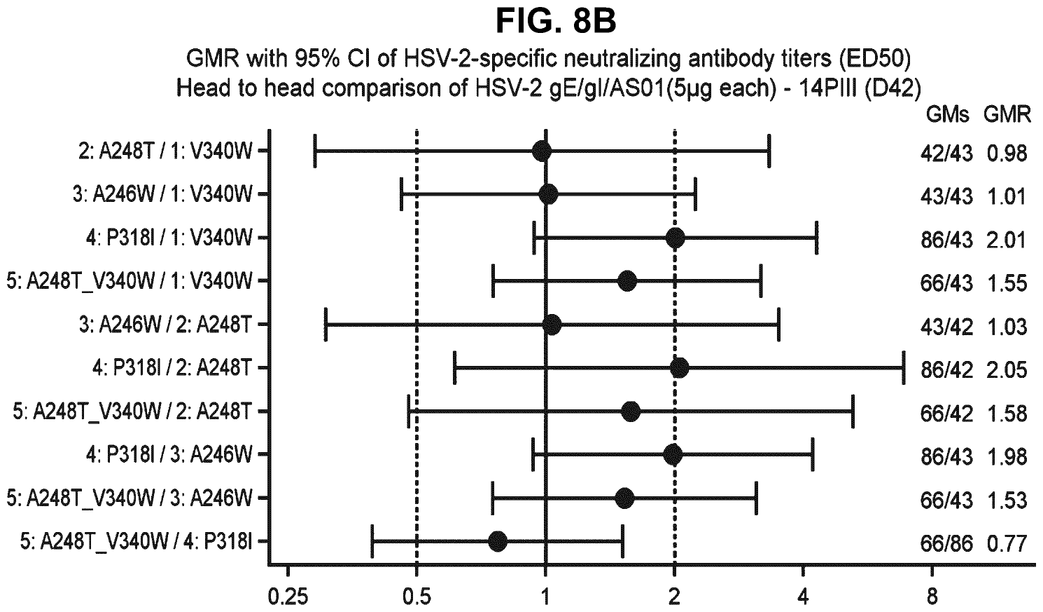

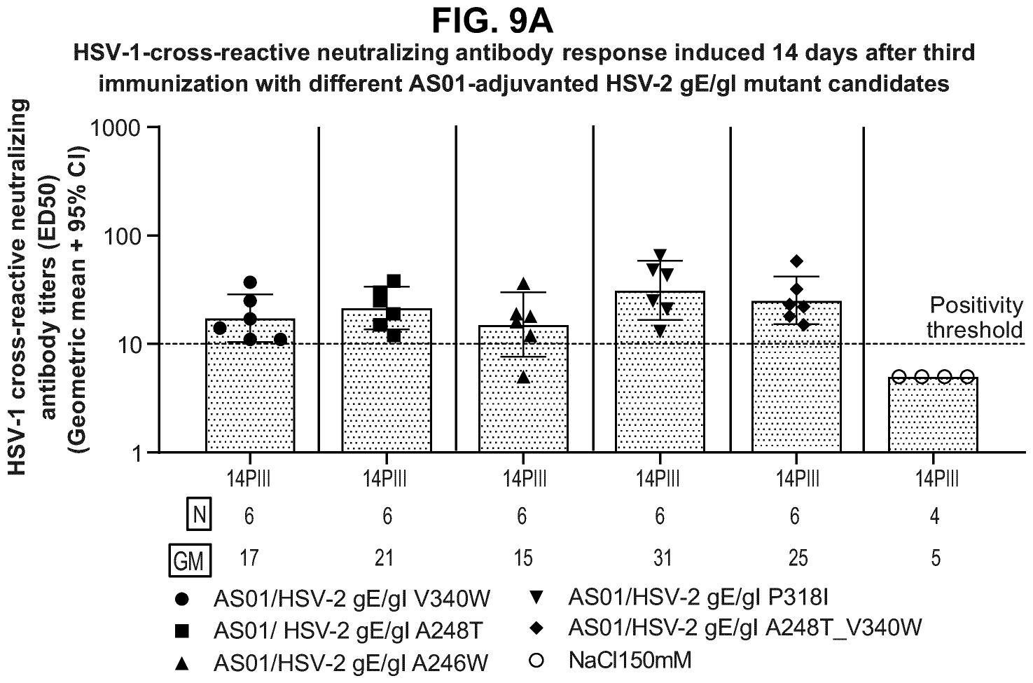

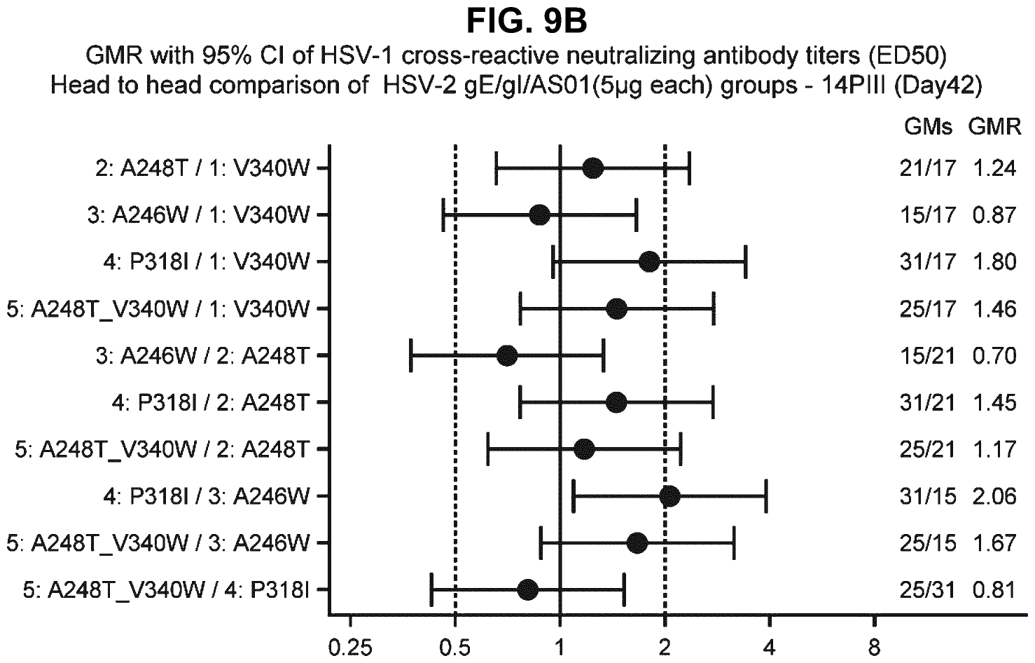

Very low but consistent neutralizing (nAb) antibody responses directed to HSV-2 MS and HSV-1 VR1789 strains were detected in all groups of mice immunized with different mutated versions of AS01-adjuvanted HSV-2 gE/gI protein. In term of anti-HSV-2 specific nAb response, no clear evidence of difference between the mutated versions of HSV-2 gE/gI protein was observed (GMRs<2-fold change with all CIs containing 1). Comparisons between P318I (group 4) versus V340W (group 1) and A246W (group3) mutants indicate a 2-fold change that just fail to reach significance (lower limits of CI just below 1) (FIG. 8A; FIG. 8B). On the side of the cross-reactive nAb response directed against HSV-1 VR1789 strain, results also suggest similar response between the different mutated versions of HSV-2 gE/gI protein. Only P318I mutant (group4) seemed to induce higher response compared to A246W mutant (group3) with a fold increase close to 2 (GMR of 2.06 with CI not containing 1). Comparison between P318I (group 4) versus V340W (group 1) mutants indicates a 1.8-fold change that just fails to reach significance (lower limit of CI just below 1). No other differences between mutants were observed (GMRs<2-fold change and CIs containing 1)(FIG. 9A & FIG. 9B).

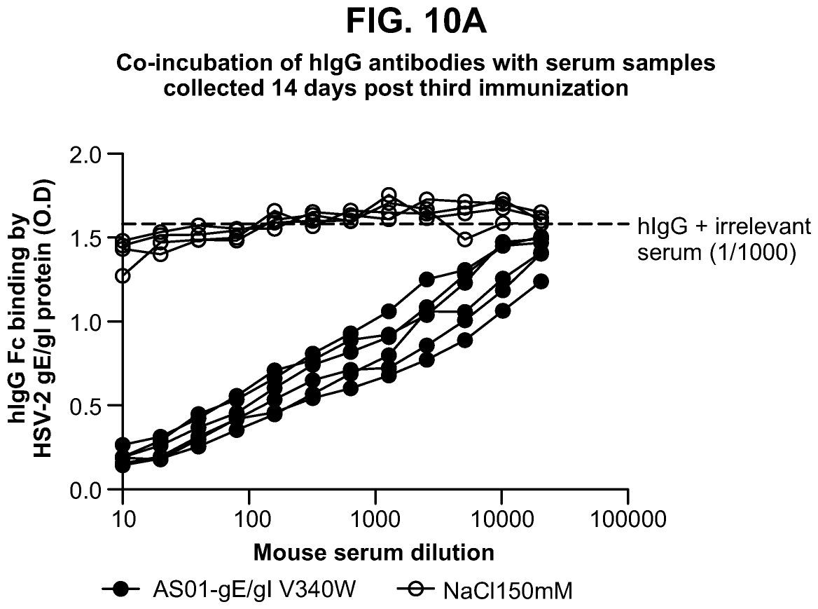

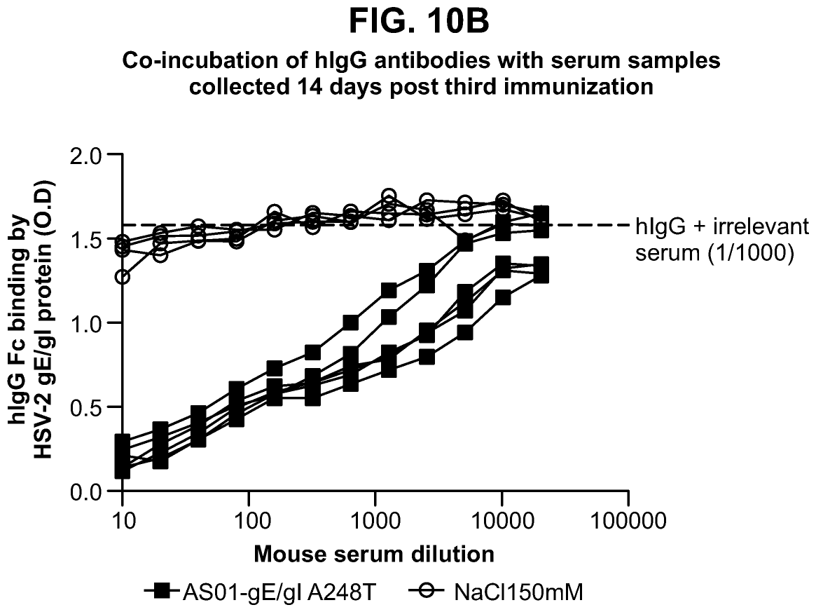

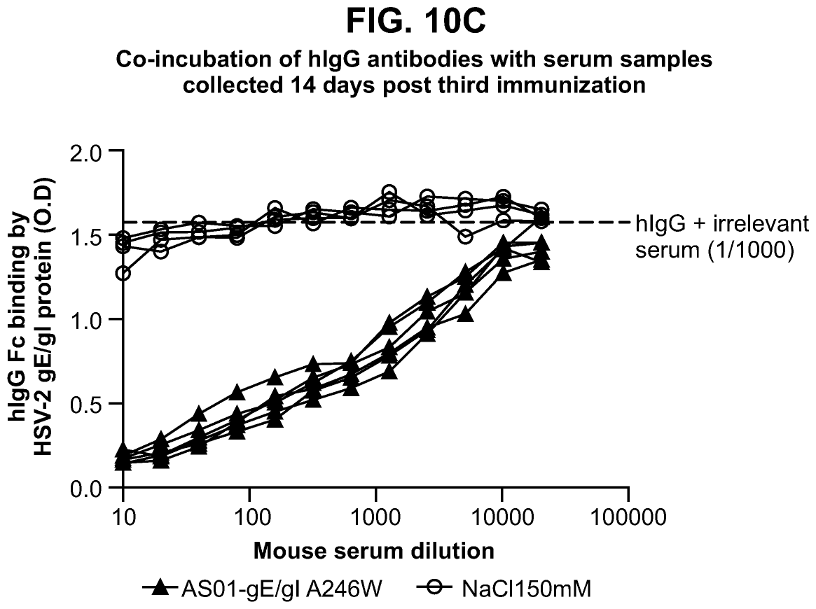

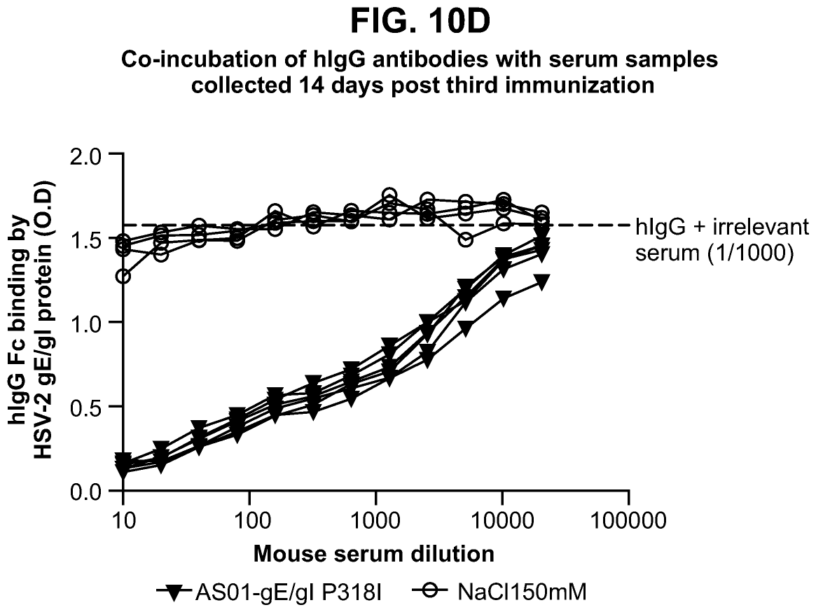

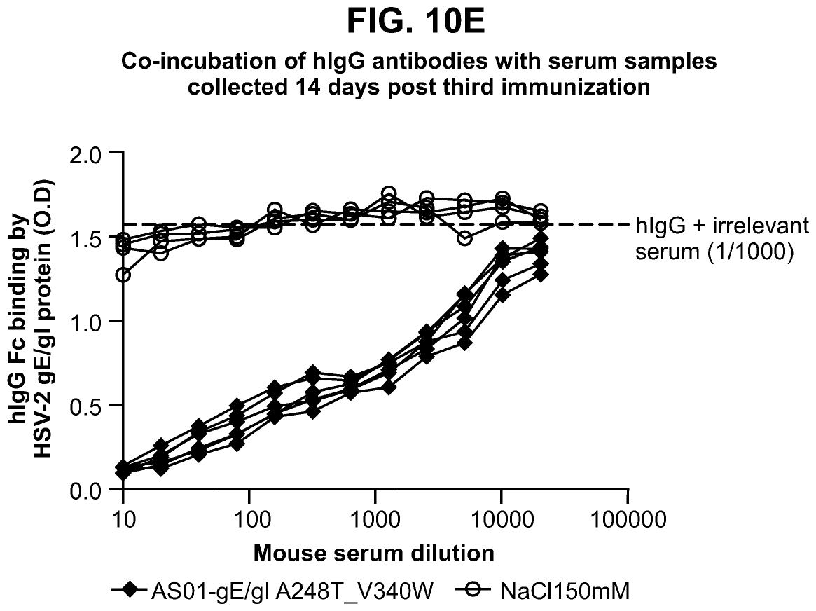

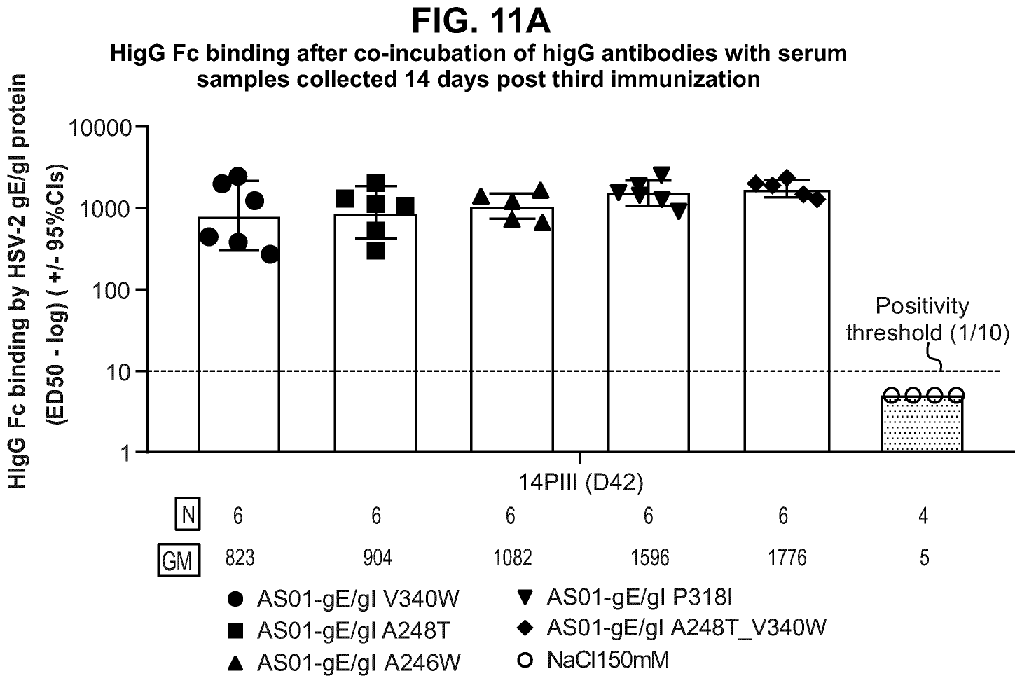

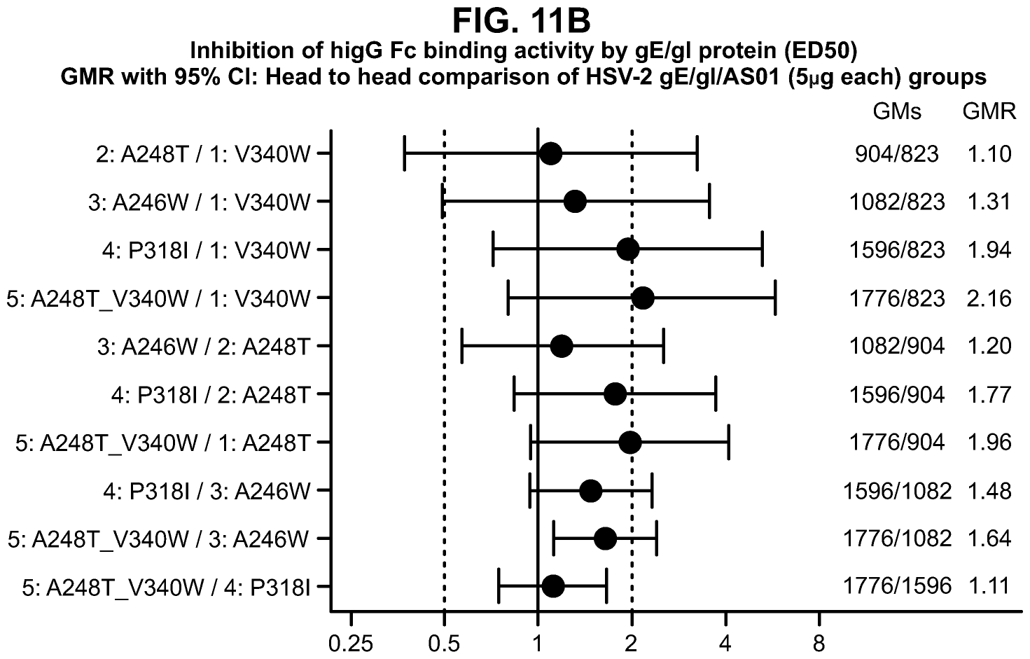

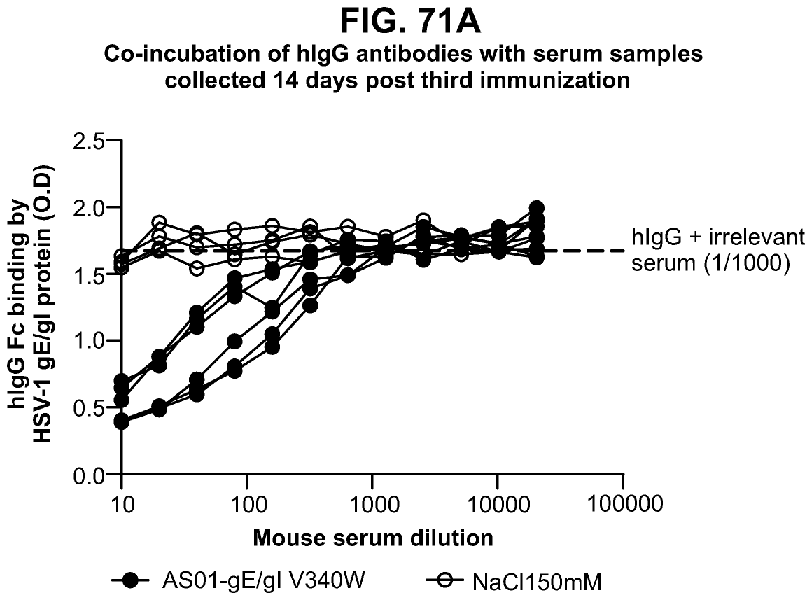

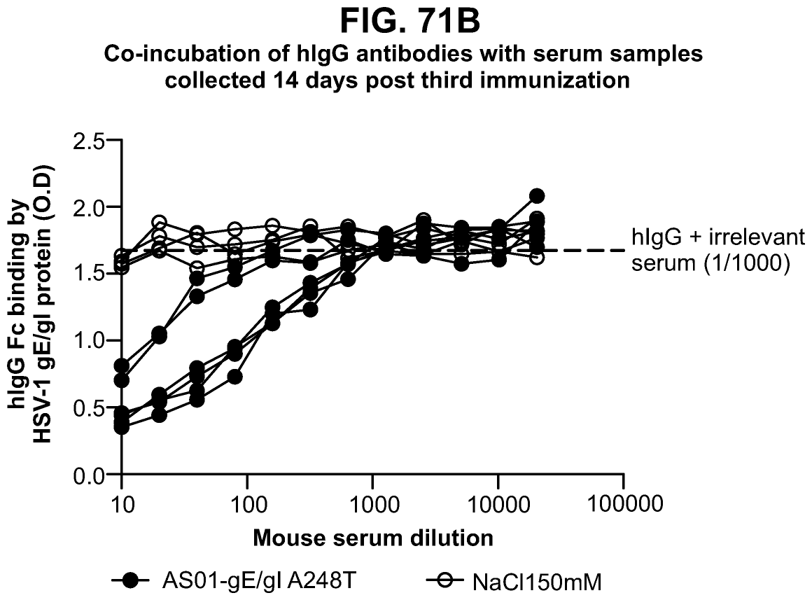

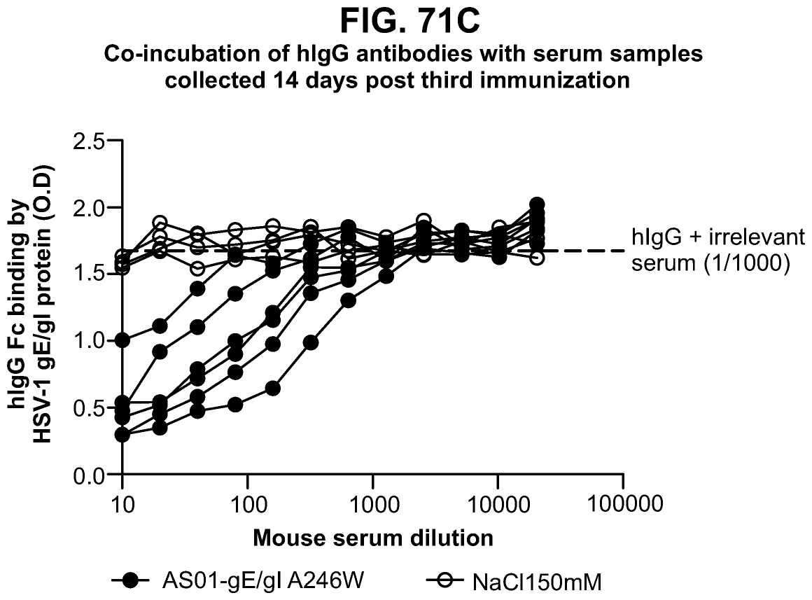

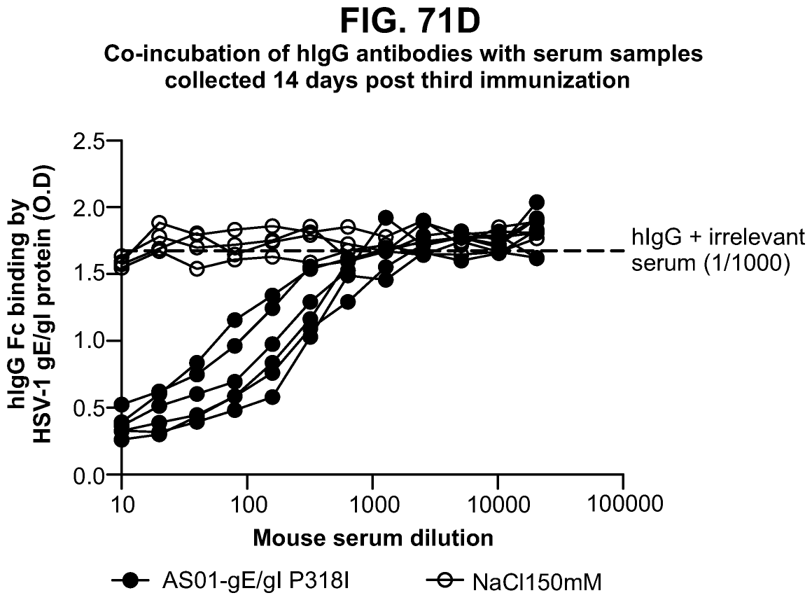

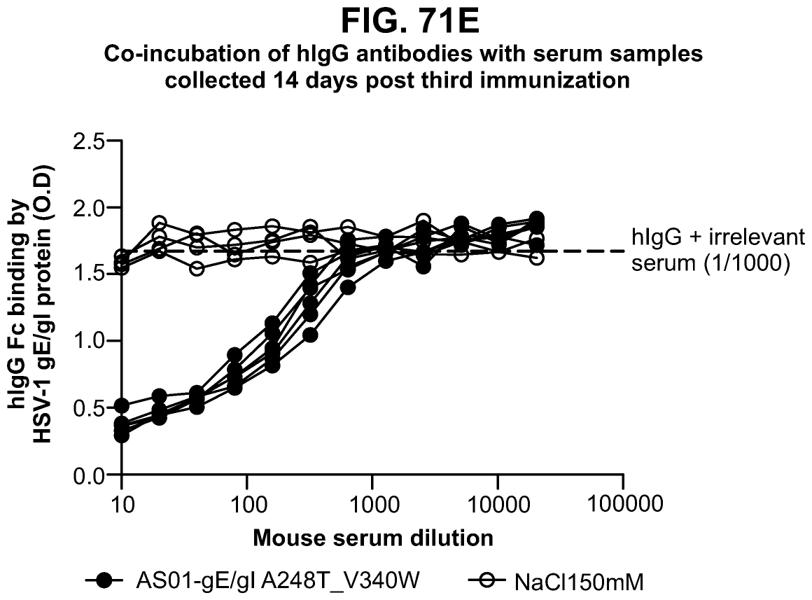

Then, the ability of sera from mice immunized with different HSV-2 gE/gI mutant candidates to compete and decrease hIgG binding by HSV-2 or HSV-1 gE/gI protein was assessed, in-vitro, by competitive ELISA. Different mutated versions of HSV-2 gE/gI protein elicited vaccine-specific and HSV-1 cross reactive polyclonal antibody response able to compete with hIgG binding by HSV-2 or HSV-1 gE/gI protein (FIG. 10 and FIG. 71). The dissociation curve of hIgG Fc binding by HSV-2 or HSV-1 gE/gI protein was quite similar between all groups of mice. However, a trend for higher inhibitory response of hIgG Fc binding towards HSV-2 gE/gI protein was observed with polyclonal antibodies from mice immunized with A248T_V340W mutant (group5) compared to A248T mutant (group 2) with a fold increase of 1.96 (and CIs almost not including 1) (FIG. 11A; FIG. 11B).

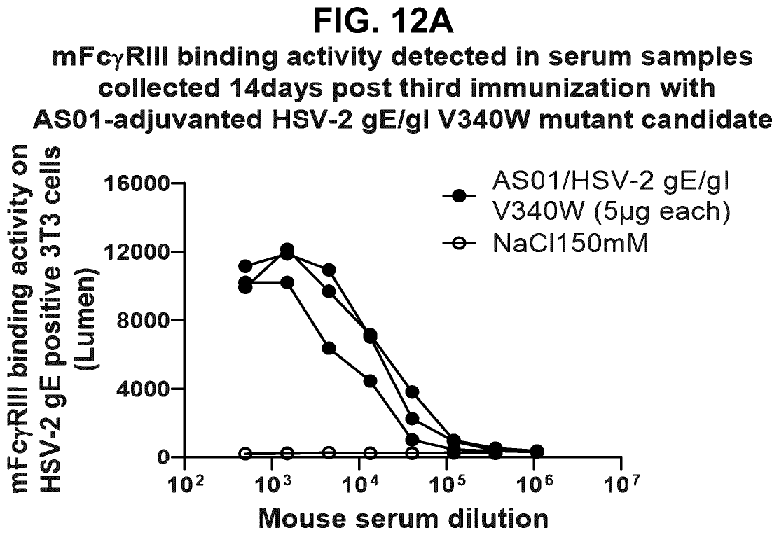

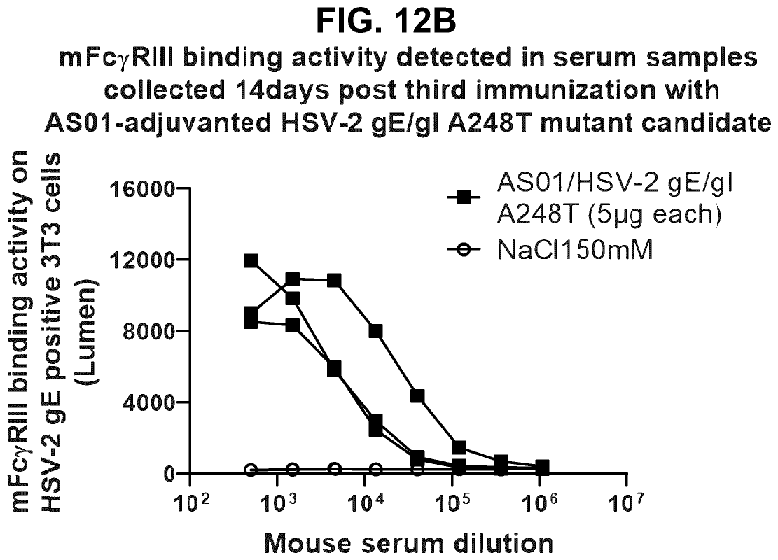

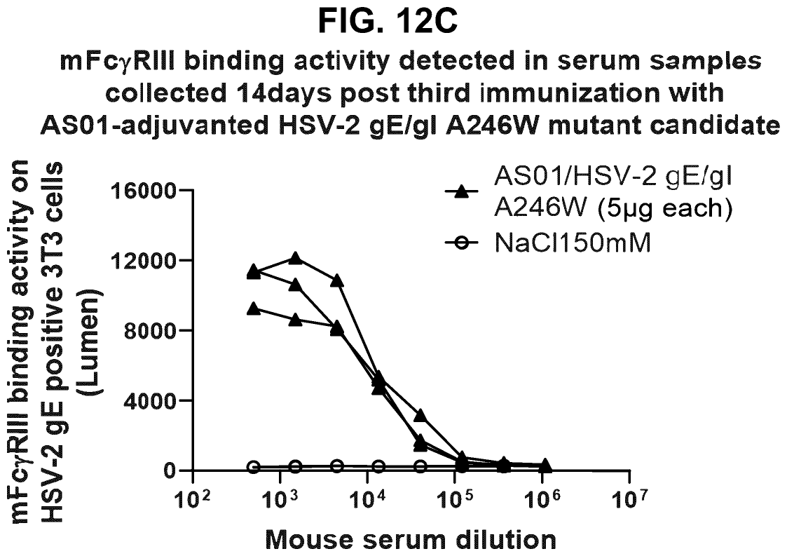

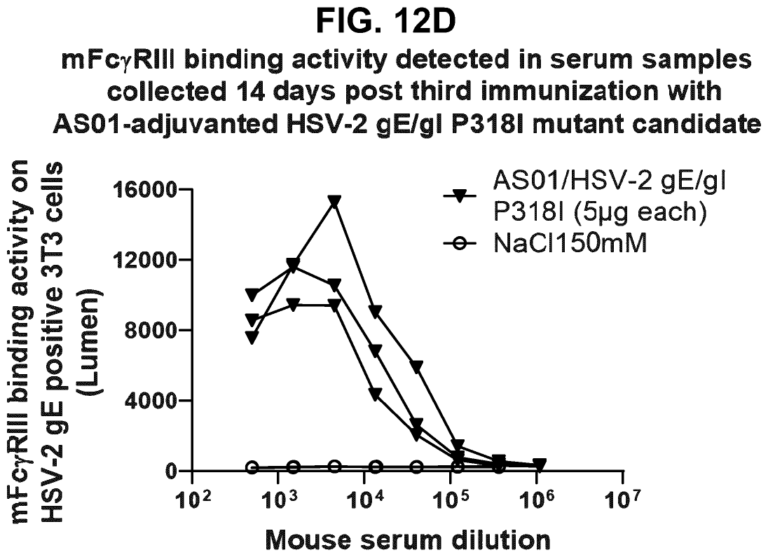

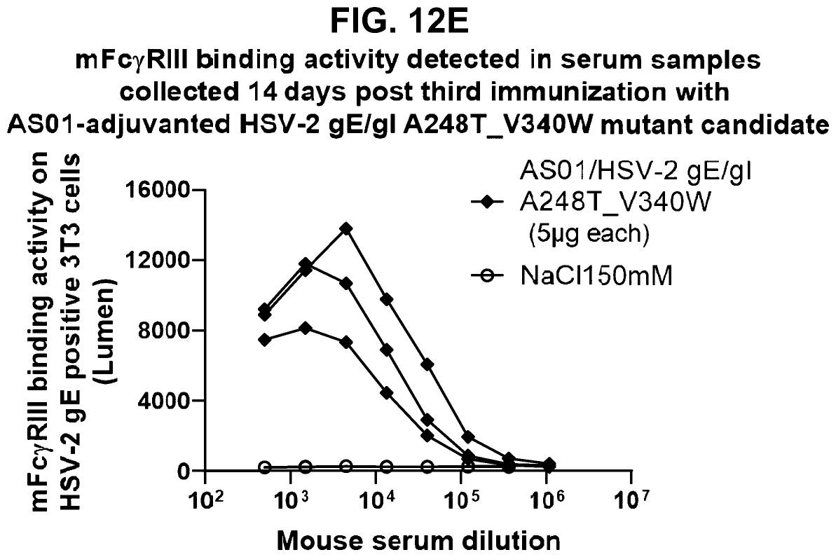

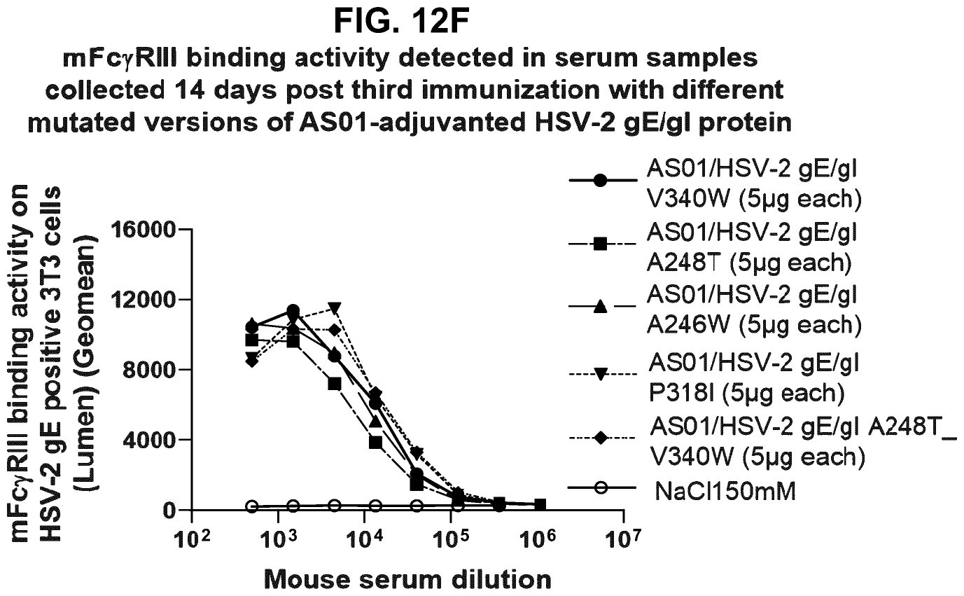

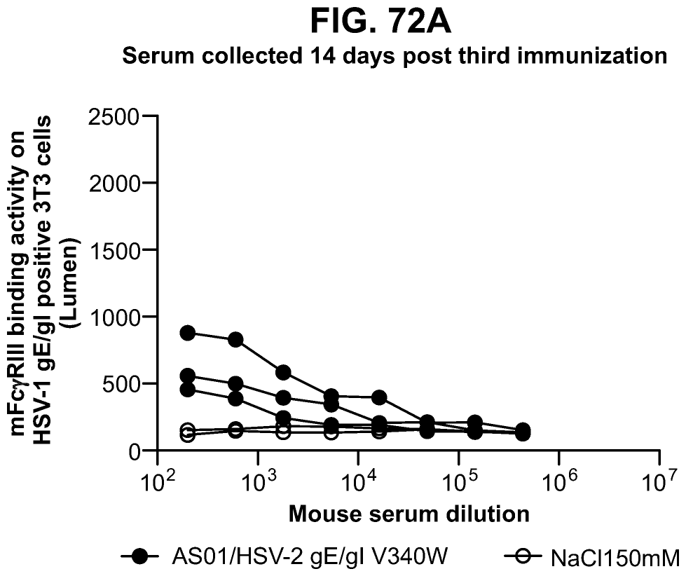

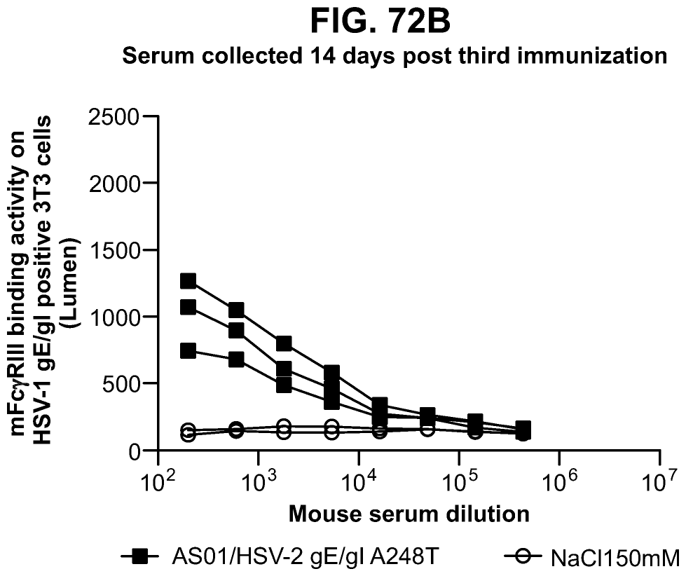

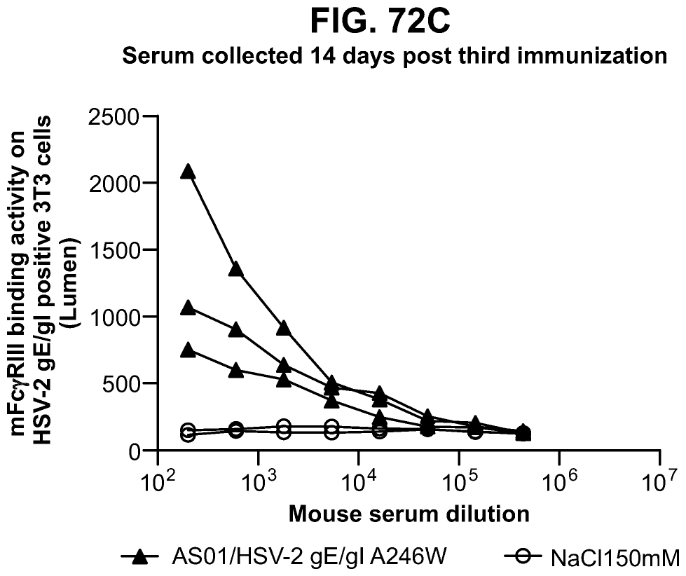

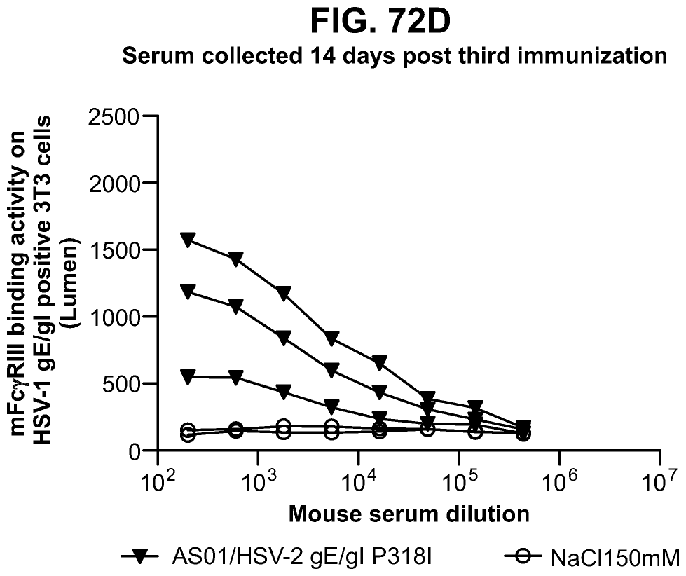

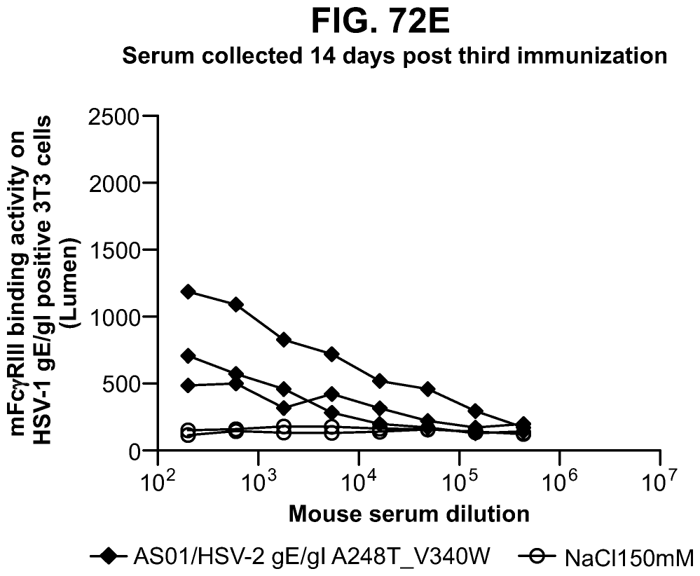

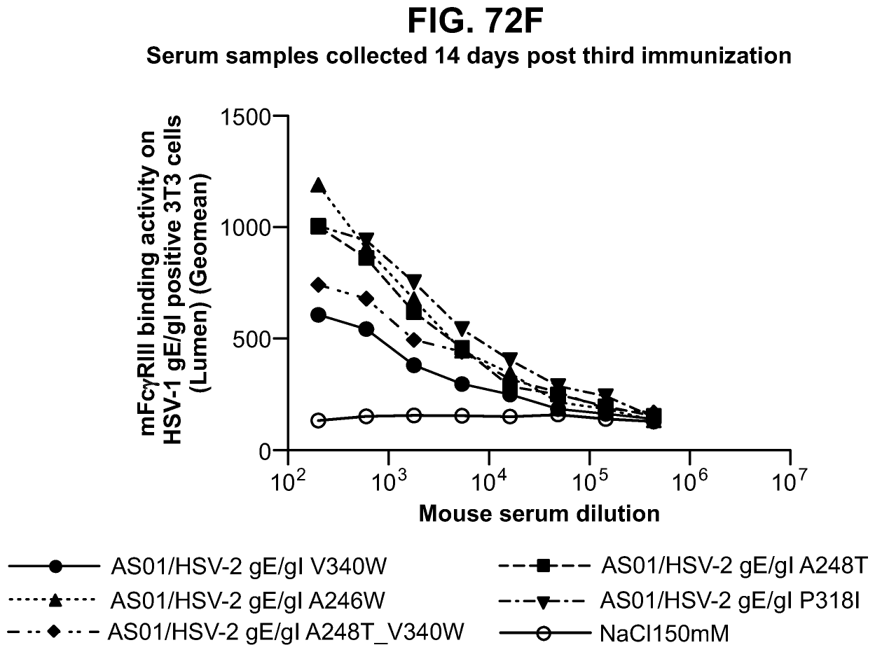

Finally, the ability of a vaccine-specific or HSV-1 gE/gI cross reactive antibody response, induced 14 days post third immunization, to bind and activate in-vitro mouse FcγRIII was investigated on HSV-2 and HSV-1 gE/gI-transfected cells. Data shown on FIGS. 12A-E and FIGS. 72A-E suggested that all groups of mice immunized with the different mutated versions of AS01-HSV-2 gE/gI protein could induce HSV-2 gE/gI-specific and HSV-1 gE/gI cross reactive antibody responses able to specifically bind and activate Jurkat reporter cells expressing FcγRIII. As expected, activation of FcγRIII was not detected with sera from unvaccinated mice. No major difference was observed between the different groups of mice in terms of FcγRIII activation after incubation with HSV-2 or HSV-1 gE/gI transfected cells (FIG. 12F and FIG. 72F).

In conclusion, these data suggest that different mutated versions of AS01/HSV-2 gE/gI protein can induce functional vaccine-specific and HSV-1 gE/gI cross reactive antibodies able to bind and activate FcγRIII after incubation with HSV-2 or HSV-1 gE/gI transfected cells, to decrease human IgG Fc binding by HSV-2 or HSV-1 gE/gI protein and to neutralize at low intensity HSV-2 and HSV-1 viruses.

All Mutated Versions of Recombinant HSV-2 gE/gI Protein can Induce Vaccine-Specific CD4+ and CD8+ T Cell Responses

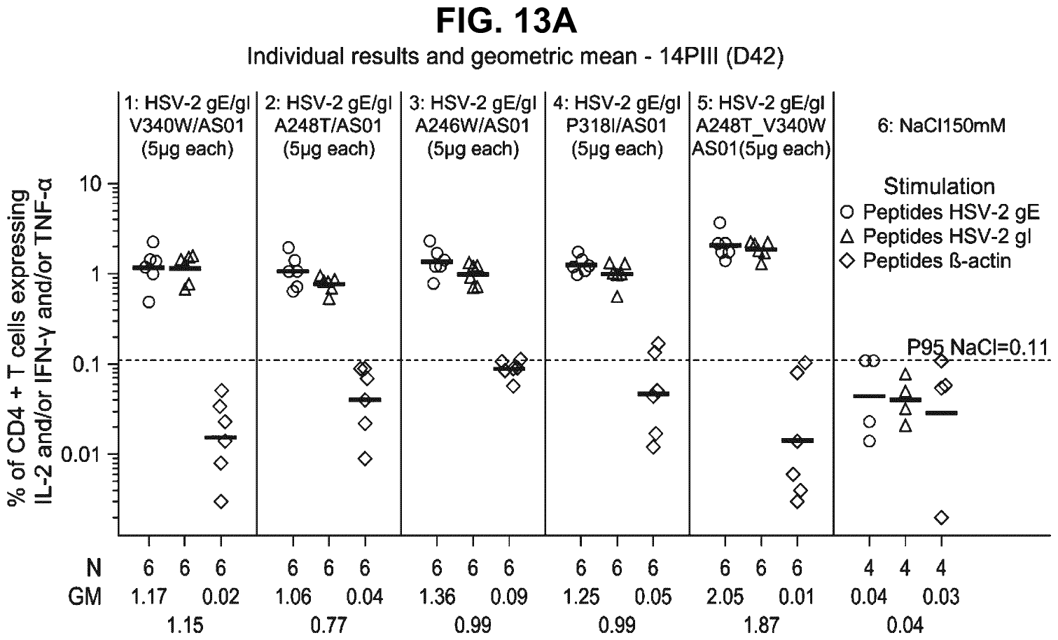

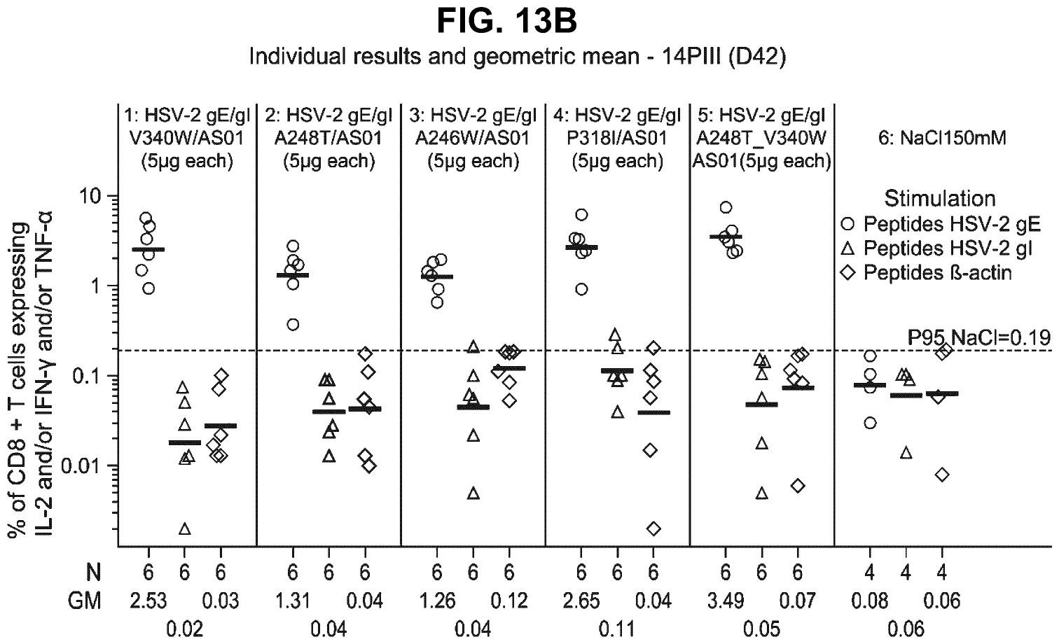

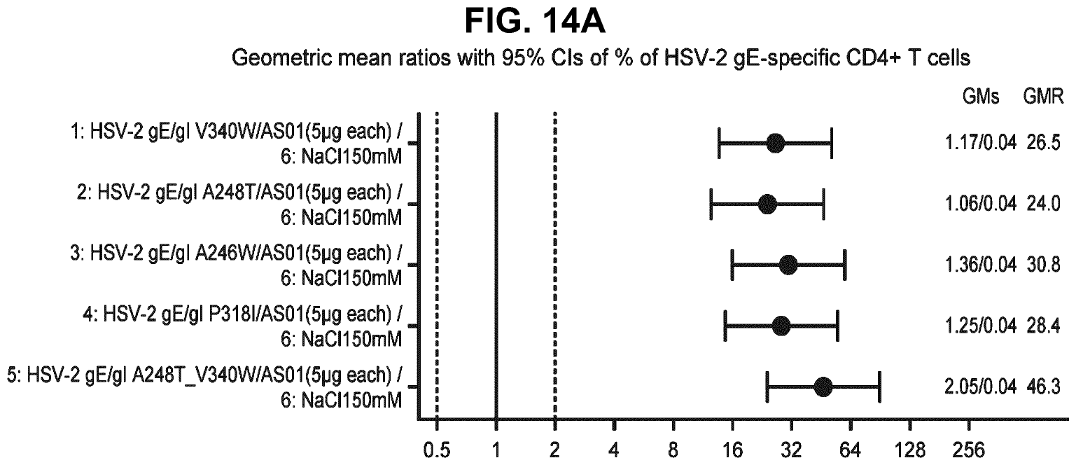

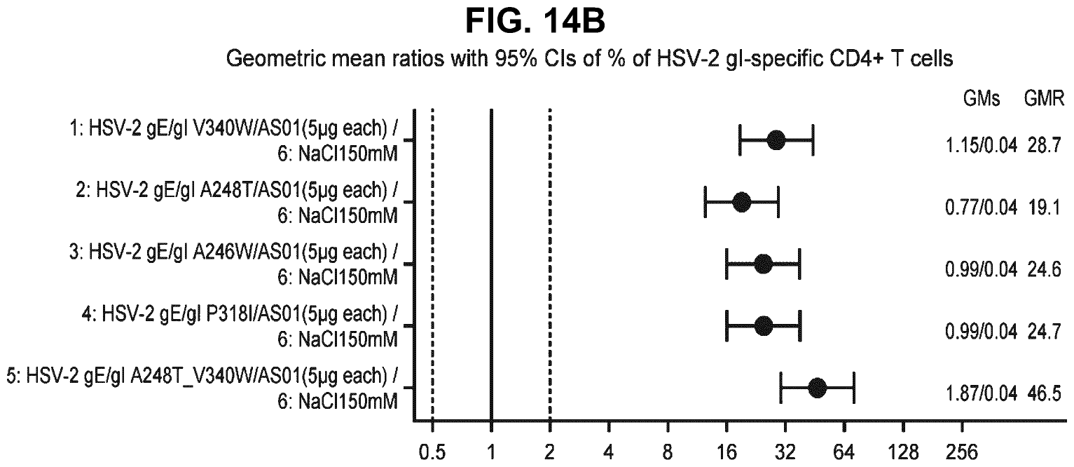

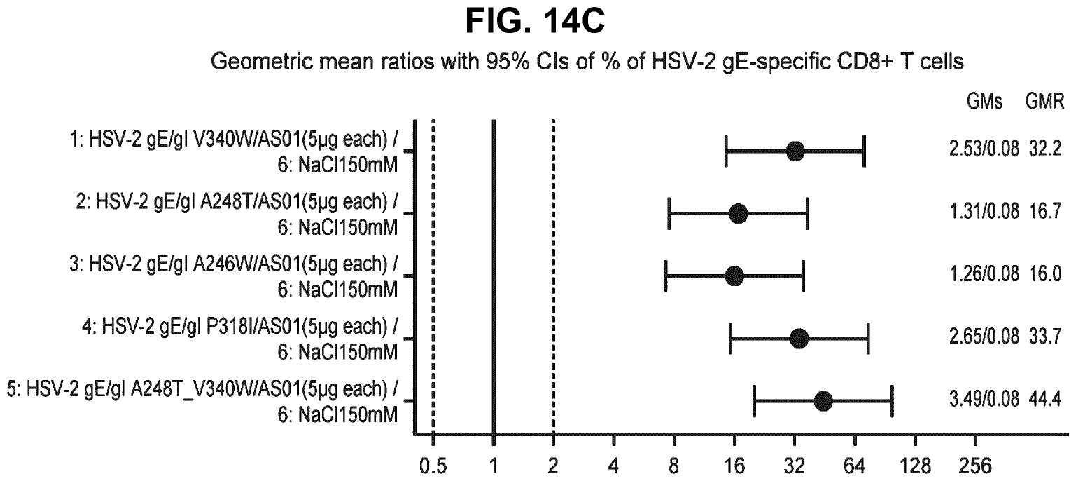

Compared to the NaCl control group, higher anti-HSV-2 gE and anti-HSV-2 gI-specific CD4+ T cell responses and anti-HSV-2 gE-specific CD8+T cell responses were detected 14 days after the third immunization in all groups of mice immunized with different mutated versions of AS01-adjuvanted HSV-2 gE/gI protein (>16-fold change between vaccine and NaCl groups with CIs not containing 1). No anti-HSV-2 gI-specific CD8+T cell response was detected in any of vaccinated groups compared to the NaCl control group (FIG. 13 & FIG. 14).

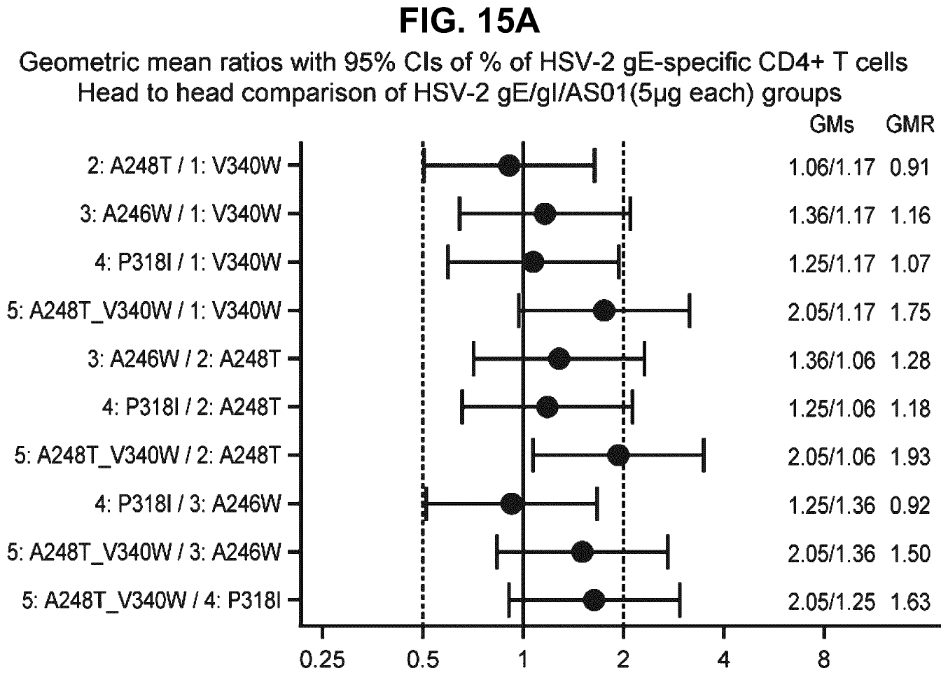

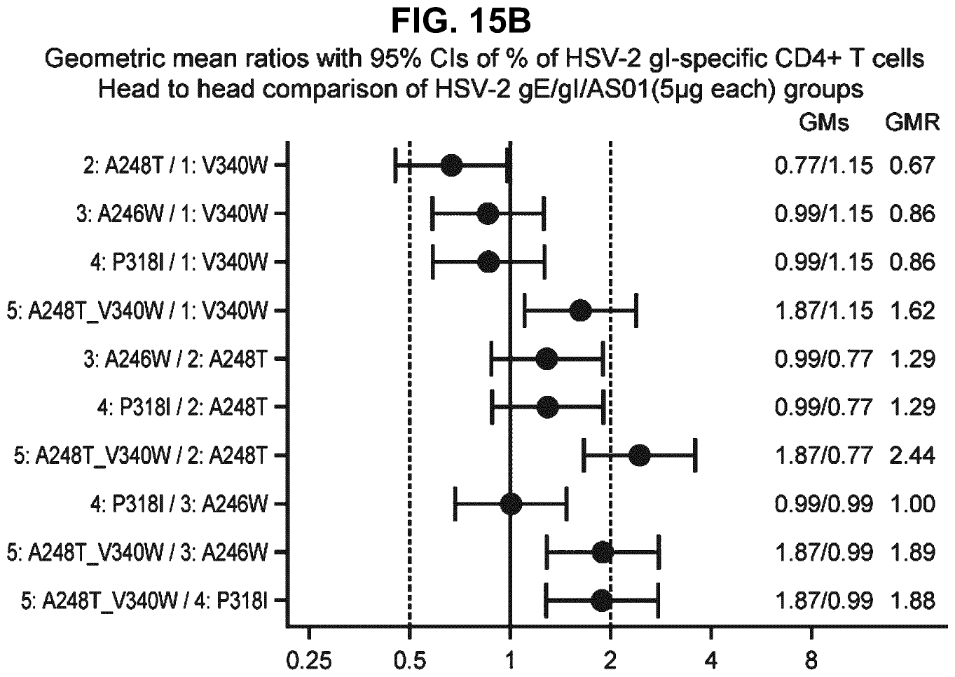

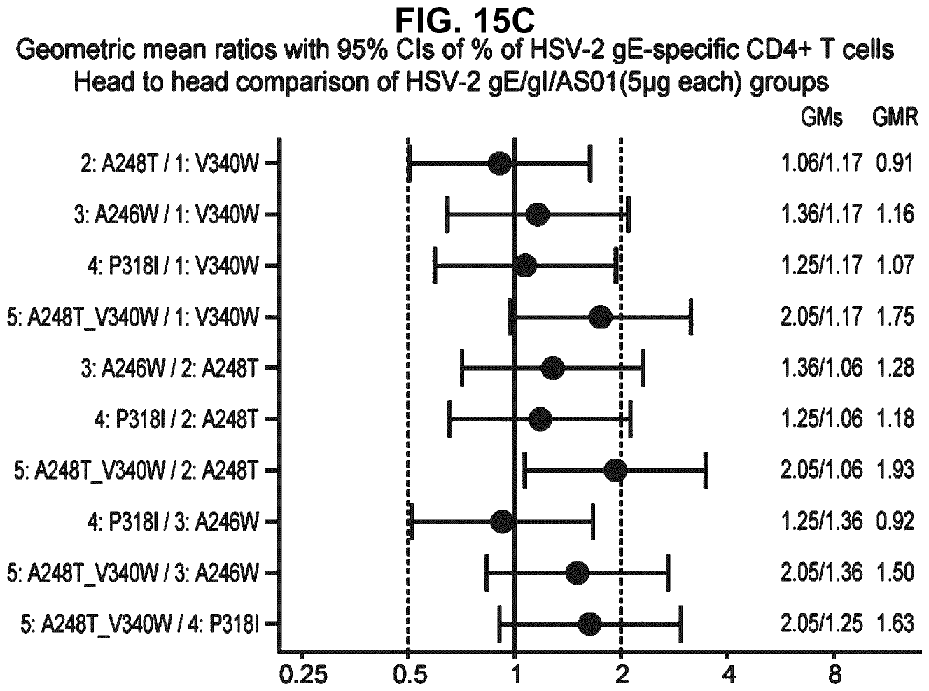

In this study, similar anti-HSV-2 gE- or anti-HSV-2 gI-specific CD4+T cell responses were detected between the different HSV-2 gE/gI mutant candidates. Only A248T_V340W mutant (group5) seemed to induce higher anti-HSV-2 gE- and anti-HSV-2 gI-specific CD4+T cell responses compared to A248T mutant (group2) with a fold increase close to 2 (GMRs of 1.93 and 2.44, respectively with CIs not containing 1) (FIG. 15A & FIG. 15B).

Similarly, for anti-HSV-2 gE-specific CD8+ T cell response, A248T_V340W mutant (group5) seemed to induce higher response compared to A248T mutant (group 2) with a fold increase of 2.66 (and CI does not include 1). In addition, response seemed to be higher with A248T_V340W (group5) compared to A246W (group3), and with P318I (group4) compared to A246W (grp3), with fold increases of 2.78 and 2.11, respectively (and CIs not including 1). There is a trend for higher response with V340W (group 1) compared to A248T (group2) or to A246W (group3), and with P318I (group4) compared to A248T (group2) with a fold increase of 2 (and CIs almost not including 1). No other differences between mutants were observed (with GMRs<2-fold change and/or CIs containing 1) (FIG. 15C).

The overall results suggest that the vaccine-specific T cell responses is slightly higher in group of mice immunized with the A248T_V340W mutant compared to group of mice immunized with the A248T mutant.

### Example 2

Objective

The general objective of this experiment is to evaluate the immunogenicity in mice of six different Lipid nanoparticle (LNP)-formulated Self Amplifying mRNA (SAM) vectors expressing new mutated versions of HSV-2 gE/gI sequence.

The primary objective was to compare to NaCl control group, the gE-specific polyclonal antibody (pAb) response induced by several LNP-formulated SAM HSV-2 gE/gI mutants 21 days post third immunization. At 21 days post third immunization, the anti-HSV-2 gI-specific polyclonal antibody response and the anti-HSV-2 gE and anti-HSV-2 gI-specific CD4+T cell responses induced by several LNP-formulated SAM HSV-2 gE/gI mutants were compared to NaCl control group. In addition, head-to-head comparison of all vaccine candidates in term of anti-HSV-2 gE and anti-HSV-2 gI-specific IgG antibody responses and CD4+ T cell responses were performed 21 days post third immunization.

Finally, the exploratory objectives were to evaluate, in different LNP-formulated SAM HSV-2 gE/gI mutants, the anti-HSV-2 gE and anti-HSV-2 gI-specific antibody responses after one and two immunizations, the anti-HSV-1 gE/gI cross-reactive antibody responses after three immunizations, the functions of vaccine-specific or anti-HSV-1 gE/gI cross reactive polyclonal antibodies and the levels of anti-HSV-2 gE- and anti-HSV-2 gI-specific CD8+T cell responses. Finally, the ability of vaccine-specific T cells, induced after three immunizations, to cross-react with HSV-1 gE antigens were also investigated in this study.

Study Design

Female CB6F1 inbred mice aged 6-8 weeks old from Harlan laboratory (OlaHsd) were randomly assigned to the study groups (n=6 gr1-6 & n=4/gr7) and kept at the institutional animal facility under specified pathogen-free conditions. CB6F1 mice (gr1-6) were intramuscularly (i.m) immunized at days 0, 21 & 42 with 0.8 μg of different versions of SAM HSV-2 gE/gI mutants formulated in Lipid nanoparticles (LNP). An additional group of mice was i.m injected with a saline solution (NaCl 150 mM) following the same schedule of immunization and used as negative control group (gr7). A total of 40 animals was used for the whole study.

Serum samples were collected at days 21, 42 & 63 post prime immunization (21PI, 21PII, 21PIII) to measure anti-HSV-2 gE- and anti-HSV-2 gI-specific and anti-HSV-1 gE/gI cross reactive IgG antibody responses and characterize the functions of vaccine-specific and HSV-1 cross reactive polyclonal antibodies. The spleens were collected at day 63 post prime immunization (21PIII) to evaluate ex-vivo systemic CD4+/CD8+ T cell responses towards HSV-2 gE and gI and HSV-1 gE antigens. Details for each group is described in the Table 4.

TABLE 4
Summary of the study design and formulation tested

| | | Treatment | | | | |
|---|---|---|---|---|---|---|
| | Number | | | Volume and | Immunization | Sample collection |
| | of | Vaccine name | Lot | route of | schedule | (organs, blood |
| Group | animals | and dose | numbers | injection | (Days) | volume) and days |
| 1 | 6 | LNP/SAM HSV-2 | N71700- | 50 μL | Days 0 & 28 | Days 21 & |
| | | gE/gI V340W | 28-1088 | i.m | | 42: Serum - |
| | | (0.8 μg) | | | | partial bleeding |
| 2 | 6 | LNP/SAM HSV-2 | N71700- | | | Day 63: Serum - |
| | | gE/gI A248T | 28-1089 | | | final bleeding + |
| | | (0.8 μg) | | | | Spleen |
| 3 | 6 | LNP/SAM HSV-2 | N71700- | | | |
| | | gE/gI A246W | 28-1095 | | | |
| | | (0.8 μg) | | | | |
| 4 | 6 | LNP/SAM HSV-2 | N7 1700- | | | |
| | | gE/gI P318I | 28-1096 | | | |
| | | (0.8 μg) | | | | |
| 5 | 6 | LNP/SAM HSV-2 | N71700- | | | |
| | | gE/gI A248T_V340W | 28-1090 | | | |
| | | (0.8 μg) | | | | |
| 6 | 6 | LNP/SAM HSV-2 | N71700- | | | |
| | | gE/gI insert ARAA | 28-1055 | | | |
| | | (0.8 μg) | | | | |
| 7 | 4 | NaCl150 mM | N/A | | | |

Materials and Methods

Investigational Products

SAM HSV-2 gE/gI mutants were generated by In vitro Transcription (IVT) according to established protocols. Preparation of LNP with SAM followed established methods. In this method, SAM-LNP is prepared by rapidly mixing ethanolic solutions of lipids with aqueous buffers that contain SAM. The rapid mixing results in a supersaturation of hydrophobic components that have ionically paired with SAM. The SAM/lipid complexes condensation and precipitate through nucleation mediated precipitation, yielding small and narrowly disperse nanoparticles. Following this mixing step, the lipid nanoparticles mature to entrap the RNA and then are transferred to a final Tris/sucrose storage buffer through a buffer exchange step. The LNP solutions are then characterized for size, lipid content, RNA entrapment, final SAM concentration, and in vitro potency. The materials can be frozen at −80° C. for over 6 months after the addition of 5% (w/v) sucrose.

Total Anti-HSV-2 gE & gI-Specific IgG Antibodies Measured by ELISA

As described for Example 1 above.

Total Anti-HSV-1 gE/gI Cross Reactive IgG Antibodies Measured by ELISA

As described for Example 1 above.

HSV-2 gE- and gI-Specific and HSV-1 gE Cross-Reactive CD4+/CD8+ T Cell Responses Measured by ICS Assay

As described for Example 1 above, except that additionally, cross reactive CD4+ and CD8+ T cell responses towards HSV-1 gE antigen were analyzed after ex-vivo stimulation with HSV-1 gE peptides pool. For this, the Cell preparation section differs in that the cells were stimulated for 6 hours (37° C., 5% CO2) with anti-CD28 (BD Biosciences, clone 37.51) and anti-CD49d antibodies (BD Biosciences, clone 9C10 (MFR4.B)) at 1 μg/mL per well, containing 100 μL of either
15 mers overlapping peptides pool covering the sequence of gE protein from HSV-2 (1 μg/mL per peptide per well).
15 mers overlapping peptides pool covering the sequence of gI protein from HSV-2 (1 μg/mL per peptide per well).
15 mers overlapping peptides pool covering the sequence of gE protein from HSV-1 (1 μg/mL per peptide per well).
15 mers overlapping peptides pool covering the sequence of Human β-actin protein (1 μg/mL per peptide per well) (irrelevant stimulation).
RPMI/additives medium (as negative control of the assay).
PMA—ionomycin solution (Sigma, P8139) at working concentrations of 0.25 μg/mL and 2.5 μg/mL respectively (as positive control of the assay).

Evaluation of the Ability of Polyclonal Sera to Bind and Activate Mouse FcγRIII after Incubation with HSV-2 gE/gI Transfected Cells (ADCC Bioassay—Promega)

As described for Example 1.

Competitive ELISA to Evaluate the Ability of Vaccine Specific Antibodies to Decrease Human IgG Fc Binding by HSV-2 gE/gI Protein

As described for Example 1.

Competitive ELISA to Evaluate the Ability of HSV-1 Cross-Reactive Antibodies to Decrease Human IgG Fc Binding by HSV-1 gE/gI Protein

As described for Example 1.

In-Vitro HSV-2 Neutralization Assay

As described for Example 1.

In-Vitro HSV-1 Neutralization Assay

As described for Example 1.

Statistical Methods

The distribution of each response was assumed to be lognormal.

For each vaccine-specific IgG antibody response (gE or gI), a two-way analysis of variance (ANOVA) model was fitted on log₁₀ titers by including groups (all except the NaCl one), time points (Day21 (21PI), Day42 (21PII) and Day63 (21PIII)) and their interactions as fixed effects. The NaCl group was not included as no response and variability was observed. Variance-covariance model selection was based on AICC criterion and individual data plot examination.

On each vaccine-specific response, the variance-covariance for time points was modelled via a Compound Symmetry matrix.

$$\begin{matrix}[ \begin{matrix}{σ}^{2} + {σ}_{1} & {σ}_{1} & {σ}_{1} \\ {σ}_{1} & {σ}^{2} + {σ}_{1} & {σ}_{1} \\ {σ}_{1} & {σ}_{1} & {σ}^{2} + {σ}_{1}\end{matrix} ] \\ CS - Compound Symmetry\end{matrix}$$

The compound symmetry considers same variance and same correlation between timepoints. The same variance-covariance matrix was modelled for each vaccine group, indicating same variance between groups.

Geometric means and their 95% CIs are derived from these models.

Despite the fact that the NaCl group was not included in the ANOVA models, the comparisons associated to the primary objective were computed as follows: geometric means and 95% CI of vaccinated groups derived from the above models were divided by titer given to all the NaCl recipients at the last timepoint. The resulting ratios should be understood as geometric mean ratios with their corresponding 95% CI.

For head to head comparison of vaccinated groups (at the last time point) and time point comparisons (PII/PI, PIII/PII, and PIII/PI) within each group, all the geometric mean ratios and their 95% CIs were derived from the models.

For gE/gI cross-reactive IgG antibody response, a one-way analysis of variance (ANOVA) model was fitted on log₁₀ titers by including groups (all except the NaCl one) as fixed effect. The NaCl group was not included as no response and variability was observed. No clear heterogeneity of variance was detected and therefore identical variances were assumed for the different groups. Geometric means and their 95% CIs are derived from this model. For comparison of vaccinated to NaCl groups, geometric means and 95% CI of vaccinated groups derived from the above model were divided by titer given to all the NaCl recipients. The resulting ratios should be understood as geometric mean ratios with their corresponding 95% CI. For head to head comparison of vaccinated groups, all the geometric mean ratios and their 95% CIs were derived from the model.

On each vaccine-specific or cross-reactive % of CD4+/CD8+ T-cell responses (gE or gI), a one-way analysis of variance (ANOVA) model was fitted on log 10 frequencies by including groups (all groups including the NaCl group) as fixed effect. No clear heterogeneity of variance was detected for % of HSV-2 gE-specific CD4+ T-cell response and therefore identical variances were assumed for the different groups. For other responses, different variances were modeled for the different groups. For both % of CD4+ and CD8+ T cell responses, the NaCl threshold was based on P95 of data across stimulation in NaCl negative control group. No modelling was performed on % of HSV-1 gE cross-reactive CD4+ T-cells and on % of HSV-2 gI-specific CD8+ T-cells since response were below the P95 NaCl threshold for all vaccine groups. Geometric means and geometric mean ratios (with their corresponding 95% CIs) were derived from these models.

For HSV-2 MS-specific neutralizing antibody titers, a one-way analysis of variance (ANOVA) model was fitted on log₁₀ values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from this model. The same model was also performed on HSV-1 cross-reactive neutralizing antibody titers.

Results

All LNP/SAM HSV-2 gE/gI Mutants Induced Vaccine-Specific IgG Antibody Responses

Note that one sample in LNP/SAM-HSV-2 gE/gI A248T_V340W group (sample 4.5) was not evaluated by ELISA 21 days post second immunization due to not enough serum available.

The anti-HSV-2 gE & gI-vaccine-specific and anti-HSV-1 gE/gI cross-reactive IgG antibody responses were investigated by ELISA.

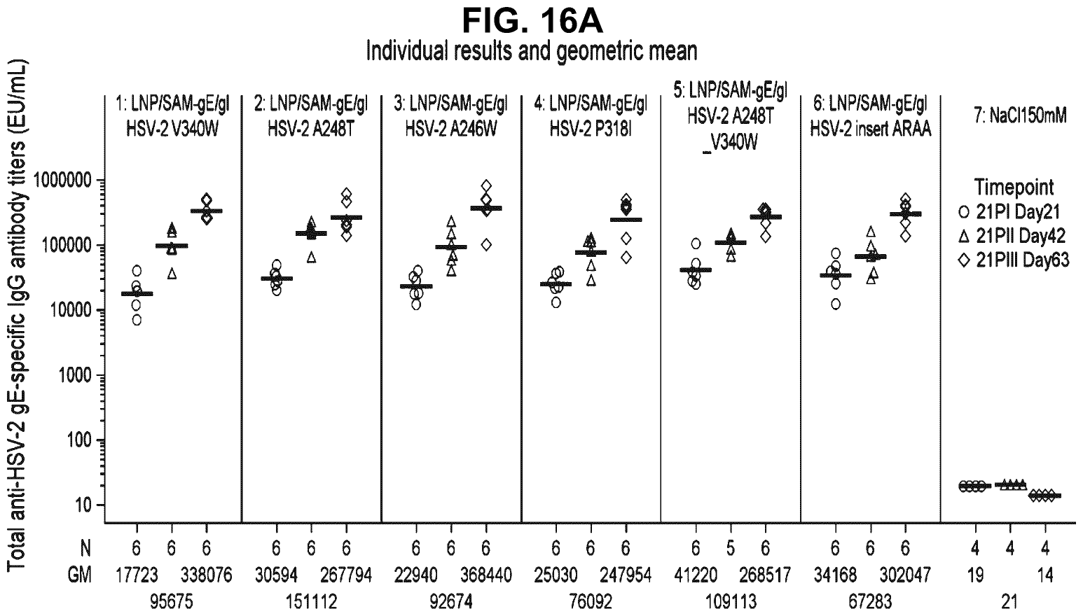

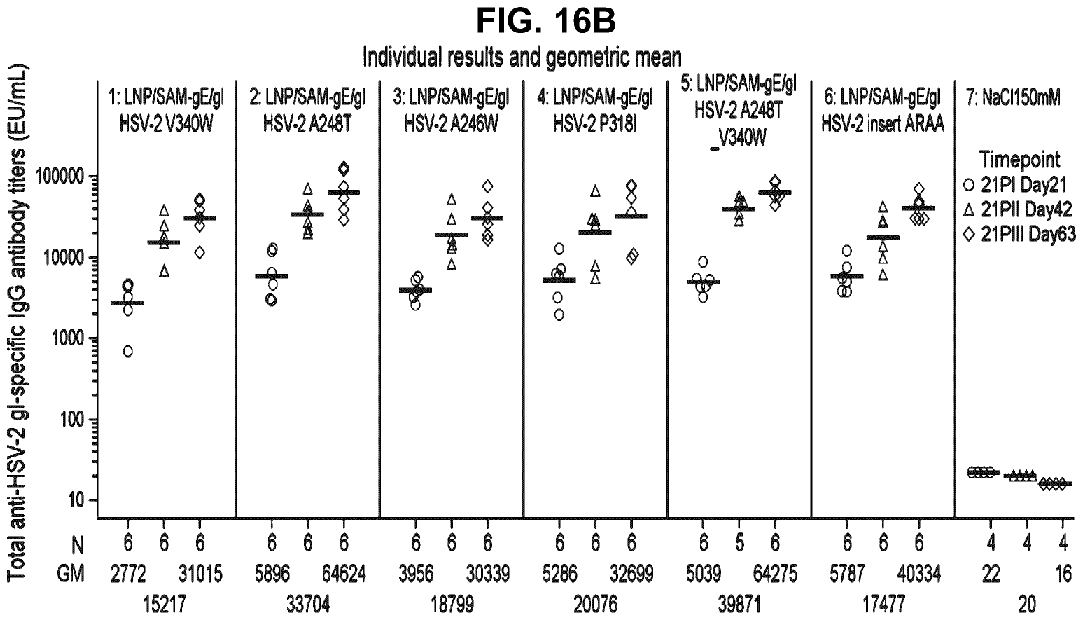

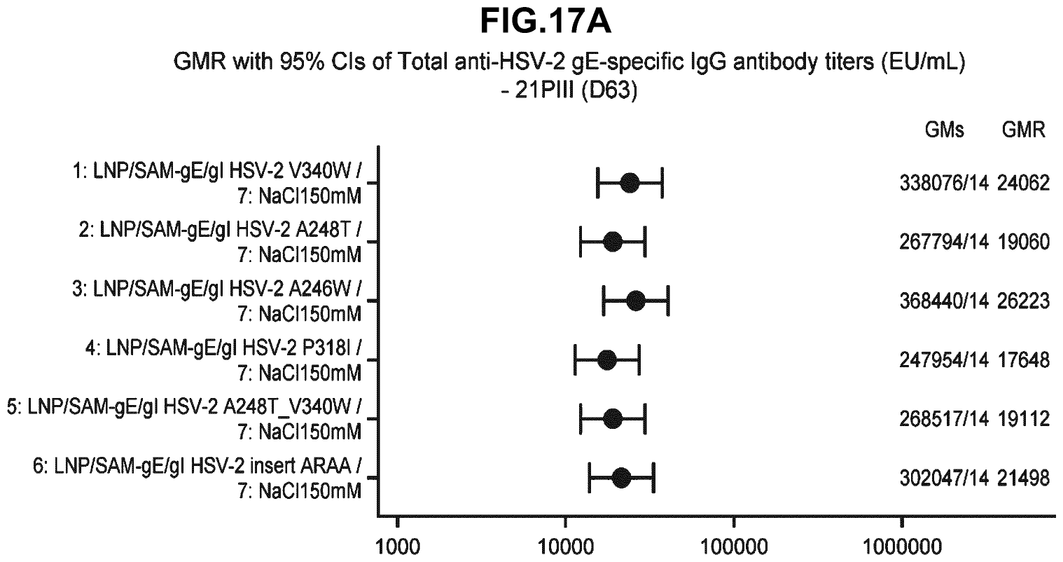

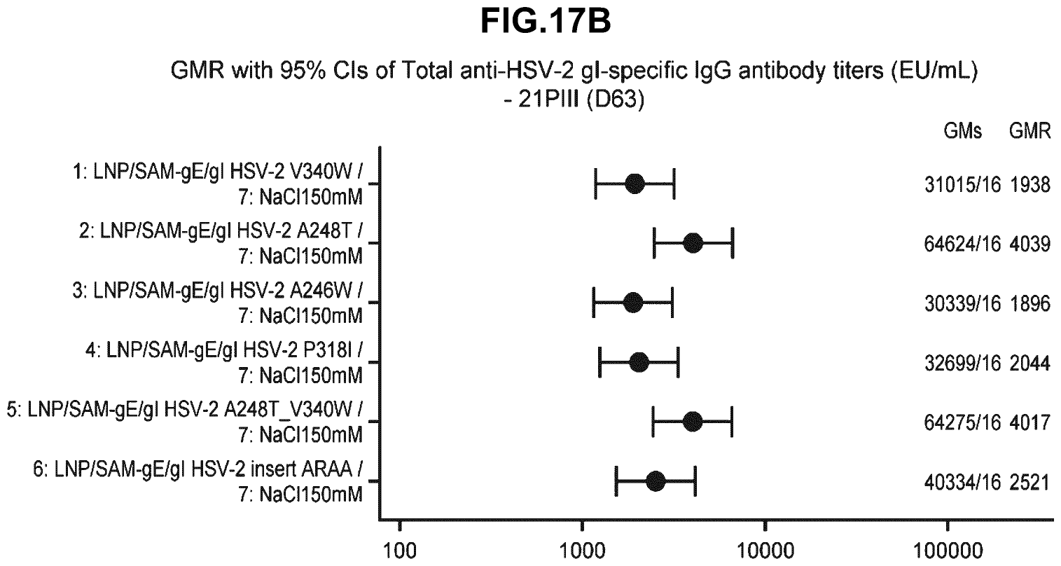

As expected, anti-HSV-2 gE-specific or anti-HSV-2 gI responses were not observed in NaCl control group (<20 EU/mL for all mice) while all the LNP-formulated SAM HSV-2 gE/gI vaccinated-mice produced a response above 30.000 EU/mL at the last time point (FIG. 16A & FIG. 16B). At 21 days post third immunization and compared to NaCl control group, high anti-HSV-2 gE or anti-HSV-2 gI- specific IgG antibody responses were induced in all groups of mice immunized with the different versions (mutated and amino acid insertion) of LNP-formulated SAM HSV-2 gE/gI vector (all GMRs>17.000 for gE and all GMRs>1.800 for gI) (FIG. 17A & FIG. 17B).

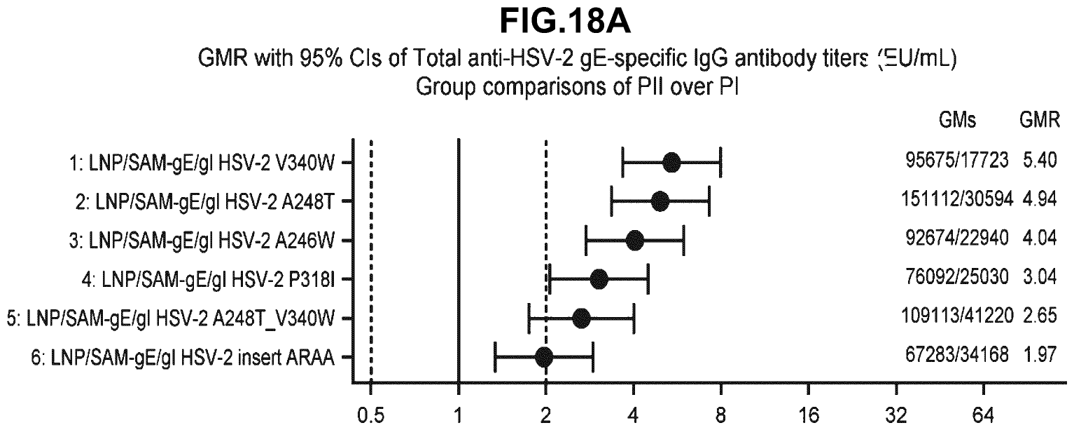

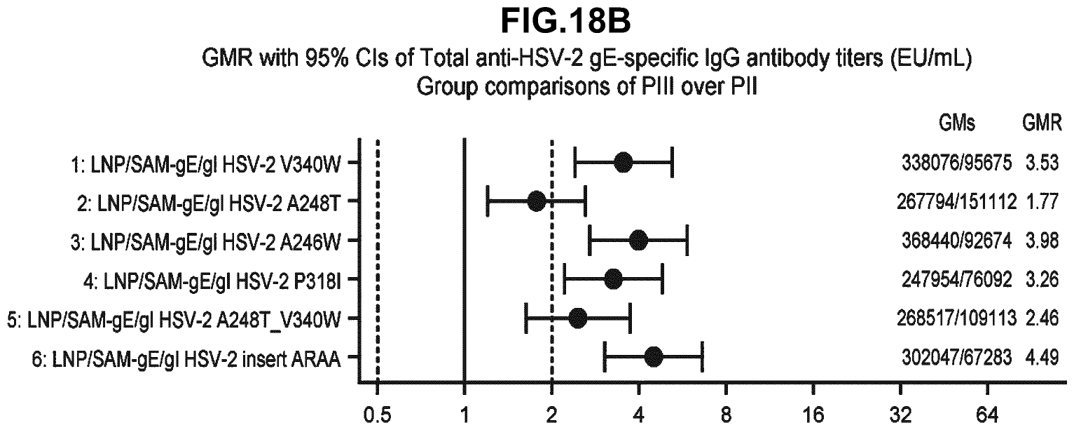

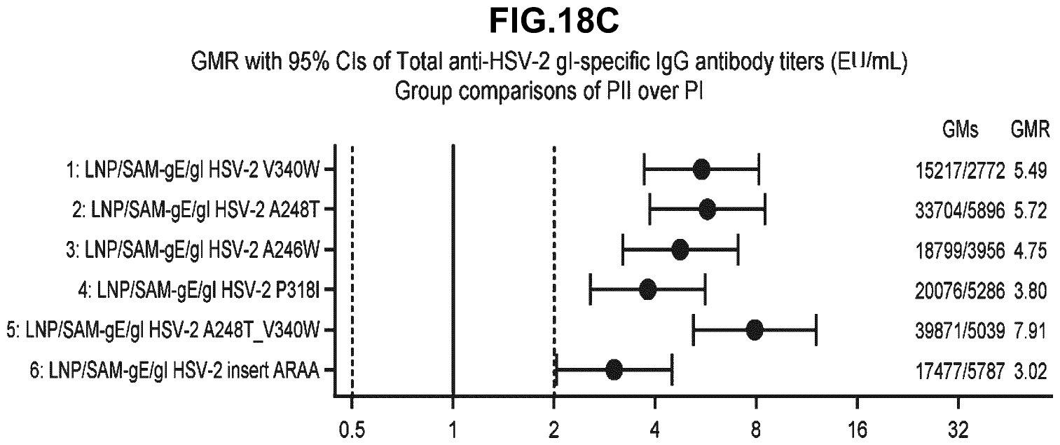

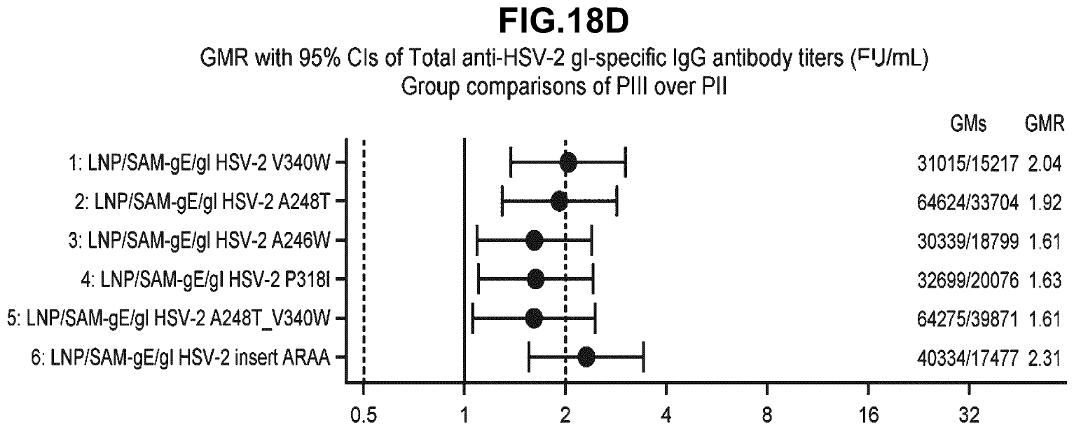

In all groups of mice immunized with LNP-formulated SAM HSV-2 gE/gI mutants, levels of anti-HSV-2 gE and anti-HSV-2 gI-specific IgG antibody responses increased after the second immunization (day42 (21PII)) compared to the first one (day21 (21PI)), with a fold increase ranging from 2 to 8 (FIG. 18A & FIG. 18C).

For all HSV-2 gE/gI mutants except A248T mutant (group2), a booster effect on HSV-2 gE-specific antibody response was also observed 21 days after the third immunization (day 63(21PIII)) compared to the second one (day42 (21PII)), with a fold increase ranging from 2.46 to 4.49 (with CI not containing 1). For A248T mutant, a fold increase of 1.77 was observed (CI not containing 1) (FIG. 18B).

Between the second and third vaccine dose, a clear booster effect on the levels of HSV-2 gI-specific antibody response was only detected for the groups of mice immunized with LNP-formulated SAM HSV-2 gE/gI V340W (group 1) and insert ARAA (group 6) mutants (2 and 2.3-fold increases with CIs not containing 1). A smaller booster effect was also observed with the other mutants with a 1.6-1.9-fold increase (CIs not containing 1) (FIG. 18D).

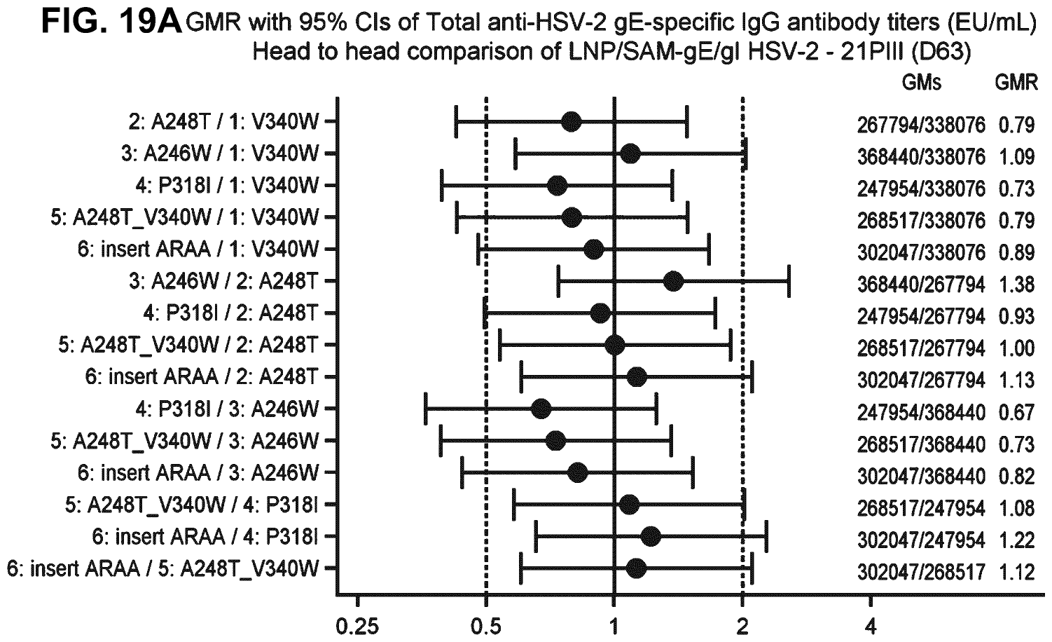

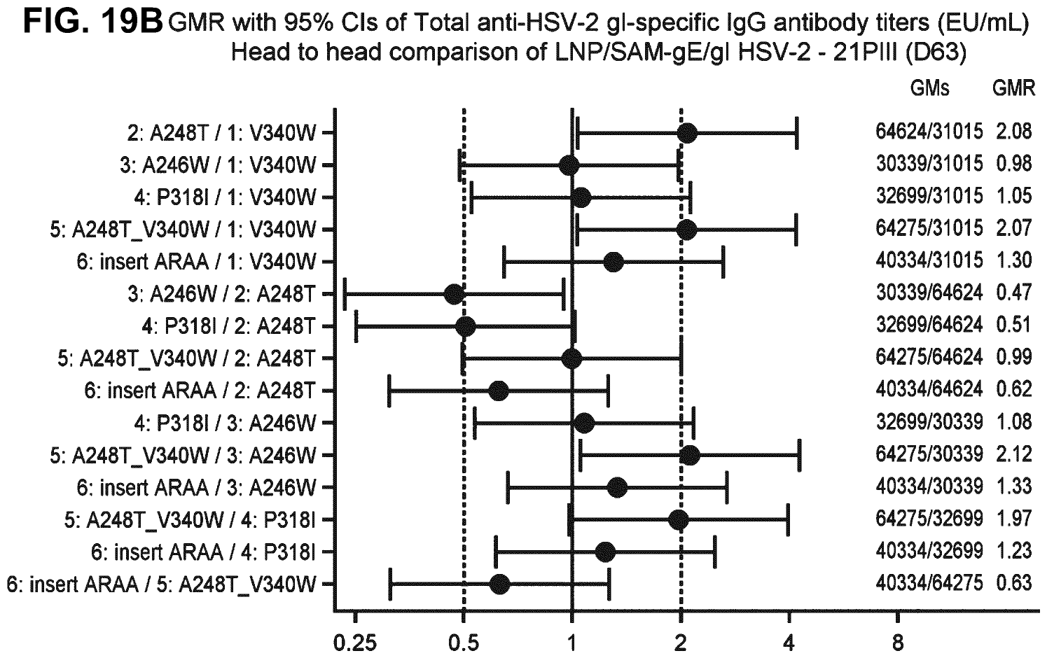

In overall, these results suggest similar HSV-2 gE-specific antibody response between the different HSV-2 gE/gI mutant candidates at 21 days post third immunization (with GMRs close to 1-fold change and CIs containing 1) (FIG. 19A). In contrast, for HSV-2 gI-specific antibody response, several differences between HSV-2 gE/gI mutant candidates seem to be observed at 21 days post third immunization. Indeed, response induced by both A248T (group 2) and A248T_V340W (group 5) mutants seemed to be twice higher than the one induced by V340W (group 1), A246W (group 3) and P318I (group 4) mutants (GMRs of 2 with CIs either not containing 1 which indicates an effect, or CIs almost not containing 1 which indicates a trend for an effect). No other differences between mutants were observed (with GMRs<2-fold change and CIs containing 1) (FIG. 19B).

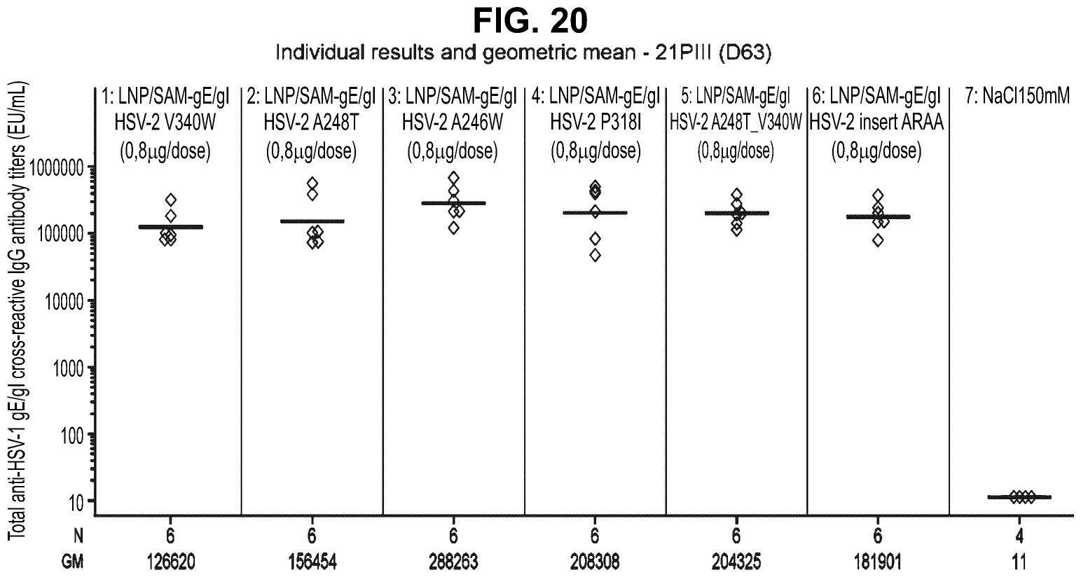

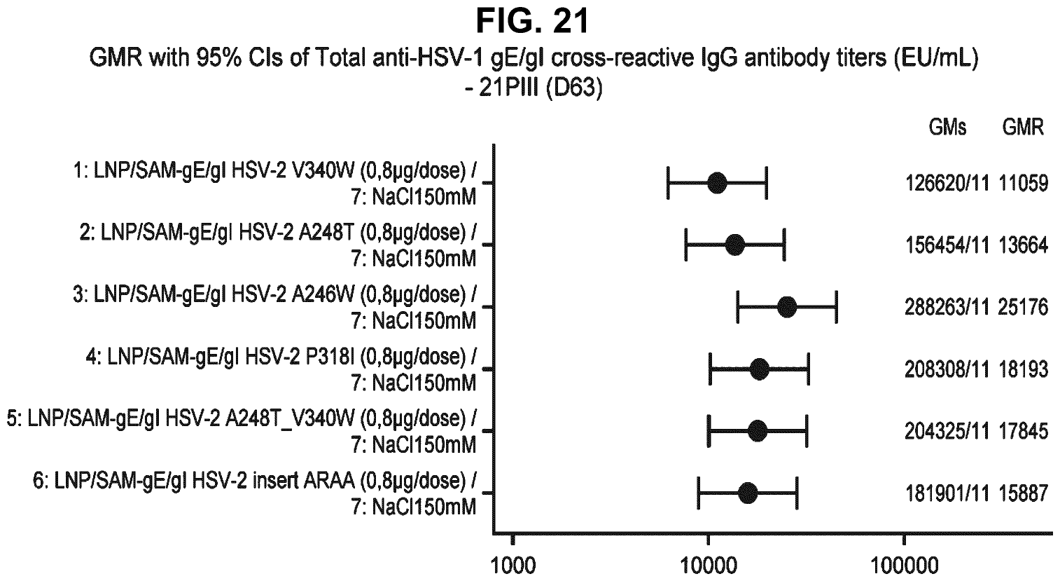

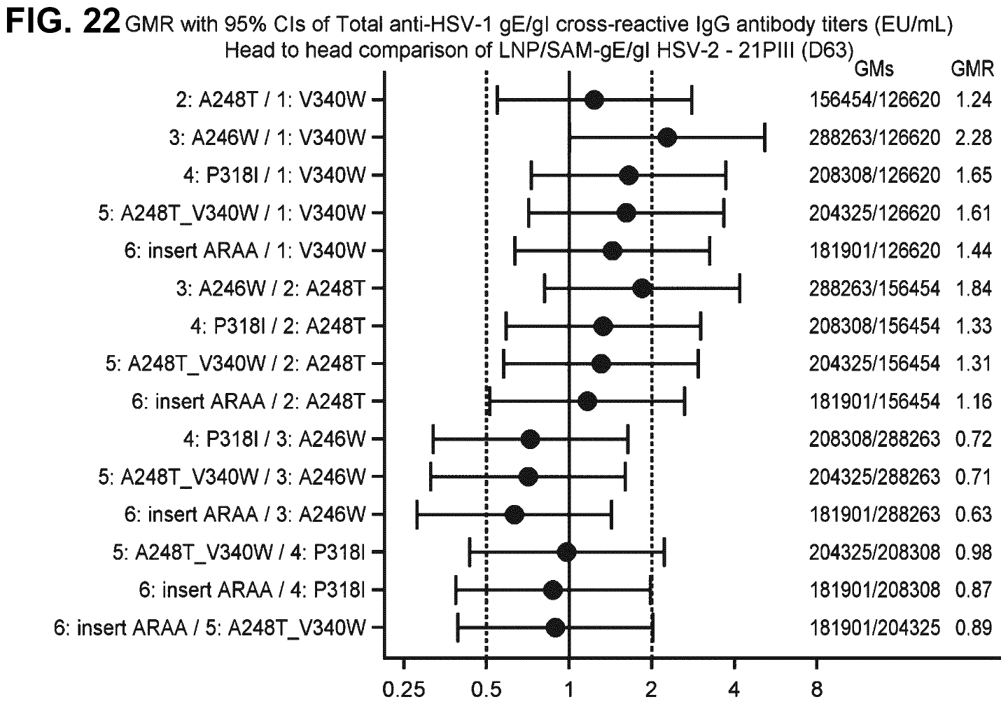

As expected, anti-HSV-1 gE/gI cross-reactive response was not observed in NaCl control group (11 EU/mL for all mice). At 21 days post third immunization and compared to NaCl control group, high anti-HSV-1 gE/gI cross-reactive IgG antibody response was induced in all groups of mice immunized by different versions (mutated and amino acid insertion) of LNP-formulated SAM HSV-2 gE/gI vector (all GMRs>11.000) (FIG. 20, FIG. 21). In overall, head to head comparison of vaccine-immunized groups of mice suggests similar anti-HSV-1 gE/gI cross-reactive IgG antibody response 21 days post third immunization (with most GMRs<2-fold change and CIs containing 1). Only A246W mutant (group 3) seemed to induce higher response compared to V340W mutant (group 1) with a fold increase of 2.28 (CI not containing 1) (FIG. 22).

Different LNP/SAM HSV-2 gE/gI Mutants can Induce Functional Vaccine-Specific and HSV-1 gE/gI Cross-Reactive Antibody Response

In this study, vaccine HSV-2 gE/gI-specific & HSV-1 gE/gI cross-reactive antibody function were only investigated in the sera collected at 21 days post third immunization. In this regard, the ability of vaccine-induced pAbs to neutralize HSV-2 MS or HSV-1 VR1789 viruses, to bind and activate mFcγRIII expressed by Jurkat reporter cell line after incubation with HSV-2 gE/gI transfected cells and to decrease in-vitro binding of human IgG Fc by HSV-2 or HSV-1gE/gI protein was investigated.

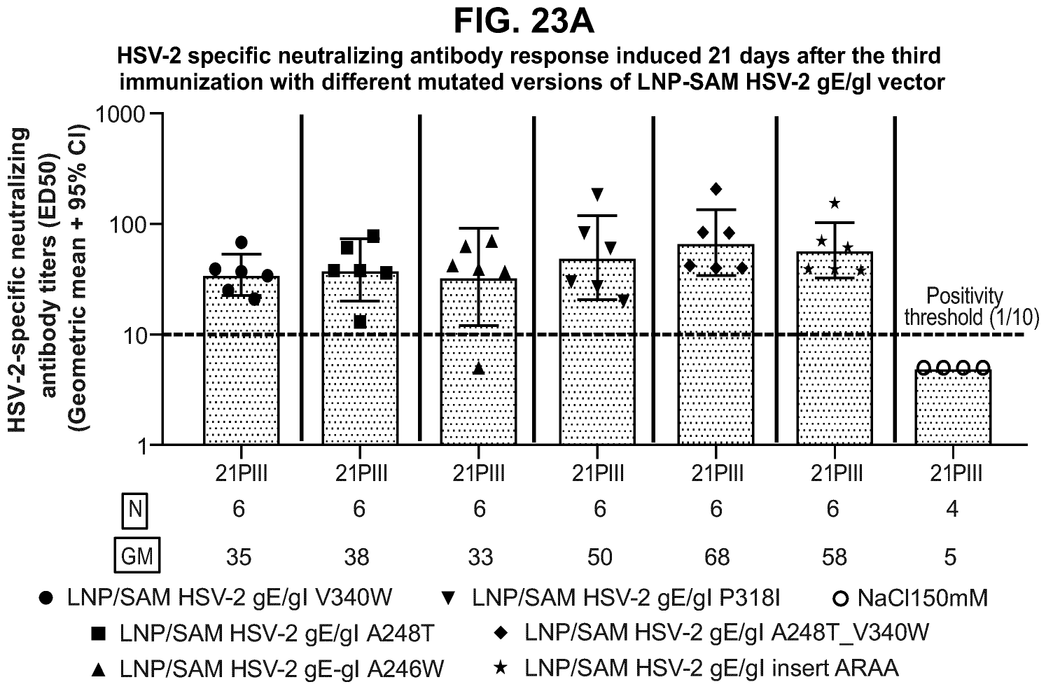

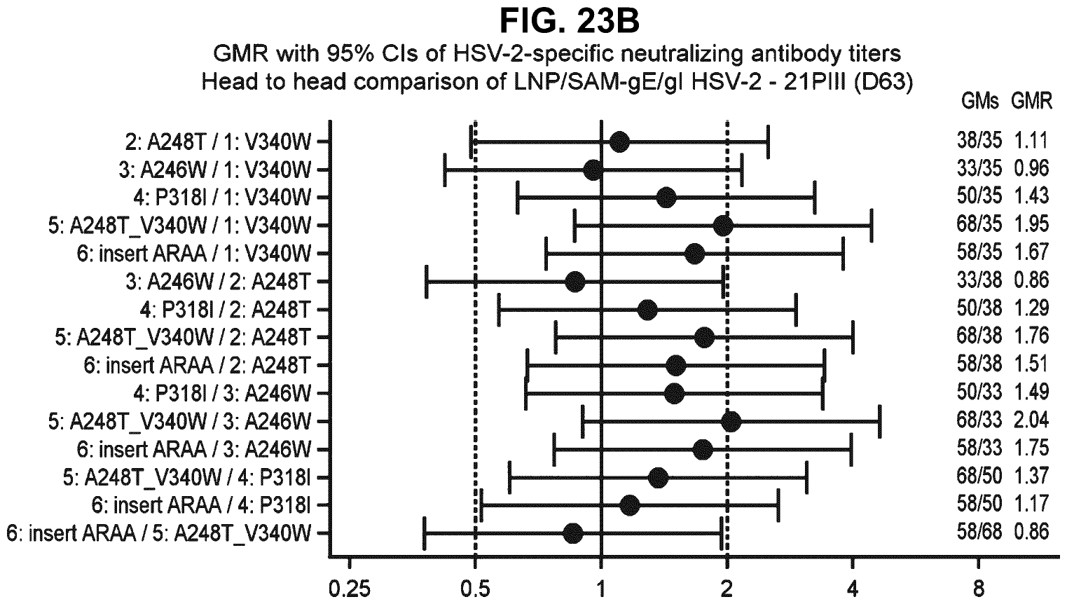

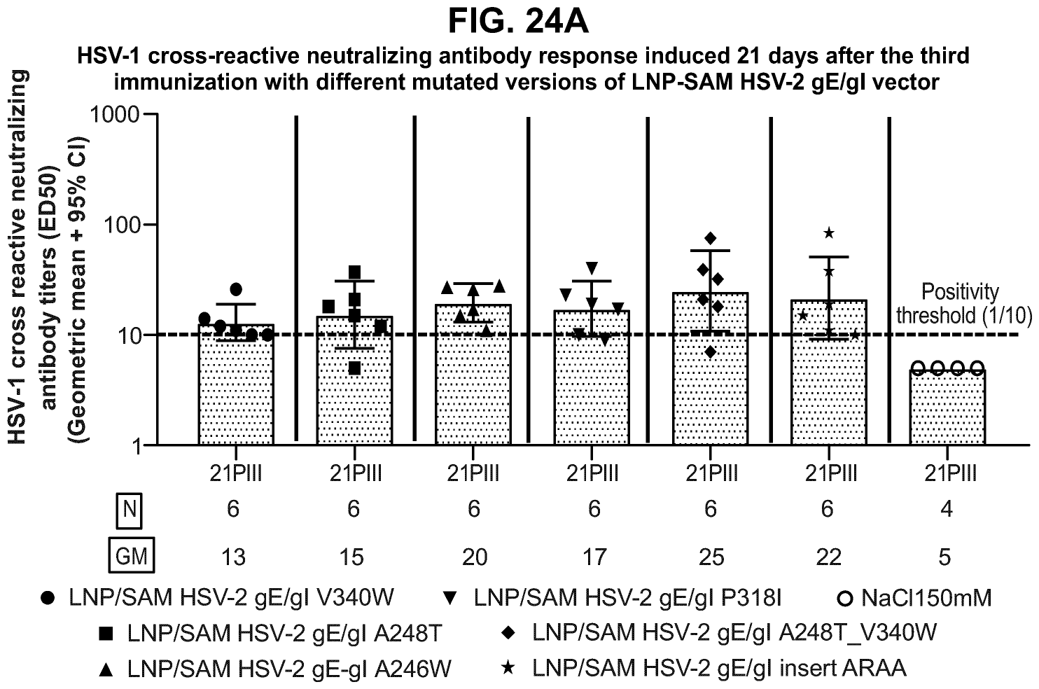

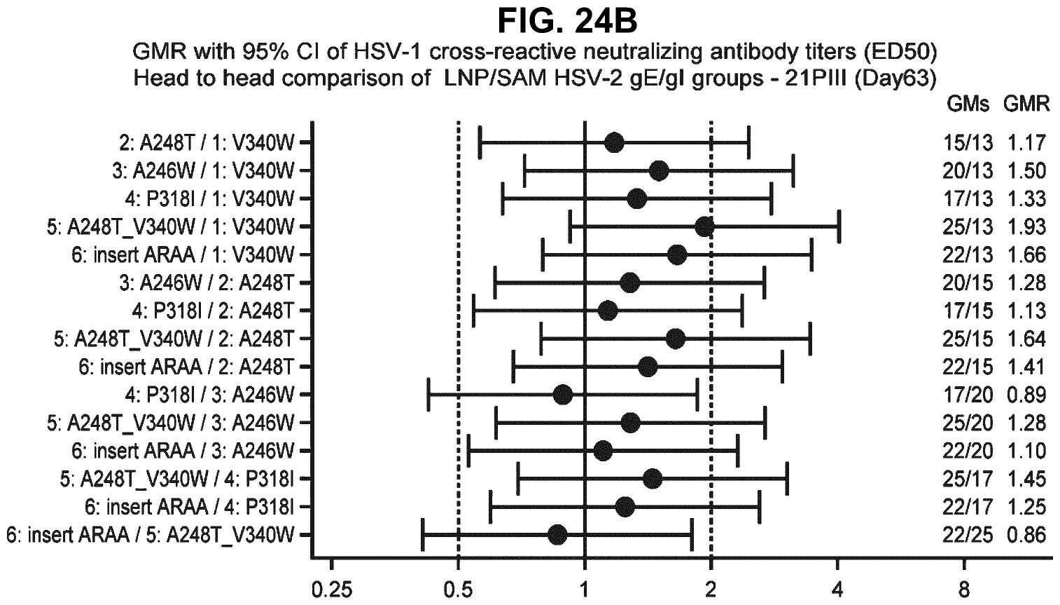

Very low specific neutralizing (nAb) antibody responses directed to HSV-2 MS or HSV-1 VR1789 strains strain was were detected in all groups of mice immunized with different versions (mutated and amino acid insertion) of LNP-formulated SAM-HSV-2 gE/gI vector. Results suggest no clear difference in term of vaccine-specific or cross-reactive antibody neutralizing activity between the different mutants (GMRs<2-fold change with all CIs containing 1). A few vaccine comparisons (in particular, A248T_V340W (group 5) versus V340W (group 1)) showed a 2-fold change but failed to reach significance due to large variability (FIG. 23A, & FIG. 23B). On the side of the cross-reactive nAb response directed against HSV-1 VR1789 strain no difference was observed between the different candidates (see FIG. 24A & FIG. 24B).

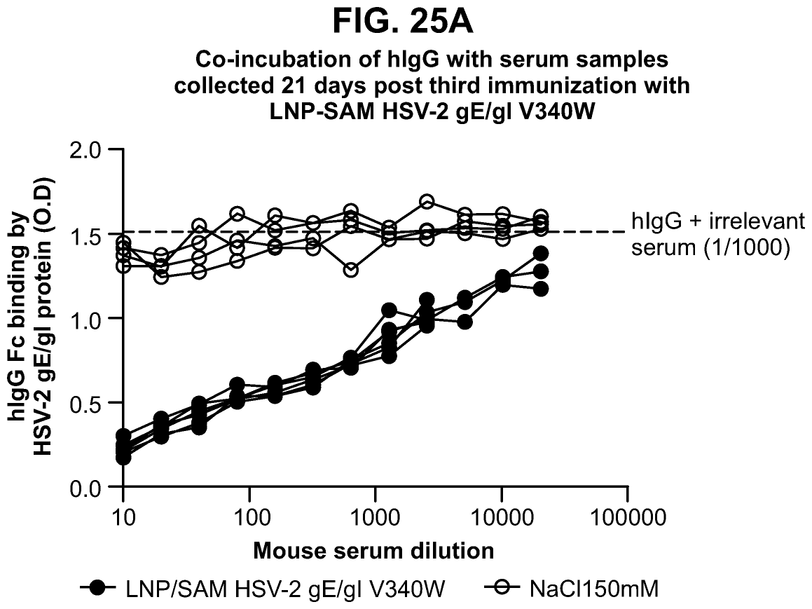

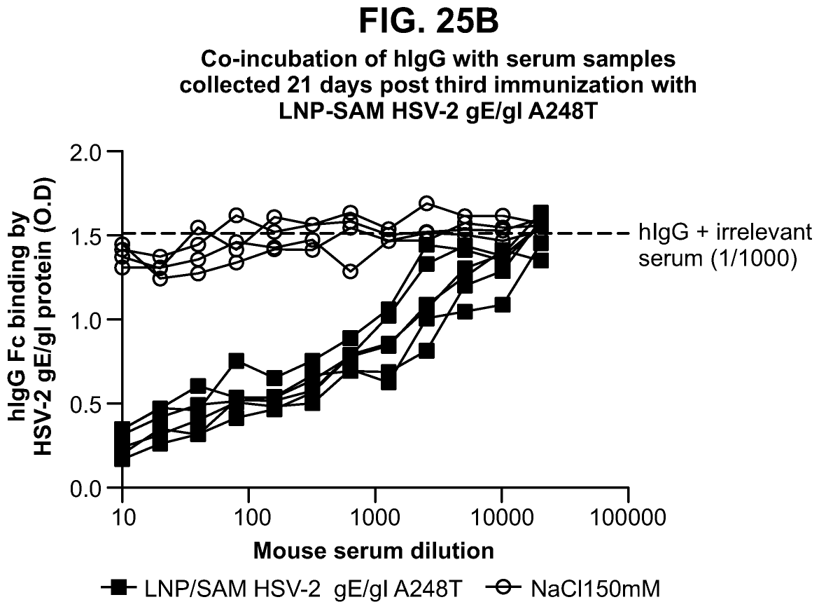

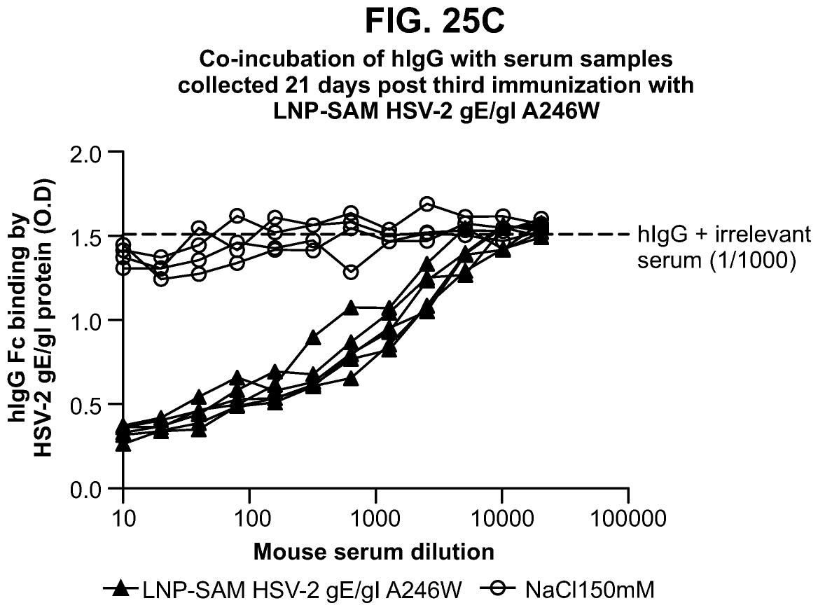

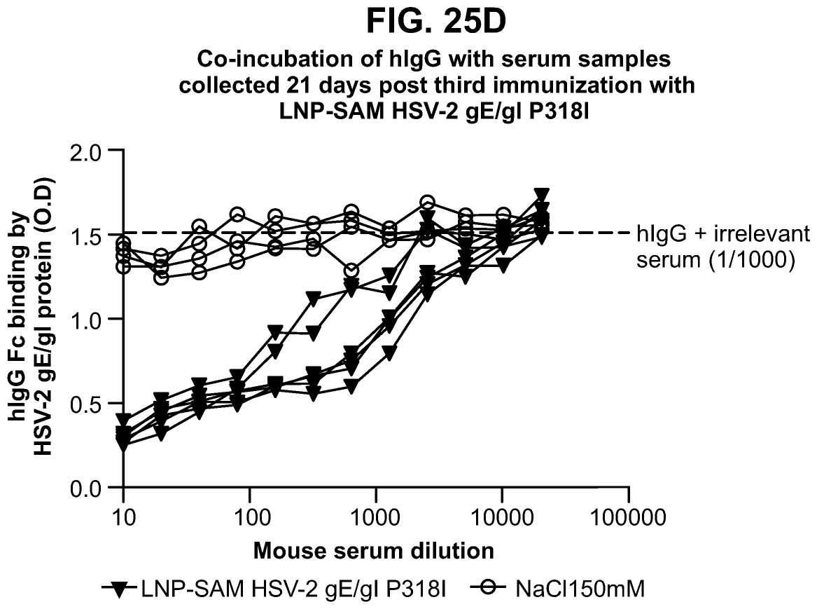

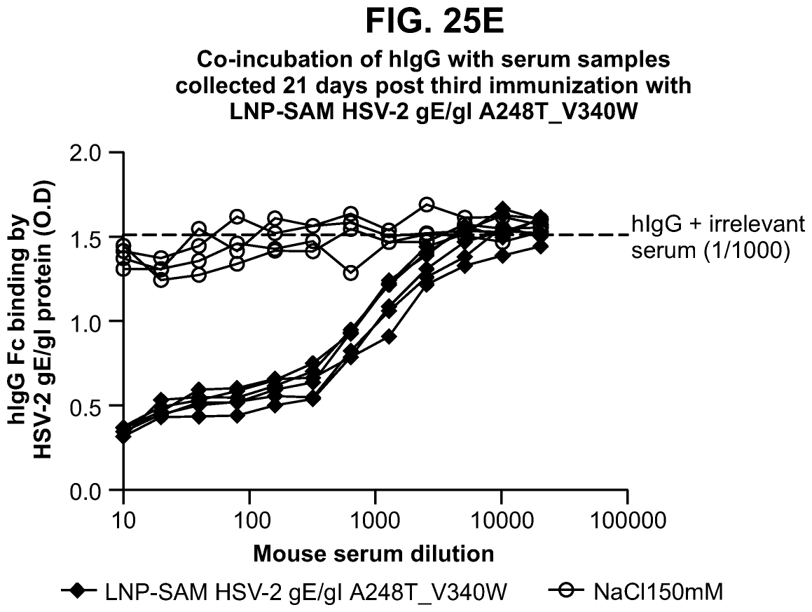

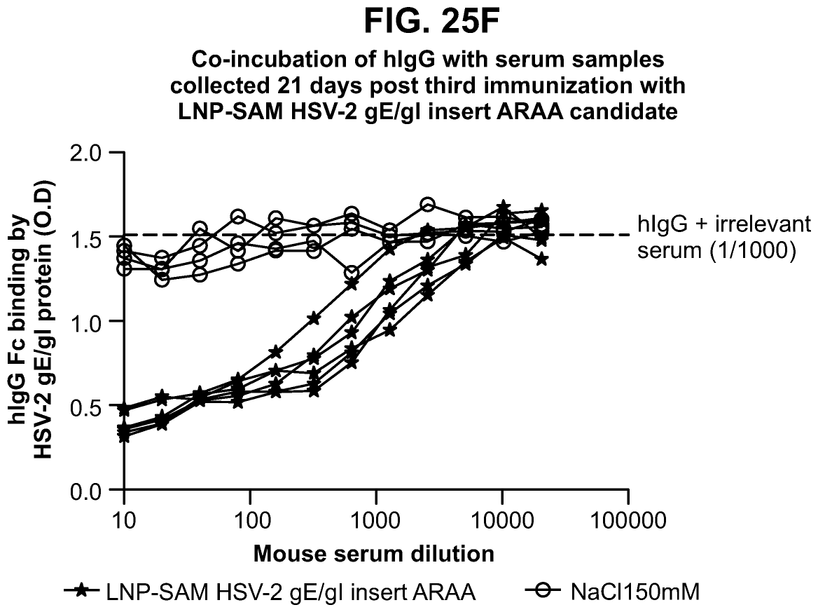

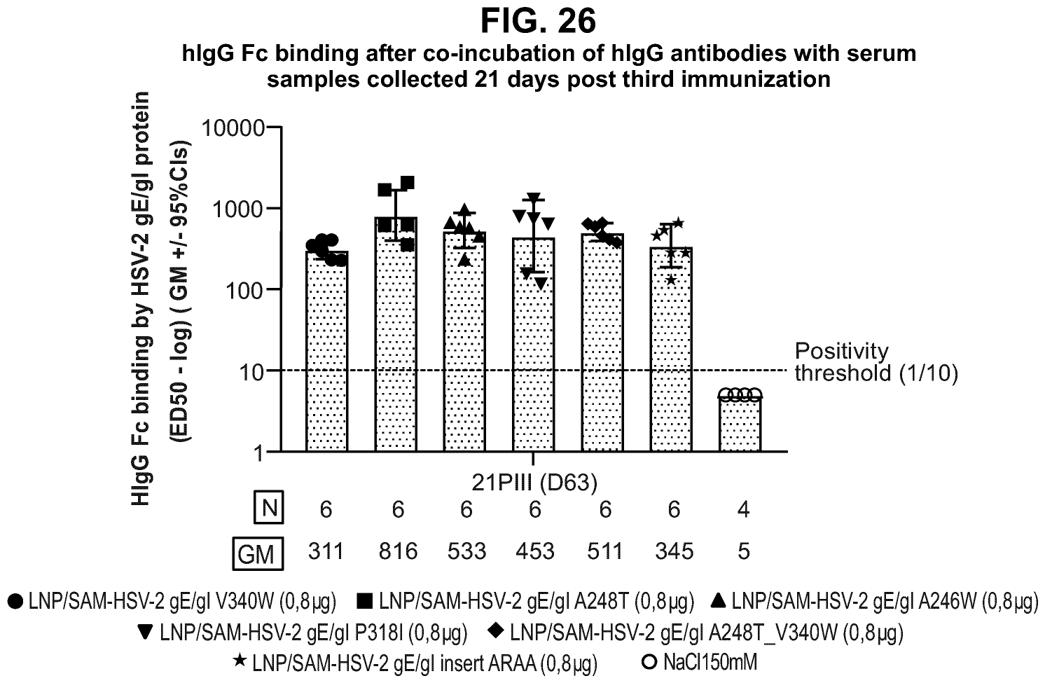

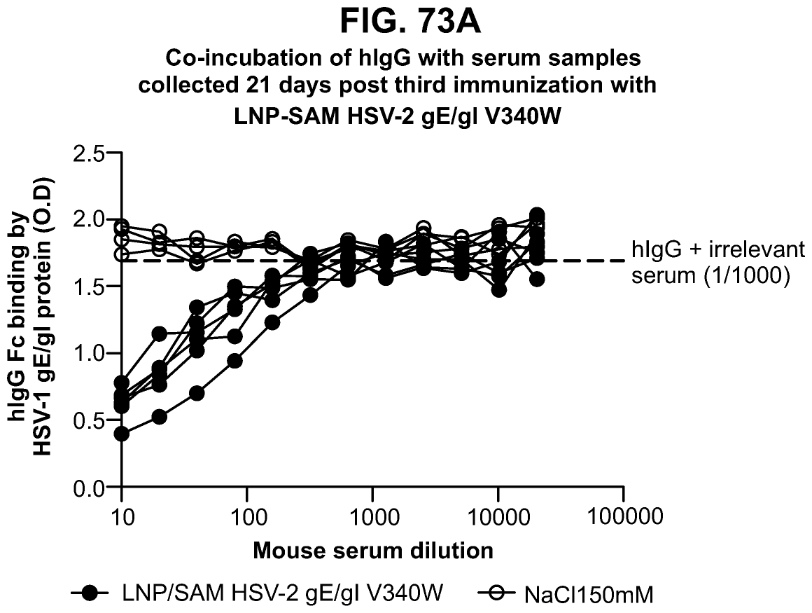

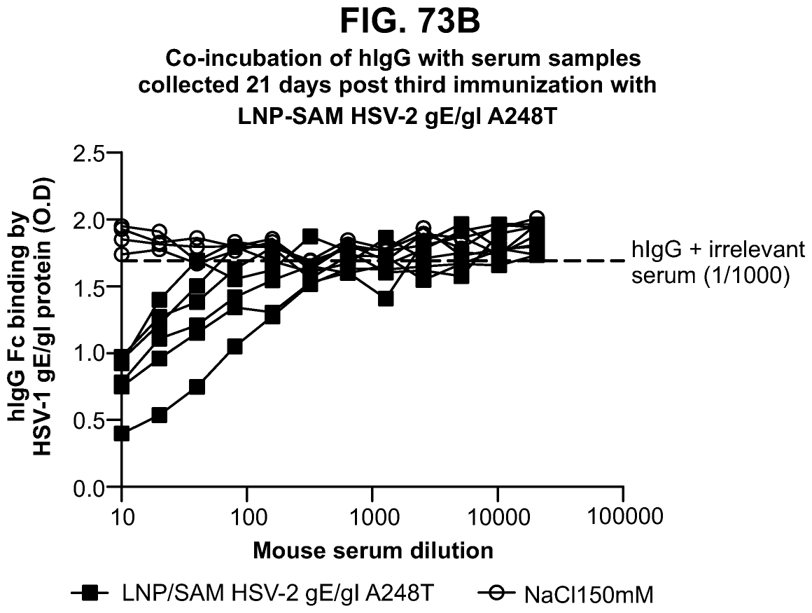

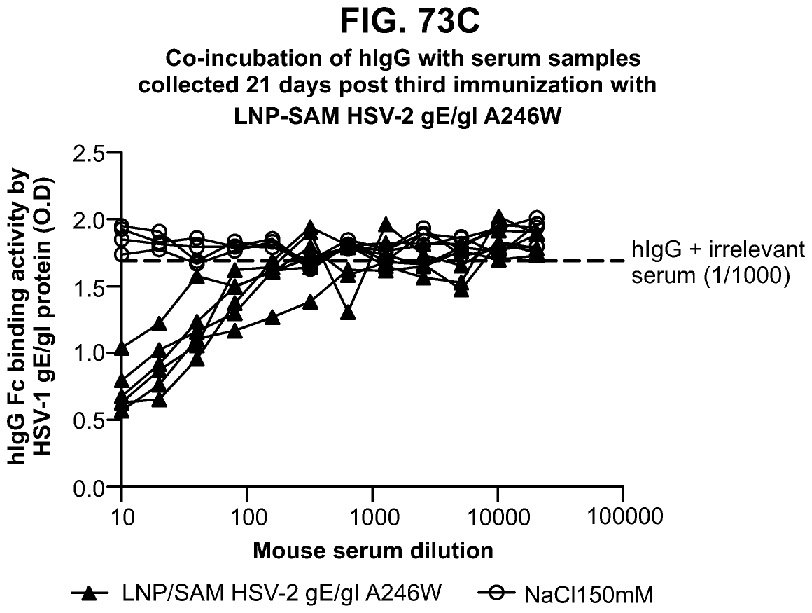

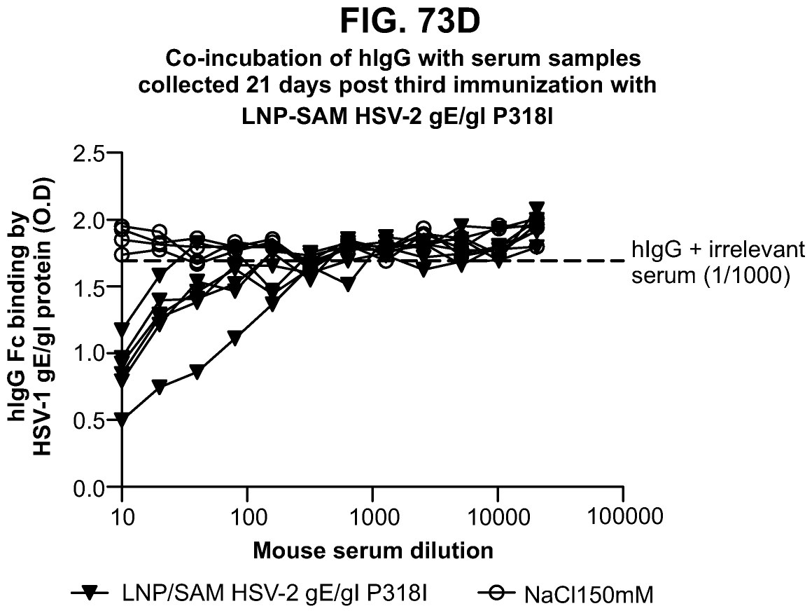

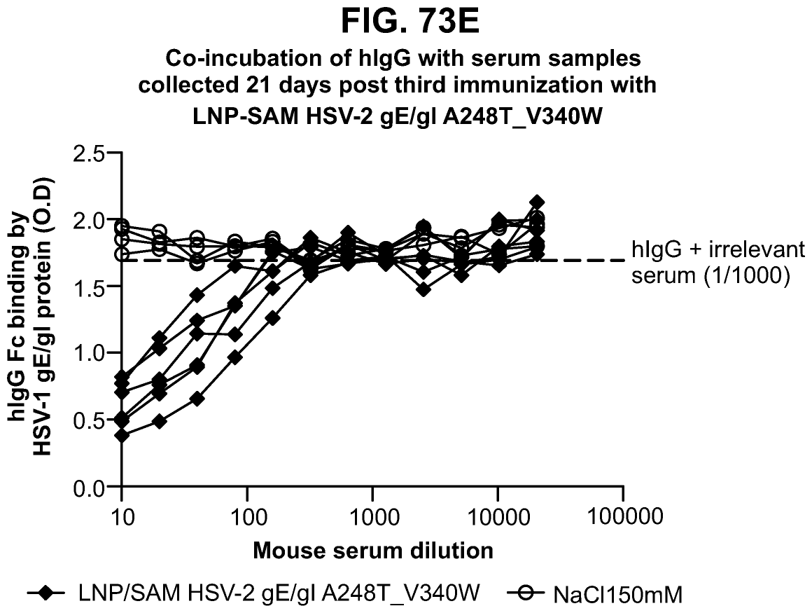

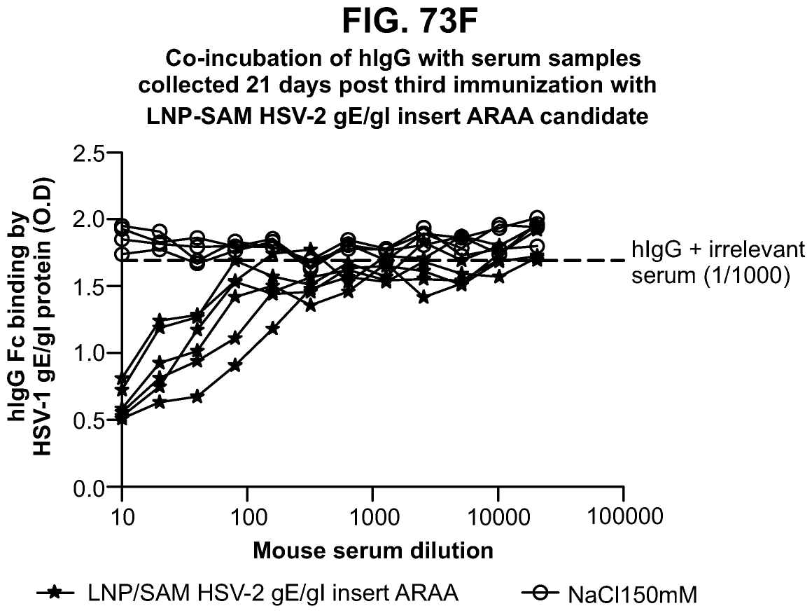

Then, the ability of sera from mice immunized with different LNP-formulated SAM-HSV-2 gE/gI mutant candidates to compete and decrease hIgG binding by HSV-2 or HSV-1 gE/gI protein was assessed in-vitro by competitive ELISA. All LNP-SAM HSV-2 gE/gI mutants elicited vaccine-specific polyclonal antibody response able to compete in-vitro with hIgG Fc binding by HSV-2 or HSV-1 gE/gI proteins. (FIG. 25 & FIG. 73). The dissociation curve of hIgG Fc binding by HSV-2 gE/gI protein was quite similar between all groups of mice and the calculation of the ED₅₀ shown similar response between group of mice (FIG. 26). In addition, the dissociation curve of hIgG Fc binding by HSV-1 gE/gI protein was also quite similar between all groups of mice suggesting no significant difference between the mutants (FIG. 73).

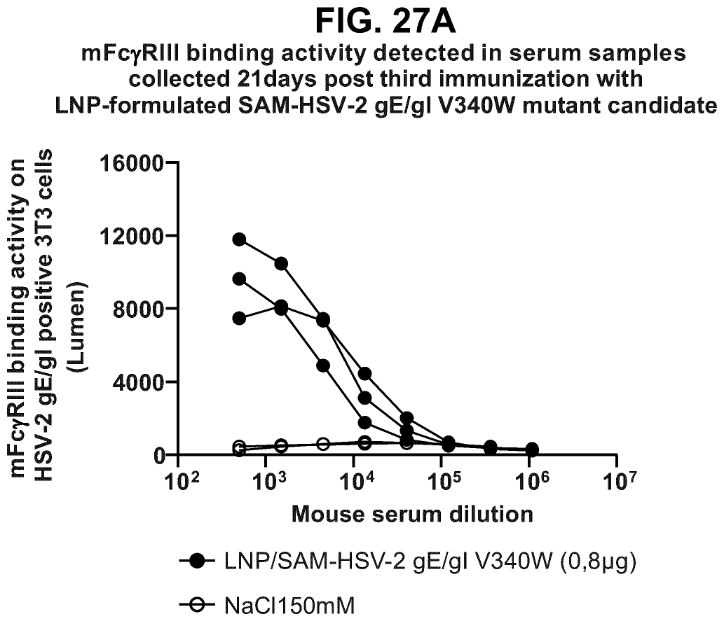

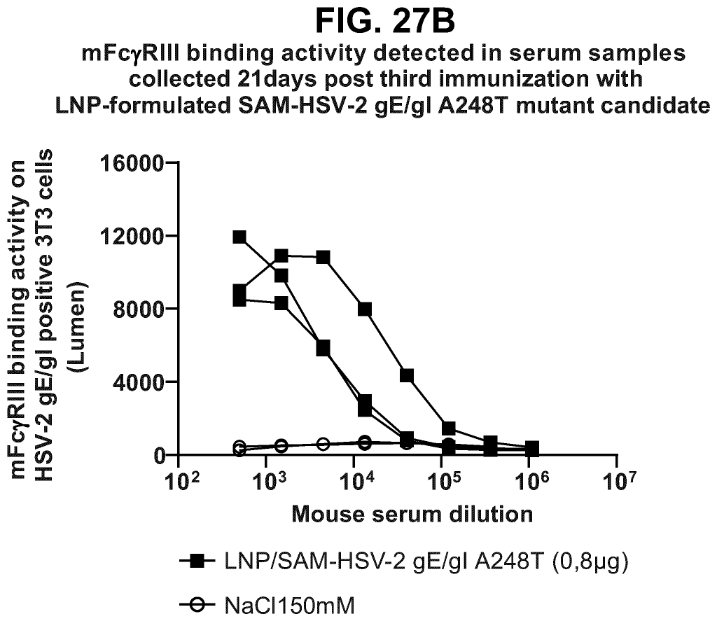

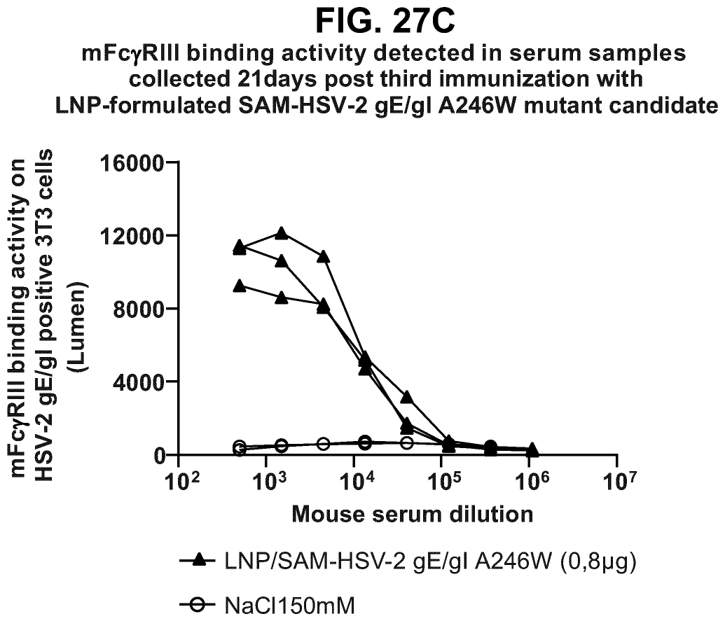

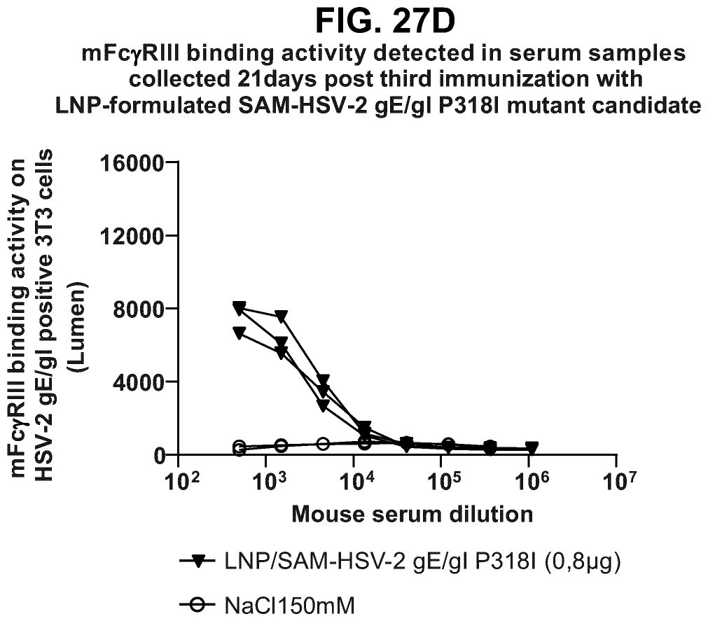

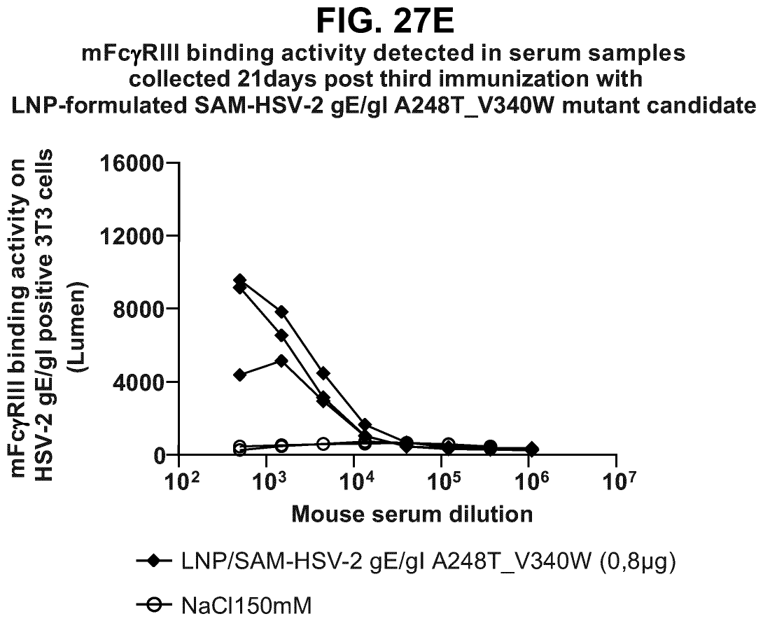

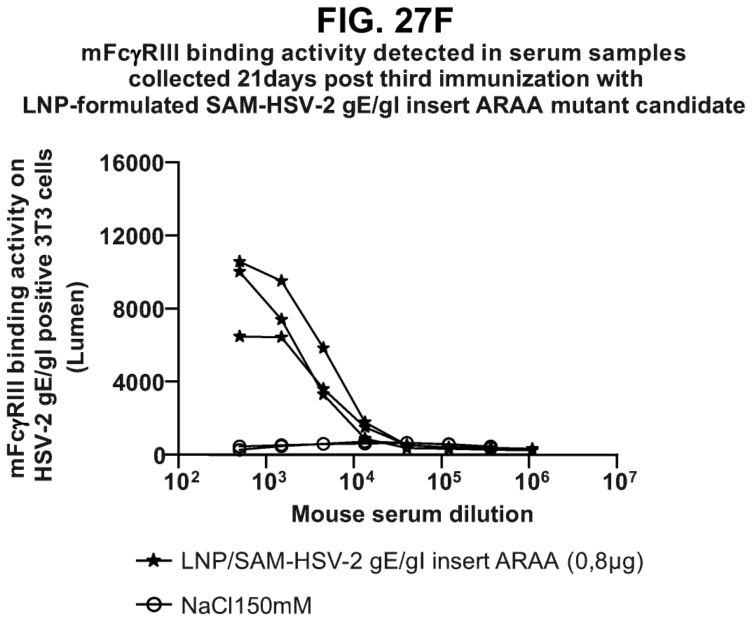

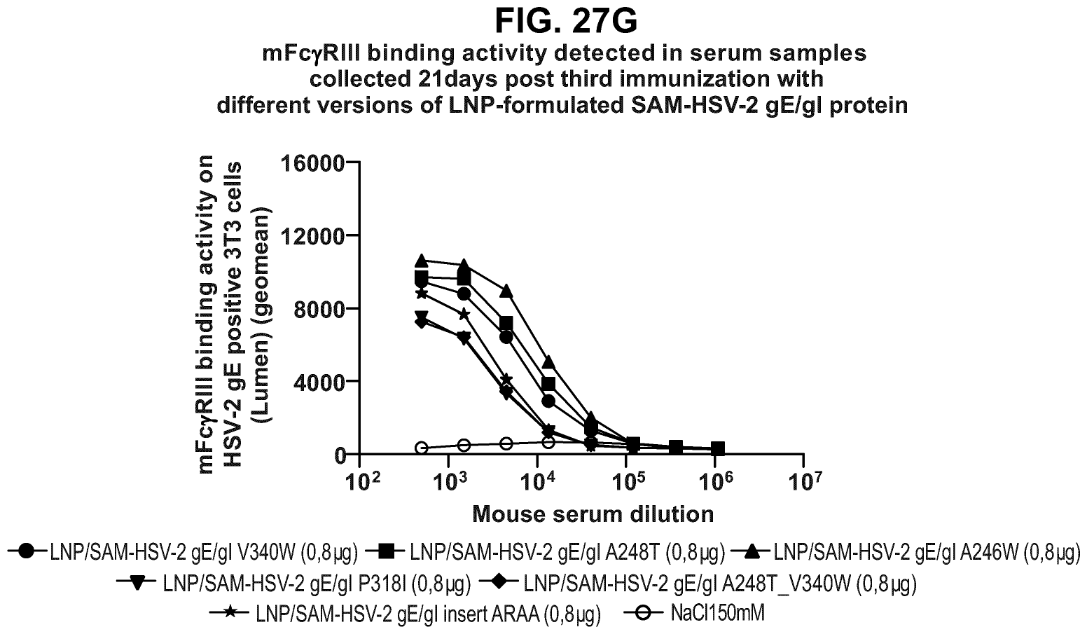

Finally, the ability of vaccine-specific antibody response, induced 14 days post third immunization, to bind and activate in-vitro mouse FcγRIII after incubation with HSV-2 gE/gI transfected cells was investigated. Data shown on the FIG. 27A-F suggested that all groups of mice immunized with the different LNP-SAM HSV-2 gE/gI mutants could induce anti-HSV-2 gE/gI-specific antibody response able to specifically bind and activate Jurkat reporter cells expressing FcγRIII. As expected, activation of FcγRIII was not detected with sera from unvaccinated mice. No major difference was observed between the different group of mice in term of FcγRIII activation (FIG. 27G).

In conclusion, these data suggest that different LNP-SAM HSV-2 gE/gI mutants can induce vaccine-specific antibodies able to bind and activate FcγRIII after incubation with HSV-2 gE/gI transfected cells, to decrease human IgG Fc binding by HSV-2 or HSV-1 gE/gI protein and to neutralize at low intensity HSV-2 MS and HSV-1 VR1789 viruses.

All LNP/SAM HSV-2 gE/gI Mutants Induced Anti-HSV-2 gE-Specific and Anti-HSV-1 gE Cross Reactive CD8+ T Cells and Low Level of Anti-HSV-2 gI-Specific CD4+ T Cells

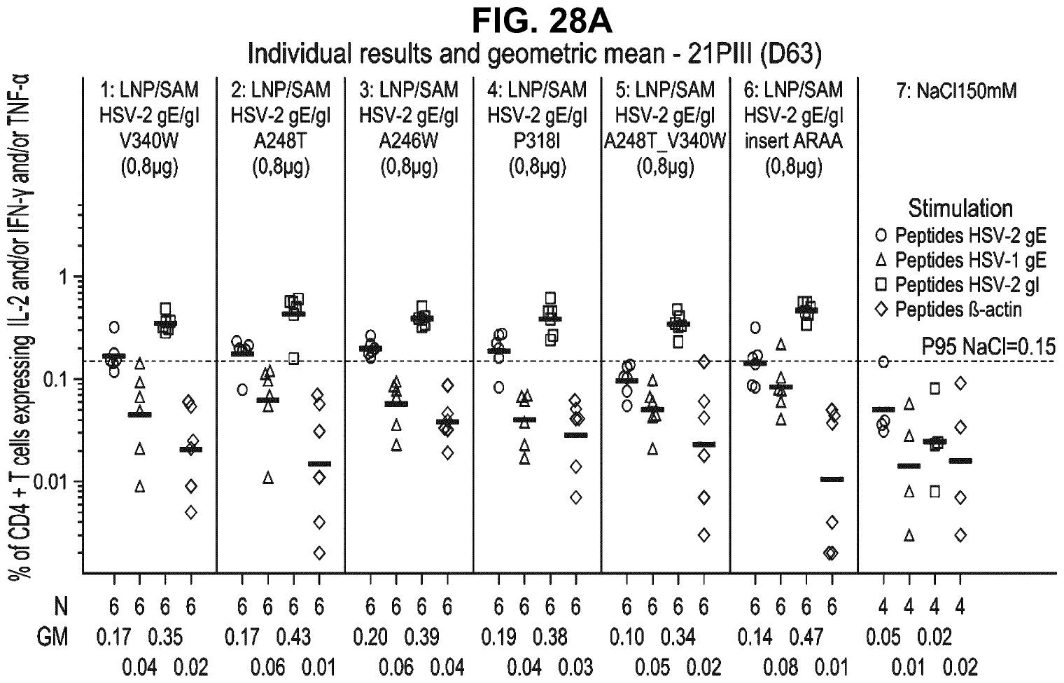

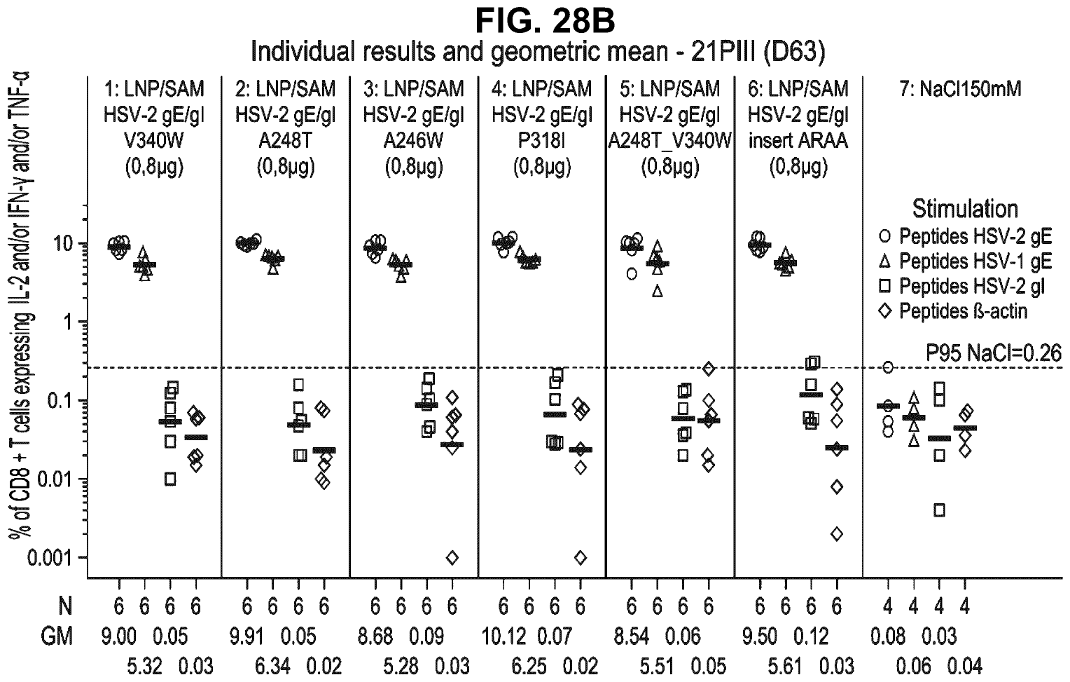

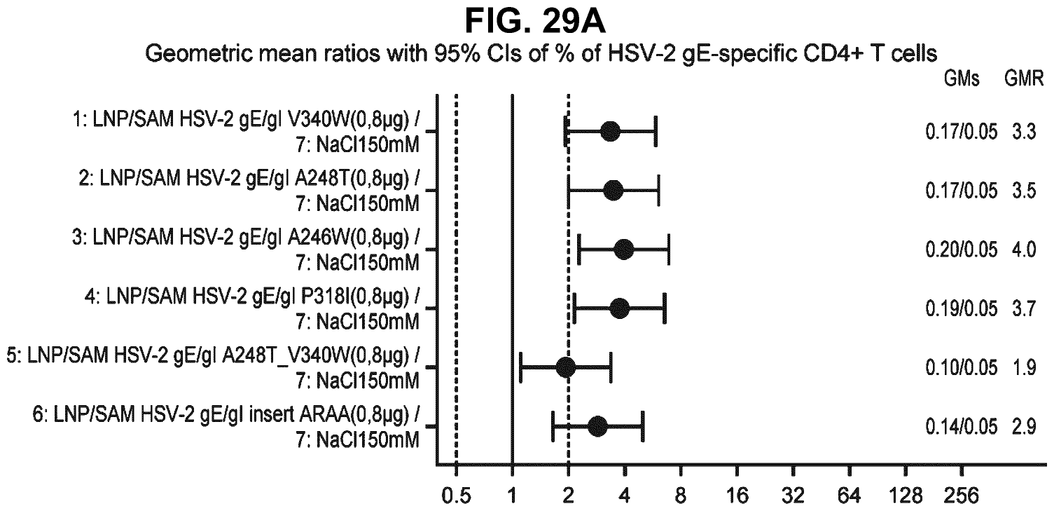

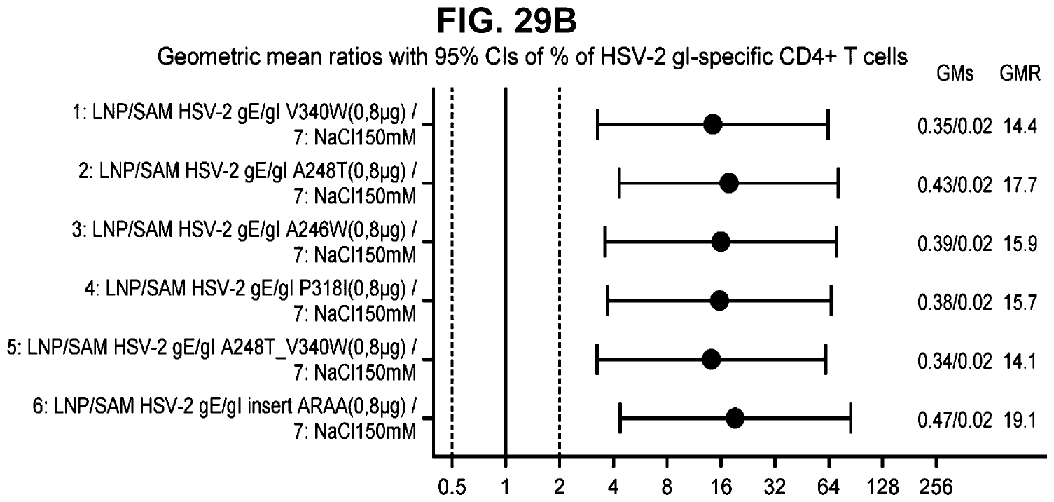

Twenty-one days after the third immunization with different LNP-SAM HSV-2 gE/gI mutants, low levels of anti-HSV-2 gE-specific CD4+ T cell response (GMs<0.2%) was detected in all mice from group 3 (A246W), most of the mice from groups 1 (V340W), 2 (A248T) and 4 (P318I), half of the mice from group 6 (insert ARAA) and none of the mice from group 5 (A248T_V340W) when compared to the P95 threshold based on NaCl control group (FIG. 28A; FIG. 29A). Compared to the NaCl control group, higher anti-HSV-2 gI-specific CD4+T cell response was detected in all groups of mice immunized with different LNP-formulated SAM HSV-2 gE/gI mutants (about 0.4%, with all mice at or above the P95 threshold, and GMRs over NaCl ranging from 14 to 19 with CIs not containing 1) (FIG. 28A & FIG. 29B). In contrast, no HSV-1 gE cross-reactive CD4+ T cell response was detected in any of the vaccinated groups compared to NaCl negative control group (FIG. 28A).

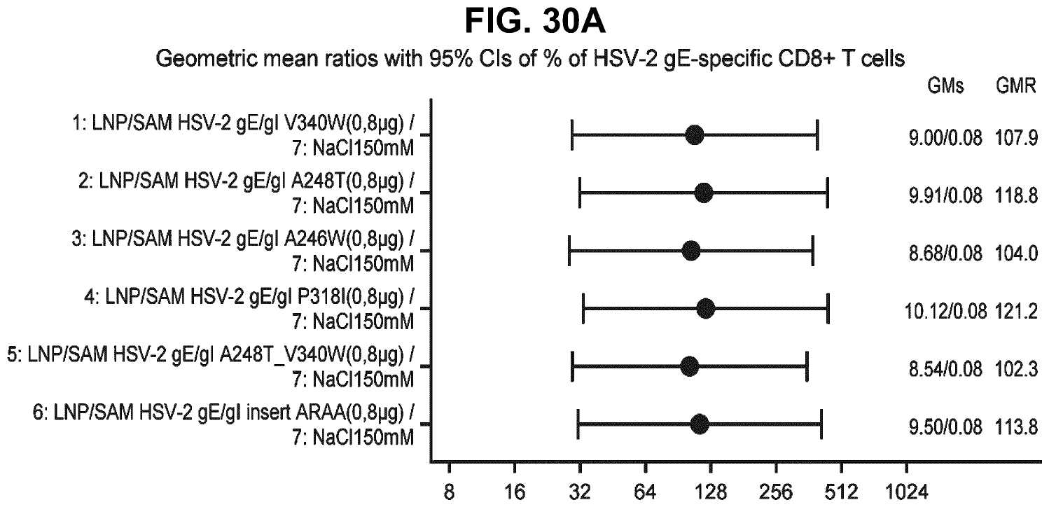

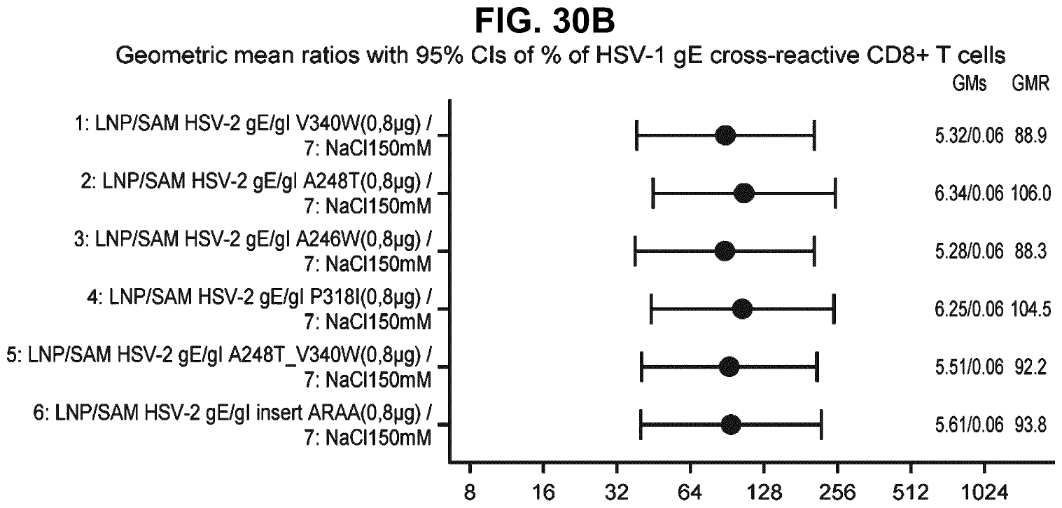

High levels of anti-HSV-2 gE-specific and anti-HSV-1 gE cross-reactive CD8+T cell response were detected in all vaccinated groups compared to the NaCl control group (GMRs of around 100 with CIs not containing 1) (FIG. 28B & FIG. 30). Anti-HSV-2 gI-specific CD8+ T cell response was not detected in any of the vaccinated groups compared to NaCl negative control (FIG. 28B).

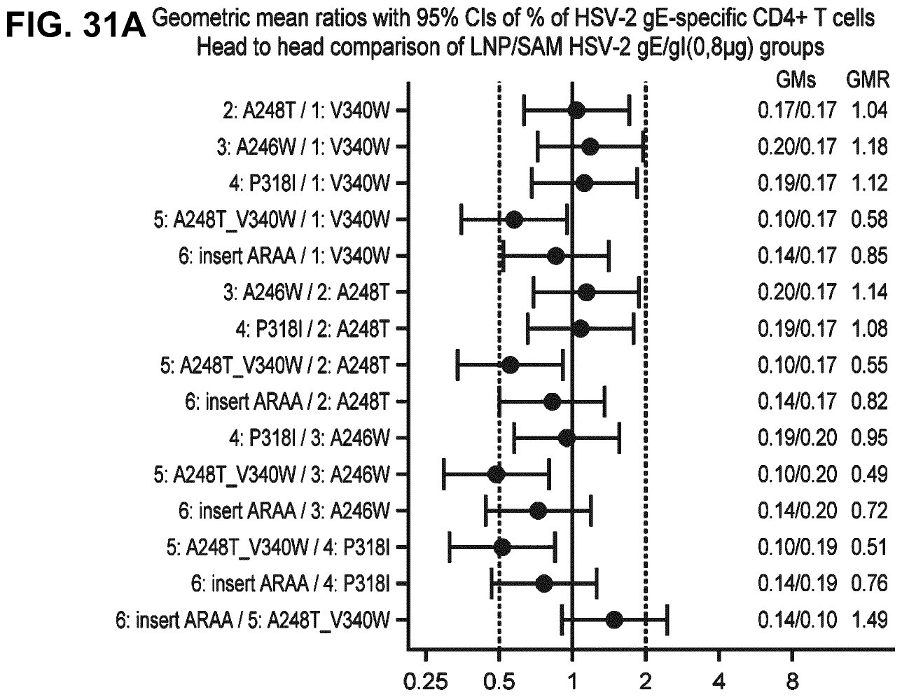

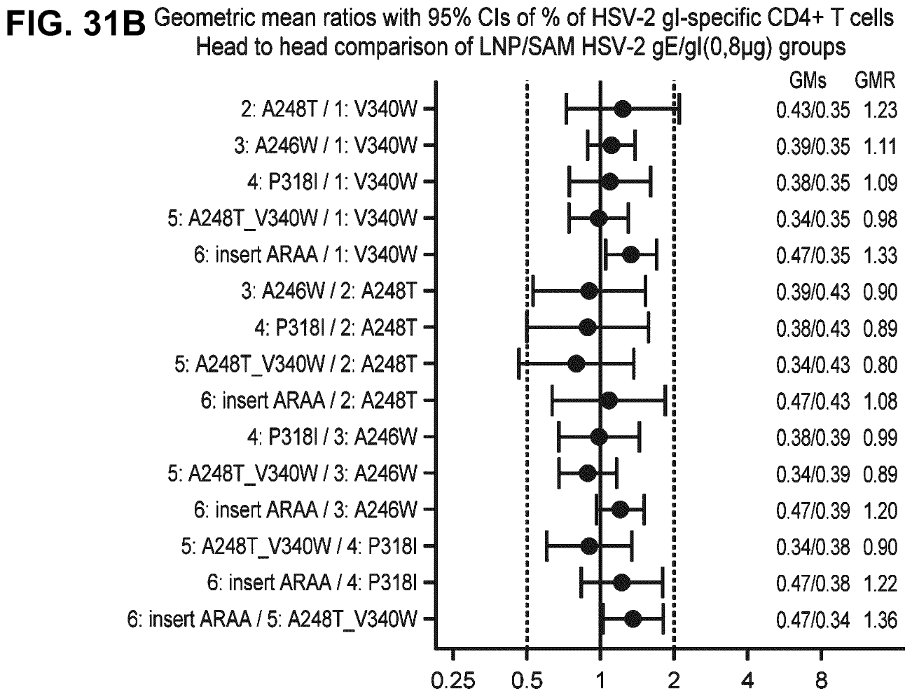

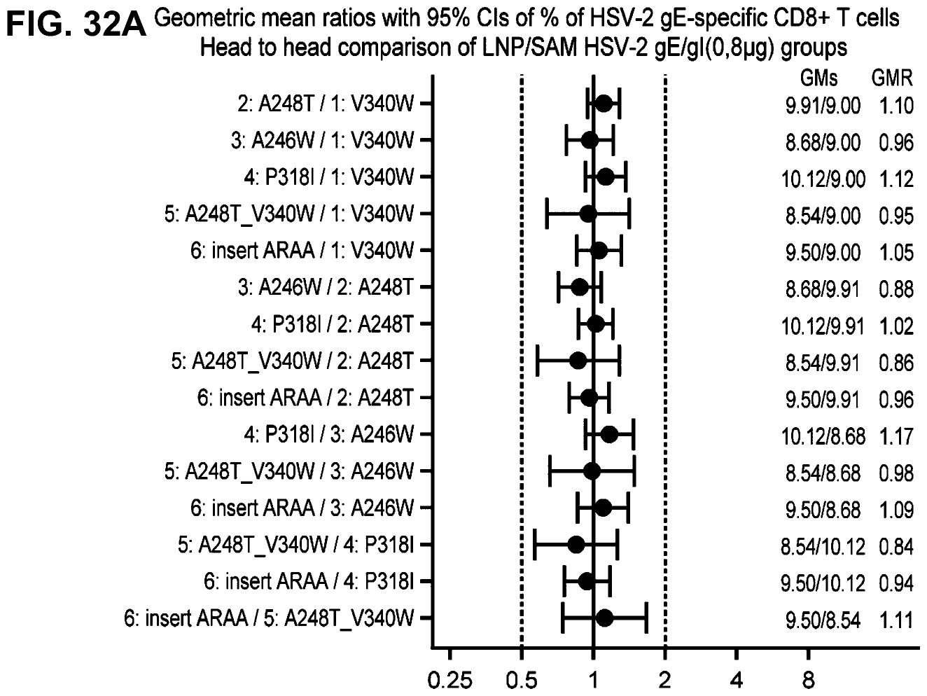

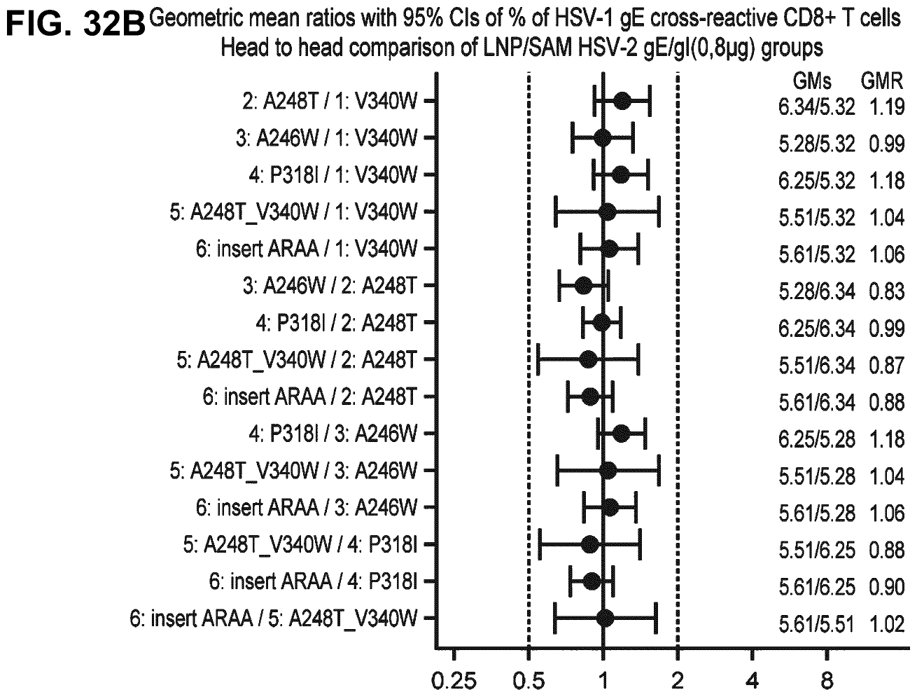

Results suggest that the different mutated versions of LNP-formulated SAM-HSV-2 gE/gI vector were all comparable in terms of a CD4+ and CD8+ T cell response (GMRs<2-fold change with most CIs totally included in [0.5,2] and/or containing 1) (FIG. 31 & FIG. 32). As there is no response detected in anti-HSV-2 gE-specific CD4+T cell response with A248T_V340W mutant (group 5), this group seems to differ from A246W (group3) and P318I mutants (group 4) on this response (fold decrease of 2 with CI not containing 1) (FIG. 31A).

### Example 3—Immuno-Evaluation of Several AS01-Adjuvanted HSV-1 gE/gI Antigens (Unmutated and Mutated) in CB6F1 Mice

Objectives

The general objective of this study was to generate immunogenicity data from different HSV-1 gE/gI proteins containing single point mutation in gE Fc binding domain to drop the avidity to human Immunoglobulin G Fc.

A comparison was carried out between the anti-HSV-1gE/gI-specific polyclonal antibody (pAb) response induced by several AS01-adjuvanted HSV-1 gE/gI antigens (unmutated or mutated) 14 days post third immunization compared to NaCl control group.

14 days post third immunization, the vaccine-specific polyclonal antibody (pAb) response and the anti-HSV-1 gE/gI-specific CD4+T cell responses induced by different mutants of HSV-1 gE/gI protein were compared to NaCl control group. In addition, head-to-head comparison of all vaccine candidates in term of anti-HSV-1 gE/gI-specific IgG antibody responses and anti-HSV-1 gE- and anti-HSV-1 gI-specific CD4+ T cell responses were performed 14 days post third immunization.

Finally, the exploratory objectives were to evaluate, in different groups of mice immunized with HSV-1 gE/gI mutants, the anti-HSV-1 gE/gI-specific antibody responses after one and two immunizations, the anti-HSV-2 gE and anti-HSV-2 gI cross-reactive antibody responses after three immunizations, the functions of vaccine-specific and HSV-2 gE/gI cross reactive polyclonal antibodies after three immunizations and the levels of anti-HSV-1 gE, anti-HSV-1 gI-specific CD8+T cell responses and anti-HSV-2 gE cross-reactive T cell responses after the third immunization.

Study Design

Female CB6F1 mice (n=6gr1-6 & n=4/gr7) were intramuscularly (i.m) immunized at days 0, 14 & 28 with 5 μg of unmutated HSV-1 gE/gI (gr1) or with different mutated versions of HSV-1 gE/gI protein (gr2-6) formulated in AS01 (5 μg). An additional group of mice was i.m injected with a saline solution (NaCl 150 mM), following the same schedule of immunization, and used as negative control group (gr7).

Serum samples were collected at days 13, 27 & 42 post prime immunization (13PI, 13PII, 14PIII) to measure both anti-HSV-1 gE/gI-specific and anti-HSV-2 gE and anti-HSV-2 gI cross reactive IgG antibody responses. The functions of vaccine-specific and HSV-2 cross reactive antibodies were also investigated in the serum samples collected 14 days after the third immunization. Spleens were collected 14 days post third immunization (14PIII) to evaluate, ex-vivo, systemic CD4+ and CD8+ T cell responses towards HSV-1 gE, HSV-1 gI & HSV-2 gE antigens.

Background and Scientific Rationale

The global prevalence of genital herpes is estimated at 417 million in individuals between the ages of 15 and 49, with a disproportionate burden of disease in Africa. Herpes Simplex Virus type 1 (HSV-1) is approximately as common as Herpes Simplex Virus type 2 (HSV-2) as the cause of first time genital herpes infections in resource-rich countries. Recurrent infections are less common after HSV-1 than HSV-2 genital infections; therefore, HSV-2 remains the predominant cause of recurrent genital herpes in resource-rich countries and the burden of HSV-2 infection is even greater in resource-limited countries. Some infected individuals have severe and frequent outbreaks of genital ulcers, while others have mild or subclinical infections, yet all risk transmitting genital herpes to their intimate partners. In infected individuals, antiviral therapy reduces the frequency of recurrent genital lesions and the risk of transmitting infection to partners. However, alternative approaches to reduce the frequency of clinical and subclinical genital recurrences by vaccination are highly demanding.

Therapeutic vaccine candidates are being developed to prevent first genital HSV-2 recurrences. Previous reports showed that vaccine compositions composed of adjuvanted recombinant HSV-2 glycoprotein D (gD) could reduce 50% of episodes of genital herpes reactivation in guinea pig model and in genital herpes patients (HSV-2 positive). Herpesviruses have evolved diverse immune evasion strategies to survive in their natural hosts. HSV encodes namely the glycoproteins E (gE), that target the humoral immune system. gE can form a noncovalent heterodimer complex with glycoprotein I (gI) that functions as an immunoglobulin G (IgG) Fc receptor (FcγR). Interactions between gE and gI increase human Fc binding affinity about 100 times. Through antibody binding, the FcγR inhibits IgG Fc-mediated activities, including complement binding and antibody-dependent cellular cytotoxicity.

In context of therapeutic HSV-2, previous experiments performed in mice showed that human IgG could bind to the gE Fc binding domain of the unmutated recombinant HSV-2 gE/gI protein and mask protective epitopes implicated in HSV-2 gE-mediated immune evasion mechanism. These results suggested that a similar situation could happen in clinical studies and negatively impact the HSV-2 vaccine therapeutic efficacy. To avoid negative immunological interference in human, point mutations have been performed within the Fc binding domain of HSV-2 gE/gI protein to reduce the affinity to hIgG Fc domain and the HSV-2 gE_P317R/gI mutant construct was selected as a vaccine candidate.

As this immune evasion mechanism is used by both HSV-1 and -2 viruses, it was decided to also generate immunogenicity data with different mutated versions of recombinant HSV-1 gE/gI protein (with a strong reduction of gE Fc binding activity).

The objective of this experiment was to evaluate the immunogenicity in mice of five different HSV-1 gE/gI mutants formulated in AS01.

Study Design

Female CB6F1 inbred mice aged 6-8 weeks old from Harlan laboratory (OlaHsd) were randomly assigned to the study groups (n=6 gr1-6 & n=4/gr7) and kept at the institutional animal facility under specified pathogen-free conditions. CB6F1 mice (gr1-6) were intramuscularly (i.m) immunized at days 0, 14 & 28 with 5 μg of unmutated (gr1) or with different mutated versions of HSV-1 gE/gI proteins (gr2-6) formulated in AS01 (5 μg). An additional group of mice was i.m injected with a saline solution (NaCl 150 mM), following the same schedule of immunization, and used as negative control group (gr7).

Serum samples were collected at days 13, 27 & 42 post prime immunization (13PI, 13PII, 14PIII) to measure both anti-HSV-1 gE/gI-specific and anti-HSV-2 gE and anti-HSV-2 gI cross reactive IgG antibody responses. The functions of vaccine-specific and HSV-2 cross reactive antibodies were also investigated in the serum samples collected 14 days after the third immunization. Spleens were collected 14 days post third immunization (14PIII) to evaluate, ex-vivo, systemic CD4+ and CD8+ T cell responses towards HSV-1 gE, HSV-1 gI & HSV-2 gE antigens. Details for each group is described in the Table 5.

TABLE 5
Summary of the study design and formulation tested

| | | Treatment | | | | | | |
|---|---|---|---|---|---|---|---|---|
| | Number | Vaccine | | | Adjuvant | Volume and | Immunization | Sample collection |
| | of | name and | Lot | Adjuvant | lot | route of | schedule | (organs, blood |
| Group | animals | dose | numbers | dose | number | injection | (Days) | volume) and days |
| 1 | 6 | HSV-1 | BMP1299 | AS01 | LIQ- | 50 μL | Days 0, 14 & 28 | Days 13 & |
| | | gE/gI | | | 2019/2014 | i.m | | 27: Serum - |
| | | (5 μg) | | | | | | partial bleeding |
| 2 | 6 | HSV-1 | BMP1281 | | | | | Day 42: Serum - |
| | | gE_P319R/gI | | | | | | final bleeding + |
| | | (5 μg) | | | | | | Spleen |
| 3 | 6 | HSV-1 | BMP1282 | | | | | |
| | | gE_P321D/gI | | | | | | |
| | | (5 μg) | | | | | | |
| 4 | 6 | HSV-1 | BMP1283 | | | | | |
| | | gE_R322D/gI | | | | | | |
| | | (5 μg) | | | | | | |
| 5 | 6 | HSV-1 | BMP1284 | | | | | |
| | | gE_N243A_R | | | | | | |
| | | 322D/gI | | | | | | |
| | | (5 μg) | | | | | | |
| 6 | 6 | HSV-1 | BMP1285 | | | | | |
| | | gE_A340G_S341G_V342G/gI | | | | | | |
| | | (5 μg) | | | | | | |
| 7 | 4 | NaCl150 mM | N/A | N/A | N/A | | | |

Materials and Methods

Investigational Products

The antigens used in this study were different versions of the purified recombinant gE/gI protein (ectodomain only). The material was expressed on ExpiCHO™ cells as research lot only.

AS01 is a liposome based adjuvant system (AS) containing QS-21 (a triterpene glycoside purified from the bark of Quillaja saponaria) and MPL (3-D Monophosphoryl lipid A), with liposomes as vehicles for these immunoenhancers and a buffer including NaCl as isotonic agent (variant 3).

A single human dose of the AS01b Adjuvant System (0.5 mL) contains 50 μg of QS-21 and 50 μg of MPL. In this study, the volume injected in mice is 1/10th of a human dose corresponding to a 5 μg QS-21 and 5 μg MPL per dose.

Animal Model

It is well accepted that small animal models are useful tools to study the immunogenicity profile of new vaccine candidates. In order to get a general overview of the capacity of the vaccine candidates to induce T cell immune responses, CB6F1 mice (hybrid of C57B1/6 and Balb/C mice) have been used in this study.

CB6F1 mice have been shown to generate potent CD4+/CD8+ T cell and humoral immune responses following vaccination with various types of immunogens, including adjuvanted proteins and viral vectors. The profile of the vaccine-induced immune responses generated in these mice compared to expected responses in humans may nevertheless be impacted by some differences pertaining to TLR expression, HLA background and antigen presentation. However, the capacity for inducing CD4+/CD8+T immune responses has shown comparable trends between these mice and humans.

Immunological Read-Outs

Total Anti-HSV-1 gE/gI-Specific IgG Binding Antibodies Measured by ELISA

Quantification of the total anti-HSV-1 gE/gI-specific IgG antibodies was performed using indirect ELISA. Recombinant gE/gI heterodimer protein (BMP1299) from HSV-1 were used as coating antigen. This protein was produced using the ExpiCHO™ expression system. The ELISA was otherwise carried out as described in Example 1.

Optical densities (OD) were captured and analysed using the SoftMaxPro GxP v5.3 software. A standard curve was generated by applying a 4-parameter logistic regression fit to the reference standard results (reference standard=20200023 14PIII—Pool of mice 1.1 to 1.20 immunized with AS01-HSV-1 gE/gI HEK (5 μg of each/dose). Antibody titer in the samples was calculated by interpolation of the standard curve. The antibody titer of the samples was obtained by averaging the values from dilutions that fell within the 20-80% dynamic range of the standard curve. Titers were expressed in EU/mL (ELISA Units per mL).

Total Anti-HSV-2 gE & gI Cross-Reactive IgG Antibodies Measured by ELISA

Quantification of the total anti-HSV-2 gE or gI cross-reactive IgG antibodies was performed using indirect ELISA. Recombinant gE (˜51 kDa) (BMP1291) or gI protein (˜46 kDa) (BMP1292) from HSV-2 were used as coating antigens. These proteins were produced using the ExpiHEK293F™ expression system. The indirect ELISA was otherwise carried out as described for this method in Example 1.

Anti-HSV-1 gE and gI-Specific and HSV-2 gE Cross-Reactive CD4+/CD8+ T Cell Responses Measured by ICS Assay

The frequencies of anti-HSV-1 gE & anti-HSV-1 gI-specific and anti-HSV-2 gE cross-reactive CD4+ and CD8+ T-cells producing IL-2 and/or IFN-γ and/or TNF-α were evaluated in splenocytes collected 14 days post third immunization after ex-vivo stimulation with HSV-1 gE or gI or HSV-2 gE peptides pools.

Isolation of Splenocytes

As described for this method in Example 1.

Cell Preparation

Fresh splenocytes were seeded in round bottom 96-well plates at 10⁶ cells/well (1004). The cells were then stimulated for 6 hours (37° C., 5% CO2) with anti-CD28 (BD biosciences, clone 37.51) and anti-CD49d antibodies (BD Biosciences, clone 9C10 (MFR4.B)) at 1 μg/mL per well, containing 100 μL of either:
15 mers overlapping peptides pool covering the sequence of gE protein from HSV-1 (1 μg/mL per peptide per well).
15 mers overlapping peptides pool covering the sequence of gI protein from HSV-1 (1 μg/mL per peptide per well).
15 mers overlapping peptides pool covering the sequence of gE protein from HSV-2 (1 μg/mL per peptide per well).
15 mers overlapping peptides pool covering the sequence of Human β-actin protein (1 μg/mL per peptide per well) (irrelevant stimulation).
RPMI/additives medium (as negative control of the assay).
PMA—ionomycin solution (Sigma, P8139) at working concentrations of 0.25 μg/mL and 2.5 μg/mL respectively (as positive control of the assay).

After 2 hours of ex vivo stimulation, Brefeldin A (Golgi plug ref 555029, BD Bioscience) diluted 1/200 in RPMI/additives supplemented with 5% FCS was added for 4 additional hours to inhibit cytokine secretion. Plates were then transferred at 4° C. for overnight incubation.

Intracellular Cytokine Staining

As described for this method in Example 1.

Cell Acquisition and Analysis

As described for this method in Example 1.

Competitive ELISA to Evaluate the Ability of Vaccine-Specific Antibodies to Decrease Human IgG Fc Binding by HSV-1 gE/gI Protein

The ability of polyclonal sera collected in different groups of mice to decrease, in-vitro, hIgG Fc binding by recombinant HSV-1 gE/gI protein was investigated by competitive ELISA.

Recombinant HSV-1 gE/gI protein (BMP1299), produced using the ExpiCHO™ expression system was used as coating antigen.

Polystyrene 96-well ELISA plate (Nunc F96 Maxisorp cat 439454) were coated with 50 μg/well of HSV-1 gE/gI protein diluted at a concentration of 2 μg/mL in free Calcium/Magnesium PBS buffer and incubated overnight at 4° C. After incubation, the coating solution was removed and the plates were blocked with 100 μL/well of PBS supplemented with 0.1% Tween-20+1% BSA (blocking buffer) for 1 h at 37° C.

In another 96-well Clear V-Bottom Polypropylene microplate (Falcon, ref 353263) a two-fold serial dilution (starting dilution 1/10) in blocking buffer for each serum was prepared in 60 μL/well and mixed with 60 μL/well of biotinylated-hIgG antibodies (Invitrogen, ref 12000C) pre-diluted at 0.7 μg/mL in blocking buffer.

Then, after 1 h of incubation with blocking buffer, the blocking solution was removed from the coated plates and 100 μL of the mixture containing both hIgG and mice sera was transferred in the corresponding HSV-1 gE/gI coated wells and incubated 24h at 37° C. Positive control of the assay was a pool of anti-HSV-2 gE/gI serum samples from previous studies. Negative control of the assay was a pool of irrelevant HPV serum samples diluted 1/1000 and mixed with the same concentration of hIgG as the samples.

After 24h of incubation, the plates were washed four times with PBS 0.1% Tween20 (washing buffer) and 50 μL/well of Steptavidin-horsedish Peroxydase AMDEX (Amersham, ref RPN4401V) diluted 2000× were added on the wells and plates were incubated for 30 min at 37° C. Plates were then washed four times with washing buffer and 50 μL/well of a solution containing 75% single-component TMB Peroxidase ELISA Substrate (ref 172-1072, Bio-Rad, USA) diluted in sodium Citrate 0.1M pH5.5 buffer were added for 10 min at room temperature. Enzymatic color development was stopped with 50 μL/well of 0.4N Sulfuric Acid 1M (H₂SO₄) and the plates were read at an absorbance of 450/620 nm using the Versamax ELISA reader. Optical densities (OD) were captured and fitted in curve in Excel program.

Titers were expressed as the effective dilution at which 50% (i.e. ED₅₀) of the signal was achieved by sample dilution.

For each plate and using a reference sample (i.e. irrelevant serum), the reference ED₅₀ value was estimated using the following formula:

ED50=OD_{0%}+0.5*(OD_{100%}−OD_{0%})

where OD_{100%} is the highest OD obtained with similar samples and OD_{0%} is the lowest achievable signal. For each plate, the former was obtained by averaging (mean) 6 replicates while the latter was set at zero.

Sample ED₅₀ titers were computed by way of linear interpolation between the left and right measurements closest to the ED₅₀ estimate within the plate. The approximation was obtained, on the untransformed OD and the logarithm base 10 transformed dilutions, with the approx function of the stats R base package.

Sample were not assigned a titer in the following cases:
no measurement was available above or below the ED₅₀,
curve crossed at least twice the ED₅₀ and
one of the dilution step (left or right) closest to the ED₅₀ was missing

Competitive ELISA to Evaluate the Ability of HSV-2 Cross Reactive Antibodies to Decrease Human IgG Fc Binding by HSV-2 gE/gI Protein

The ability of polyclonal sera collected in different groups of mice to decrease in-vitro hIgG Fc binding by recombinant HSV-2 gE/gI protein was investigated by competitive ELISA. Recombinant HSV-2 gE/gI protein (BMP1063), produced using the ExpiHEK293F™ expression system was used as coating antigen.

Polystyrene 96-well ELISA plate (Nunc F96 Maxisorp cat 439454) were coated with 50 μL/well of HSV-2 gE/gI protein diluted at a concentration of 4 μg/mL in free Calcium/Magnesium PBS buffer and incubated overnight at 4° C. After incubation, the coating solution was removed and the plates were blocked with 100 μL/well of PBS supplemented with 0.1% Tween-20+1% BSA (blocking buffer) for 1 h at 37° C.

The blocking solution was removed from the coated plates and 60 μL of two serial dilution of individual mouse serum (starting dilution 1/10 in blocking buffer) was prepared into 96-well Clear V-Bottom Polypropylene microplate (Falcon, ref 353263) and mixed with 60 μL/well of biotinylated-hIgG antibodies (Invitrogen, ref 12000C) pre-diluted at 0.7 μg/mL in blocking buffer. The blocking buffer from HSV-2 gE/gI coated plates was removed and 100 μL of the mixture was then transferred in the corresponding wells for an incubation period of 24h at 37° C. Positive control serum of the assay was a pool of HSV-2 serum samples from previous studies. Negative control serum of the assay was a pool of irrelevant HPV serum samples diluted 1/100 and mix with hIgG too.

After incubation, the plates were washed four times with PBS+0.1% Tween20 (washing buffer) and 50 μL/well of Steptavidin-horsedish Peroxydase AMDEX (Amersham, ref RPN4401V) diluted 2000× were added on the well and plates were incubated for 30 min at 37° C. After incubation, plates were washed four times with washing buffer and 50 μL/well of a solution containing 75% single-component TMB Peroxidase ELISA Substrate (ref 172-1072, Bio-Rad, USA) diluted in sodium Citrate 0.1M pH5.5 buffer were added for 10 min at room temperature. Enzymatic color development was stopped with 50 μL/well of 0.4N Sulfuric Acid 1M (H₂SO₄) and the plates were read at an absorbance of 450/620 nm using the Versamax ELISA reader. Optical densities (OD) were captured and fitted in curve in Excel program.

Titers were expressed as the effective dilution at which 50% (i.e. ED₅₀) of the signal was achieved by sample dilution.

For each plate and using a reference sample (i.e. irrelevant serum), the reference ED₅₀ value was estimated using the following formula:

ED50=OD_{0%}+0.5*(OD_{100%}−OD_{0%})

where OD_{100%} is the highest OD obtained with similar samples and OD_{0%} is the lowest achievable signal. For each plate, the former was obtained by averaging (mean) 6 replicates while the latter was set at zero.

Samples ED50 titers were computed by way of linear interpolation between the left and right measurements closest to the ED₅₀ estimate within the plate. The approximation was obtained, on the untransformed OD and the logarithm base 10 transformed dilutions, with the approx function of the stats R base package.

Sample were not assigned a titer in the following cases:
no measurement was available above or below the ED₅₀,
curve crossed at least twice the ED₅₀ and
one of the dilution step (left or right) closest to the ED₅₀ was missing

Evaluation of the Ability of HSV-1-gE/gI Specific and HSV-2 gE/gI Cross-Reactive Antibodies to Bind and Activate mFcγRIII after Incubation with HSV-1 or HSV-2 gE/gI Transfected Cells (Mouse FcγRIII ADCC Bioassay—Promega)

The mouse FcγRIII Antibody Dependent Cell Cytotoxicity (ADCC) Reporter Bioassay (Cat. #M1201), developed by Promega laboratory, is a bioluminescent cell-based assay which can be used to measure the ability of antibodies to specifically bind and activate the mouse FcγRIII expressed by modified Jurkat reporter cells. The method used was as described for Example 1.

Samples titer in the mouse ADCC-like assay were computed by means of the area under the curve (AUC) as described in Huang and Pang (Assay Drug Dev Technol. 2012 10(1):88-96), with minor modifications. Briefly, log-transformed responses (i.e. RLU) were fitted, on log 3-transformed sample serial dilutions, with a 5-parameter logistic model for each sample on the plate. A negative control sample (i.e. irrelevant mouse serum diluted 1/1000) was used to evaluate, in each plate, the background signal threshold estimated with its geometric mean plus 3 standard deviations (i.e. exp[mean(logRLU)+3*sd(logRLU)]). The area below the fitted curve of each sample and above the plate background (i.e. AUC) was computed using the “integrate” function in “R” software.

In-Vitro HSV-1 Neutralization Assay

An in-vitro neutralization assay was developed to detect and quantify HSV-1 specific neutralizing antibody titers in serum samples from different animal species. The method used was as described for Example 1.

In-Vitro HSV-2 Neutralization Assay

The method used was as described for Example 1.

Statistical Methods

The distribution of each response was assumed to be lognormal.

For anti-HSV-1 gE/gI-specific antibody (pAb) response, a two-way analysis of variance (ANOVA) model was fitted on log₁₀ titers by including groups (all except the NaCl one), time points (Day13 (13PI), Day27 (13PII) and Day42 (14PIII)) and their interaction as fixed effects. The NaCl group was not included as no response and variability was observed. Variance-covariance model selection was based on AICC criterion and individual data plot examination.

The variance-covariance for time points was modelled via an Unstructured matrix:

$$\begin{matrix}[ \begin{matrix}{σ}_{1}^{2} & {σ}_{21} & {σ}_{31} \\ {σ}_{21} & {σ}_{2}^{2} & {σ}_{32} \\ {σ}_{31} & {σ}_{32} & {σ}_{3}^{2}\end{matrix} ] \\ UN - Unstructured\end{matrix}$$

The unstructured matrix considers different variances and estimates unique correlations for each pair of time points. The same variance-covariance matrix is modelled for each vaccine group, indicating same variance between groups.

Geometric means and their 95% CIs are derived from this model.

Despite the fact that the NaCl group was not included in the ANOVA model, the comparisons associated to the primary objective were computed as follows: geometric means and 95% CI of vaccinated groups derived from the above model were divided by titer and given to all the NaCl recipients at the last timepoint. The resulting ratios should be understood as geometric mean ratios with their corresponding 95% CI.

For head to head comparison of vaccinated groups (at the last time point) and time point comparisons (PII/PI, PIII/PII, and PIII/PI) within each group, all the geometric mean ratios and their 95% CIs were derived from the model.

For each HSV-2 gE and gI cross-reactive IgG antibody response, a one-way analysis of variance (ANOVA) model was fitted on log 10 titers by including groups (all except the NaCl one) as fixed effect. The NaCl group was not included as no response and variability was observed. For gE cross-reactive IgG antibody titers, heterogeneity of variance was detected and therefore a different variance was assumed for each group. For gI cross-reactive IgG antibody titers, no clear heterogeneity of variance was detected and therefore identical variances were assumed for the different groups. Geometric means and their 95% CIs are derived from these models. For comparison of vaccinated to NaCl groups, geometric means and 95% CI of vaccinated groups derived from the above model were divided by titer given to all the NaCl recipients. The resulting ratios should be understood as geometric mean ratios with their corresponding 95% CI. For head to head comparison of vaccinated groups, all the geometric mean ratios and their 95% CIs were derived from the model.

On each vaccine-specific or cross-reactive % of CD4+/CD8+ T cell responses (gE or gI), a one-way analysis of variance (ANOVA) model was fitted on log 10 frequencies by including groups (all groups including the NaCl group) as fixed effect. No clear heterogeneity of variance was detected and therefore identical variances were assumed for the different groups. For both % of CD4+ and CD8+ T cell responses, the NaCl threshold was based on P95 of data across stimulation in NaCl negative control group. No modelling was performed on % of HSV-1 gE-specific CD8+ T cells and on % of HSV-2 gE cross-reactive CD8+ T cells since response were below the P95 NaCl threshold for all vaccine groups. Geometric means and geometric mean ratios (with their corresponding 95% CIs) were derived from these models.

For the evaluation of the dissociation of the human IgG Fc binding by the pAbs, ED50 response was calculated for each sample. On each response (HSV-1 or HSV-2), a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

For HSV-1-specific neutralizing antibody titers, a one-way analysis of variance (ANOVA) model was fitted on log₁₀ values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

Results

Different Versions of AS01-HSV-1 gE/gI Protein can Induce Vaccine-Specific and Cross Reactive IgG Antibodies

The anti-HSV-1 gE/gI-vaccine-specific and anti-HSV-2 gE or anti-HSV-2 gI cross-reactive IgG antibody responses were investigated by ELISA.

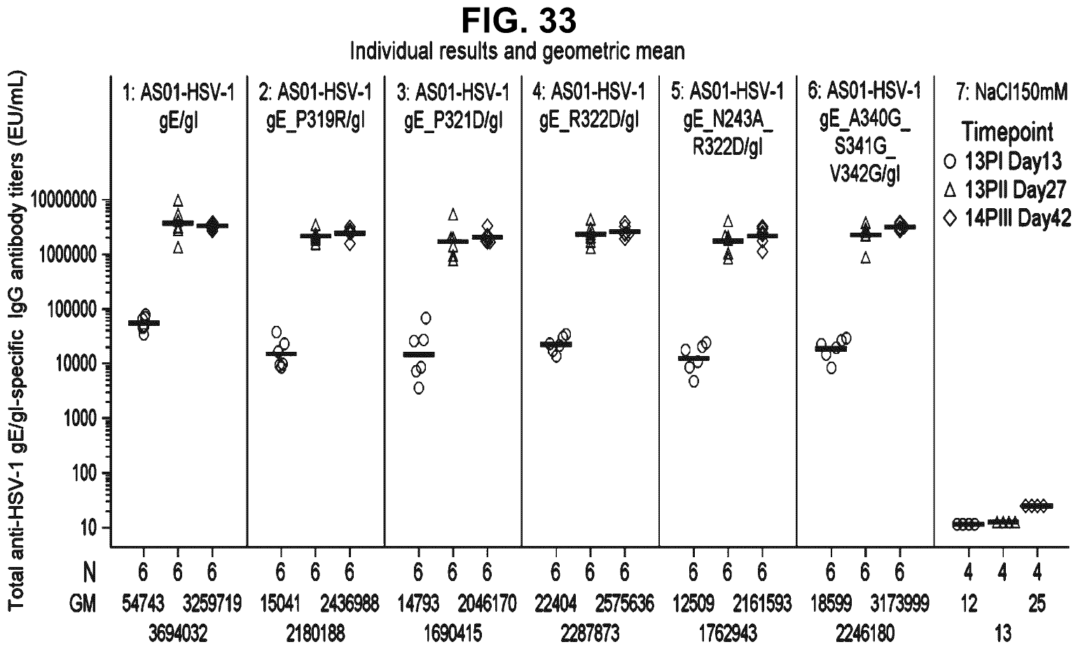

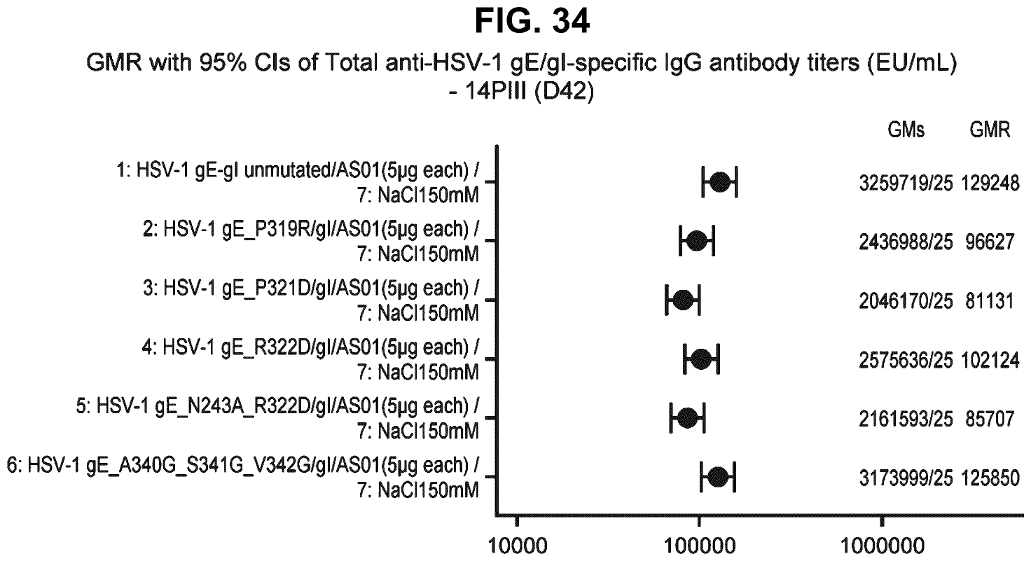

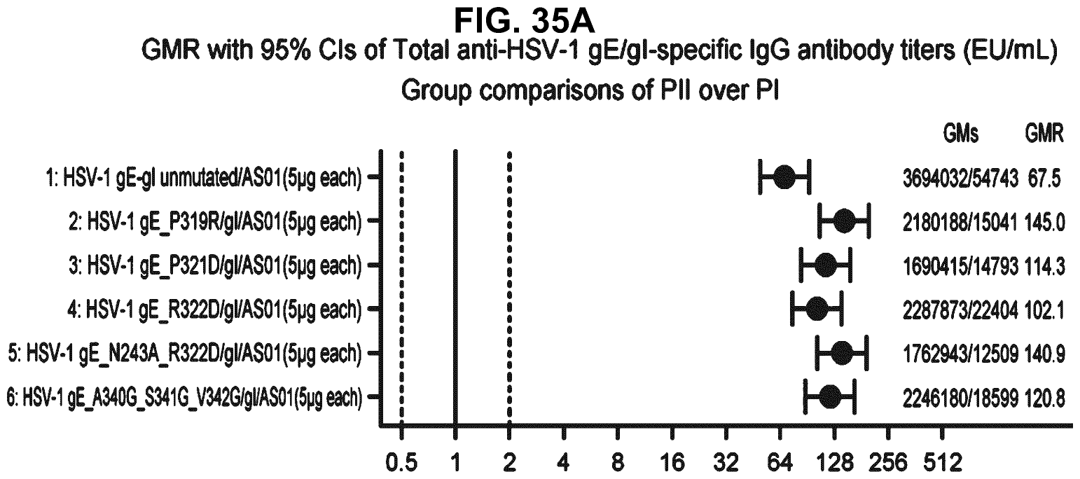

As expected, no anti-HSV-1-specific gE/gI response was observed in NaCl control group (<30 EU/ml for all mice) while all the AS01-adjuvanted HSV-1 gE/gI vaccinated-mice (gr2-6) produced a response above 2.000.000 EU/mL at the last time point. Compared to NaCl control group, high anti-HSV-1 gE/gI- vaccine-specific IgG antibody response was induced by all AS01-adjuvanted HSV-1 gE/gI proteins tested (unmutated and mutated versions) in this study at 14 days post third immunization (all GMRs>80.000) (FIG. 33 & FIG. 34).

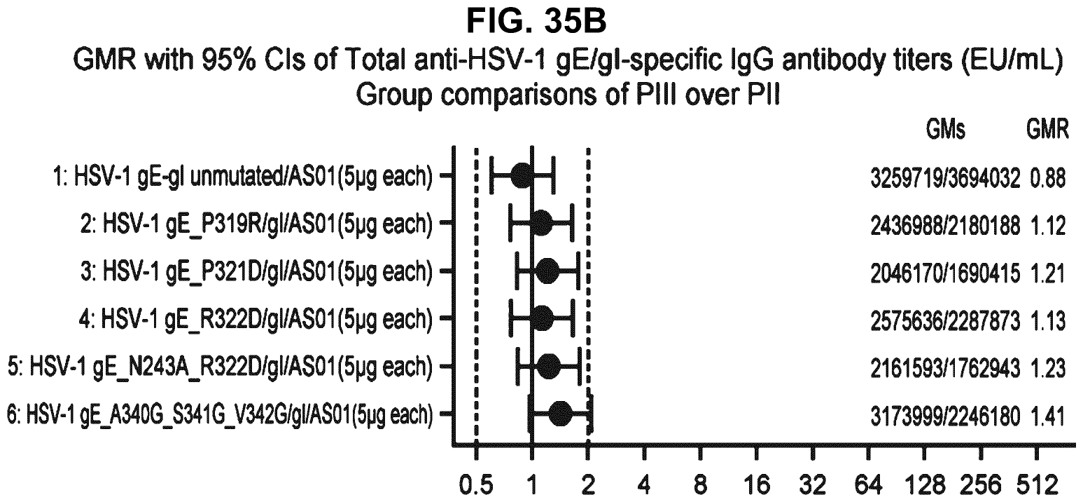

In all groups of mice immunized with different mutated versions of HSV-1 gE/gI protein, anti-HSV-1 gE/gI-specific IgG antibody responses increased after the second immunization (day27 (13PII)) compared to the first one (day13 (13PI)), with a fold increase ranging from 68 to 145 (FIG. 35A).

For all HSV-1 gE/gI mutant candidates, similar anti-HSV-1 gE/gI-specific antibody response was observed 14 days after the third immunization (day42(14PIII)) compared to the second one (day27 (13PII)), with a fold increase close to 1 (with CIs containing 1), indicating no booster effect of the third dose (FIG. 35B).

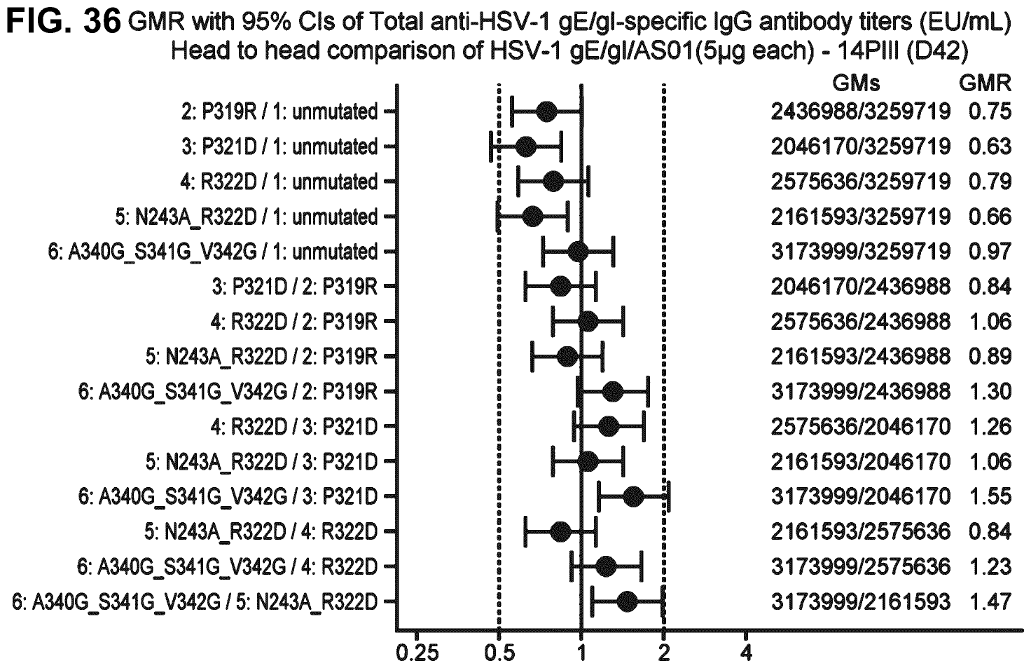

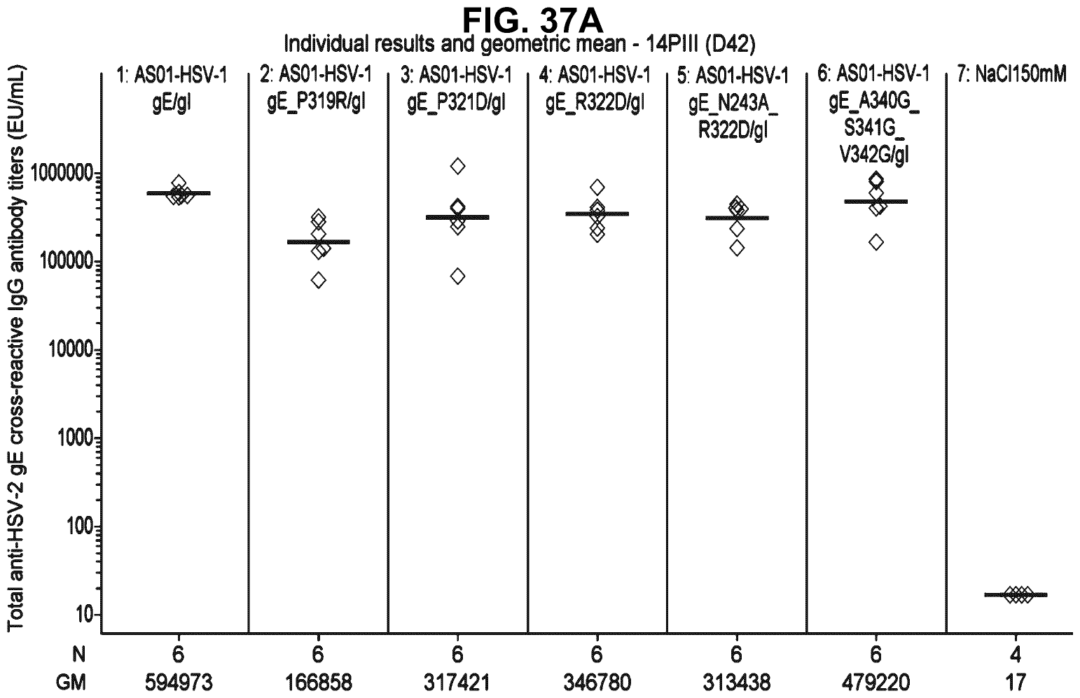

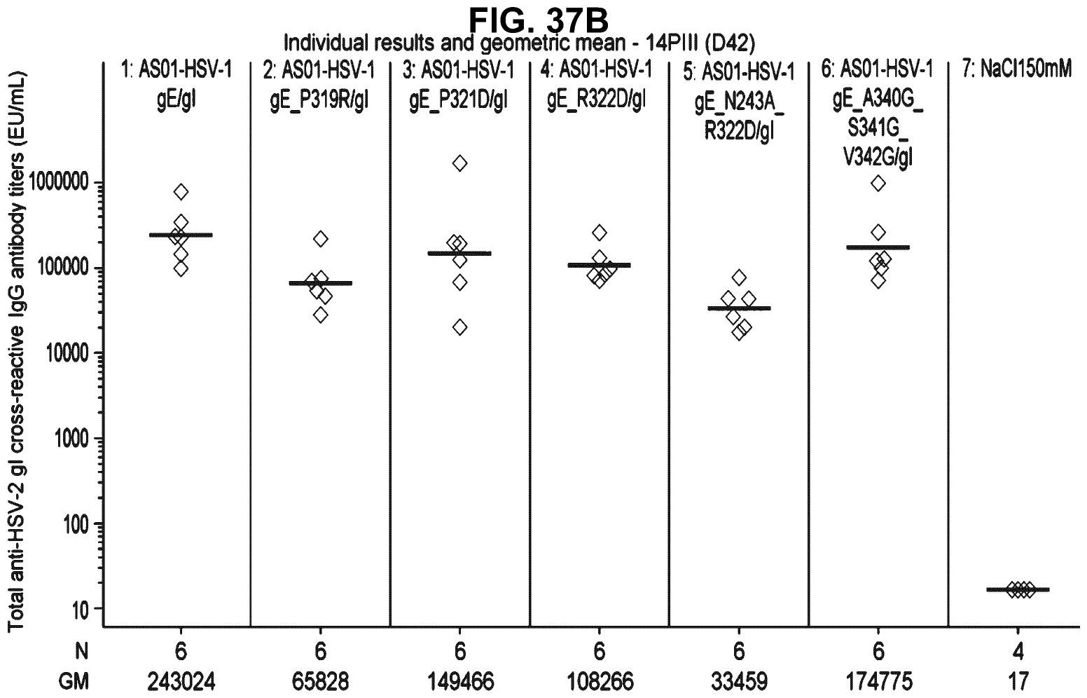

Altogether, results suggest very similar anti-HSV-1 gE/gI-specific IgG antibody responses between the different mutated versions of HSV-1 gE/gI mutant candidates 14 days post third immunization (with GMRs close to 1-fold change and most CIs are totally included in [0.5,2]) and between the different mutated versions of HSV-1 gE/gI protein and the unmutated one (FIG. 36).

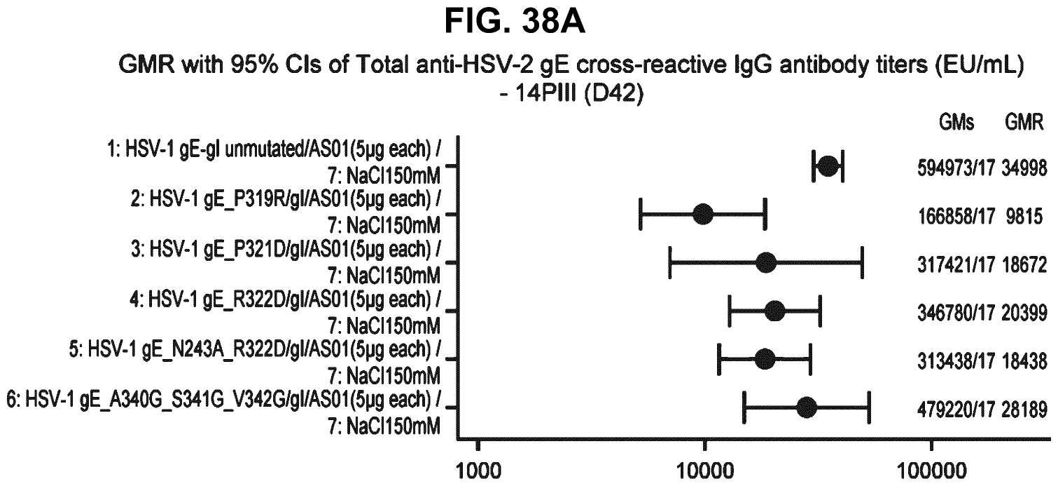

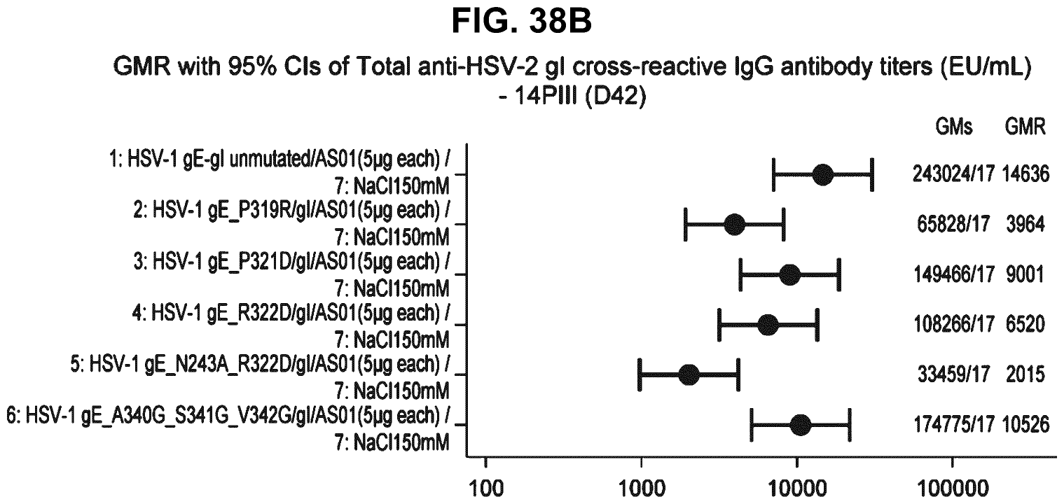

As expected, anti-HSV-2 gE and anti-HSV-2 gI cross-reactive responses were not observed in NaCl control group (17 EU/mL for all mice). At 14 days post third immunization and compared to NaCl control group, high anti-HSV-2 gE and anti-HSV-2 gI cross-reactive IgG antibody responses were induced in all groups of mice immunized by different unmutated and different mutated versions of AS01-adjuvanted HSV-1 gE/gI protein (all GMRs>9.000 for gE and >2.000 for gI) (FIG. 37, FIG. 38).

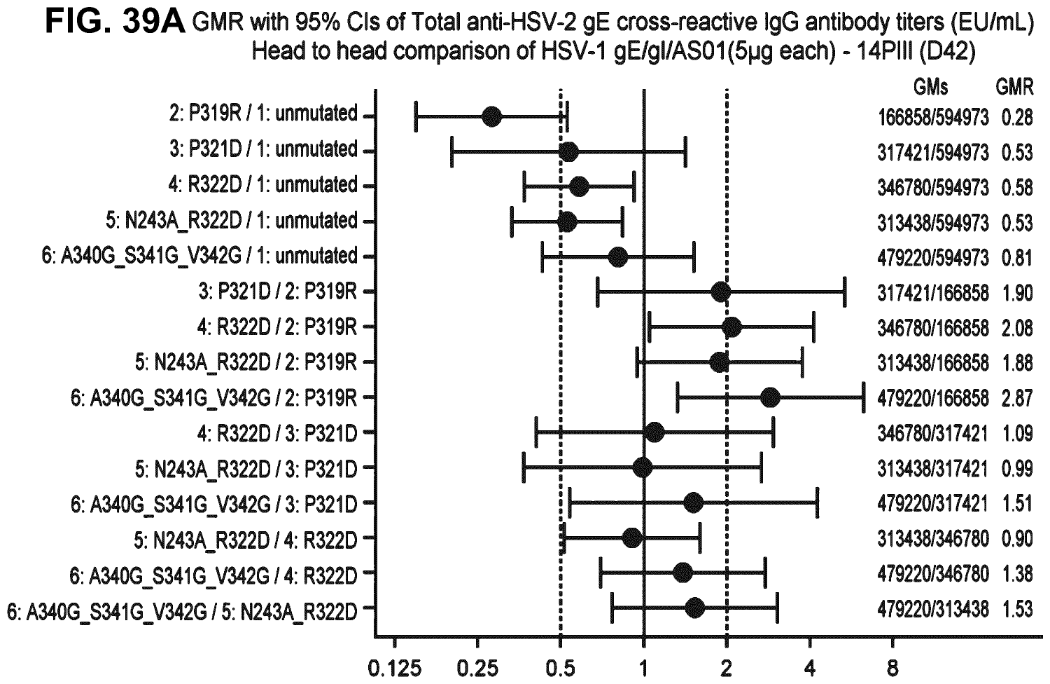

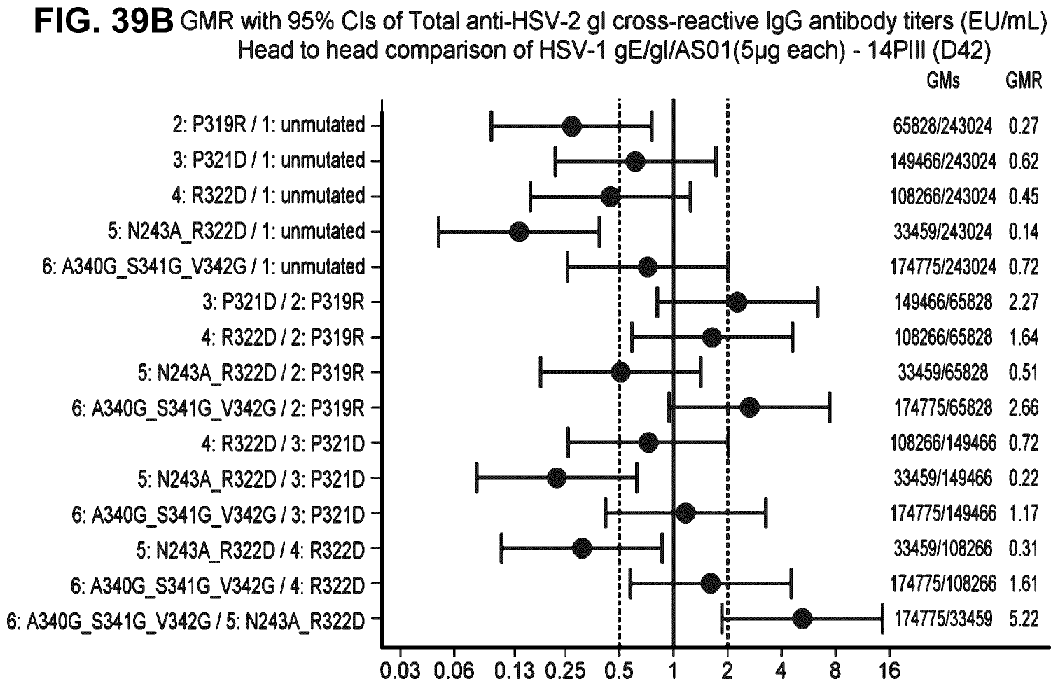

Several differences between HSV-1 gE/gI unmutated and mutant candidates are observed on both anti-HSV-2 gE and anti-HSV-2 gI cross-reactive antibody responses at 14 days post third immunization. Indeed, both anti-HSV-2 gE and anti-HSV-2 gI cross-reactive responses induced from unmutated vaccine protein (group 1) seemed to be higher than the ones induced by P319R mutant (group 2) and N243A_R322D mutant (group 5) (fold changes ranging from 2 to 7 with CIs not containing 1). Both anti-HSV-2 HSV-2 gE and anti-HSV-2 gI responses seemed to be also higher with A340G_S341G_V342G mutant (group 6) than with P319R mutant (group 2) (GMRs of 2.9 and 2.7 with CIs not or almost not containing 1, respectively). In addition, anti-HSV-2 gE response induced by R322D mutant (group 4) seemed to be twice higher than the one induced by P319R mutant (group 2) (with CI not containing 1). Finally, anti-HSV-2 gI response induced by P321D (group 3), R322D (group 4) and A340G_5341 G_V342G (group 6) mutants seemed to be higher than the one induced by N243A_R322D mutant (group 5) (fold changes ranging from 2 to 5, with CIs not containing 1). No other differences between mutants seemed to be observed (with GMRs<2-fold change and/or CIs containing 1) (FIG. 39).

Different Versions of AS01-HSV-1 gE/gI Proteins can Induce Functional Vaccine-Specific and HSV-2 gE/gI Cross Reactive Antibody Responses

In this study, HSV-1-specific & HSV-2 gE/gI cross-reactive antibody functions were only investigated in the sera collected at 14 days post third immunization. In this regard, the ability of vaccine-induced pAbs to neutralize HSV-1 & HSV-2 viruses, to decrease in-vitro binding of human IgG Fc by HSV-1 or HSV-2 gE/gI protein and to cross-react and activate mFcγRIII expressed on modified Jurkat cells after incubation with HSV-2 or HSV-1 gE/gI positive 3T3 cells was investigated.

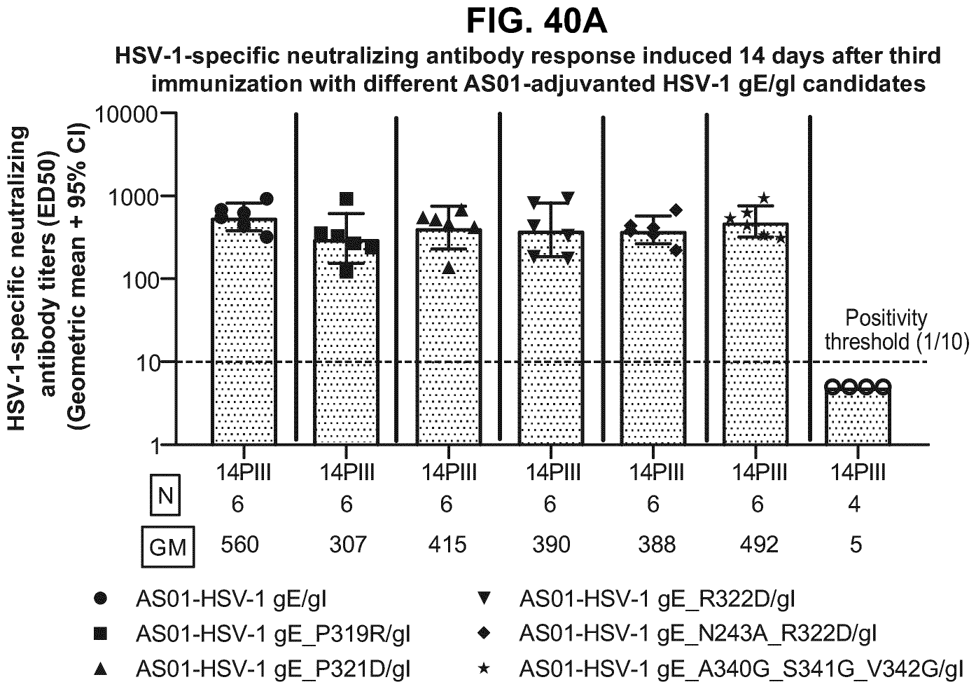

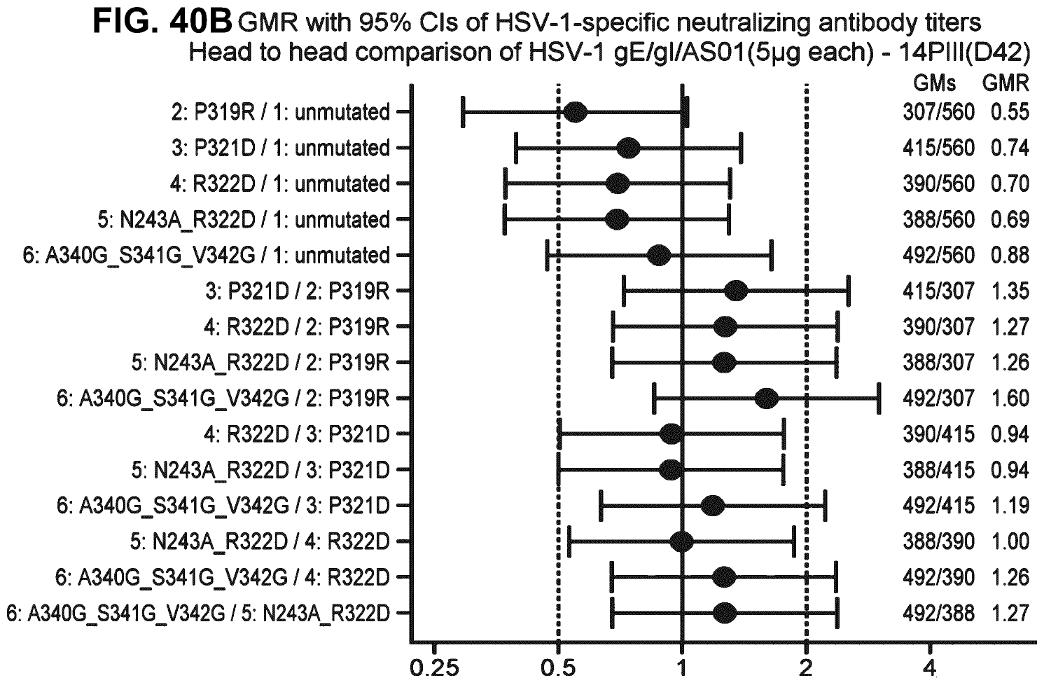

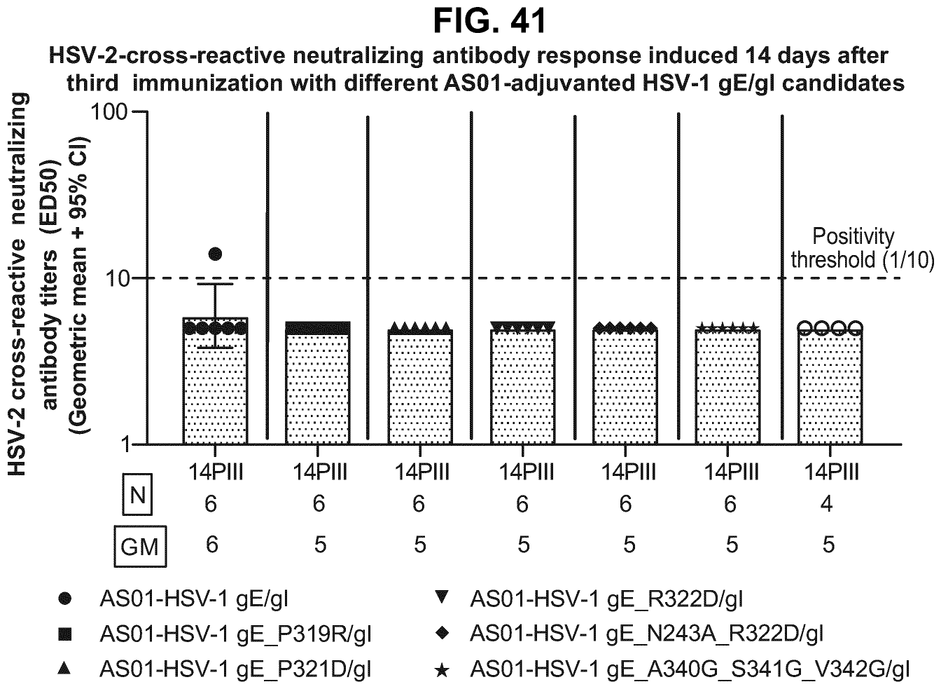

Consistent moderate levels of neutralizing antibody (nAb) response directed to HSV-1 VR-1789 strain was detected in all groups of mice immunized with the different versions of AS01-adjuvanted HSV-1 gE/gI protein (FIG. 40A). These results suggest no difference in term of HSV-1 neutralizing activity between the different groups of mice (GMRs<2-fold change with all CIs containing 1). However, a trend for higher response in unmutated group compared to P319R mutant was observed (GMR P319R/unmutated=0.55, with upper limit of CI just above 1) (FIG. 40B). In contrast, no anti-HSV-2 cross-reactive nAb response directed to HSV-2 MS strain was detected in any of the groups immunized with AS01-adjuvanted HSV-1 gE/gI proteins (FIG. 41).

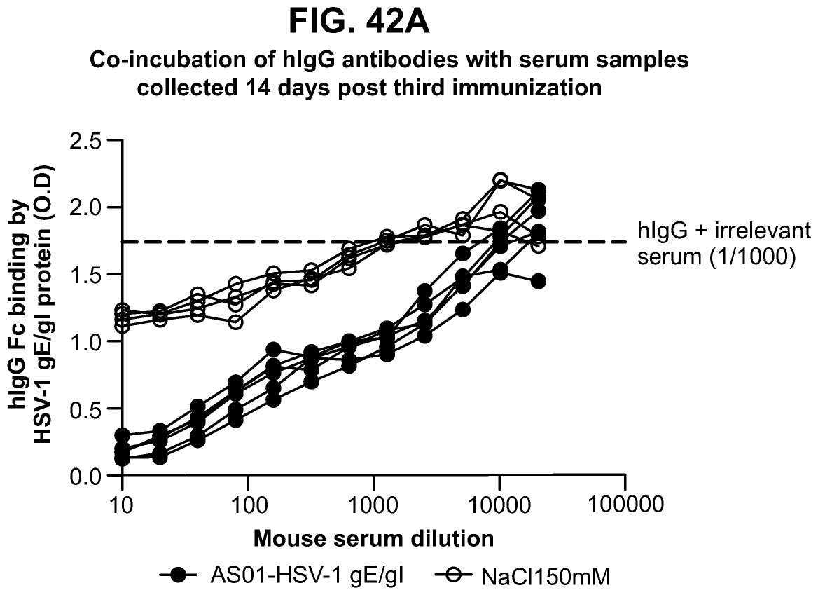

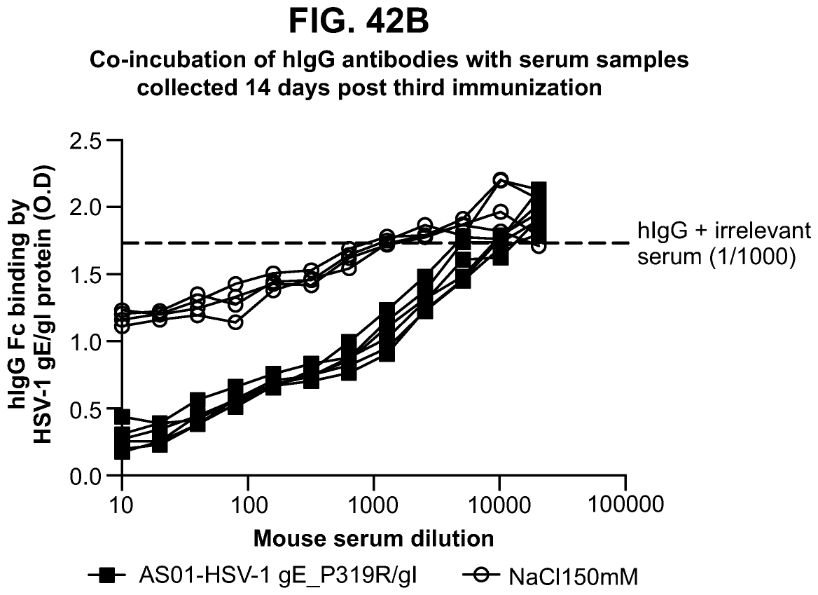

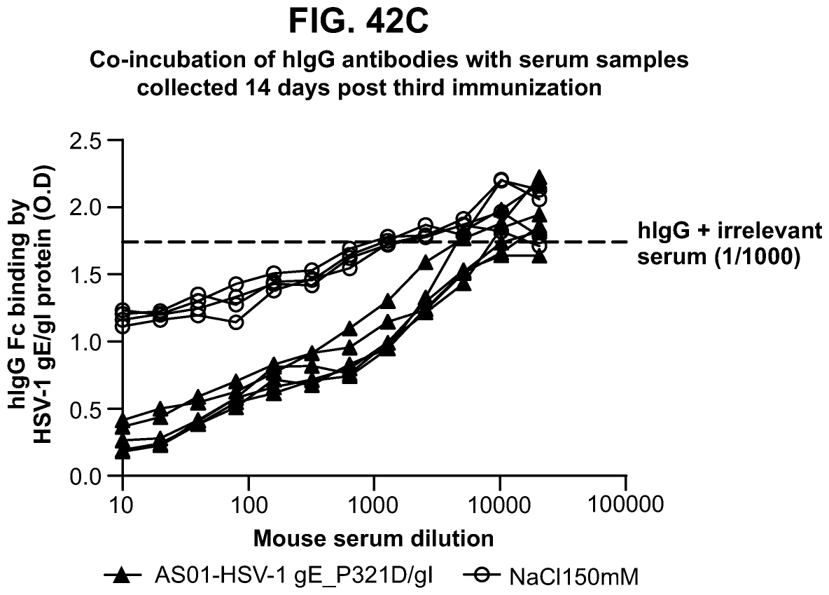

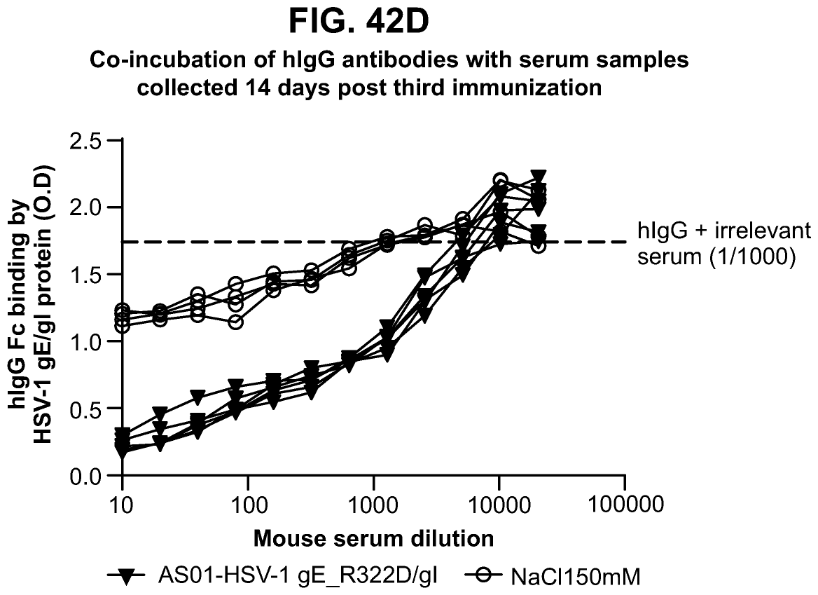

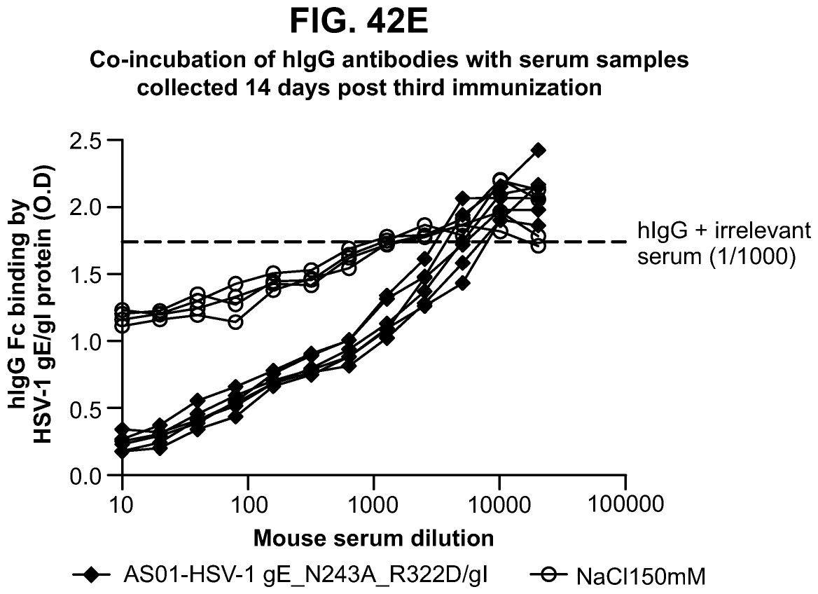

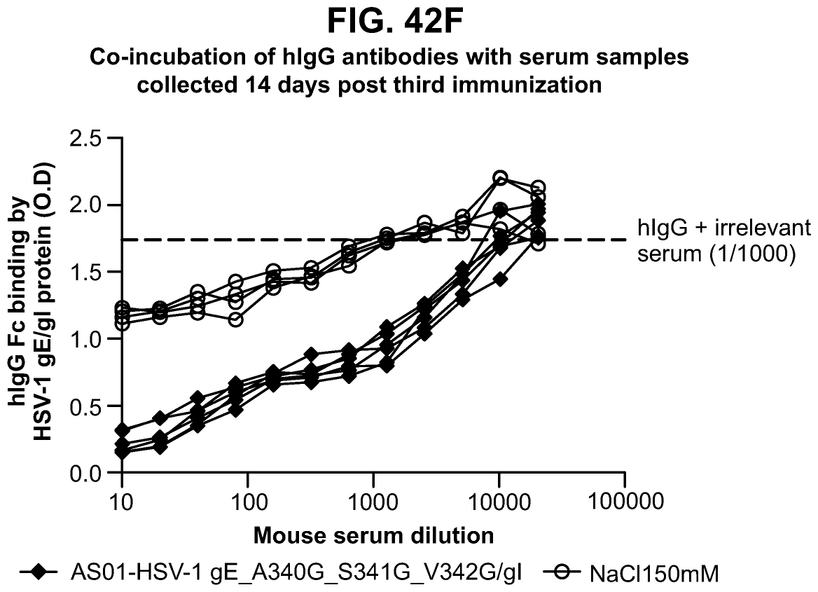

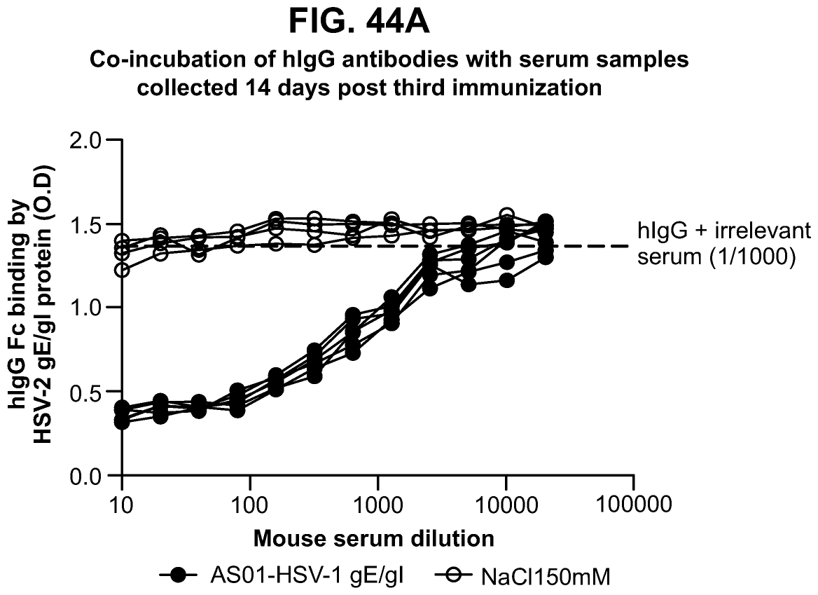

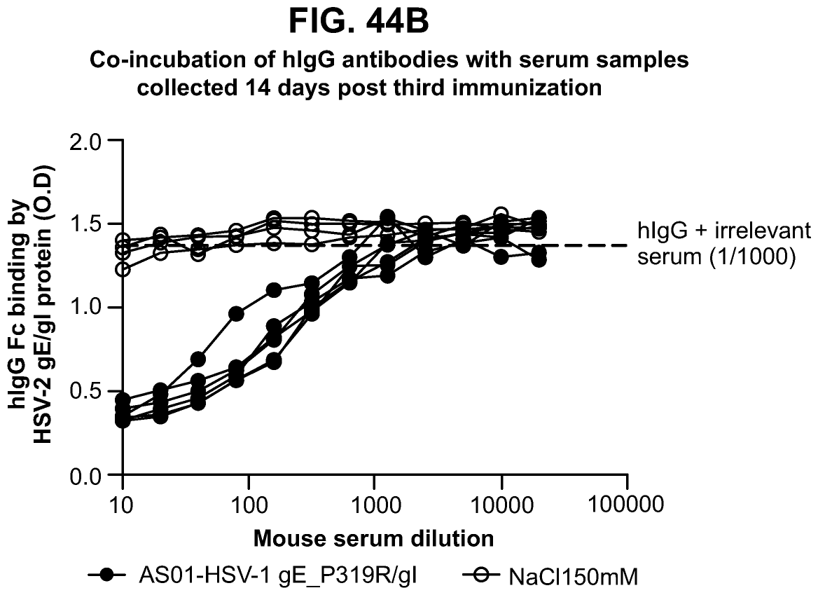

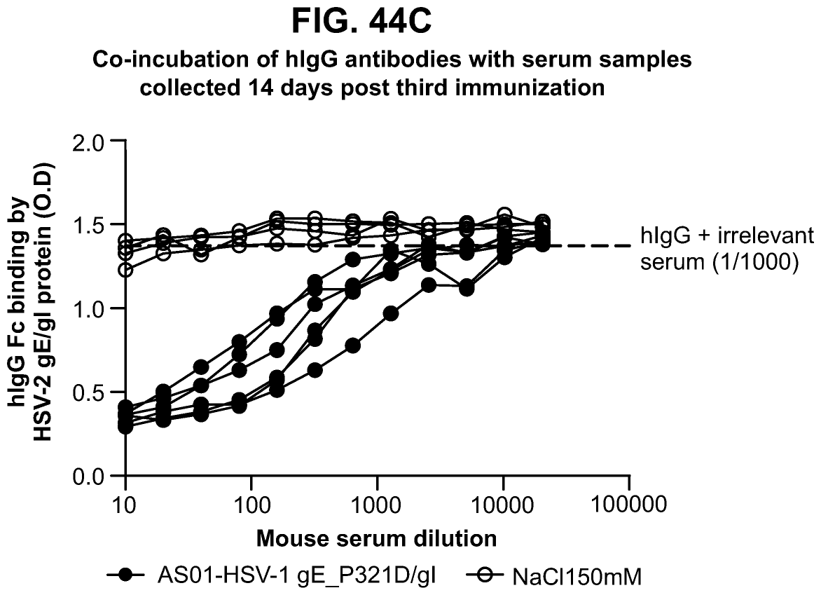

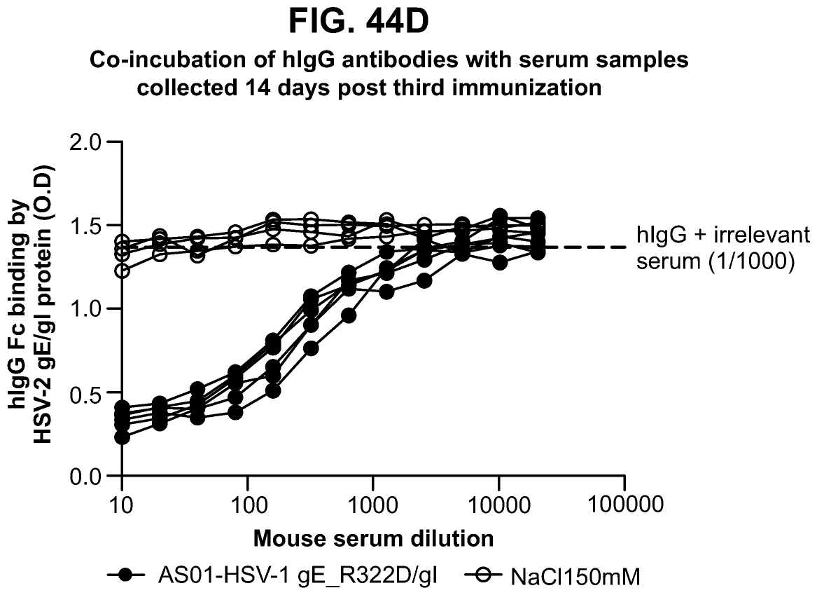

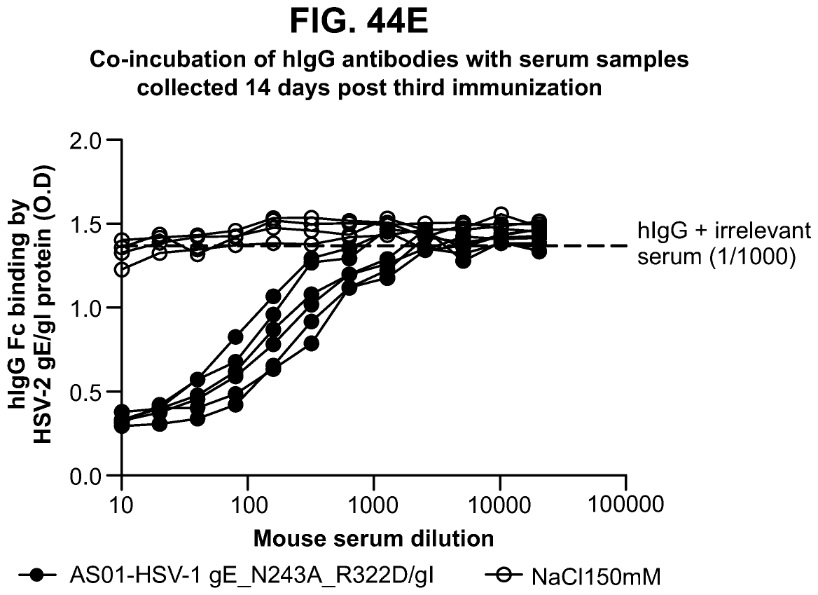

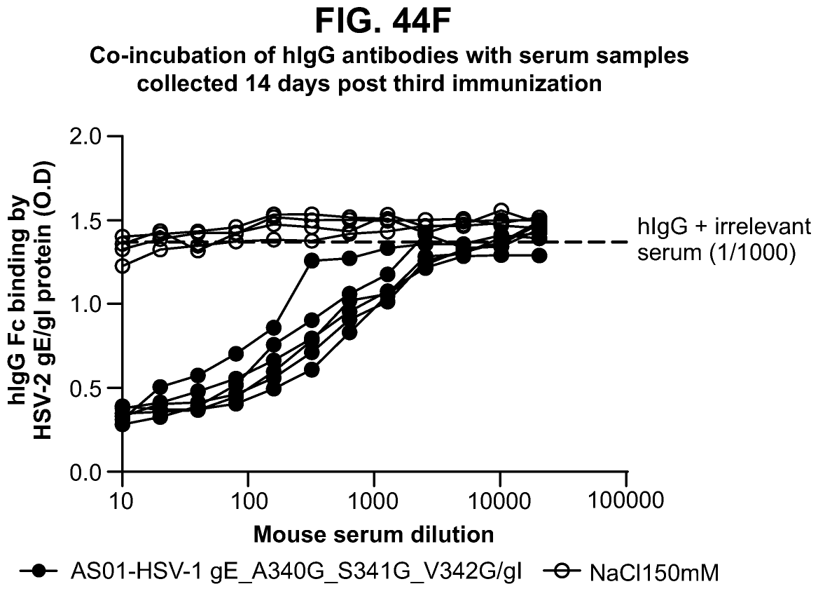

Then, the ability of sera from mice immunized with different versions of HSV-1 gE/gI protein to compete and decrease hIgG Fc binding by HSV-1 or HSV-2 gE/gI protein was assessed, in-vitro, by competitive ELISA. All HSV-1 gE/gI proteins (mutated and unmutated) elicited vaccine-specific and HSV-2 cross reactive polyclonal antibodies able to decrease hIgG Fc binding by HSV-1 or HSV-2 gE/gI protein (FIG. 42; FIG. 44).

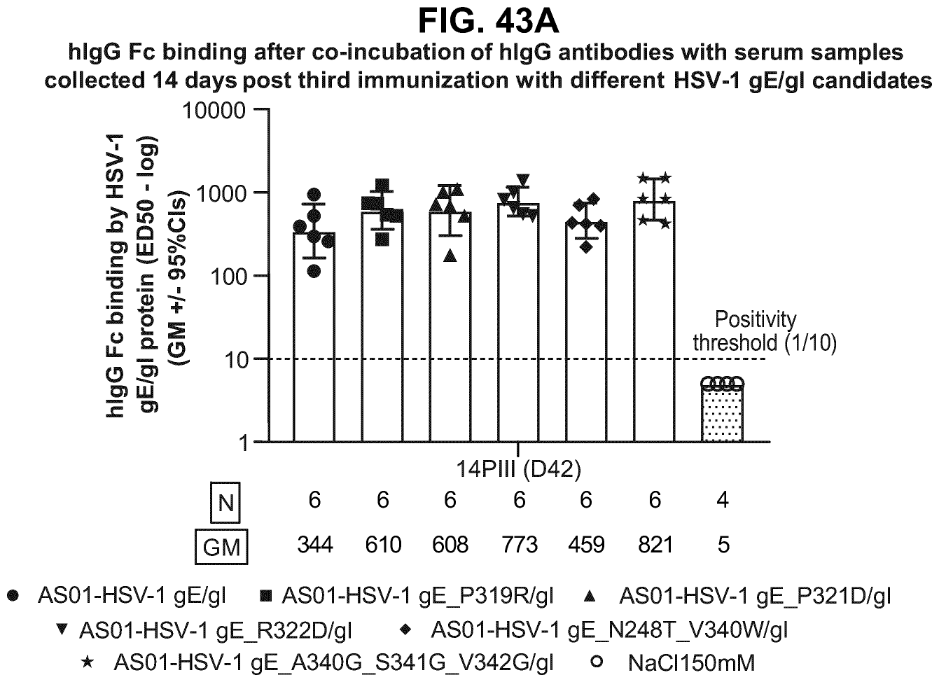

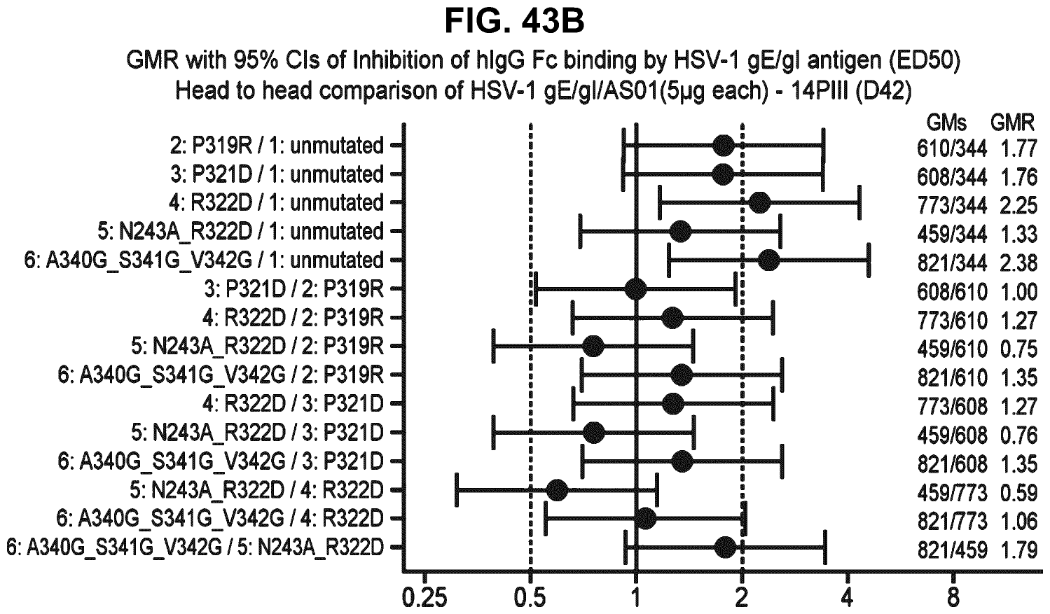

At same serum dilution, the level of hIgG Fc binding on HSV-1 gE/gI protein was quite similar between different groups of HSV-1 gE/gI immunized mice. The calculation of ED50 shown that only R322D (group4) and A340G_S341G_V342G (group 6) mutants seemed to induce higher response compared to the unmutated one (group 1) with fold-increases of 2.2 and 2.4, respectively (and CIs not containing 1). No other differences were observed between the different groups (GMRs<2-fold change and CIs containing 1) (FIG. 43A; FIG. 43B).

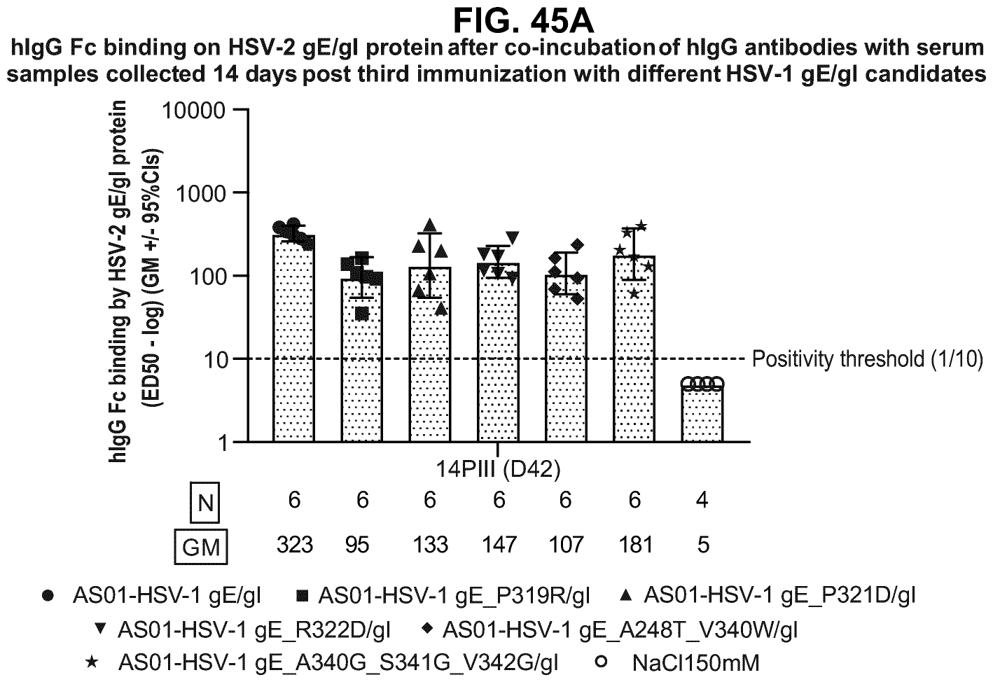

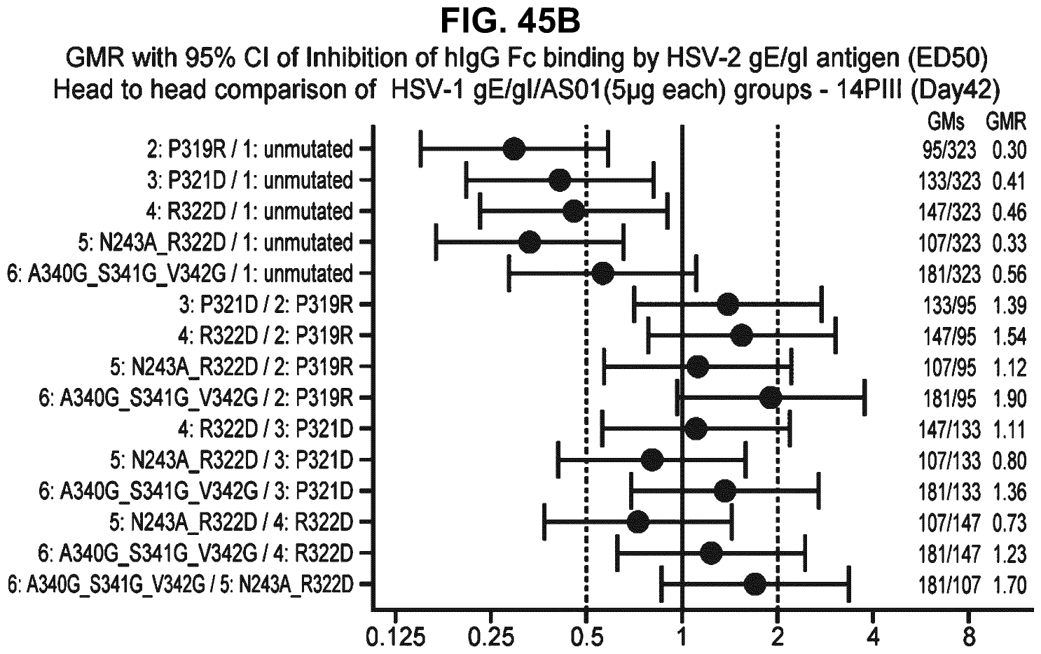

At same serum dilution, a few differences between groups of HSV-1 gE/gI immunized mice seem to be observed on the level of hIgG Fc binding on HSV-2 gE/gI protein. Indeed, response induced from unmutated protein (group 1) seemed to be higher than the one induced by P319R (group 2), P321D (group 3), R322D (group 4) and N243A_R322D (group 5) mutants (fold changes ranging from 2 to 3 with CIs not containing 1). A trend for higher response with A340G_S341G_V342G mutant (group 6) than with P319R mutant (group 2) seems also observed (GMR of 1.90 and CI almost not containing 1). No other differences between groups seemed to be observed (with GMRs<2-fold change and CIs containing 1). (FIG. 45A & FIG. 45B).

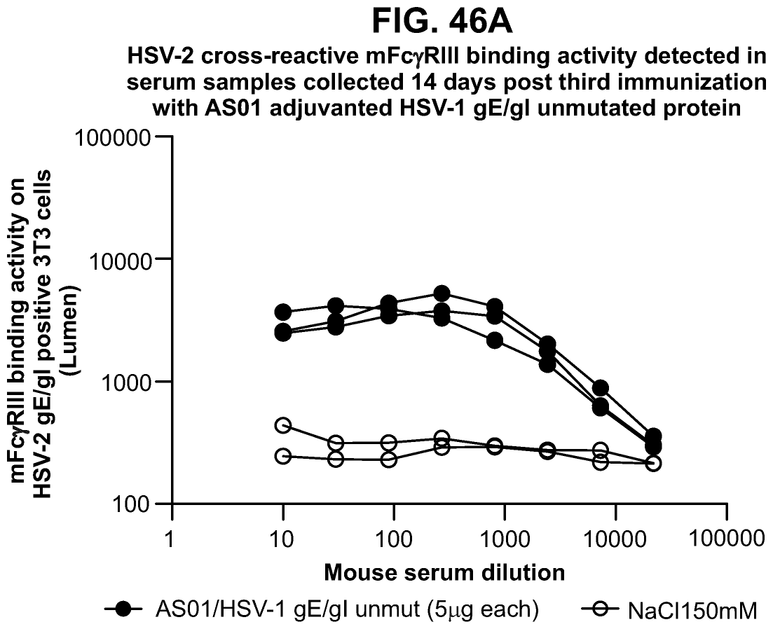

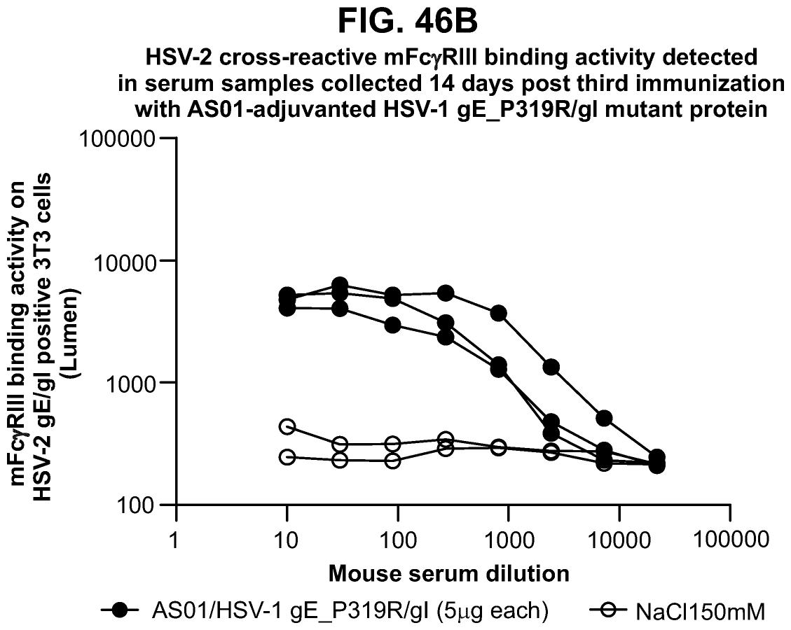

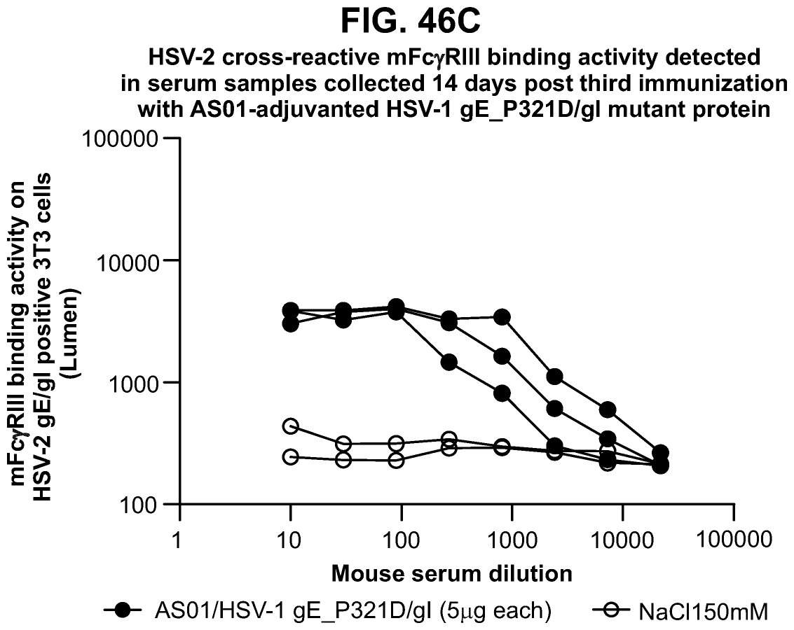

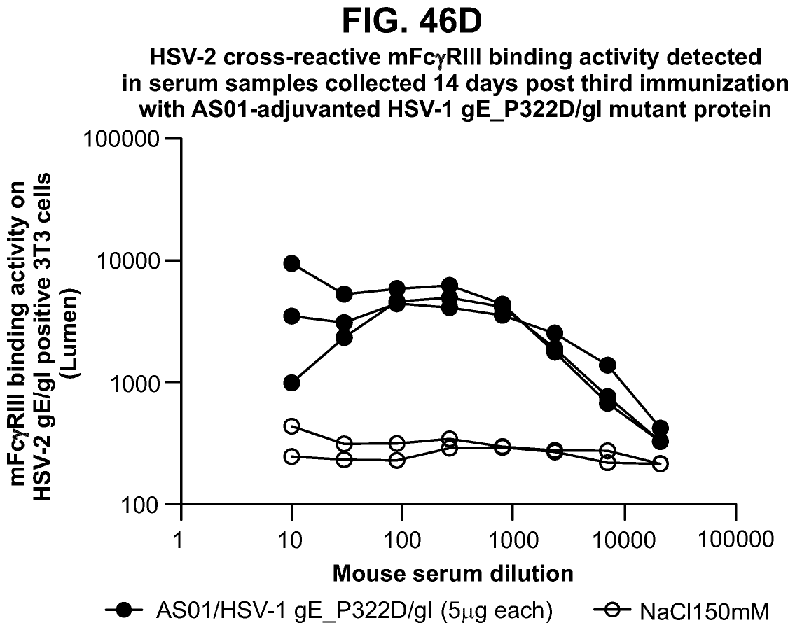

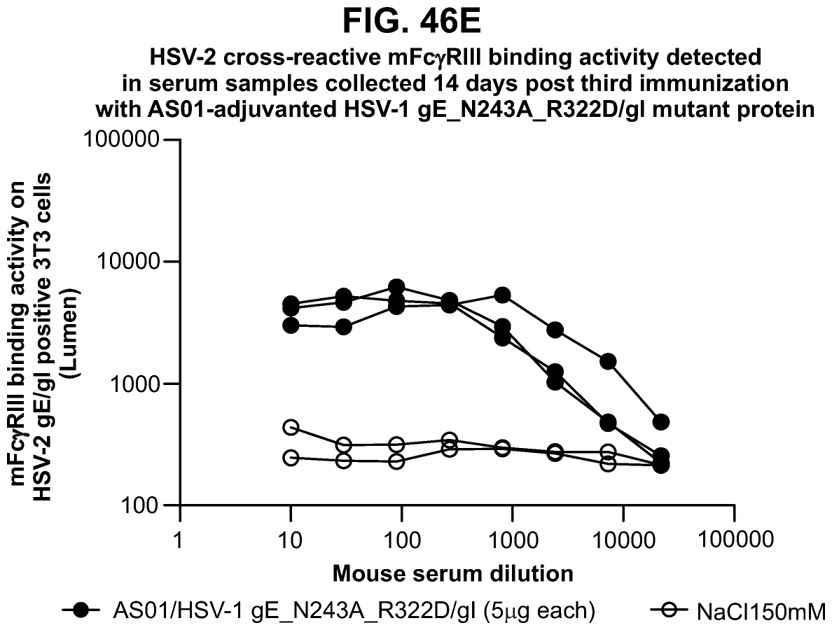

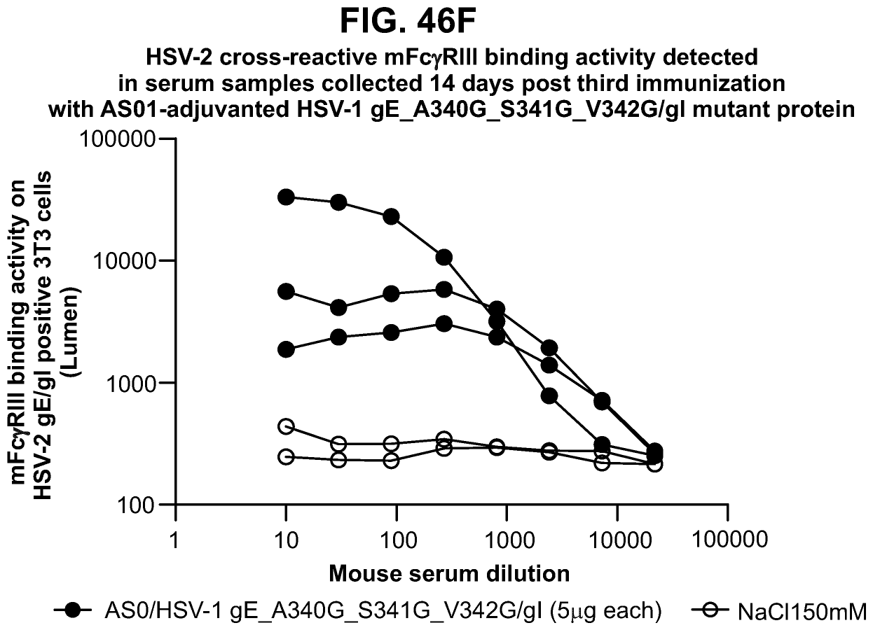

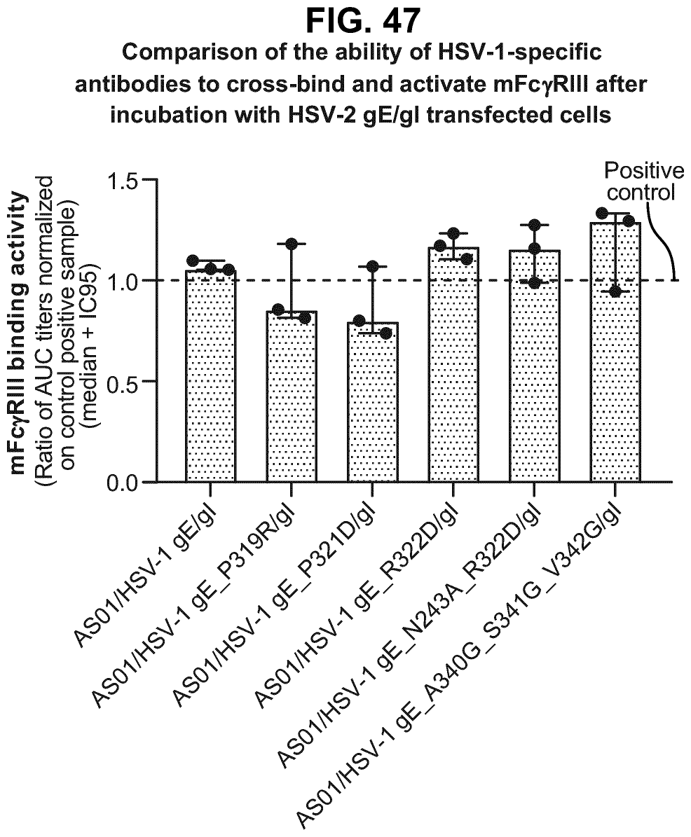

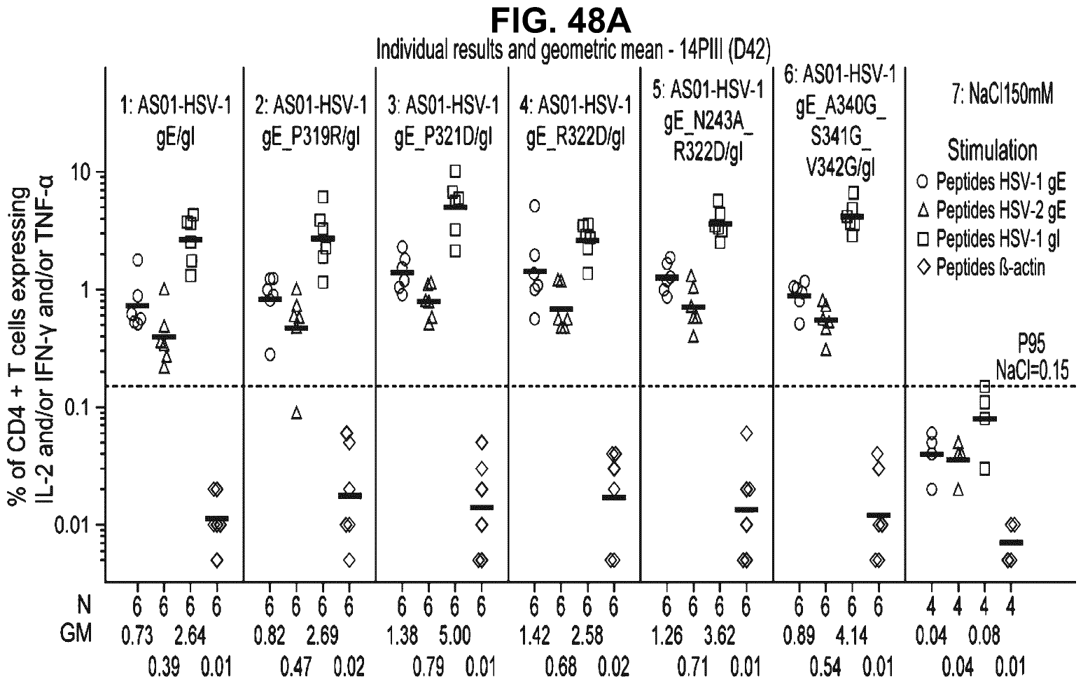

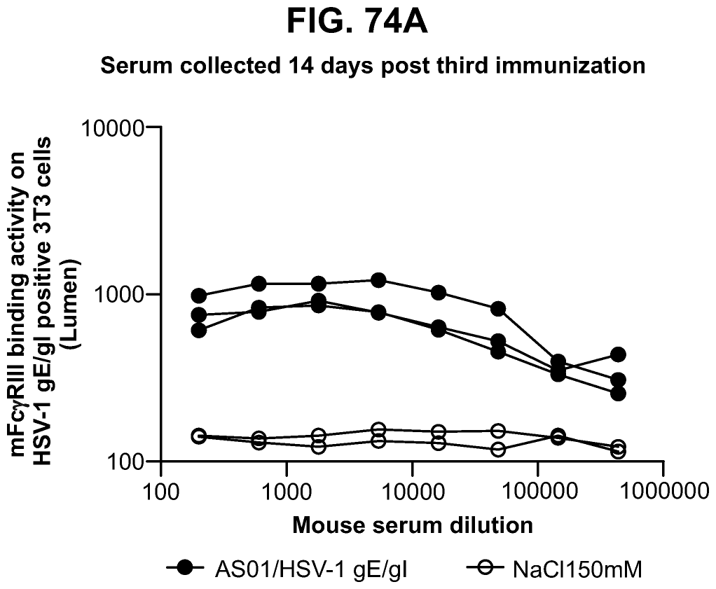

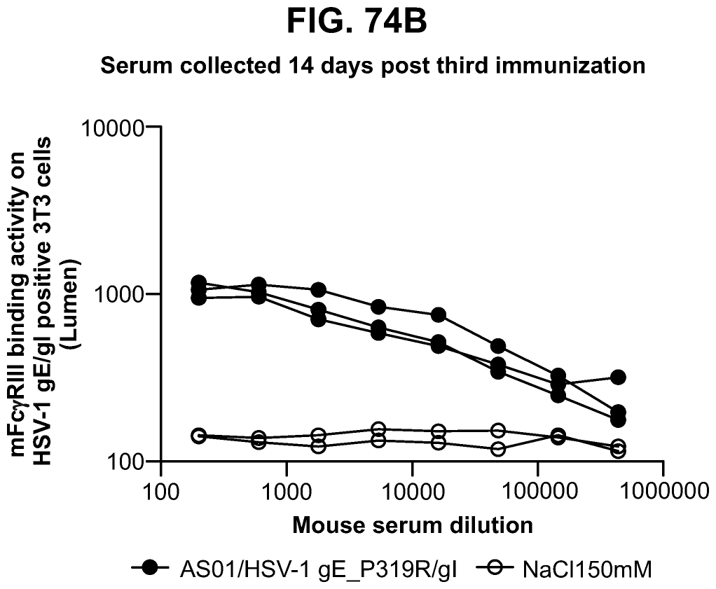

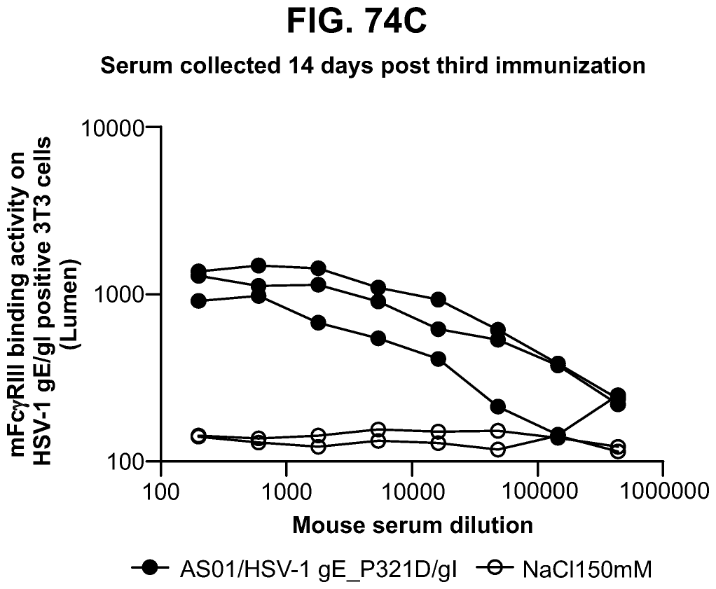

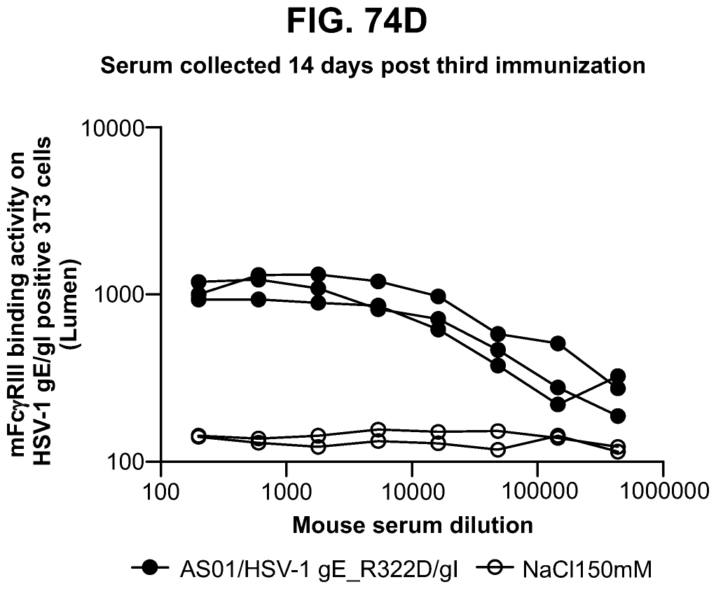

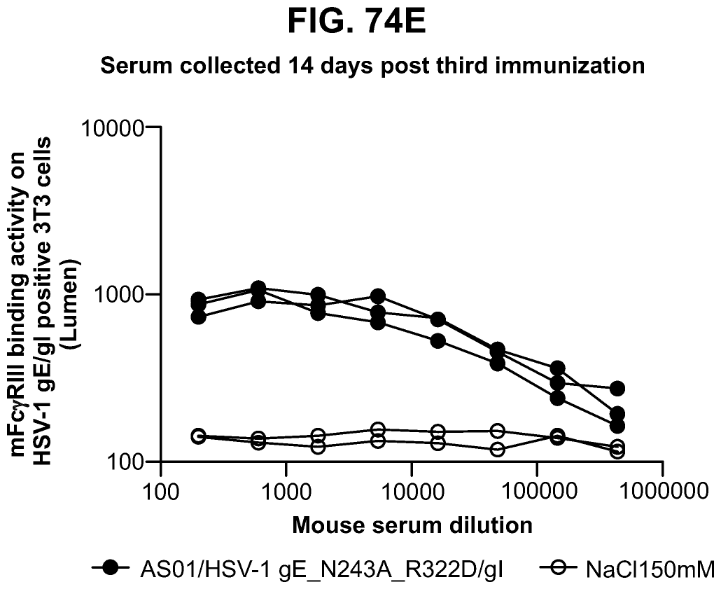

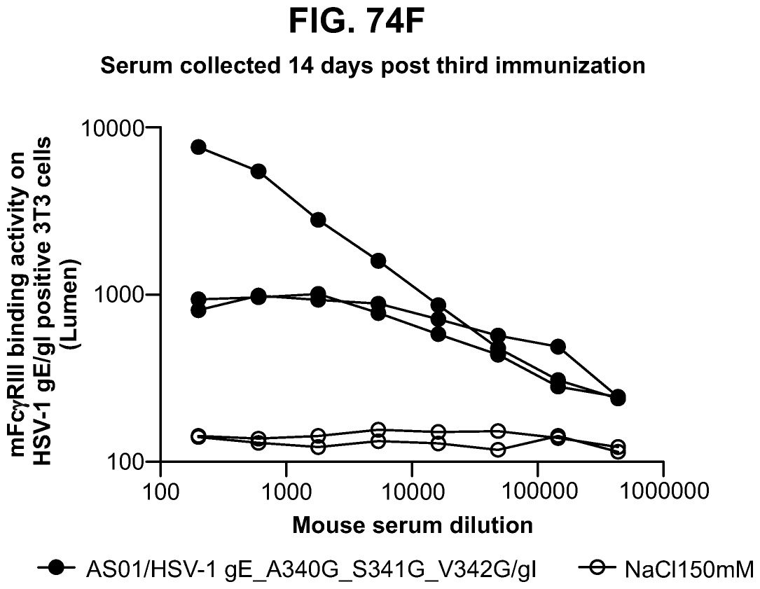

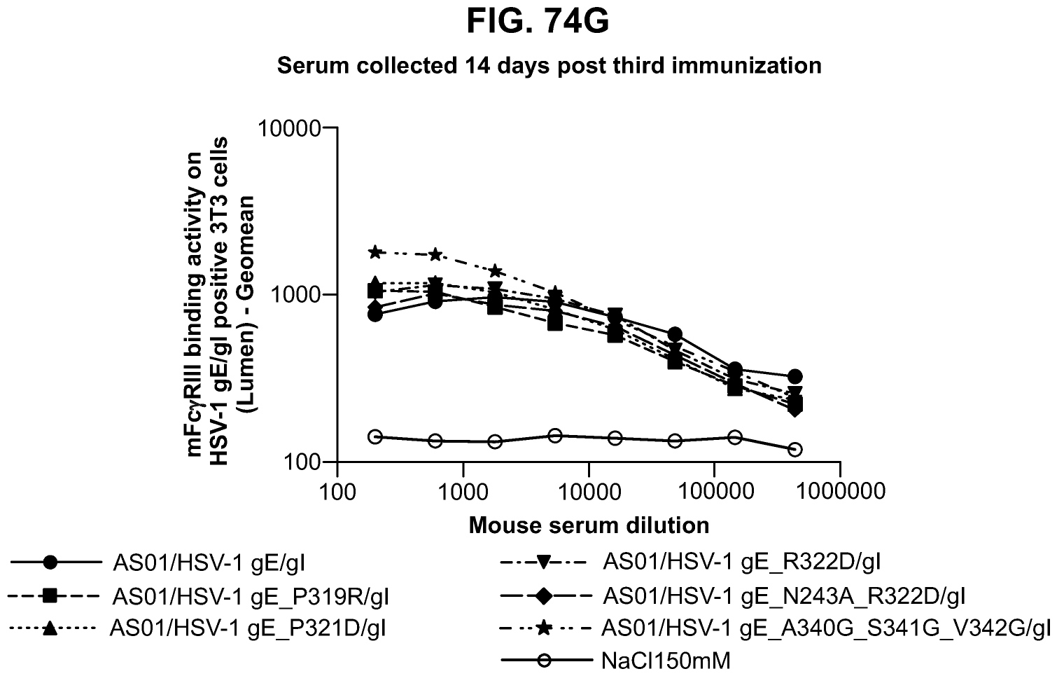

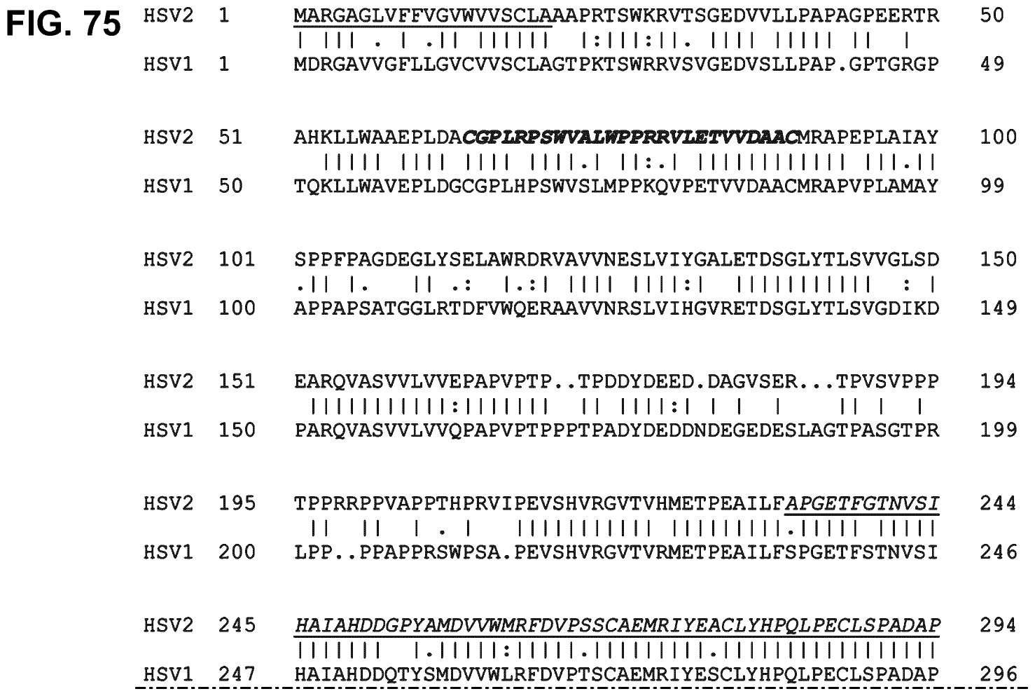

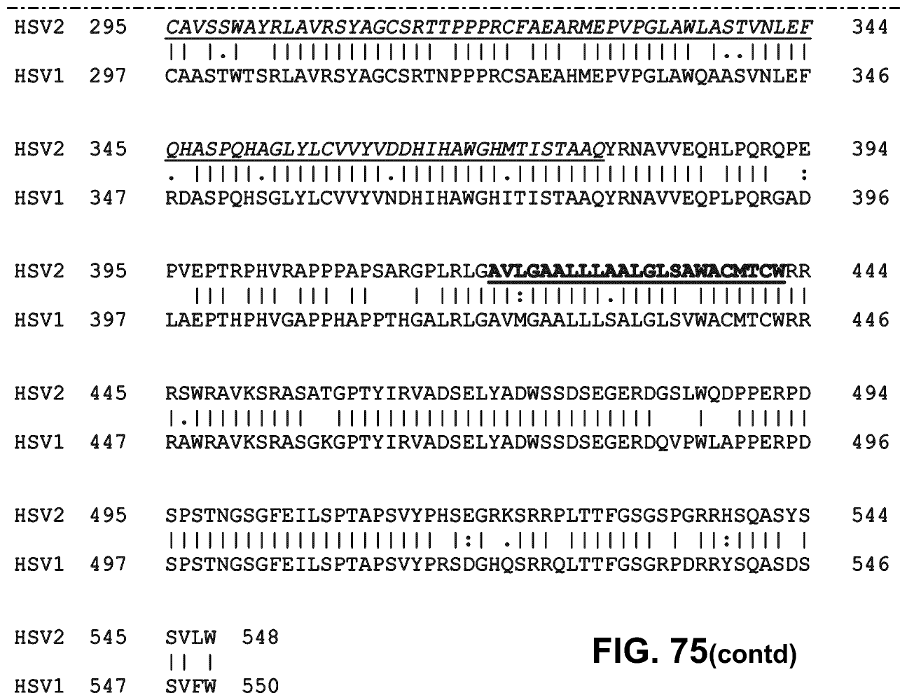

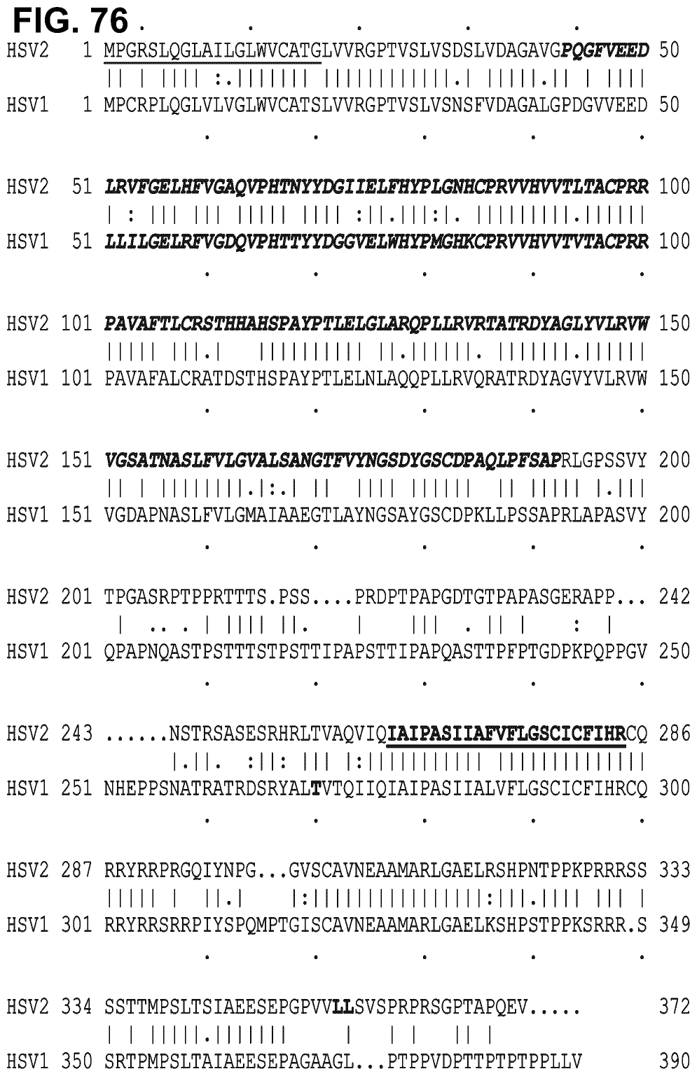

Finally, the ability of HSV1-gE/gI-specific or HSV-2 gE/gI cross reactive antibodies to bind and activate in-vitro, mouse FcγRIII after incubation with HSV-1 or HSV-2 gE/gI transfected 3T3 cells, was investigated 14 days after the third immunization. Data shown on the FIG. 46, FIG. 47 and FIG. 74 suggested that all mice immunized with the different HSV-1 gE/gI mutant candidates could induce vaccine-specific or HSV-2 gE/gI cross reactive antibodies able to bind and activate Jurkat reporter cells expressing FcγRIII after incubation with HSV-1 or HSV-2 gE/gI transfected cells. As expected, activation of FcγRIII was not detected with sera from unvaccinated NaCl control mice (FIG. 46 and FIG. 74). All these data suggest that different mutated versions of HSV-1 gE/gI protein could induce functional vaccine-specific or HSV-2 gE/gI cross-reactive antibody response against HSV-1 or HSV-2 gE/gI protein.

Different Versions of AS01-HSV-1 gE/gI Protein Induced Anti-HSV-1 gE/gI-Specific and Anti-HSV-2 gE Cross-Reactive CD4+ T Cell Responses

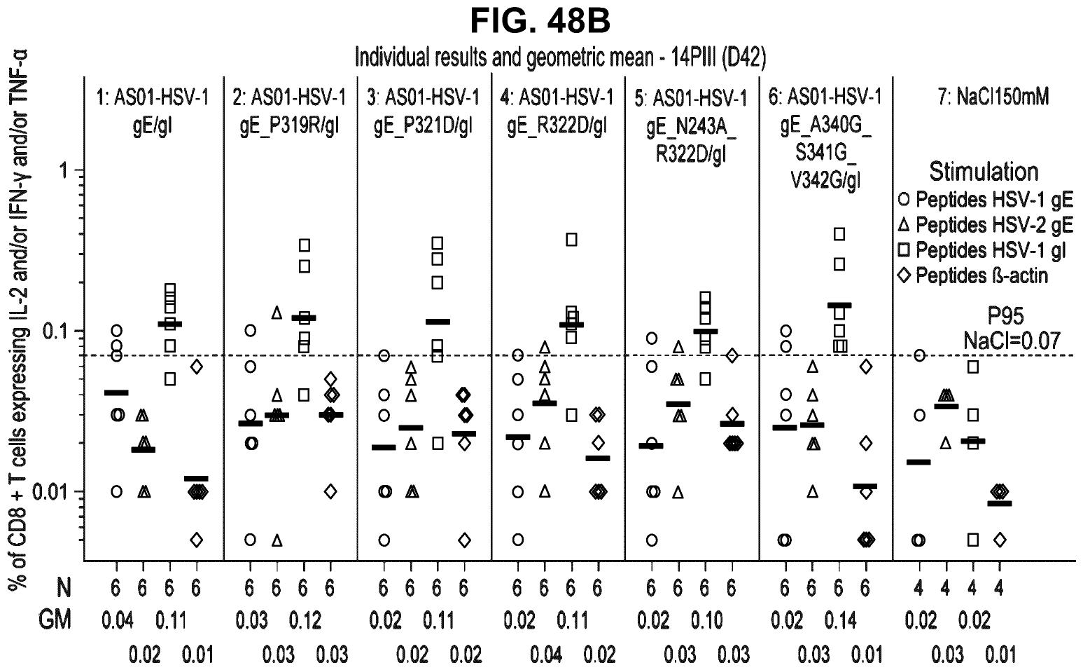

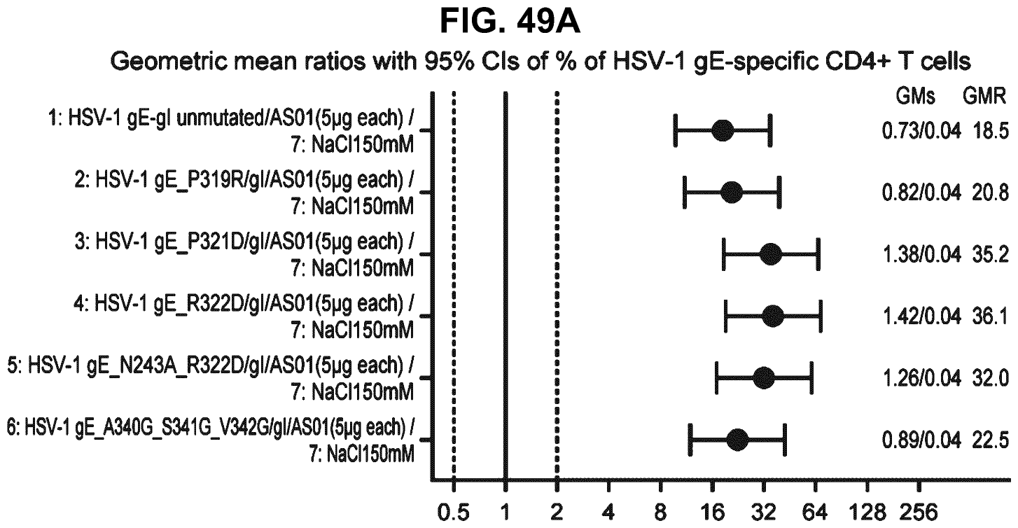

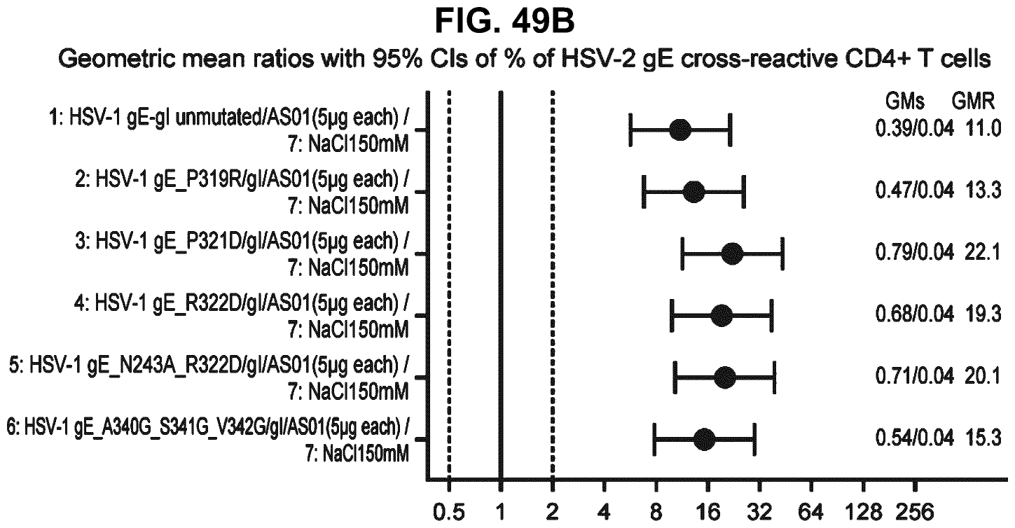

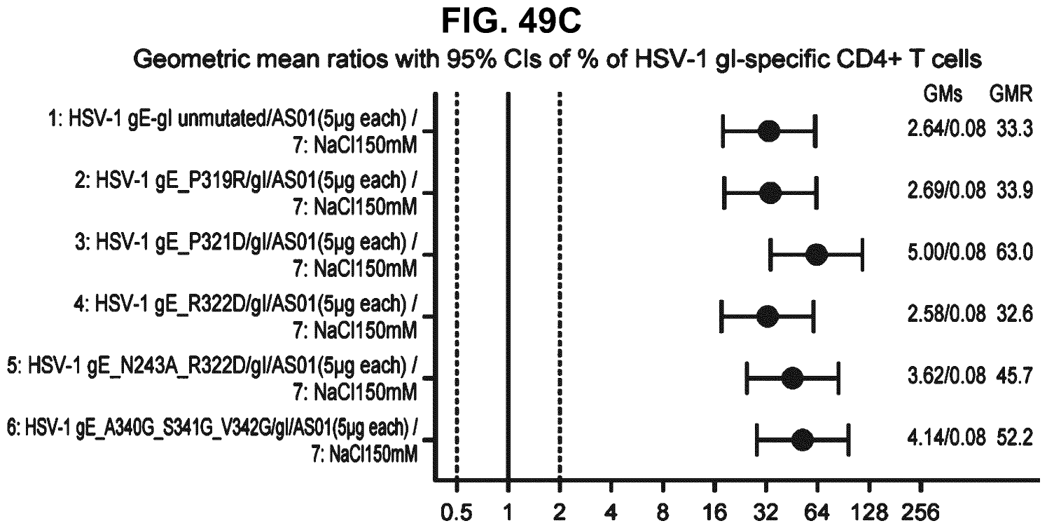

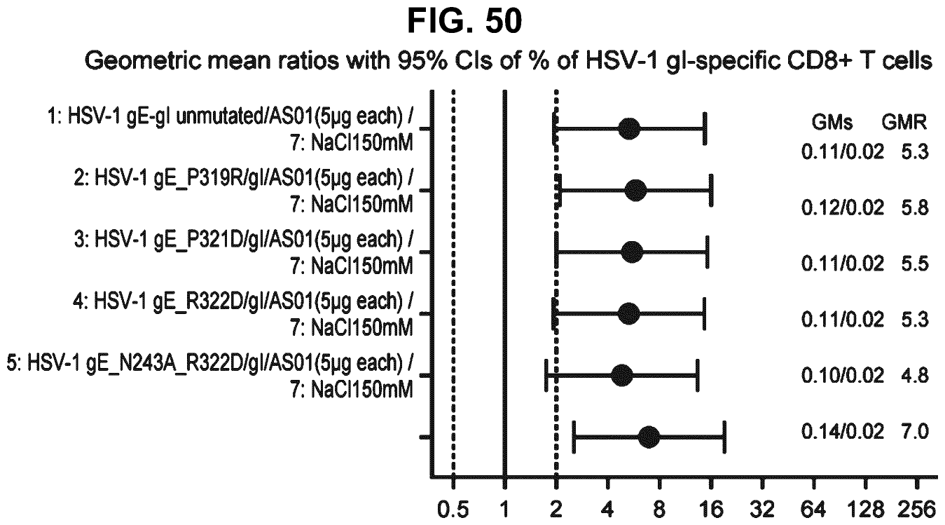

Compared to the NaCl control group, higher anti-HSV-1 gE and gI-specific and anti-HSV-2 gE cross-reactive CD4+T cell responses were detected 14 days after the third immunization in all groups of mice immunized with AS01-adjuvanted HSV-1 gE/gI proteins (unmutated and mutated versions), (FIG. 48A) with a fold increase ranging from 11 to 63 between vaccinated and NaCl groups (CIs not containing 1) (FIG. 49A, FIG. 49B & FIG. 49C). However, anti-HSV-1 gI-specific CD8+T cell response was only inconsistently detected in all vaccinated groups compared to the NaCl control group (GMRs ranging from 5 to 7 with CI not containing 1). The anti-HSV-1 gI-specific CD8+T cell response was considered as not relevant from a biological point of view (0.1 to 0.14%) (FIG. 49C & FIG. 50). No anti-HSV-1 gE-specific or anti-HSV-2 gE cross-reactive CD8+T cell responses was detected in any of vaccinated groups compared to the NaCl control group (data not shown).

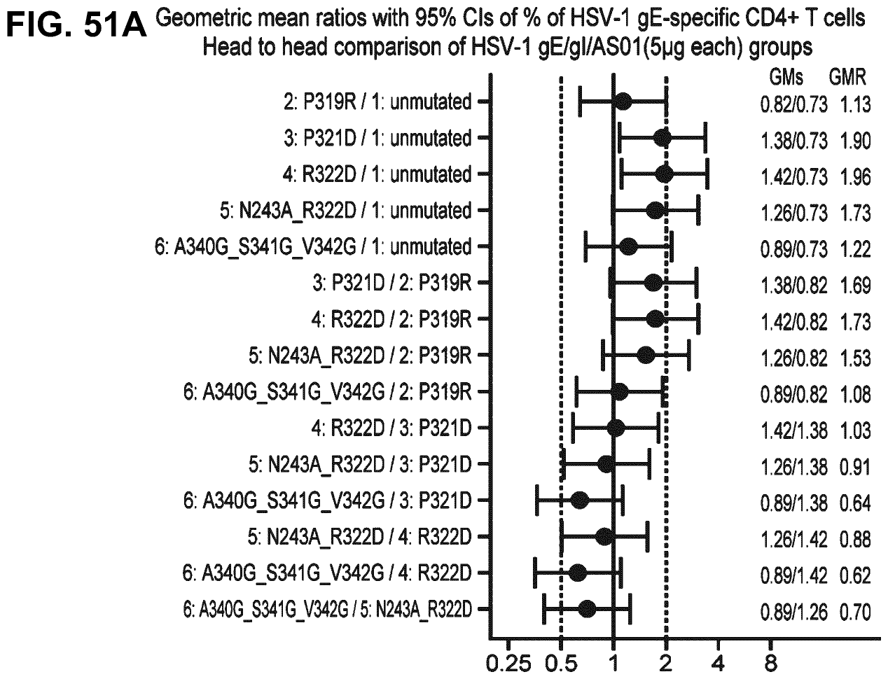

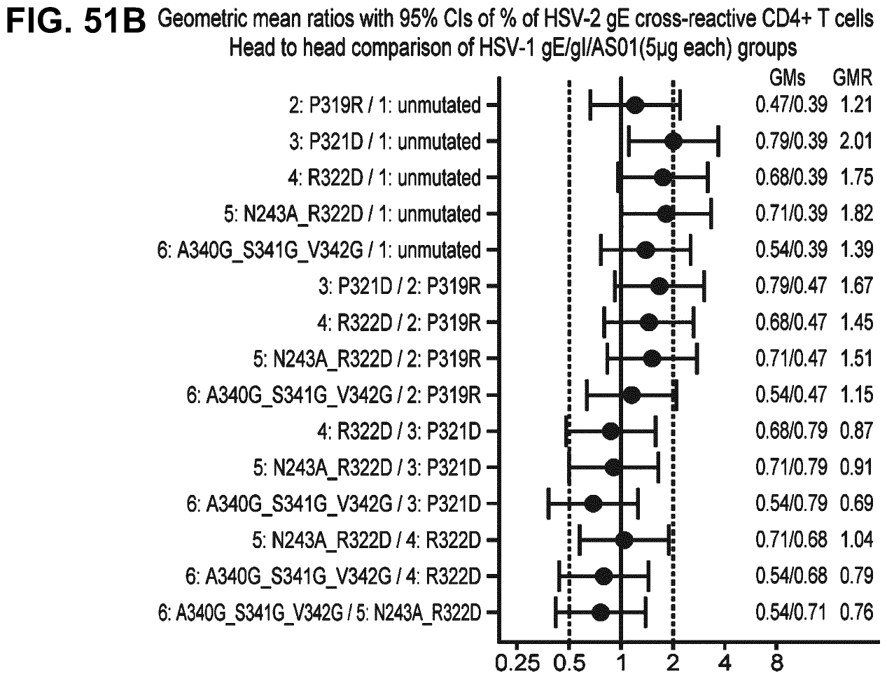

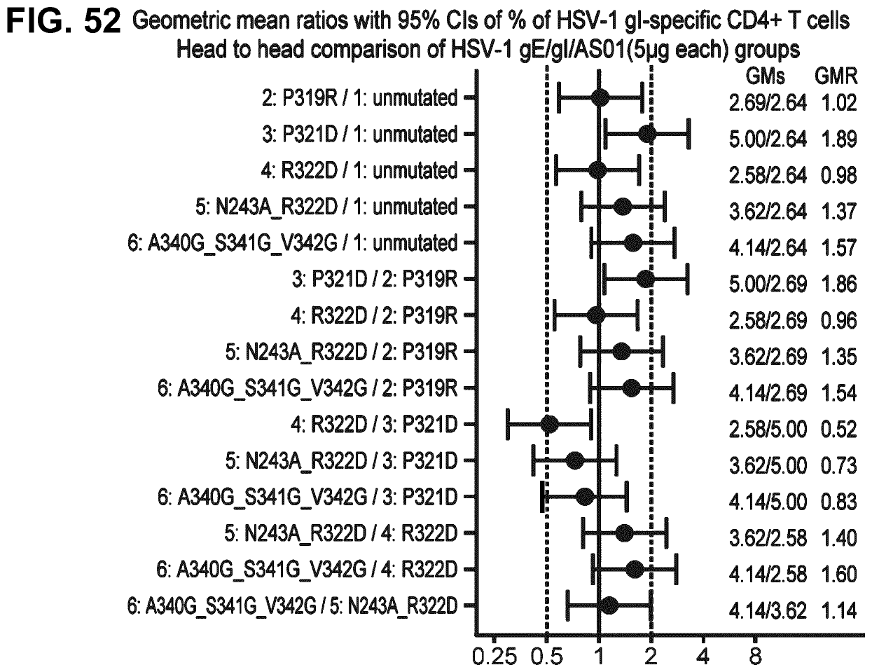

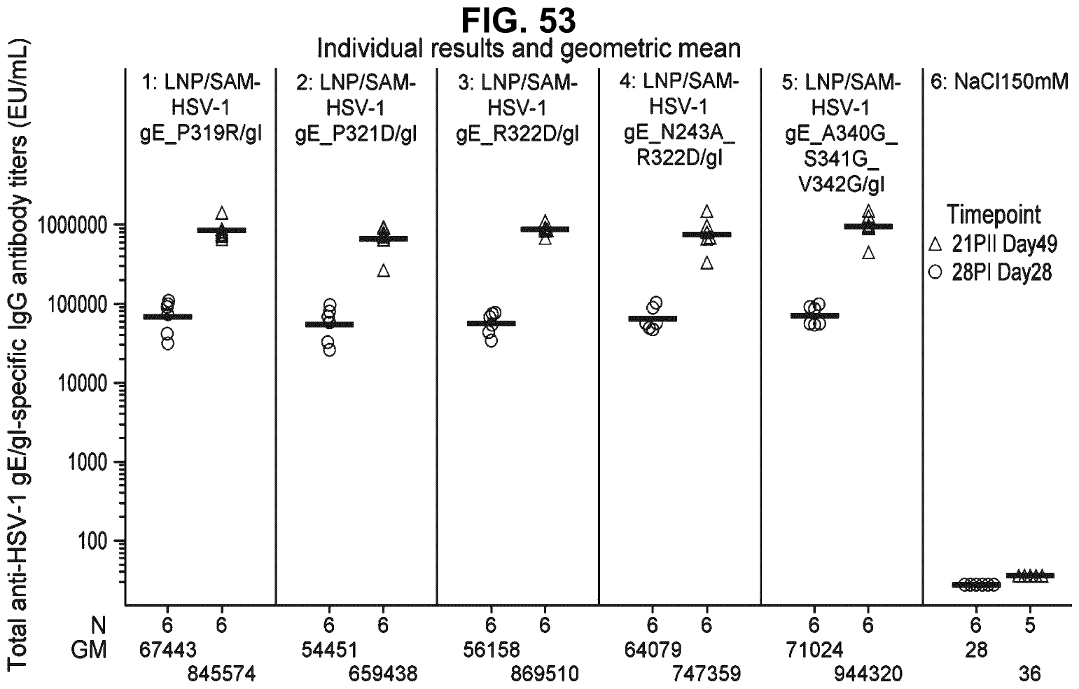

Overall, results suggest very similar vaccine-specific and cross-reactive CD4+T cell responses between the different mutated versions of HSV-1 gE/gI protein and with the unmutated HSV-1 gE/gI candidate. Only P321D mutant (group3) seemed to induce higher CD4+T cell response compared to the unmutated one (group1) with a fold increase close to 2 (GMR of 1.9 for anti-HSV-1 gE-specific CD4+T cells, GMR of 2 for HSV-2 gE cross-reactive CD4+T cells, and GMR of 1.89 for anti-HSV-1 gI-specific CD4+T cells, with CIs not containing 1) (FIG. 51A & FIG. 52). In addition, a trend for higher anti-HSV-1 gE-specific CD4+T cell response was also observed with R322D mutant (group4) compared to the unmutated protein (group1) (GMR of 1.96 and CI not containing 1) (FIG. 51B). Finally, slightly higher anti-HSV-1 gI-specific CD4+T cell response was observed with P321D mutant (group3) compared to R322D mutant (group4) (fold increase of almost 2 and CI not containing 1) (FIG. 52). No other differences between mutants were observed (with GMRs<2-fold change and/or CIs containing 1).

### Example 4—Immuno-Evaluation of Several LNP-Formulated SAM HSV-1 gE/gI Mutants in CB6F1 Mice

Objectives

The objective of this study was to generate immunogenicity data from different LNP-formulated SAM-HSV-1 gE/gI constructs containing mutation(s) in gE Fc binding domain to drop the avidity to human Immunoglobulin G Fc domain.

Twenty-one days post second immunization, the vaccine-specific polyclonal antibody (pAb) response and the anti-HSV-1 gE/gI-specific CD4+T cell responses induced by different mutants of LNP-formulated SAM HSV-1 gE/gI construct were compared to NaCl control group. In addition, head-to-head comparison of all vaccine candidates in term of anti-HSV-1 gE/gI-specific IgG antibody responses and anti-HSV-1 gE- and anti-HSV-1 gI-specific CD4+ T cell responses were performed 21 days post second immunization.

Finally, the exploratory objectives were to evaluate, in different groups of mice immunized with different mutated versions of LNP-formulated SAM HSV-1 gE/gI construct, the anti-HSV-1 gE/gI-specific antibody responses after one immunization, the anti-HSV-2 gE and anti-HSV-2gI cross-reactive antibody responses after two immunization, the functions of vaccine-specific and HSV-2 gE/gI cross-reactive polyclonal antibodies after two immunization and the levels of anti-HSV-1 gE-specific and anti-HSV-1 gI-specific CD8+T cell responses and anti-HSV-2 gE cross-reactive T cell response after the second immunization.

Overview of the Study Design

Female CB6F1 inbred mice aged 6-8 weeks old from Harlan laboratory (OlaHsd) were randomly assigned to the study groups (n=6 gr1-5; n=6 gr6) and kept at the institutional animal facility under specified pathogen-free conditions. CB6F1 mice (gr1-5) were intramuscularly (i.m) immunized at days 0 & 28 with 1 μg of different versions of SAM-HSV-1 gE/gI mutants formulated in Lipid nanoparticles (LNP). An additional group of mice was i.m injected with a saline solution (NaCl150 mM) following the same schedule of immunization and used as negative control group (gr6).

Serum samples were collected at days 28 & 49 post prime immunization (28PI, 21PII) to measure both anti-HSV-1 gE/gI-specific and cross reactive anti-HSV-2 gE and anti-HSV-2 gI IgG antibody responses. The functions of vaccine-specific and anti-HSV-1 gE/gI cross-reactive antibodies were also investigated in the serum samples collected 21 days after the second immunization. Spleens were collected 21 days post second immunization (21PII) to evaluate, ex-vivo, systemic CD4+ and CD8+ T cell responses towards HSV-1 gE, HSV-1 gI & HSV-2 gE antigens. Details for each group is described in the Table 6. A total of 36 animals was used for the whole study.

TABLE 6
Summary of the study design and formulation tested

| | | Treatment | | | | |
|---|---|---|---|---|---|---|
| | Number | | | Volume and | Immunization | Sample collection |
| | of | Vaccine name | Lot | route of | schedule | (organs, blood |
| Group | animals | and dose | numbers | injection | (Days) | volume) and days |
| 1 | 6 | LNP/SAM HSV-1 | 1700-29- | 50 μL | Days 0 & 28 | Day 28: Serum - |
| | | gE_P319R/gI | 1203 | i.m | | partial bleeding |
| | | (1 μg) | | | | Day 49: Serum - |
| 2 | 6 | LNP/SAM HSV-1 | 1700-29- | | | final bleeding + |
| | | gE_P321D/gI | 1204 | | | Spleen |
| | | (1 μg) | | | | |
| 3 | 6 | LNP/SAM HSV-1 | 1700-29- | | | |
| | | gE_R322D/gI | 1204 | | | |
| | | (1 μg) | | | | |
| 4 | 6 | LNP/SAM HSV-1 | 1700-29- | | | |
| | | gE_N243A_R322D/gI | 1205 | | | |
| | | (1 μg) | | | | |
| 5 | 6 | LNP/SAM HSV-1 | 1700-29- | | | |
| | | gE_A340G_S341G_V342G/gI | 1206 | | | |
| | | (1 μg) | | | | |
| 6 | 6 | NaCl150 mM | N/A | | | |

Materials and Methods

Investigational Products

Different mutated versions of SAM HSV-1 gE/gI vector were generated by In vitro Transcription (IVT) according to established protocols. Preparation of LNP with SAM followed established methods as follows. In this method, LNP-SAM is prepared by rapidly mixing ethanolic solutions of lipids with aqueous buffers that contain SAM. The rapid mixing results in a supersaturation of hydrophobic components that have ionically paired with SAM.

The SAM/lipid complexes condensate and precipitate through nucleation mediated precipitation, yielding small and narrowly disperse nanoparticles. Following this mixing step, the lipid nanoparticles mature to entrap the RNA and then are transferred to a final Tris/sucrose storage buffer through a buffer exchange step. The LNP solutions are then characterized for size, lipid content, RNA entrapment, final SAM concentration, and in vitro potency. The materials can be frozen at −80C for over 6 months after the addition of 5% (wt/v) sucrose.

Immunological Read-Outs

Total Anti-HSV-1 gE/gI-Specific IgG Antibodies Measured by ELISA

This was performed as described for Example 3.

Total Anti-HSV-2 gE & Anti-HSV-2 gI Cross-Reactive IgG Antibodies Measured by ELISA

This was performed as described for Example 3.

Anti-HSV-1 gE and Anti-HSV-1 gI-Specific & Anti-HSV-2 gE Cross-Reactive CD4+/CD8+ T Cell Responses Measured by ICS Assay

This was performed as described for Example 3.

Competitive ELISA to Evaluate the Ability of Vaccine-Specific Antibodies to Decrease Human IgG Fc Binding by HSV-1 gE/gI Protein

This was performed as described for Example 3.

Competitive ELISA to Evaluate the Ability of HSV-1 Cross Reactive Antibodies to Decrease Human IgG Fc Binding by HSV-2 gE/gI Protein

This was performed as described for Example 3.

Evaluation of the Ability of HSV-2 Cross-Reactive Antibodies to Bind and Activate mFcγRIII after Incubation with HSV-2 gE/gI Transfected Cells (Mouse FcγRIII ADCC Bioassay—Promega)

This was performed as described for Example 3.

In-Vitro HSV-1 Neutralization Assay

This was performed as described for Example 3.

In-Vitro HSV-2 MS Neutralization Assay

This was performed as described for Example 3.

Statistical Methods

The distribution of each response was assumed to be lognormal.

For anti-HSV-1 gE/gI-specific polyclonal antibody (pAb) response, a two-way analysis of variance (ANOVA) model is fitted on log₁₀ titers by including groups (all except the NaCl one), time points (Day28 (28PI) and Day49 (21PII)) and their interaction as fixed effects. The NaCl group was not included as no response and variability was observed. Variance-covariance model selection was based on AICC criterion and individual data plot examination.

The variance-covariance for time points was modelled via a Compound Symmetry matrix:

$$\begin{matrix}[ \begin{matrix}{σ}^{2} + {σ}_{1} & {σ}_{1} \\ {σ}_{1} & {σ}^{2} + {σ}_{1}\end{matrix} ] \\ CS - Compound Symmetry\end{matrix}$$

The compound symmetry considers same variance and same correlation between timepoints. The same variance-covariance matrix was modelled for each vaccine group, indicating same variance between groups.

Geometric means and their 95% CIs are derived from this model.

Despite the fact that the NaCl group was not included in the ANOVA model, the comparisons associated to the primary objective were computed as follows: geometric means and 95% CI of vaccinated groups derived from the above model were divided by titer given to all the NaCl recipients at the last timepoint. The resulting ratios should be understood as geometric mean ratios with their corresponding 95% CI.

For head to head comparison of vaccinated groups (at the last time point) and time point comparison (PII/PI) within each group, all the geometric mean ratios and their 95% CIs were derived from the model.

For each HSV-2 cross-reactive IgG antibody response (gE or gI), a one-way analysis of variance (ANOVA) model was fitted on log₁₀ titers by including groups (all except the NaCl one) as fixed effect. The NaCl group was not included as no response and variability was observed. No clear heterogeneity of variance was detected and therefore identical variances were assumed for the different groups. Geometric means and their 95% CIs are derived from these models. For comparison of vaccinated to NaCl groups, geometric means and 95% CI of vaccinated groups derived from the above model were divided by titer given to all the NaCl recipients. The resulting ratios should be understood as geometric mean ratios with their corresponding 95% CI. For head to head comparison of vaccinated groups, all the geometric mean ratios and their 95% CIs were derived from the model.

On each vaccine-specific or cross-reactive % of CD4+/CD8+ T cell responses (gE-, or gI-specific), a one-way analysis of variance (ANOVA) model was fitted on log 10 frequencies by including groups (all groups including the NaCl group) as fixed effect. No clear heterogeneity of variance was detected for % of HSV-2 gE cross-reactive CD4+ T cell response and therefore identical variances were assumed for the different groups. For other responses, different variances were modeled for the different groups. For both % of CD4+ and CD8+ T cell responses, the NaCl threshold is based on P95 of data across stimulation in NaCl negative control group. No modelling was performed on % of HSV-1 gE-specific or HSV-2 gE cross-reactive CD8+ T cells since most of the response were below the P95 NaCl threshold for all vaccine groups. Geometric means and geometric mean ratios (with their corresponding 95% CIs) were derived from these models.

For the evaluation of the dissociation of the human IgG Fc binding by the pAbs (HSV-1 or HSV-2), ED50 response was calculated for each sample. On each response (HSV-1 or HSV-2), a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

For HSV-1-specific neutralizing antibody titers, a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from this model.

Results

Different LNP/SAM HSV-1 gE/gI Mutants Induced Vaccine-Specific and Anti-HSV-2 gE and Anti-HSV-2 gI Cross-Reactive IgG Antibody Responses

The level of anti-HSV-1 gE/gI-specific IgG antibody response was investigated by ELISA. Note that one sample in NaCl group (sample 6.2) was not evaluated 21 days after the second immunization due to lack of serum availability.

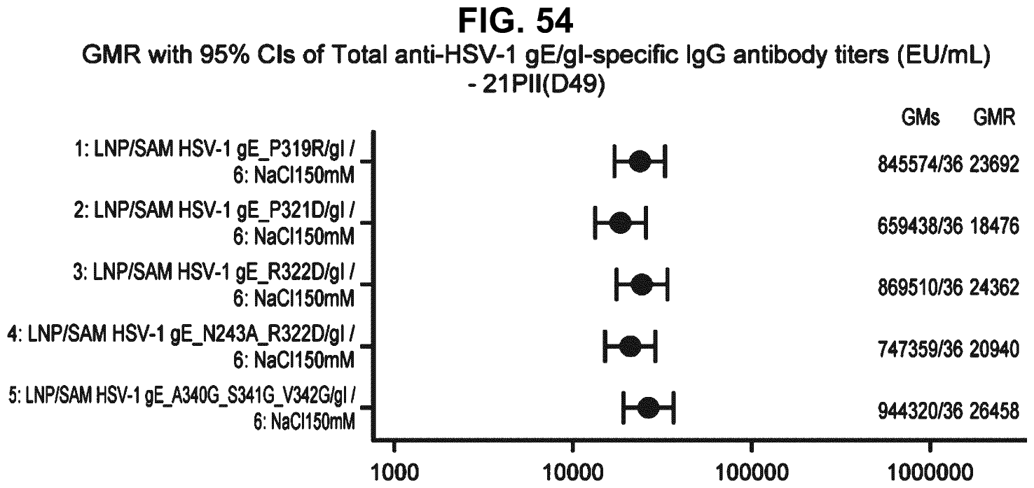

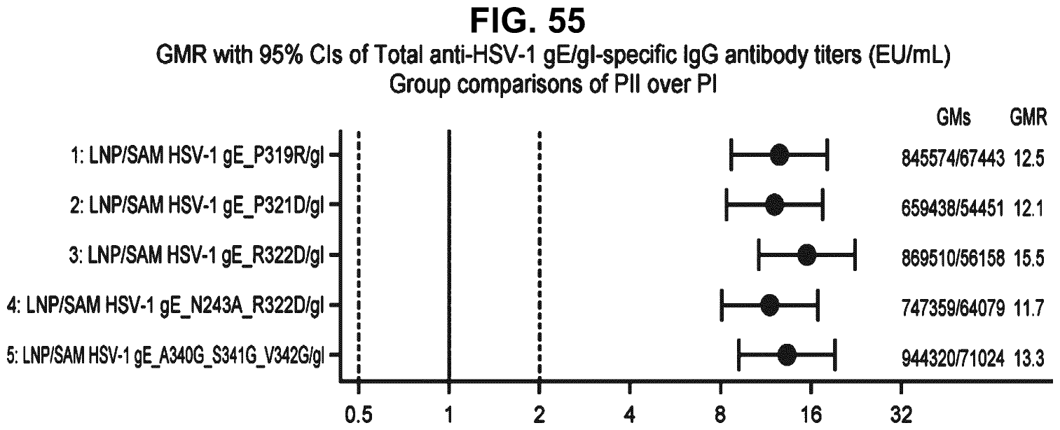

Twenty-one day post the second immunization, all LNP/SAM-HSV-1 gE/gI vaccinated groups developed strong anti-HSV-1 gE/gI-specific antibody response compared to the NaCl control group (response above 600.000 EU/mL; all GMRs>18.000) (FIG. 53 & FIG. 54). As expected, no anti-HSV-1 gE/gI-specific response was observed in the NaCl control group (<40 EU/ml for all mice). In all groups of mice immunized with different mutated versions of LNP-formulated SAM-HSV-1 gE/gI vector, anti-HSV-1 gE/gI-specific IgG antibody responses increased after the second immunization (day49 (21PII)) compared to the first immunisation (day28 (28PI)), with a fold increase ranging from 12 to 16 (FIG. 55).

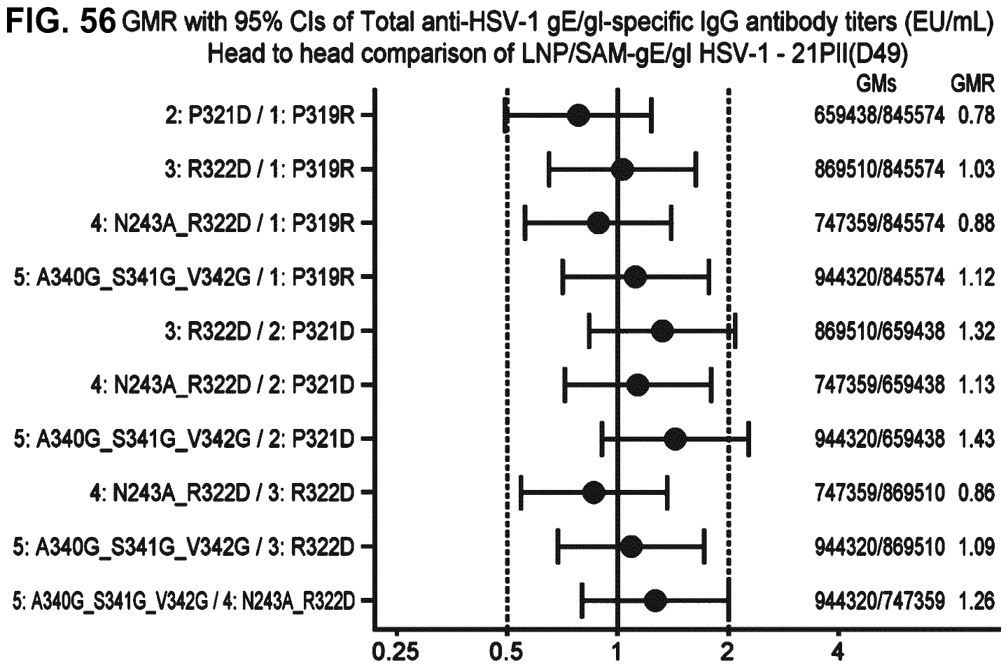

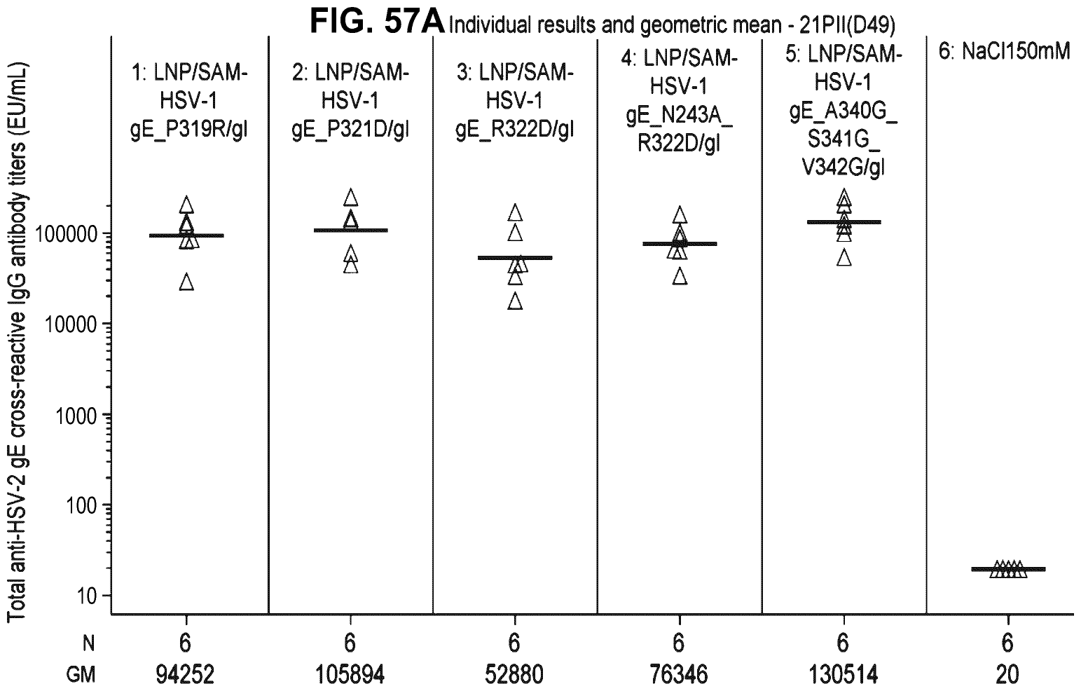

Finally, head to head comparison of all vaccine-immunized groups of mice suggests the induction of a similar anti-HSV-1 gE/gI-specific IgG antibody response between the different mutated versions of LNP-formulated SAM-HSV-1 gE/gI vector 21 days post second immunization (with GMRs close to 1-fold change and most CIs are totally included in [0.5,2] and contain 1) (FIG. 56).

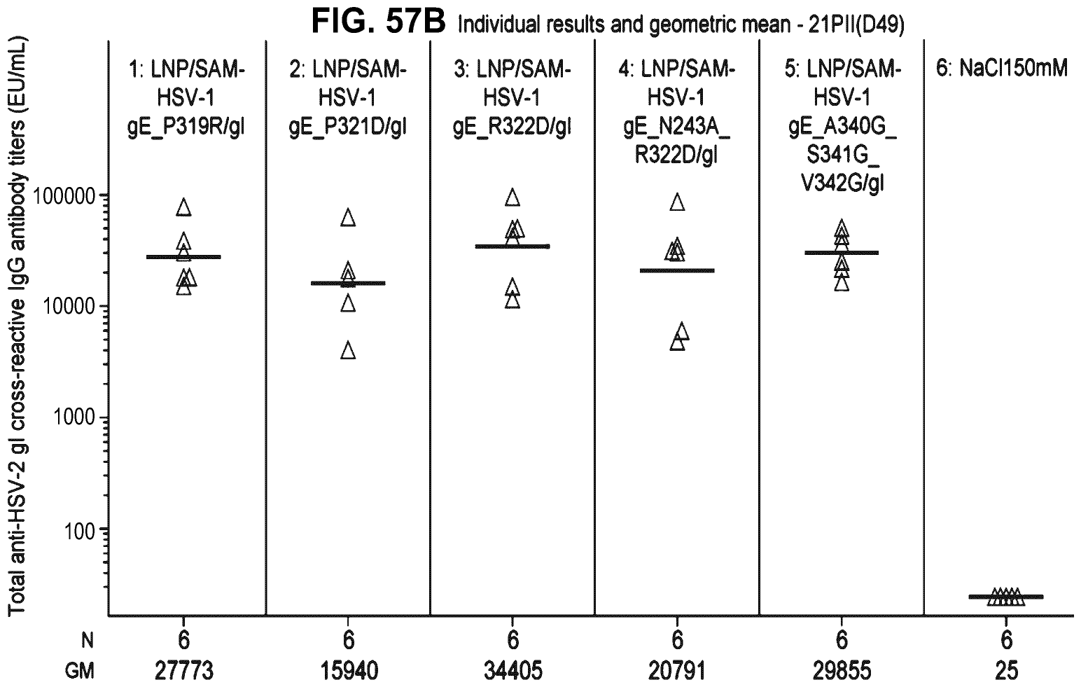

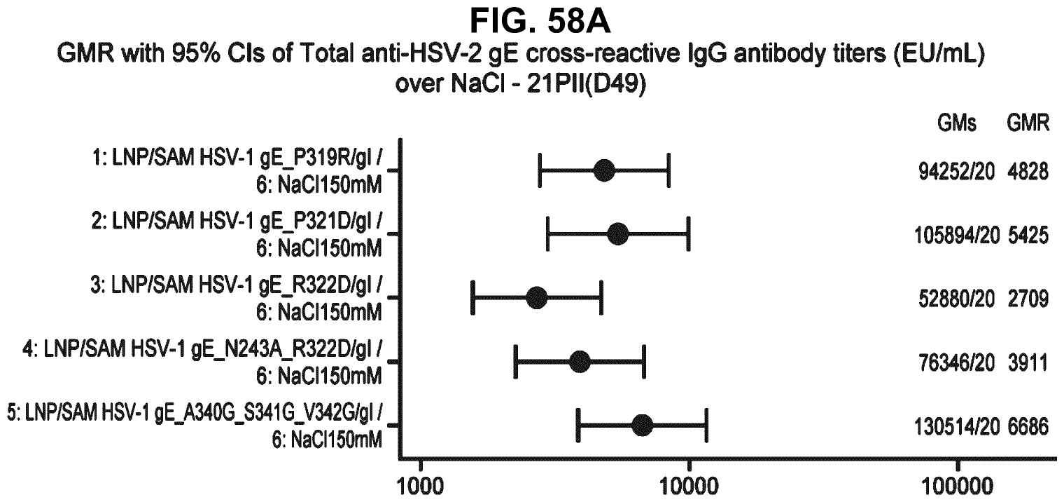

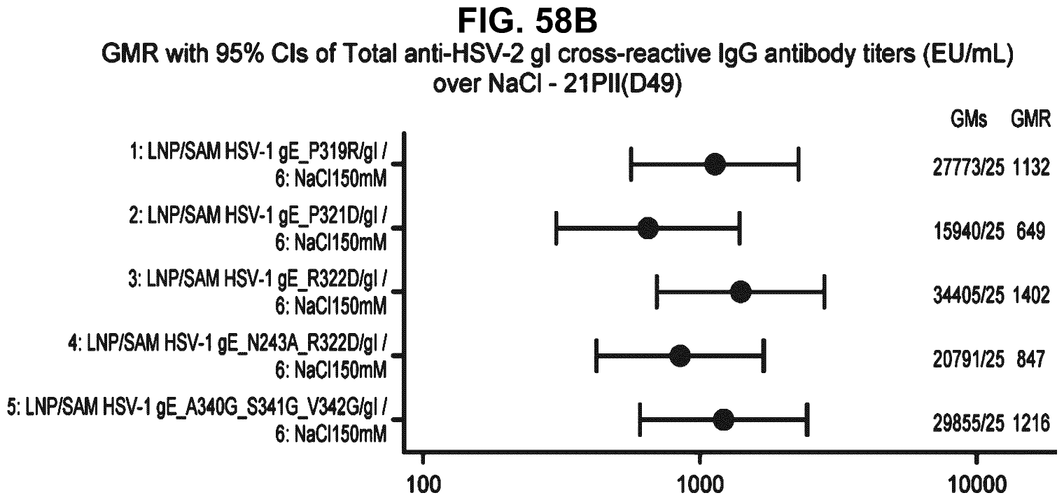

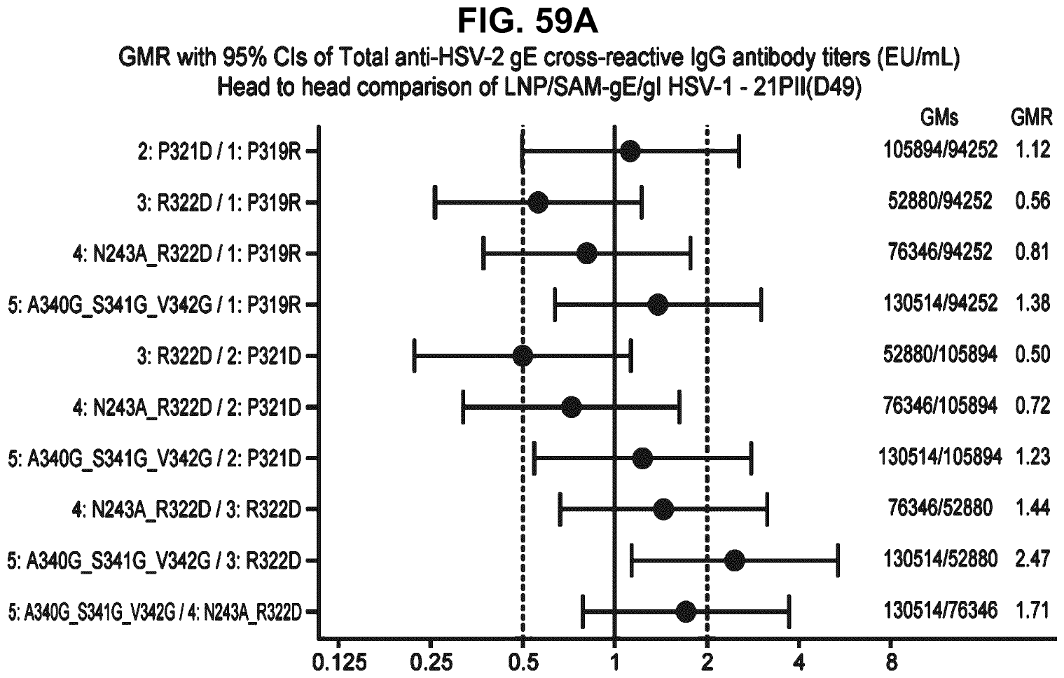

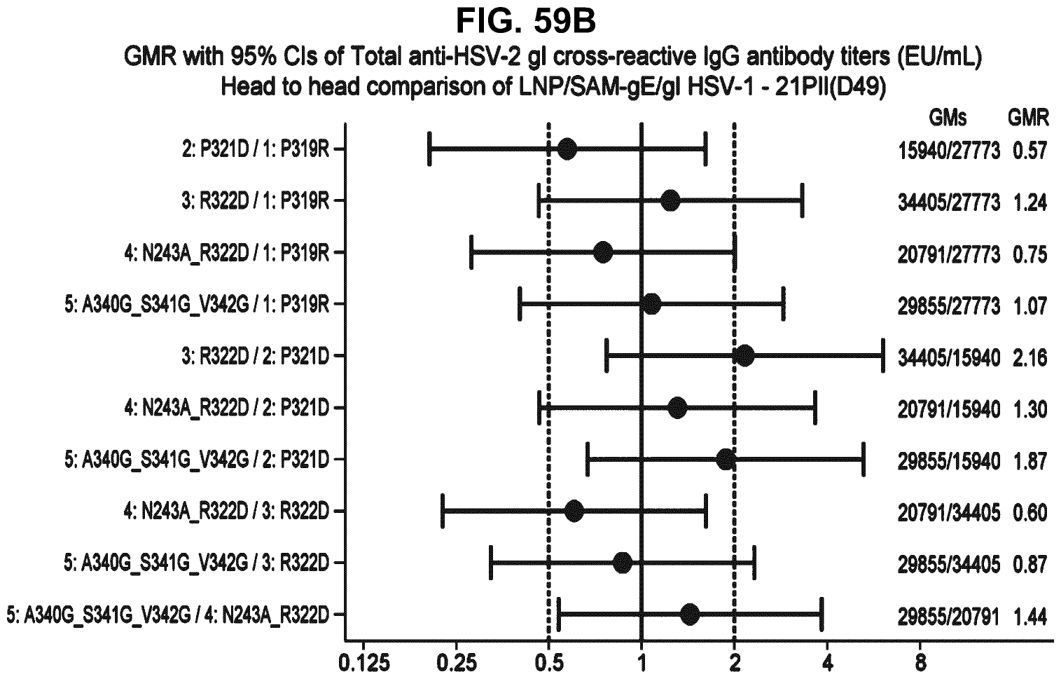

As expected, anti-HSV-2 gE and anti-HSV-2 gI cross-reactive responses were not observed in NaCl control group (<30 EU/mL for all mice). At 21 days post second immunization and compared to NaCl control group, high anti-HSV-2 gE and anti-HSV-2 gI cross-reactive IgG antibody responses were induced in all groups of mice immunized with different mutated versions of LNP-formulated SAM-HSV-1 gE/gI vector (all GMRs>2.000 for gE and >600 for gI) (FIG. 57, FIG. 58). In overall, head to head comparison of vaccine-immunized groups of mice suggests similar anti-HSV-2 gE or anti-HSV-2 gI cross-reactive IgG antibody responses 21 days post second immunization (with most GMRs<2-fold change and/or CIs containing 1). Only A340G_S341G_V342G mutant (group 5) seemed to induce higher gE response compared to R322D mutant (group 3) with a fold increase of 2.47 (CI not containing 1) (FIG. 59).

Different Mutated Versions of LNP/SAM HSV-1 gE/gI Vector can Induce Functional HSV-1 gE/gI Specific and HSV-2 gE/gI Cross-Reactive Antibody Response

In this study, HSV-1-specific and HSV-2 cross-reactive antibody functions were only investigated in the sera collected at 21 days post second immunization. In this regard, the ability of polyclonal Abs to neutralize HSV-1 or HSV-2 viruses, to decrease, in-vitro, binding of human IgG Fc by HSV-1 or HSV-2 gE/gI protein and to bind and activate mFcγRIII expressed on modified Jurkat cells after incubation with HSV-2 gE/gI positive 3T3 cells was investigated for each group of mice.

Note that one sample in NaCl group (sample 6.2) was not evaluated in HSV-1/HSV-2 neutralization assays and in hIgG competitive ELISA on HSV-1/HSV-2 gE/gI protein due to lack of serum availability. In addition, one additional sample in LNP/SAM-HSV-1 gE_P321D/gI group (sample 2.3) was also not evaluated in HSV-2 neutralization assay and hIgG competitive ELISA on HSV-1/HSV-2 gE/gI protein for the same reason.

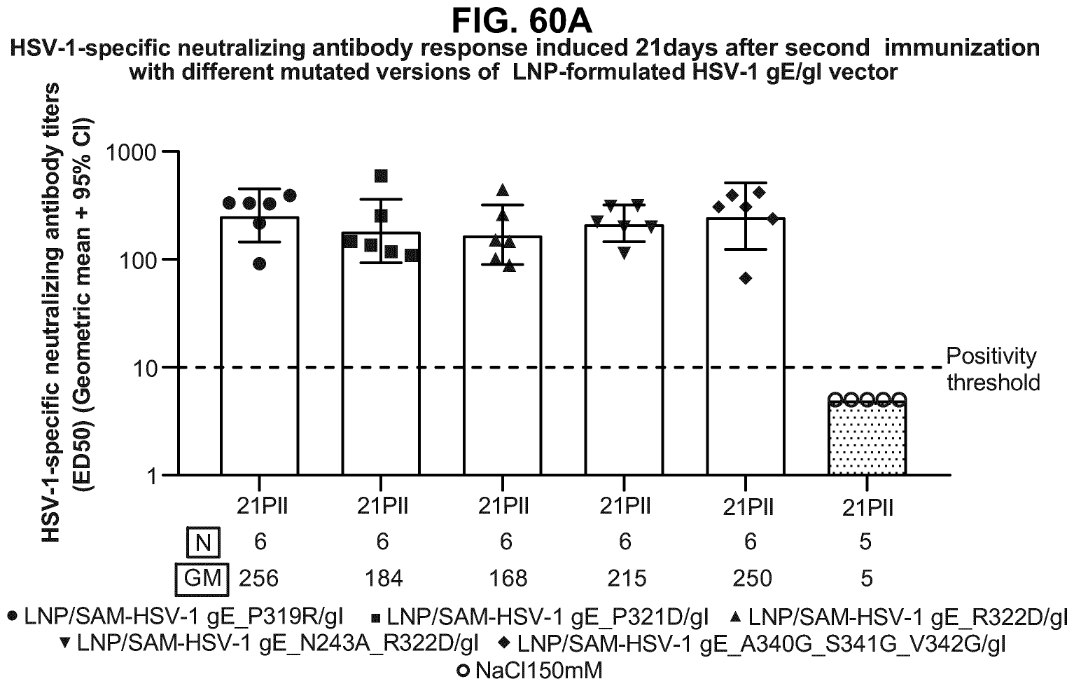

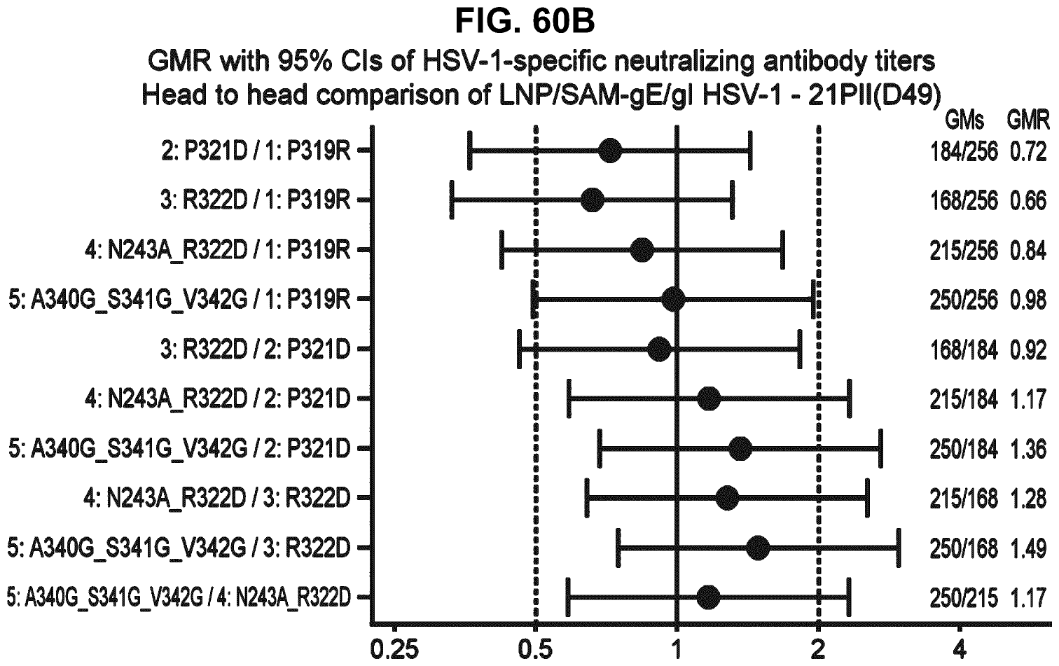

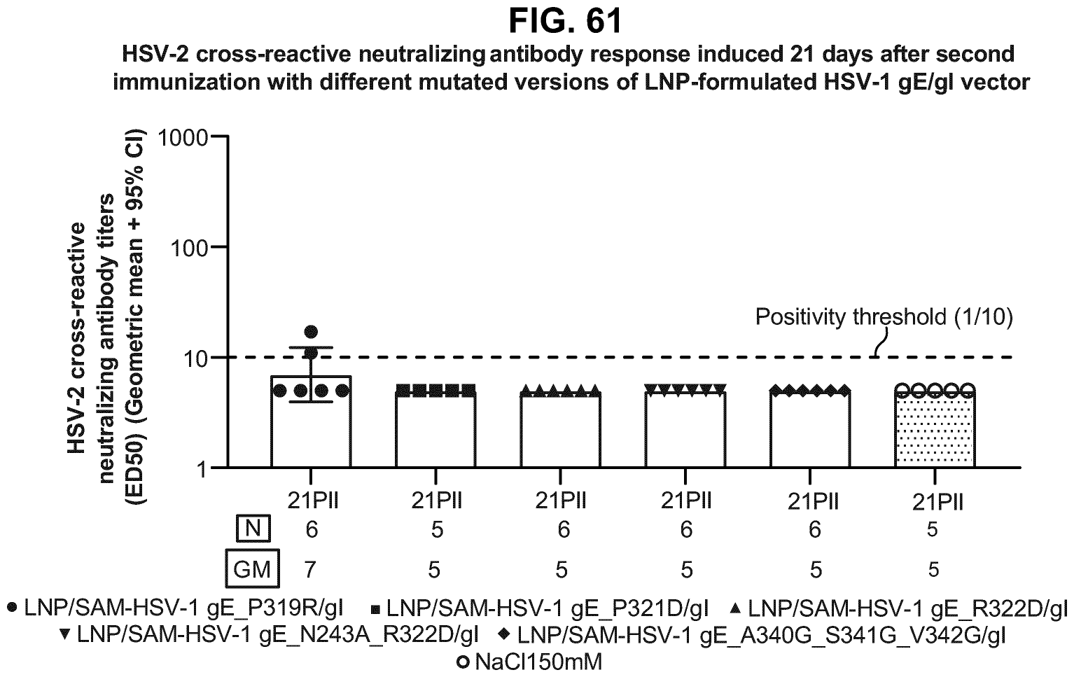

Low but consistent vaccine-specific neutralizing antibody (nAb) response directed to HSV-1 VR-1789 strain was detected in all groups of mice immunized with different mutated versions of LNP-formulated SAM-HSV-1 gE/gI vector (FIG. 60A). No difference in the intensity of the response induced by the different candidates was detected after group comparisons (GMRs close to 1 with all CIs containing 1) (FIG. 60B). However, no cross-reactive nAb response directed to HSV-2 MS strain was detected in any vaccinated groups of mice (FIG. 61).

Then, the ability of sera from mice immunized with different LNP-formulated SAM-HSV-1 gE/gI mutants to compete and decrease, in-vitro, hIgG Fc binding by HSV-1 or HSV-2 gE/gI protein was assessed by competitive ELISA. All LNP/SAM HSV-1 gE/gI mutants elicited vaccine-specific polyclonal antibody response able to decrease hIgG Fc binding by HSV-1 or HSV-2 gE/gI protein.

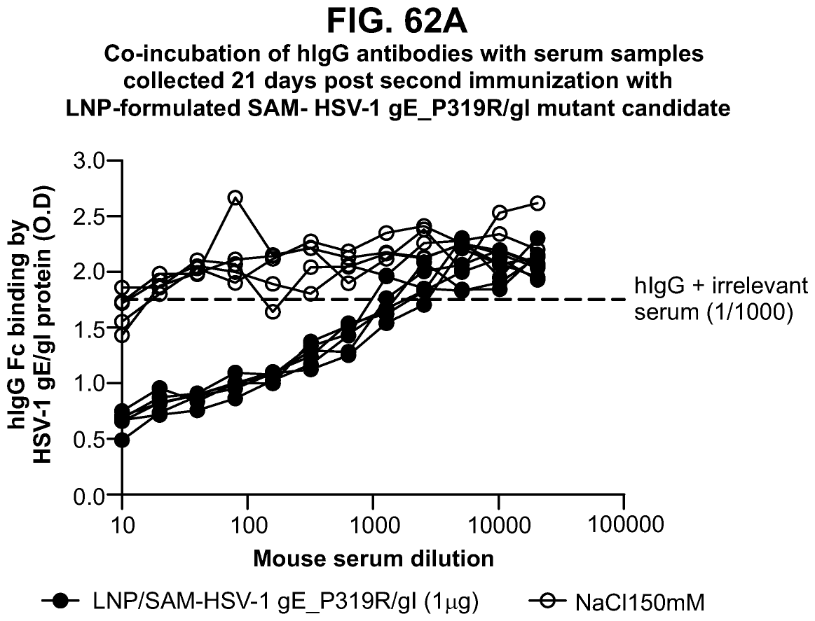

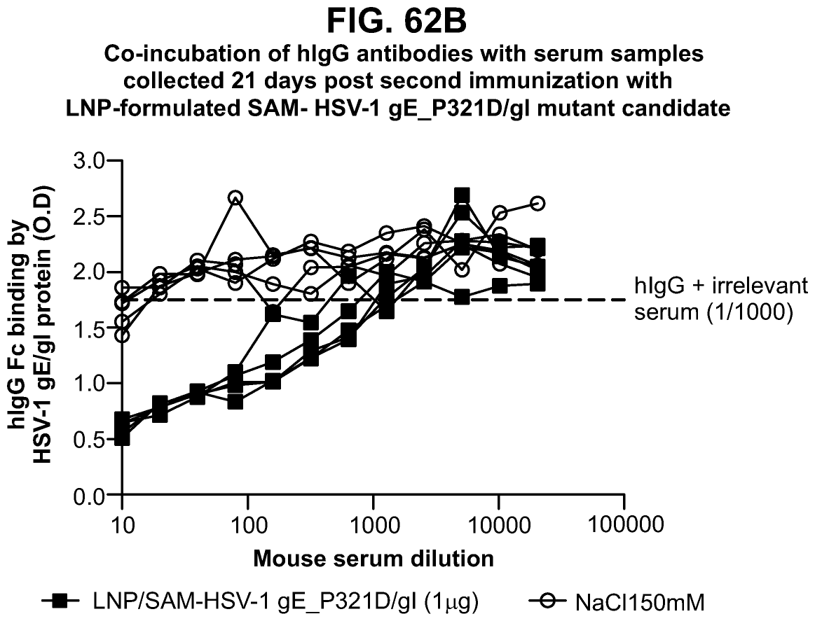

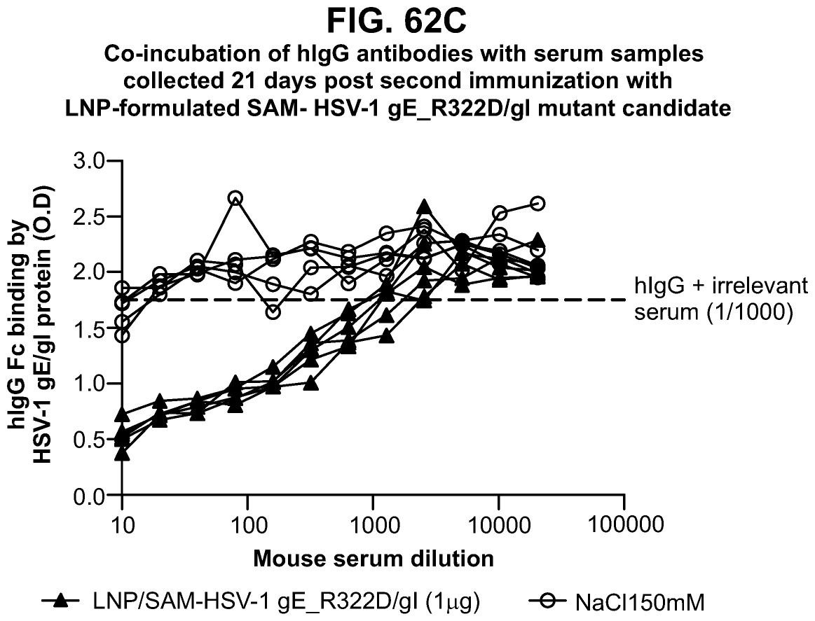

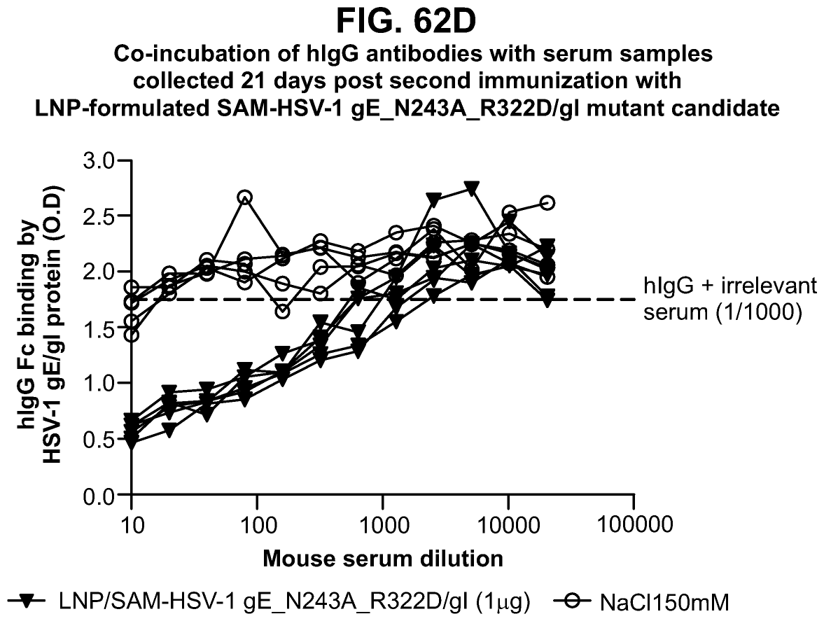

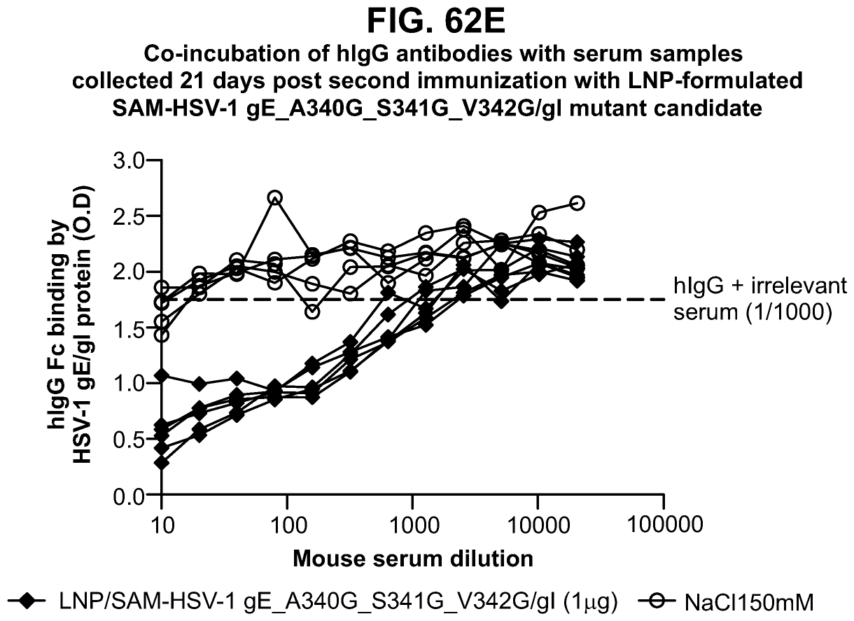

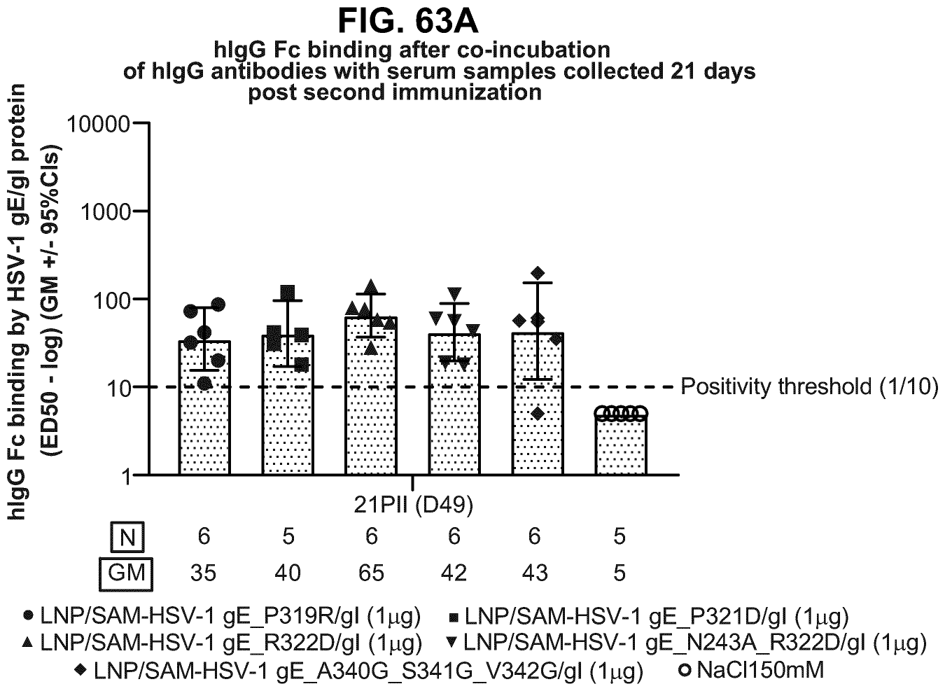

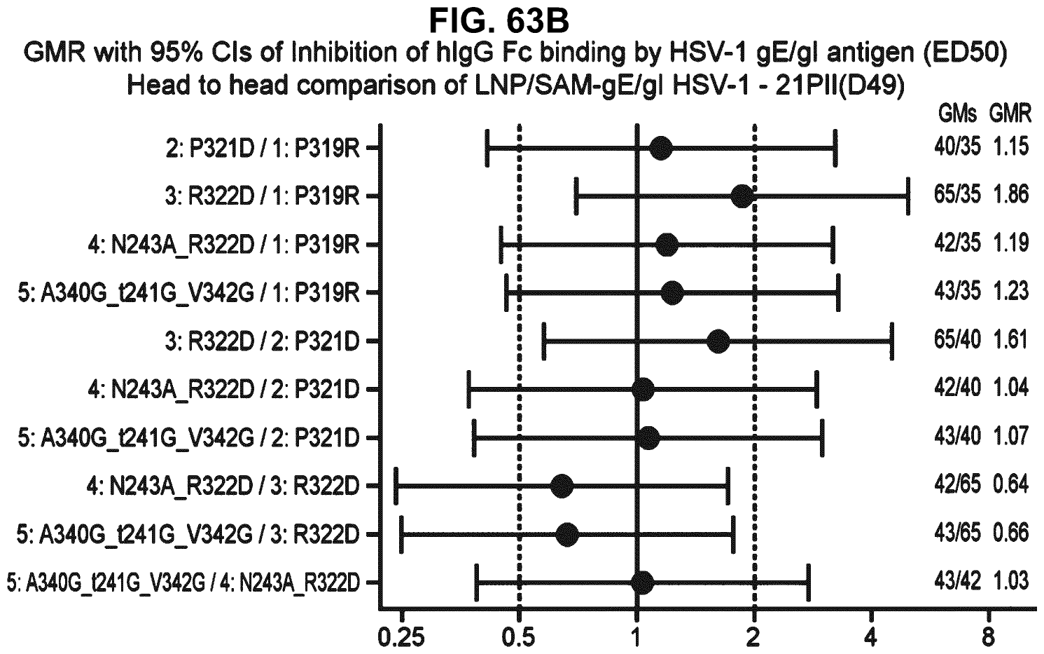

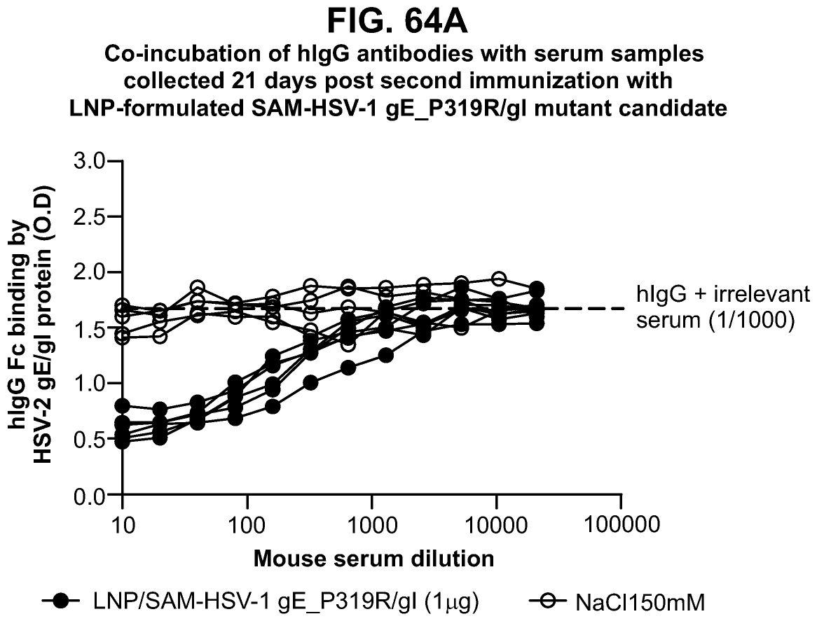

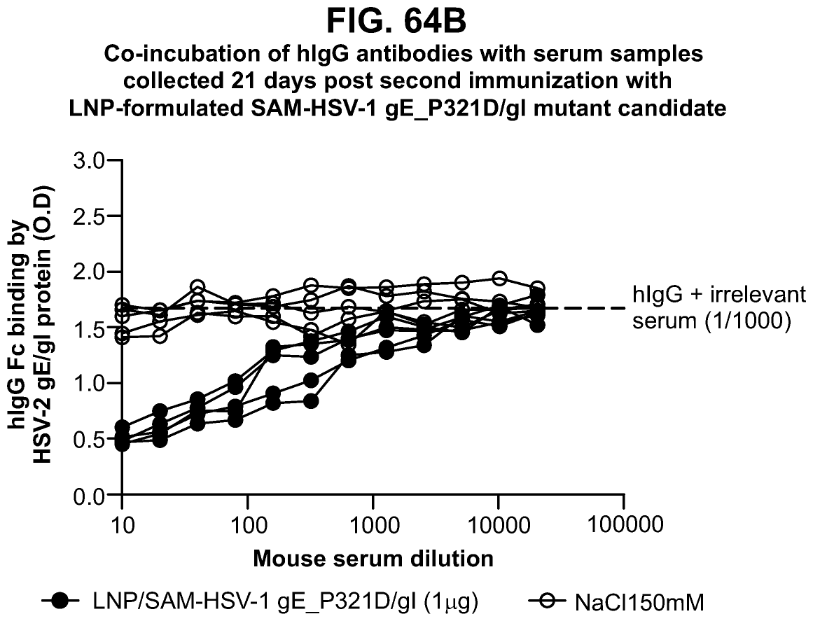

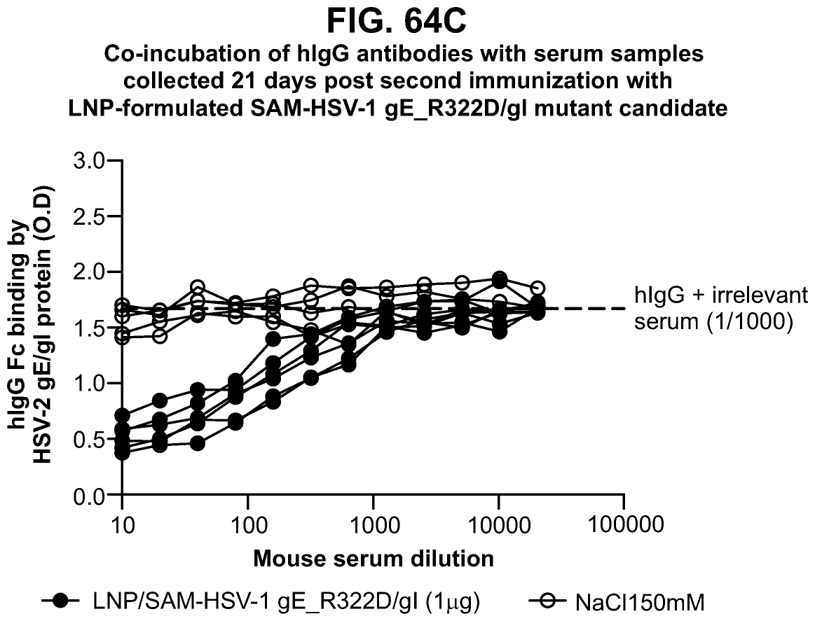

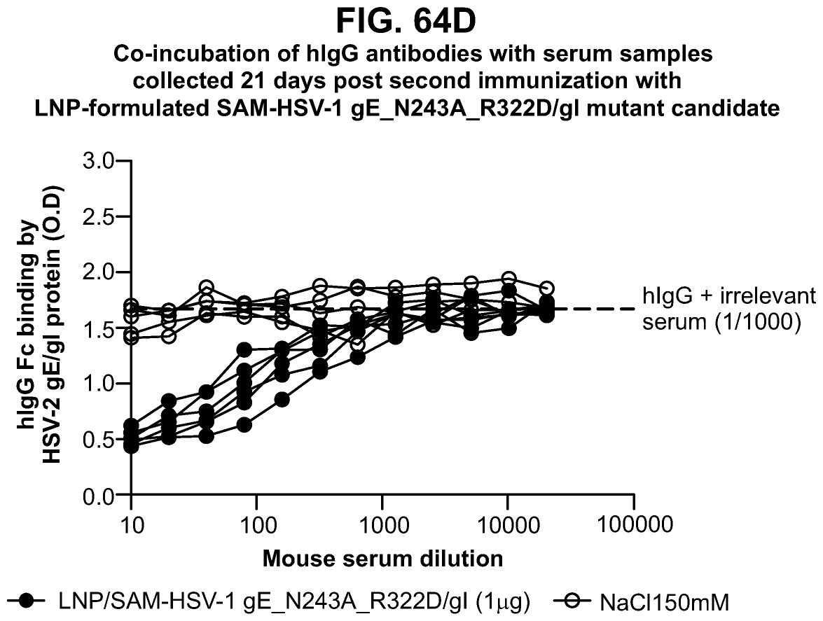

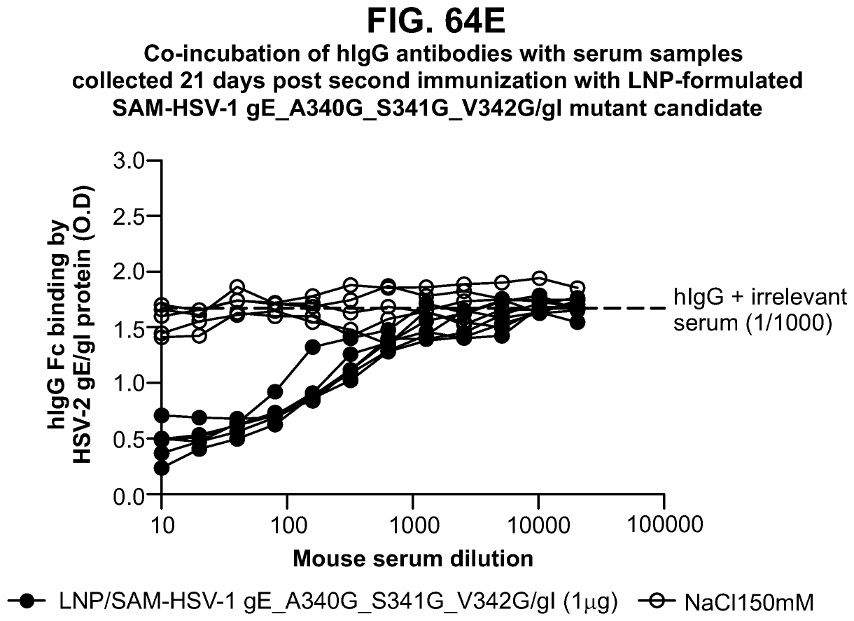

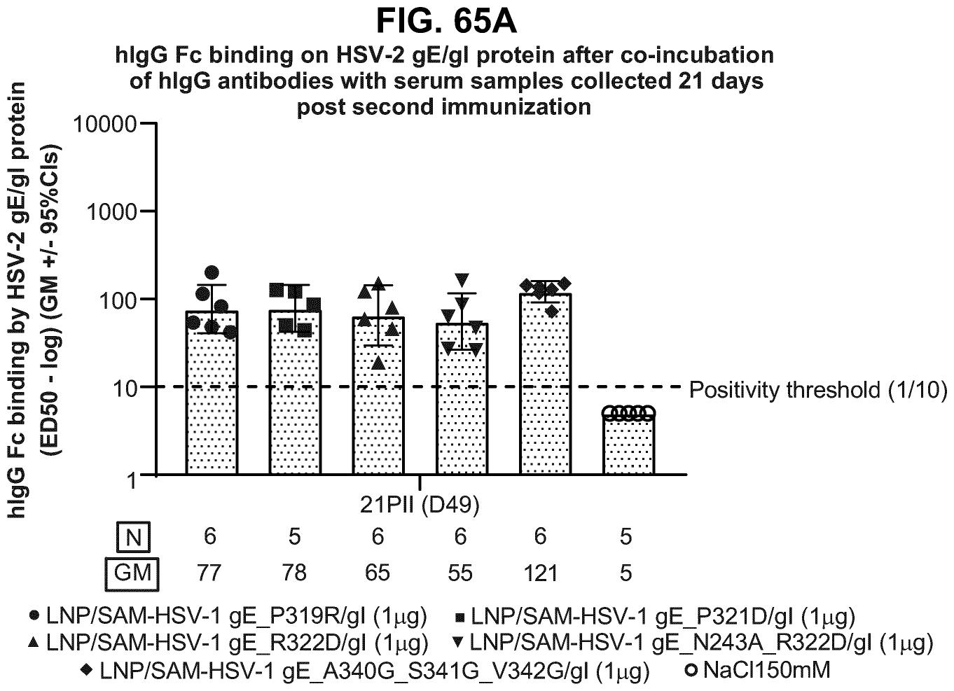

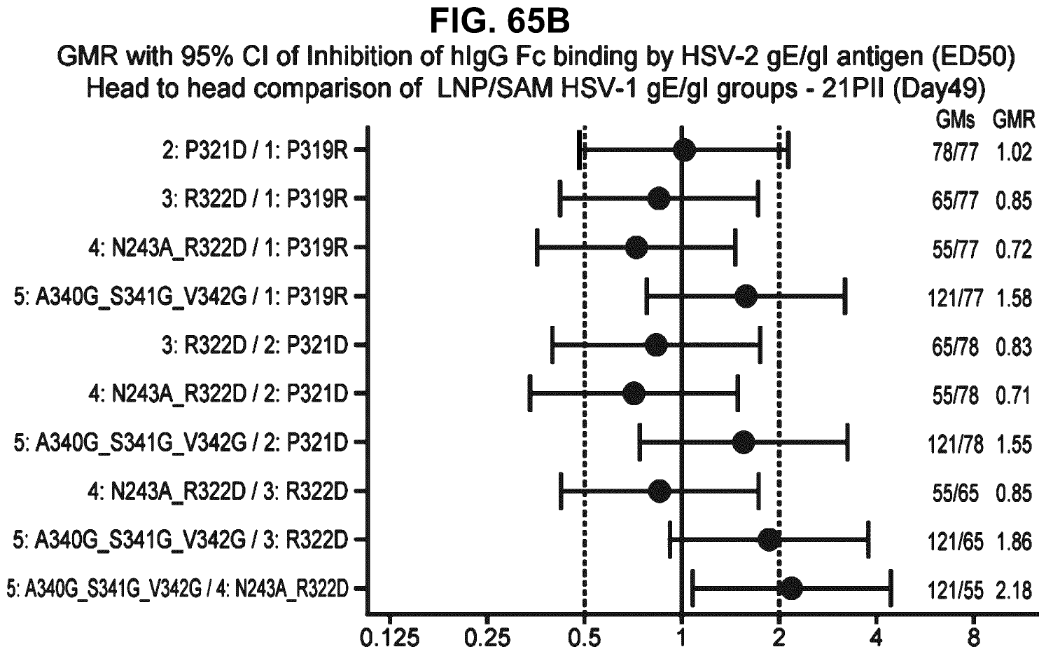

No clear difference in the dissociation curve of hIgG Fc binding by HSV-1 gE/gI protein was observed with the polyclonal sera collected in mice immunized with the different LNP/SAM HSV-1 gE/gI mutants (GMRs close to 1 with all CIs containing 1), which suggest that all candidates induce vaccine specific functional antibodies able to inhibit hIgG Fc binding by HSV-1 gE/gI protein (FIG. 62 & FIG. 63). In addition, no significant difference in the dissociation curve of hIgG Fc binding by HSV-2 gE/gI protein was observed with polyclonal sera collected in mice immunized with the different LNP/SAM HSV-1 gE/gI mutants (with most GMRs<2-fold change and/or CIs containing 1). Only A340G_5341 G_V342G mutant (group 5) seemed to induce higher cross reactive HSV-2 hIgG FC binding response compared to N243A_R322D mutant (group 4) with a fold increase of 2.18 (CI not containing 1). A trend for higher response with A340G_S341G_V342G mutant (group 5) than with R322D mutant (group 3) seems also observed (GMR of 1.86 and CI almost not containing 1) (FIG. 64, FIG. 65).

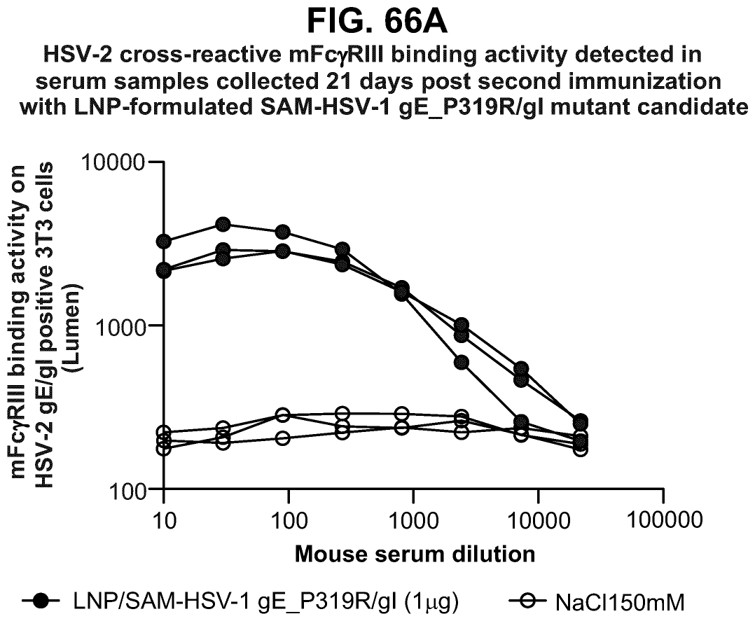

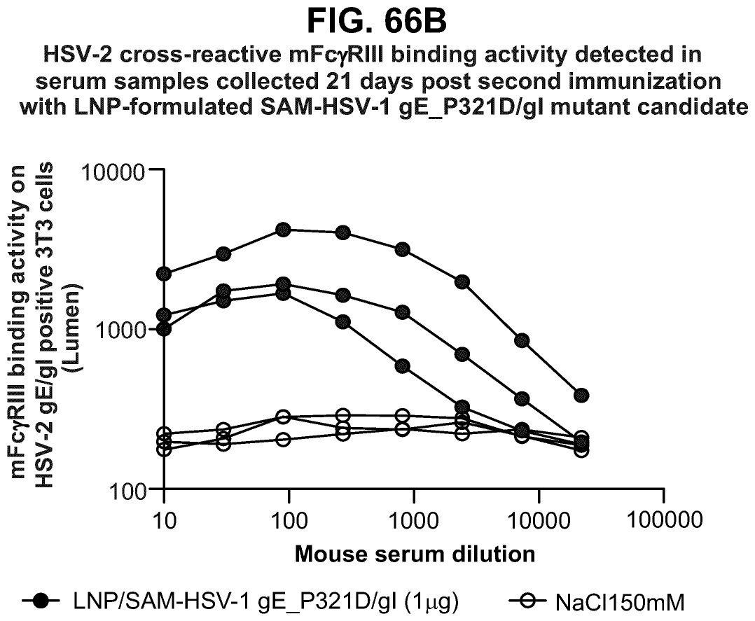

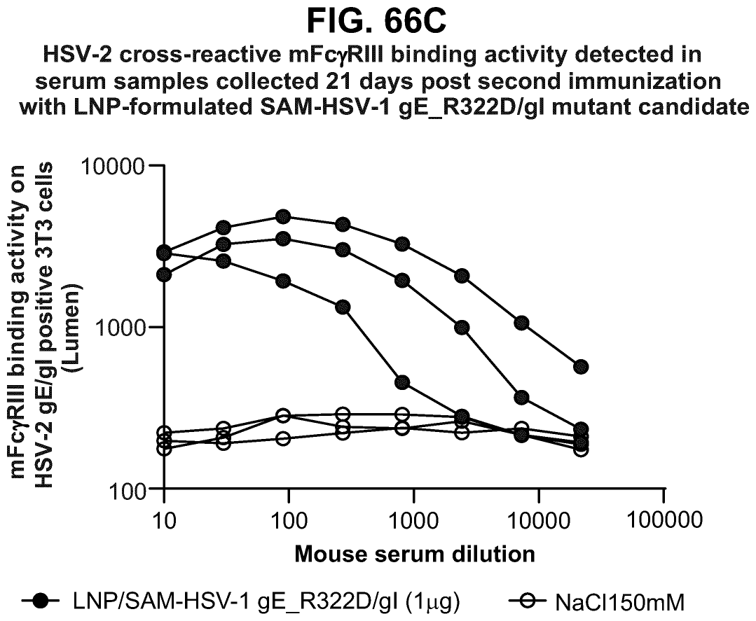

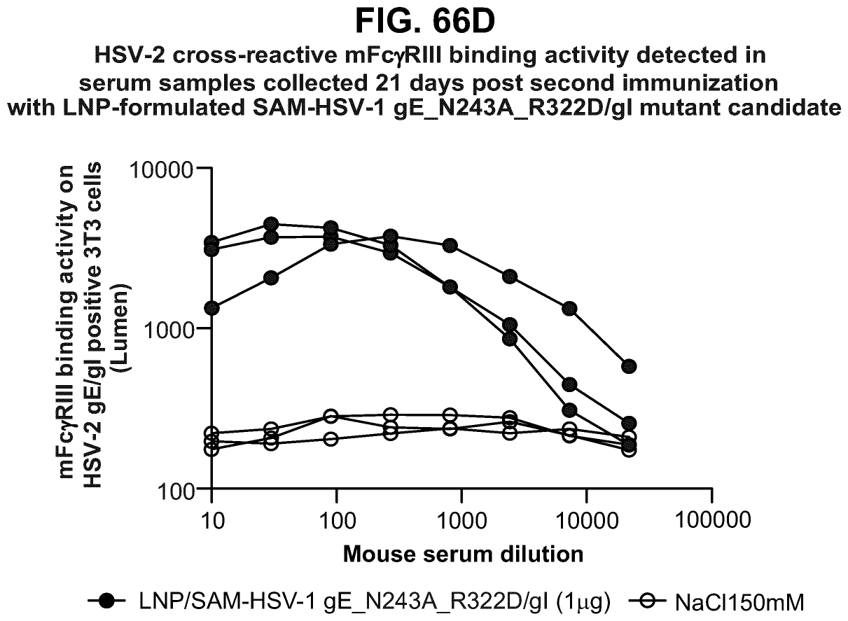

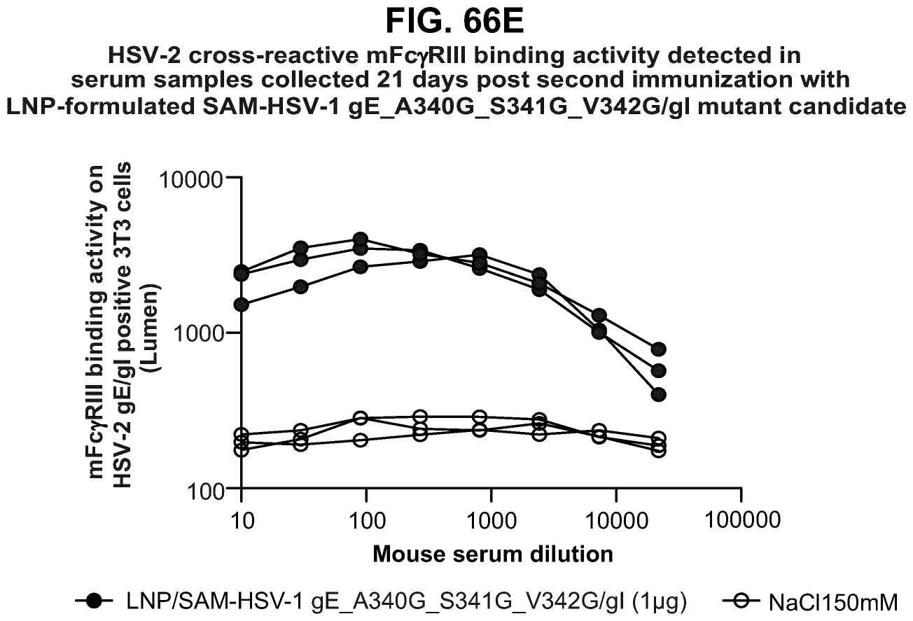

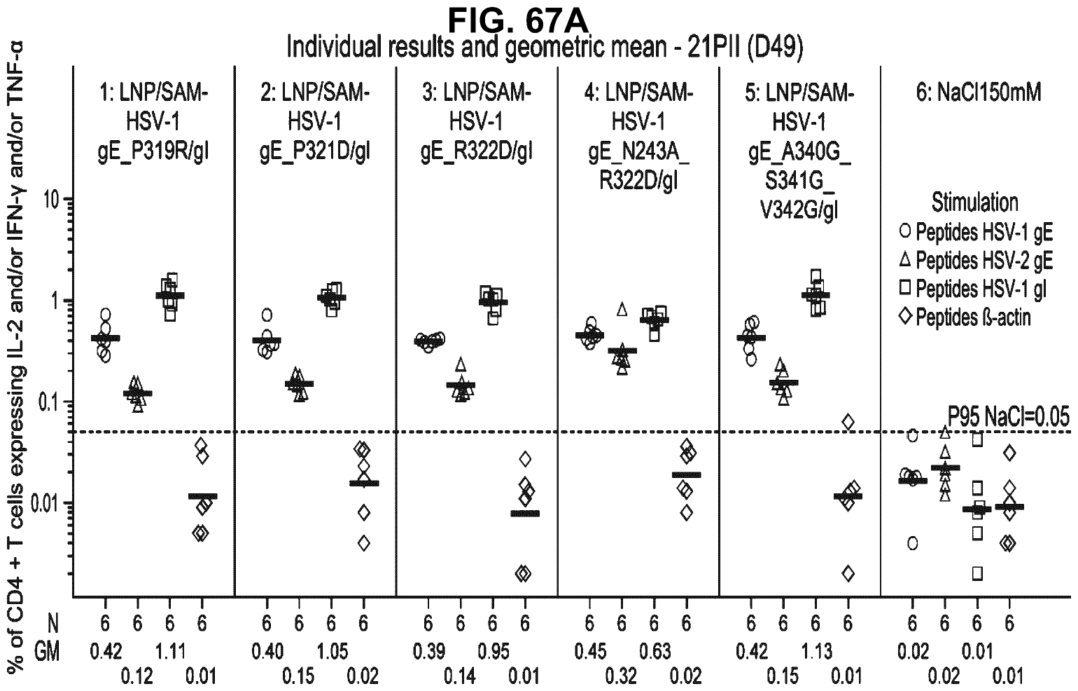

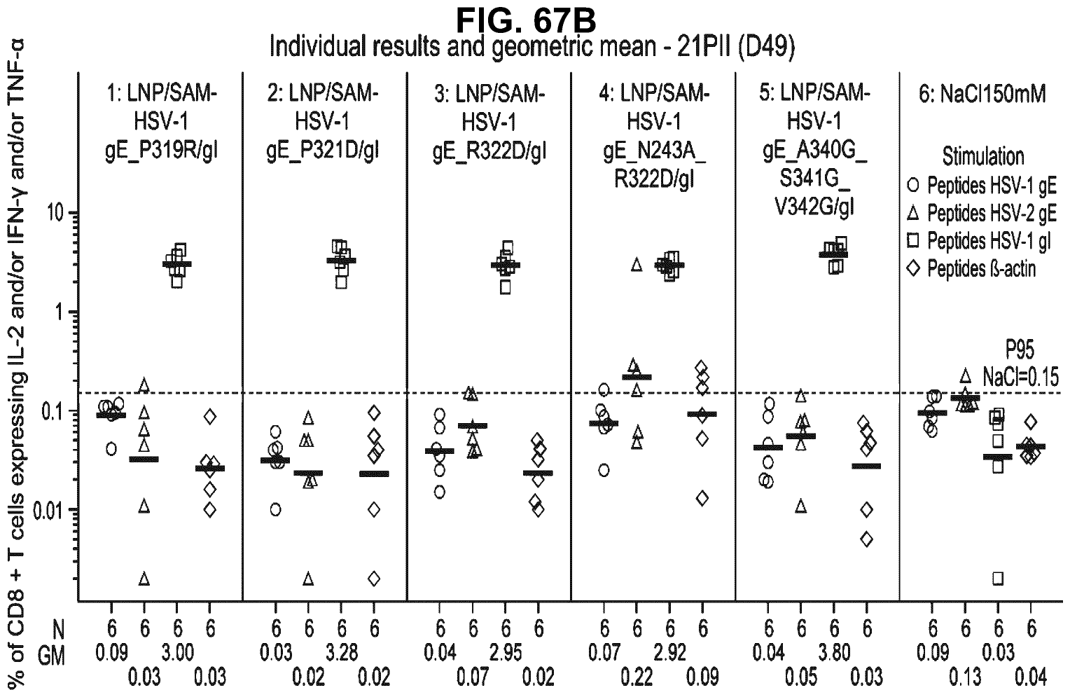

Finally, the ability of vaccine-specific pAbs to bind and activate, in-vitro, mouse FcγRIII after incubation with HSV-2 gE/gI transfected 3T3 cells, was investigated 21 days after the second immunization. Data shown in FIG. 66 suggested that all mice immunized with the different LNP-SAM-HSV-1gE/gI mutant candidates could induce HSV-2 cross reactive antibodies able to specifically bind and activate Jurkat reporter cells expressing FcγRIII after incubation with HSV-2 gE/gI transfected cells. As expected, activation of FcγRIII was not detected with sera from unvaccinated NaCl control mice (FIG. 66).

Altogether, these results suggest that all mutated versions of LNP-SAM HSV-1 gE/gI can induce functional vaccine-specific and cross-reactive polyclonal antibody response 21 days after the second immunization in mice.

Different Mutated Versions of LNP/SAM HSV-1 gE/gI Vector Induced Vaccine Specific and Cross-Reactive T Cell Responses

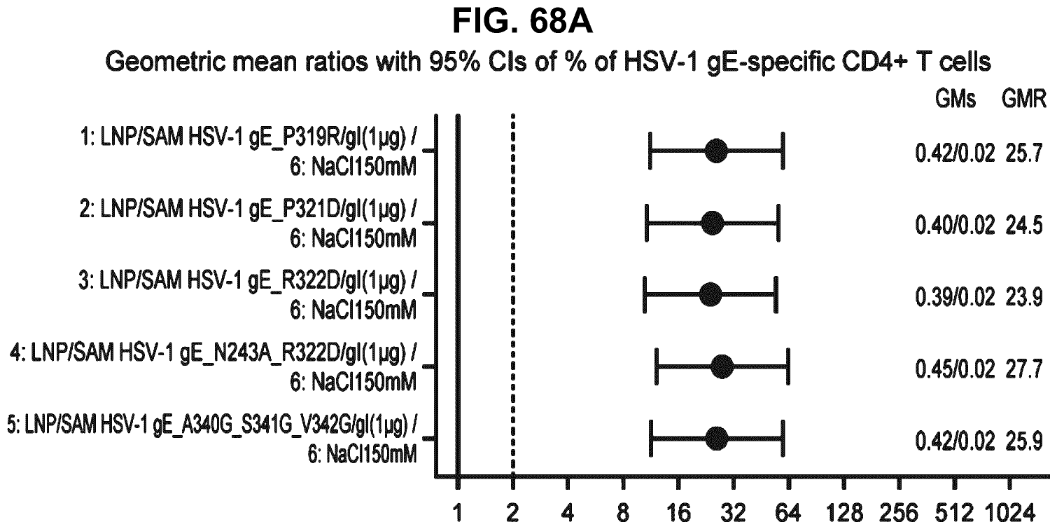

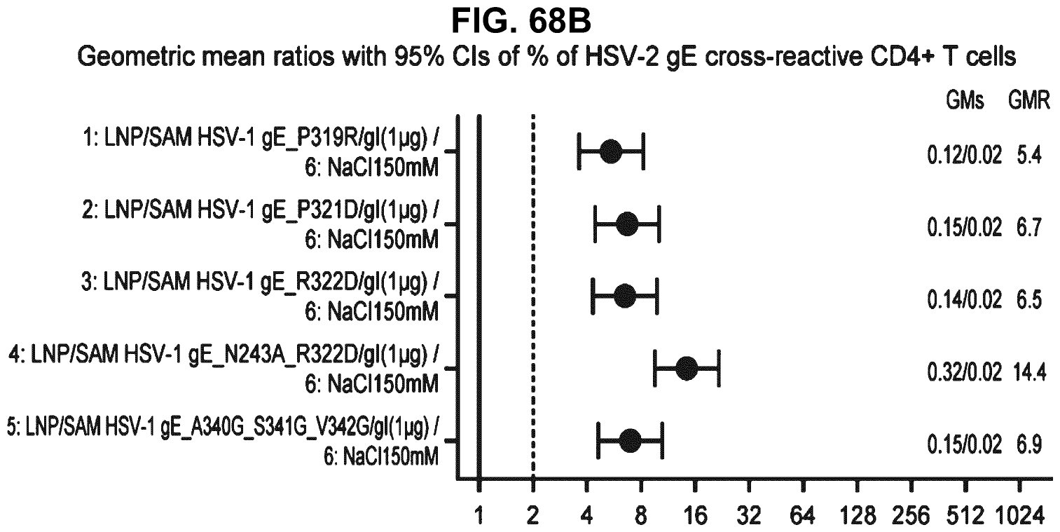

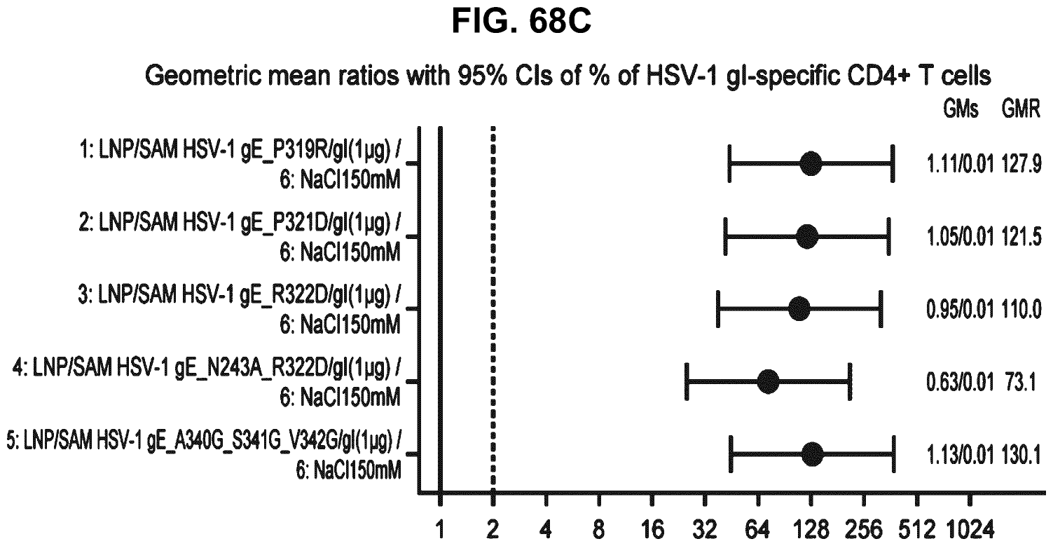

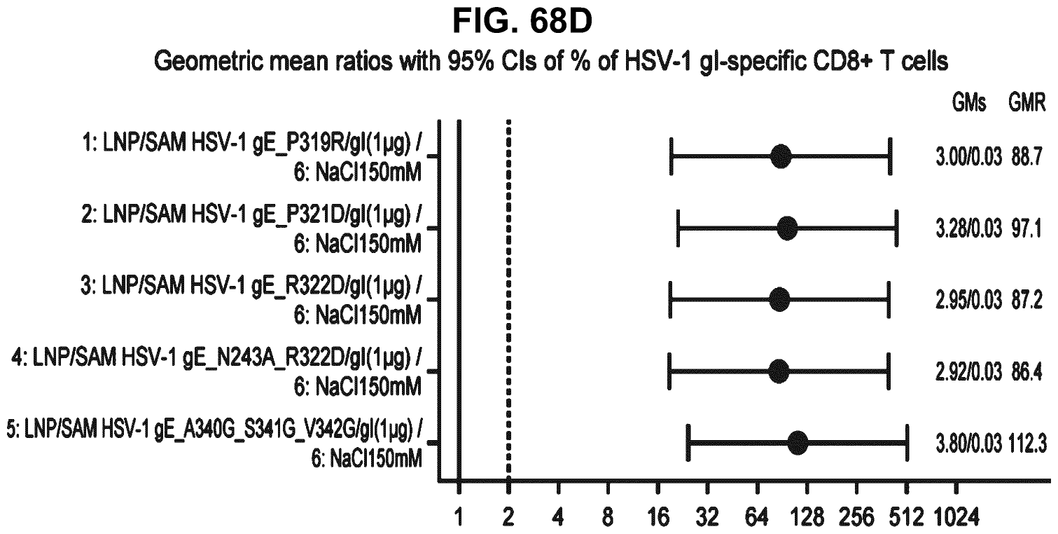

Compared to the NaCl control group, higher anti-HSV-1 gE & anti-HSV-1 gI specific and HSV-2 gE cross-reactive CD4+T cell responses were detected 21 days after the second immunization in all groups of mice immunized with different mutated versions of LNP-formulated SAM-HSV-1 gE/gI vector (FIG. 67A). Note that only moderate level of anti-HSV-1 gE-specific CD4+T cell response was observed (about 0.4%, with GMRs over NaCl around 25) (FIG. 67A; FIG. 68A). Cross-reactive HSV-2 gE CD4+T cell response was also detected in all vaccinated groups compared to the NaCl control group (GMRs ranging from 5 to 14 with CI not containing 1), but this response was low (about 0.15%) (FIG. 67A; FIG. 68B). Compared to the NaCl control group, high anti-HSV-1 gI-specific CD4+/CD8+T cell responses were detected in all groups of mice immunized with different mutated versions of LNP-formulated SAM HSV-1 gE/gI vector (about 1% for anti-HSV-1 gI-CD4+ T cells and 3% for anti-HSV-1 gI-CD8+ T cells, with GMRs over NaCl around 100) (FIG. 67A; FIG. 67B; FIG. 68C; FIG. 68D). However, compared to NaCl group, there is no clear evidence that either anti-HSV-1 gE-specific or anti-HSV-2 gE cross-reactive CD8+T cell responses were detected in any of vaccine groups (most of the vaccinated animals had a response below the P95 NaCl threshold) (FIG. 67B).

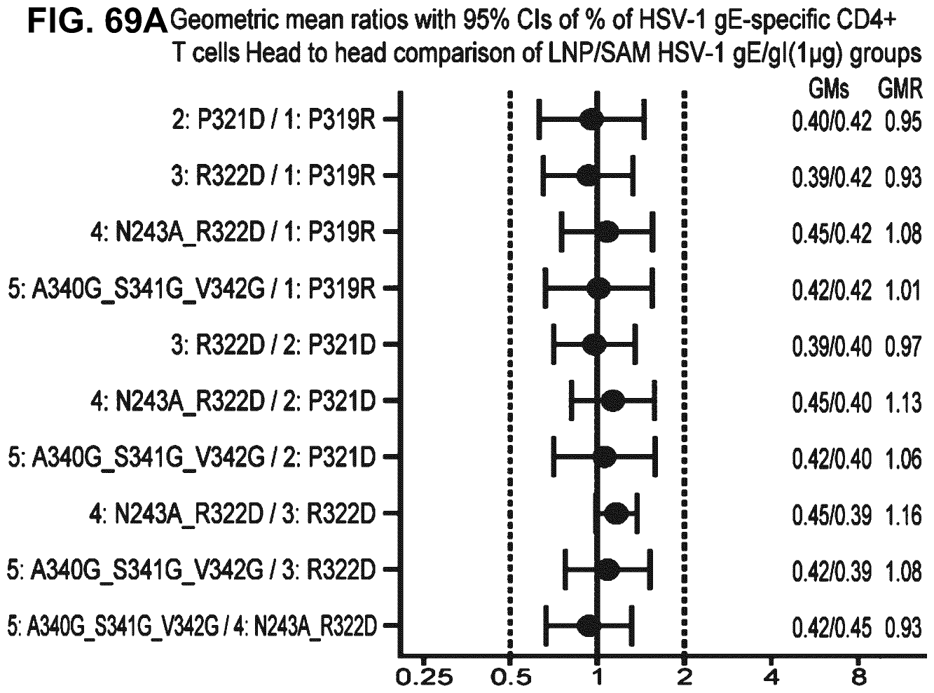

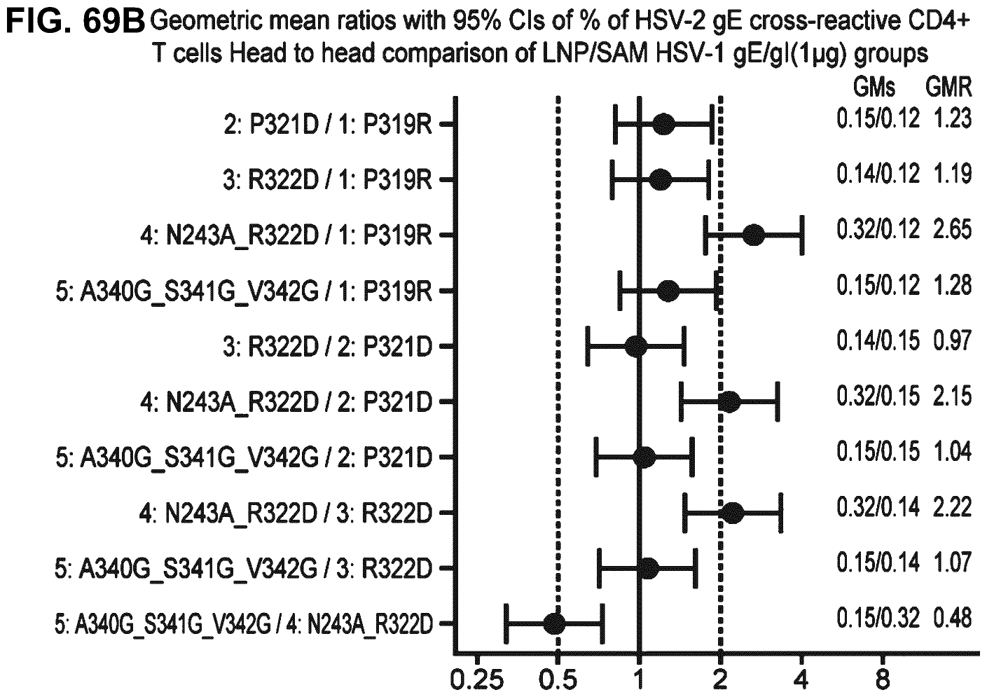

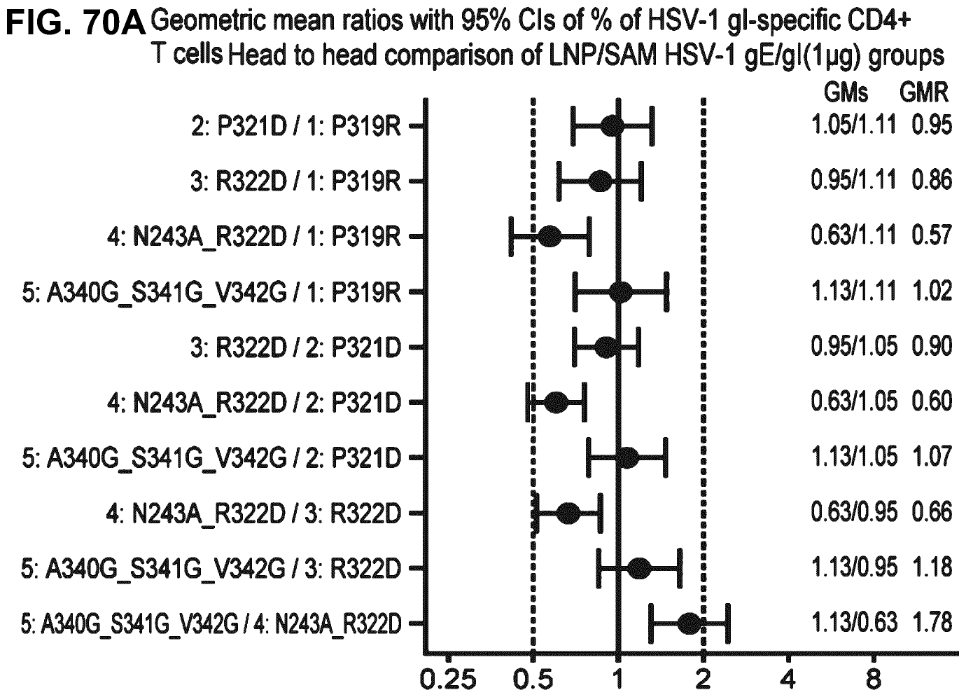

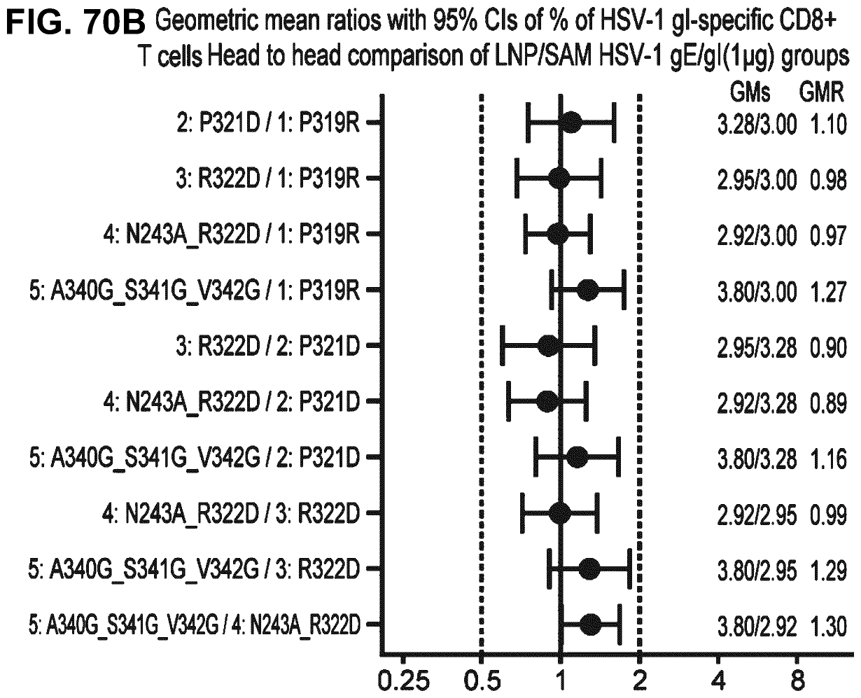

A head to head comparison of different LNP/SAM HSV-1 gE/gI mutants have suggested similar vaccine-specific and anti-HSV-2 gE cross reactive CD4+T cell responses and anti-HSV-1 gI-specific CD4+/CD8+ T cell responses. Only N243A_R322D mutant (group4) seemed to induce higher anti-HSV-2 gE cross-reactive CD4+T cell response compared to other mutants (FIG. 69B). However, this difference seemed to be mainly triggered by one extremely high responder in this group 4 (0.82%). No differences in the intensity of vaccine-specific CD4+ and CD8+T cell responses was observed between the different mutants (GMRs<2-fold change with most CIs totally included in [0.5,2] and/or containing 1) (FIG. 69A; FIG. 70A; FIG. 70B).

1. A nucleic acid encoding a HSV2 Fc receptor or an immunogenic fragment or variant thereof for use in inducing a cross reactive immune response against HSV1 when administered to a subject.

2. A nucleic acid encoding a HSV1 Fc receptor or an immunogenic fragment or variant thereof for use in inducing a cross reactive immune response against HSV2 when administered to a subject.

3-21. (canceled)

22. A method of treating a herpes virus infection or herpes virus related disease in a subject in need thereof comprising administering an immunologically effective amount of a nucleic acid encoding a HSV1 Fc receptor or immunogenic fragment or variant thereof, to the subject wherein the HSV1 Fc receptor or immunogenic fragment or variant thereof can induce a cross reactive immune response against HSV2 when administered to a subject.

1. A nucleic acid encoding a HSV2 Fc receptor or an immunogenic fragment or variant thereof for use in inducing a cross reactive immune response against HSV1 when administered to a subject.

2. A nucleic acid encoding a HSV1 Fc receptor or an immunogenic fragment or variant thereof for use in inducing a cross reactive immune response against HSV2 when administered to a subject.

3. The nucleic acid for use according to claim 1 or 2 wherein the Fc receptor or immunogenic fragment or variant thereof is administered to a subject for use in treating a subject infected with HSV or for preventing HSV infection in a subject, optionally wherein the Fc receptor or immunogenic fragment or variant thereof is administered to a subject for use in treating a subject infected with HSV1 and/or HSV2 or for preventing HSV1 and/or HSV2 infection in a subject.

4. The nucleic acid for use according to any previous claim wherein the cross-reactive immune response comprises a functional cross-reactive immune response.

5. The nucleic acid for use according to claim 4 wherein the functional cross-reactive immune response comprises a cross serotype cytotoxic antibody response, a cross reactive T cell response and/or generation of antibodies that can inhibit the immune evasion activity of HSV, optionally wherein the antibodies that can inhibit the immune evasion activity of HSV, inhibit binding of human IgG to the HSV Fc receptor.

6. The nucleic acid for use according to any one of claims 1 or 3 to 5 wherein said Fc receptor or immunogenic fragment or variant thereof is from a HSV2 gE2 ectodomain.

7. The nucleic acid for use according to any one of claims 2 to 5 wherein said Fc receptor or immunogenic fragment or variant thereof is from a HSV1 gE1 ectodomain.

8. The nucleic acid for use according to any preceding claim wherein said Fc receptor or immunogenic fragment or variant thereof is part of a heterodimer with a binding partner from HSV or a fragment thereof.

9. The nucleic acid for use according to any one of claim 1, 3 to 6 or 8 wherein said Fc receptor or immunogenic fragment or variant thereof is a HSV2 gE2 ectodomain having the amino acid sequence shown at SEQ ID NO: 7, or a variant thereof which is at least 90% identical thereto.

10. The nucleic acid for use according to claim 8 wherein the Fc receptor is HSV2 gE2 or an immunogenic fragment thereof, and the binding partner is HSV2 gI2 or a fragment thereof.

11. The nucleic acid for use according to claim 7 wherein said Fc receptor or immunogenic fragment or variant thereof is part of a heterodimer with a binding partner from HSV or a fragment thereof, preferably wherein i) the Fc receptor is HSV1 gE1 or an immunogenic fragment thereof, and the binding partner is HSV1 gI1 or a fragment thereof, and/or ii) said binding partner or fragment thereof is a HSV1 gI1 ectodomain.

12. The nucleic acid for use according to claim 9 or 10 wherein said binding partner or fragment thereof is a HSV2 gI2 ectodomain, optionally wherein the HSV2 gI2 ectodomain has the amino acid sequence shown at SEQ ID NO: 8, or a variant thereof which is at least 90% identical thereto.

13. The nucleic acid for use according to any preceding claim wherein:—
(i) said use does not comprise administration of a nucleic acid encoding an immunodominant viral antigen to the subject, in particular when the Fc receptor is HSV2 gE2 or HSV1 gE1, the Fc receptor or immunogenic fragment thereof is not administered to the subject together with nucleic acid encoding HSV2 gD2 or HSV1 gD1 (respectively), or a fragment thereof comprising immunodominant regions,
(ii) said Fc receptor is administered to the subject together with nucleic acid encoding HSV2 gC2 or an immunodominant fragment thereof, or nucleic acid encoding HSV1 gC1 or an immunogenic fragment thereof and/or
(iii) the ability of said Fc receptor or immunogenic fragment or variant thereof to bind to a human antibody Fc domain is reduced or abolished compared to the corresponding native HSV Fc receptor.

14. The nucleic acid for use according to any one of claim 1, 3 to 6, 8 to 10, 12 or 13 wherein:
(i) the ability of said Fc receptor or immunogenic fragment or variant thereof to bind to a human antibody Fc domain is reduced or abolished compared to the corresponding native HSV Fc receptor;
(ii) said HSV Fc receptor or immunogenic fragment or variant thereof is a HSV2 gE2 or immunogenic fragment thereof and
(iii) said HSV2 gE2 or immunogenic fragment thereof comprises a mutation or a combination of mutations with respect to the sequence shown in SEQ ID NO: 1 selected from 289_insert ADIGL; 338_insert ARAA; H245K; P317R; P319R; P319G; P319K; H245A_P319R; H245A_P319G; H245A_P319K; H245A_P319T; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248T; P318S; P317K; P317S_V340W; H245D; R314P_V340W; R314L_318D; P319L_V340W; P317F; P318D_S338G; R314G_V340W; P317K_S338H; R314L_V340W; P318R; P318Q; P317F_S338G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R320T; R314P_P318R; A248G_F322I; R320N; P317N; R314D; R314Y; R314P_F322I; P319G_V340M; P317S_V340R; R314V; P317R_P319D; P317R_R320D; P319D_R320D; Δ319_Δ320; P317G_P318G_Δ319_Δ320; P318E_Δ319_Δ320; P318G_Δ319_Δ320; P318K_Δ319_Δ320; P317R_P318E_Δ319_320; P317R_P318G_Δ319_Δ320 and P317G_P318K_Δ319_Δ320.

15. The nucleic acid for use according to any one of claim 2 to 5, 7, 8, 11 or 13 wherein:
(i) the Fc receptor or immunogenic fragment or variant thereof is a recombinant Fc receptor or immunogenic fragment or variant thereof;
(ii) the ability of said Fc receptor or immunogenic fragment or variant thereof to bind to a human antibody Fc domain is reduced or abolished compared to the corresponding native HSV Fc receptor;
(iii) said Fc receptor or immunogenic fragment or variant thereof is a HSV1 gE1 or immunogenic fragment thereof and
(iv) said HSV1 gE1 or immunogenic fragment thereof comprises a mutation or a combination of mutations with respect to the sequence shown in SEQ ID NO: 3 selected from H247A, H247K, P319R, P321A, P321R, P321G, P321K, P321T, A339G, P321D, P321S, A340D, N243A and R322D, H247A/P321A, H247A/P321R, H247A/P321G, H247A/P321K, H247A/P321T, N243A/R322D, N243A/P321D, H247G/P319G, P319G/P321G, A340G/S341G/V342G.

16. A pharmaceutical composition comprising the nucleic acid of any one of claim 1, 3-6, 8-10 or 12-14 and a pharmaceutically acceptable carrier, for use in inducing a cross reactive immune response against HSV1 when administered to a subject.

17. A pharmaceutical composition comprising the nucleic acid of any one of claim 2-5, 7-8, 13 or 15 and a pharmaceutically acceptable carrier, for use in inducing a cross reactive immune response against HSV2 when administered to a subject.

18. The nucleic acid of claim 8, wherein the sequences encoding the HSV Fc receptor or immunogenic fragment or variant thereof and its binding partner or fragment thereof are separated by an internal ribosomal entry site (IRES) sequence.

19. The nucleic acid for use according to any preceding claim, wherein said nucleic acid is an RNA molecule, optionally wherein said RNA molecule is a self-amplifying RNA molecule.

20. The nucleic acid for use according to claim 19, wherein said RNA molecule or self-amplifying RNA molecule is associated with a non-viral delivery material, such as to form a cationic nanoemulsion (CNE) or a lipid nanoparticle (LNP).

21. A method of treating a herpes virus infection or herpes virus related disease in a subject in need thereof comprising administering an immunologically effective amount of a nucleic acid encoding a HSV2 Fc receptor or immunogenic fragment or variant thereof, to the subject wherein the HSV2 Fc receptor or immunogenic fragment or variant thereof can induce a cross reactive immune response against HSV1 when administered to a subject.

22. A method of treating a herpes virus infection or herpes virus related disease in a subject in need thereof comprising administering an immunologically effective amount of a nucleic acid encoding a HSV1 Fc receptor or immunogenic fragment or variant thereof, to the subject wherein the HSV1 Fc receptor or immunogenic fragment or variant thereof can induce a cross reactive immune response against HSV2 when administered to a subject.


SEQUENCE LISTING (135 sequences)

SEQ ID NO: 1 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 2 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 3 - protein, 550 aa, herpes simplex virus 1
MDRGAVVGFLLGVCVVSCLAGTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIHGVRETDSGLYTLSVGDIKDPARQVASVVLVVQPAPVPTPPPTPADYDEDDNDEGEDESLAGTPASGTPRLPPPPAPPRSWPSAPEVSHVRGVTVRMETPEAILFSPGETFSTNVSIHAIAHDDQTYSMDVVWLRFDVPTSCAEMRIYESCLYHPQLPECLSPADAPCAASTWTSRLAVRSYAGCSRTNPPPRCSAEAHMEPVPGLAWQAASVNLEFRDASPQHSGLYLCVVYVNDHIHAWGHITISTAAQYRNAVVEQPLPQRGADLAEPTHPHVGAPPHAPPTHGALRLGAVMGAALLLSALGLSVWACMTCWRRRAWRAVKSRASGKGPTYIRVADSELYADWSSDSEGERDQVPWLAPPERPDSPSTNGSGFEILSPTAPSVYPRSDGHQSRRQLTTFGSGRPDRRYSQASDSSVFW

SEQ ID NO: 4 - protein, 390 aa, herpes simplex virus 1
MPCRPLQGLVLVGLWVCATSLVVRGPTVSLVSNSFVDAGALGPDGVVEEDLLILGELRFVGDQVPHTTYYDGGVELWHYPMGHKCPRVVHVVTVTACPRRPAVAFALCRATDSTHSPAYPTLELNLAQQPLLRVQRATRDYAGVYVLRVWVGDAPNASLFVLGMAIAAEGTLAYNGSAYGSCDPKLLPSSAPRLAPASVYQPAPNQASTPSTTTSTPSTTIPAPSTTIPAPQASTTPFPTGDPKPQPPGVNHEPPSNATRATRDSRYALTVTQIIQIAIPASIIALVFLGSCICFIHRCQRRYRRSRRPIYSPQMPTGISCAVNEAAMARLGAELKSHPSTPPKSRRRSSRTPMPSLTAIAEESEPAGAAGLPTPPVDPTTPTPTPPLLV

SEQ ID NO: 5 - protein, 234 aa, Human cytomegalovirus
MQTYSTPLTLVIVTSLFLFTTQGSSSNAVEPTKKPLKLANYRATCEDRTRTLVTRLNTSHHSVVWQRYDIYSRYMRRMPPLCIITDAYKETTRQGGAAFACTRQNLTLYNLTVKDTGVYLLQDQYTGDVEAFYLIIHPRSFCRALETRRCFYPGPGRVVVTDSQEADRAIISDLKRQWSGLSLHCAWVSGMMIFVGALVICFLRSQRIGEQDAEHLRTDLDTEPLLLTVDGDLQ

SEQ ID NO: 6 - protein, 345 aa, Human cytomegalovirus
MCSVLAIALVVALLGDMHPGVKSSTTSAVTSPSNTTVTSTTSISTSNNVTSAVTTTVQTSTSSASTSVIATTQKEGHLYTVNCEASYSHDQVSLNATCKVILLNNTKNPDILSVTCYARTDCKGPFTQVGYLSAFPPDNEGKLHLSYNATAQELLISGLRPQETTEYTCSFFSWGRHHNATWDLFTYPIYAVYGTRLNATTMRVRVLLQEHEHCLLNGSSLYHPNSTVHLHQGNQLIPPWNISNVTYNGQRLREFVFYLNGTYTVVRLHVQIAGRSFTTTYVFIKSDPLFEDRLLAYGVLAFLVFMVIILLYVTYMLARRRDWSYKRLEEPVEEKKHPVPYFKQW

SEQ ID NO: 7 - protein, 419 aa, Artificial Sequence
  HSV2 gE ectodomain
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLG

SEQ ID NO: 8 - protein, 256 aa, Artificial Sequence
  HSV2 gI ectodomain
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLT

SEQ ID NO: 9 - protein, 256 aa, Artificial Sequence; identical to SEQ ID NO: 8
  HSV2 gI ectodomain

SEQ ID NO: 10 - protein, 270 aa, Artificial Sequence
  HSV1 gI ectodomain
MPCRPLQGLVLVGLWVCATSLVVRGPTVSLVSNSFVDAGALGPDGVVEEDLLILGELRFVGDQVPHTTYYDGGVELWHYPMGHKCPRVVHVVTVTACPRRPAVAFALCRATDSTHSPAYPTLELNLAQQPLLRVQRATRDYAGVYVLRVWVGDAPNASLFVLGMAIAAEGTLAYNGSAYGSCDPKLLPSSAPRLAPASVYQPAPNQASTPSTTTSTPSTTIPAPSTTIPAPQASTTPFPTGDPKPQPPGVNHEPPSNATRATRDSRYALT

SEQ ID NO: 11 - protein, 180 aa, Artificial Sequence
  HCMV gp34 ectodomain
MQTYSTPLTLVIVTSLFLFTTQGSSSNAVEPTKKPLKLANYRATCEDRTRTLVTRLNTSHHSVVWQRYDIYSRYMRRMPPLCIITDAYKETTRQGGAAFACTRQNLTLYNLTVKDTGVYLLQDQYTGDVEAFYLIIHPRSFCRALETRRCFYPGPGRVVVTDSQEADRAIISDLKRQWSG

SEQ ID NO: 12 - protein, 291 aa, Artificial Sequence
  HCMV gp68 ectodomain
MCSVLAIALVVALLGDMHPGVKSSTTSAVTSPSNTTVTSTTSISTSNNVTSAVTTTVQTSTSSASTSVIATTQKEGHLYTVNCEASYSHDQVSLNATCKVILLNNTKNPDILSVTCYARTDCKGPFTQVGYLSAFPPDNEGKLHLSYNATAQELLISGLRPQETTEYTCSFFSWGRHHNATWDLFTYPIYAVYGTRLNATTMRVRVLLQEHEHCLLNGSSLYHPNSTVHLHQGNQLIPPWNISNVTYNGQRLREFVFYLNGTYTVVRLHVQIAGRSFTTTYVFIKSDPLFE

SEQ ID NO: 13 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPATPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 14 - protein, 548 aa, herpes simplex virus 2; identical to SEQ ID NO: 1

SEQ ID NO: 15 - protein, 548 aa, herpes simplex virus 2; identical to SEQ ID NO: 1

SEQ ID NO: 16 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLRAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 17 - protein, 548 aa, herpes simplex virus 2; identical to SEQ ID NO: 1

SEQ ID NO: 18 - protein, 548 aa, herpes simplex virus 2; identical to SEQ ID NO: 1

SEQ ID NO: 19 - protein, 545 aa, herpes simplex virus 2
  misc_feature (109)..(109): Xaa can be any naturally occurring amino acid
  misc_feature (146)..(195): Xaa can be any naturally occurring amino acid
  misc_feature (340)..(340): Xaa can be any naturally occurring amino acid
  misc_feature (475)..(475): Xaa can be any naturally occurring amino acid
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLXSELAWRDRVAVVNESLVIYGALETDSGLYTLSVVGLXXXXXXXXXXXXXXXXXXXXXXXXXXXXXXXXXXXXXXXXXXXXXXXXXXRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLXFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSXGERDGSLWQDPPERPDSPSTNGSGFEILSPTAPSVYPHSEGRKSRRPLTTFGSGSPGRRHSQASYPSVLW

SEQ ID NO: 20 - protein, 545 aa, herpes simplex virus 2
  misc_feature (44)..(46): Xaa can be any naturally occurring amino acid
  misc_feature (58)..(58): Xaa can be any naturally occurring amino acid
  misc_feature (69)..(69): Xaa can be any naturally occurring amino acid
  misc_feature (81)..(206): Xaa can be any naturally occurring amino acid
  misc_feature (387)..(397): Xaa can be any naturally occurring amino acid
MARGAGLVFFVGVWVVSCLAAAPRTSWKRVTSGEDVVVLPAPAXXXRAHKLLWAAEPXDACGPLRPSWXALWPPRRVLETXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPXXXXXXXXXXXPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 21 - protein, 548 aa, herpes simplex virus 2
  misc_feature (181)..(203): Xaa can be any naturally occurring amino acid
  misc_feature (377)..(405): Xaa can be any naturally occurring amino acid
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDXXXXXXXXXXXXXXXXXXXXXXXPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAXXXXXXXXXXXXXXXXXXXXXXXXXXXXXPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 22 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDKARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 23 - protein, 548 aa, herpes simplex virus 2; identical to SEQ ID NO: 1

SEQ ID NO: 24 - protein, 548 aa, herpes simplex virus 2; identical to SEQ ID NO: 22

SEQ ID NO: 25 - protein, 548 aa, herpes simplex virus 2
  misc_feature (120)..(120): Xaa can be any naturally occurring amino acid
  misc_feature (141)..(198): Xaa can be any naturally occurring amino acid
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDXVAVVNESLVIYGALETDSGLXXXXXXXXXXXXXXXXXXXXXXXXXXXXXXXXXXXXXXXXXXXXXXXXXXXXXXXXXXRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHMRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 26 - protein, 548 aa, herpes simplex virus 2; identical to SEQ ID NO: 1

SEQ ID NO: 27 - protein, 545 aa, herpes simplex virus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 28 - protein, 545 aa, herpes simplex virus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVTNARRSAFPPQPPPRRPPVAPPTHPRVIPEVSHVRGVTVHMETLEAILFAPGETFGTNVSIHAIAHDDGPYAMDVVWMRFDVPSSCADMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 29 - protein, 545 aa, herpes simplex virus 2; identical to SEQ ID NO: 27

SEQ ID NO: 30 - protein, 548 aa, herpes simplex virus 2; identical to SEQ ID NO: 1

SEQ ID NO: 31 - protein, 548 aa, herpes simplex virus 2; identical to SEQ ID NO: 1

SEQ ID NO: 32 - protein, 548 aa, herpes simplex virus 2; identical to SEQ ID NO: 13

SEQ ID NO: 33 - protein, 548 aa, herpes simplex virus 2
MARGAGLVFFVGVWVVSCLGAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 34 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 35 - protein, 548 aa, herpes simplex virus 2; identical to SEQ ID NO: 1

SEQ ID NO: 36 - protein, 548 aa, herpes simplex virus 2; identical to SEQ ID NO: 1

SEQ ID NO: 37 - protein, 548 aa, herpes simplex virus 2; identical to SEQ ID NO: 1

SEQ ID NO: 38 - protein, 548 aa, herpes simplex virus 2; identical to SEQ ID NO: 34

SEQ ID NO: 39 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALDTDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 40 - protein, 548 aa, herpes simplex virus 2; identical to SEQ ID NO: 1

SEQ ID NO: 41 - protein, 539 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGARETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHS

SEQ ID NO: 42 - protein, 545 aa, herpes simplex virus 2; identical to SEQ ID NO: 27

SEQ ID NO: 43 - protein, 543 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASY

SEQ ID NO: 44 - protein, 544 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRHPLTTFGSGSPGRRHSQASYS

SEQ ID NO: 45 - protein, 545 aa, herpes simplex virus 2; identical to SEQ ID NO: 27

SEQ ID NO: 46 - protein, 543 aa, herpes simplex virus 2; identical to SEQ ID NO: 43

SEQ ID NO: 47 - protein, 542 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAGLGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQAS

SEQ ID NO: 48 - protein, 538 aa, herpes simplex virus 2
MARGAGLVFFVGVWVVSCLAAAPRTSWKRVTSGEDVVLLPAPAGPEERTRVHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRH

SEQ ID NO: 49 - protein, 534 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSP

SEQ ID NO: 50 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTLVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 51 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGARETDSGLYTLSVVGLSDEARQVASVVLVVEPAPVPTPTPDDYDEEDDAGVSERTPVSVPPTTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 52 - protein, 548 aa, herpes simplex virus 2; identical to SEQ ID NO: 51

SEQ ID NO: 53 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGARETDSGLYTLSVVGLSDEARQVASVVLVVEPAPVPTPTPDDYDEEDDAGVSERTPVSVPPPTPPRRPPVAPQTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 54 - protein, 548 aa, herpes simplex virus 2
MARGAGLVFFVGVWVVSCLAAAPRTSWKRVTSGEDVVLLPAPAGPEERTRTHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPATPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSIYPHSEGRKSRRPLTTFGSGSPGRRHSQASYSSVLW

SEQ ID NO: 55 - protein, 548 aa, herpes simplex virus 2; identical to SEQ ID NO: 34

SEQ ID NO: 56 - protein, 548 aa, herpes simplex virus 2; identical to SEQ ID NO: 34

SEQ ID NO: 57 - protein, 548 aa, herpes simplex virus 2; identical to SEQ ID NO: 34

SEQ ID NO: 58 - protein, 548 aa, herpes simplex virus 2
MARGAGLVFFVGVWVVSCLAAAPRTSWKRVTSGEDVVLLPAPTGPEERTRAHKLLWAAEPLDACGPLRPSWVALWPPRRVLETVVDAACMRAPEPLAIAYSPPFPAGDEGLYSELACRDRVAVVNESLVIYGARETDSGLYTLSVVGLSDEARQVASVVLVVEPAPVPTPTPDDYDEEDDAGVSERTPVSVPPPTPPRRPPVAPQTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 59 - protein, 548 aa, herpes simplex virus 2; identical to SEQ ID NO: 58

SEQ ID NO: 60 - protein, 548 aa, herpes simplex virus 2; identical to SEQ ID NO: 53

SEQ ID NO: 61 - protein, 548 aa, herpes simplex virus 2; identical to SEQ ID NO: 51

SEQ ID NO: 62 - protein, 372 aa, herpes simplex virus 2; identical to SEQ ID NO: 2

SEQ ID NO: 63 - protein, 372 aa, herpes simplex virus 2; identical to SEQ ID NO: 2

SEQ ID NO: 64 - protein, 372 aa, herpes simplex virus 2; identical to SEQ ID NO: 2

SEQ ID NO: 65 - protein, 372 aa, herpes simplex virus 2; identical to SEQ ID NO: 2

SEQ ID NO: 66 - protein, 372 aa, herpes simplex virus 2; identical to SEQ ID NO: 2

SEQ ID NO: 67 - protein, 372 aa, herpes simplex virus 2; identical to SEQ ID NO: 2

SEQ ID NO: 68 - protein, 372 aa, herpes simplex virus 2; identical to SEQ ID NO: 2

SEQ ID NO: 69 - protein, 372 aa, herpes simplex virus 2; identical to SEQ ID NO: 2

SEQ ID NO: 70 - protein, 372 aa, herpes simplex virus 2; identical to SEQ ID NO: 2

SEQ ID NO: 71 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 72 - protein, 372 aa, herpes simplex virus 2; identical to SEQ ID NO: 71

SEQ ID NO: 73 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 74 - protein, 372 aa, herpes simplex virus 2; identical to SEQ ID NO: 73

SEQ ID NO: 75 - protein, 372 aa, herpes simplex virus 2; identical to SEQ ID NO: 71

SEQ ID NO: 76 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGLDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 77 - protein, 372 aa, herpes simplex virus 2; identical to SEQ ID NO: 71

SEQ ID NO: 78 - protein, 372 aa, herpes simplex virus 2; identical to SEQ ID NO: 73

SEQ ID NO: 79 - protein, 372 aa, herpes simplex virus 2; identical to SEQ ID NO: 71

SEQ ID NO: 80 - protein, 372 aa, herpes simplex virus 2; identical to SEQ ID NO: 73

SEQ ID NO: 81 - protein, 372 aa, herpes simplex virus 2; identical to SEQ ID NO: 73

SEQ ID NO: 82 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTLPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 83 - protein, 372 aa, herpes simplex virus 2; identical to SEQ ID NO: 73

SEQ ID NO: 84 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 85 - protein, 372 aa, herpes simplex virus 2; identical to SEQ ID NO: 84

SEQ ID NO: 86 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARPPLLRVRTATRDYAGLYVLRVWVGSATNASLFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 87 - protein, 372 aa, herpes simplex virus 2; identical to SEQ ID NO: 84

SEQ ID NO: 88 - protein, 372 aa, herpes simplex virus 2; identical to SEQ ID NO: 84

SEQ ID NO: 89 - protein, 374 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTSPSSPRDPTPAPGDTGTPAPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 90 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGLDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 91 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVSQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 92 - protein, 372 aa, herpes simplex virus 2
MPGRSLQGLVILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTLPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 93 - protein, 372 aa, herpes simplex virus 2; identical to SEQ ID NO: 92

SEQ ID NO: 94 - protein, 372 aa, herpes simplex virus 2; identical to SEQ ID NO: 92

SEQ ID NO: 95 - protein, 372 aa, herpes simplex virus 2; identical to SEQ ID NO: 90

SEQ ID NO: 96 - protein, 372 aa, herpes simplex virus 2; identical to SEQ ID NO: 91

SEQ ID NO: 97 - protein, 372 aa, herpes simplex virus 2; identical to SEQ ID NO: 92

SEQ ID NO: 98 - protein, 372 aa, herpes simplex virus 2; identical to SEQ ID NO: 92

SEQ ID NO: 99 - protein, 371 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVITLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTSPSSPRDPTPAPGDTGTPAPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAP

SEQ ID NO: 100 - protein, 372 aa, herpes simplex virus 2
  misc_feature (222)..(222): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTPPSSPRDXTPAPGDTGTPAPASGEIAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 101 - protein, 372 aa, herpes simplex virus 2 [sequence omitted]

SEQ ID NO: 102 - protein, 372 aa, herpes simplex virus 2 [sequence omitted]

SEQ ID NO: 103 - protein, 372 aa, herpes simplex virus 2 [sequence omitted]

SEQ ID NO: 104 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 105 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEELEPGPVVLLSVSPRPRSGPTAPQEV

SEQ ID NO: 106 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGEKAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 107 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHRAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 108 - protein, 372 aa, herpes simplex virus 2; identical to SEQ ID NO: 104

SEQ ID NO: 109 - protein, 372 aa, herpes simplex virus 2; identical to SEQ ID NO: 104

SEQ ID NO: 110 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVRVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 111 - protein, 372 aa, herpes simplex virus 2 [sequence omitted]

SEQ ID NO: 112 - protein, 372 aa, herpes simplex virus 2 [sequence omitted]

SEQ ID NO: 113 - protein, 372 aa, herpes simplex virus 2; identical to SEQ ID NO: 104

SEQ ID NO: 114 - protein, 372 aa, herpes simplex virus 2; identical to SEQ ID NO: 104

SEQ ID NO: 115 - protein, 372 aa, herpes simplex virus 2; identical to SEQ ID NO: 104

SEQ ID NO: 116 - protein, 372 aa, herpes simplex virus 2
MPGRSLQGLVILGLWVCTTGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTLPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 117 - protein, 372 aa, herpes simplex virus 2; identical to SEQ ID NO: 104

SEQ ID NO: 118 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHRAHSPAYPTLELGLARQPLLRVRTATRDYAGLYVLRVWVGSATNASRFVLGVALSVNGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 119 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVITLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 120 - protein, 372 aa, herpes simplex virus 2
  misc_feature (215)..(219): Xaa can be any naturally occurring amino acid
MPGRSLQGLAMLGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTXXXXXRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 121 - protein, 372 aa, herpes simplex virus 2
  misc_feature (215)..(224): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTXXXXXXXXXXAPGDTGTPAPASGEIAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 122 - protein, 372 aa, herpes simplex virus 2
  misc_feature (212)..(238): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRXXXXXXXXXXXXXXXXXXXXXXXXXXXIAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 123 - protein, 424 aa, herpes simplex virus 2
  misc_feature (201)..(231): Xaa can be any naturally occurring amino acid
  misc_feature (309)..(396): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHMITLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYXXXXXXXXXXXXXXXXXXXXXXXXXXXXXXXPAPASGERAPPNSTRSASESRHRLTVAQVIQIAIPASIIAFVFLGSCICFIHRCQRRYRRPRGQIYNPGGVSCAVNEXXXXXXXXXXXXXXXXXXXXXXXXXXXXXXXXXXXXXXXXXXXXXXXXXXXXXXXXXXXXXXXXXXXXXXXXXXXXXXXXXXXXXXXXNKPMLFAYMRVLDPFVIVRHSPDGMGGG

SEQ ID NO: 124 - DNA, 66 bp, Artificial Sequence
  GSG-P2A
ggaagcggagctactaacttcagcctgctgaagcaggctggagacgtggaggagaaccctggacct

SEQ ID NO: 125 - DNA, 99 bp, Artificial Sequence
  F2A long
gaggcccggcacaagcagaagatcgtggcccccgtgaagcagaccctgaacttcgacctgctgaagctggccggcgacgtggagagcaaccccggcccc

SEQ ID NO: 126 - DNA, 24 bp, Artificial Sequence
  Subgenomic promoter
ctctctacggctaacctgaatgga

SEQ ID NO: 127 - DNA, 750 bp, Artificial Sequence
  IRES EV71
tgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgacc

SEQ ID NO: 128 - DNA, 1257 bp, Artificial Sequence
  gE P317R ectodomain codon-optimized
atggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggc

SEQ ID NO: 129 - DNA, 768 bp, Artificial Sequence
  gI ectodomain codon-optimized
atgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgaca

SEQ ID NO: 130 - DNA, 9993 bp, Artificial
  SAM backbone
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 131 - DNA, 7561 bp, Artificial
  5′ region of SEQ ID NO130
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 132 - DNA, 2439 bp, Artificial
  3′ region of SEQ ID NO130
tctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 133 - RNA, 7747 nt, Artificial
  SAM backbone
auaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagacggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaa

SEQ ID NO: 134 - RNA, 7561 nt, Artificial
  5′ region of SEQ ID NO133
auaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaag

SEQ ID NO: 135 - RNA, 186 nt, Artificial
  3′ region of SEQ ID NO133
ucuagacggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaa